SEQUENCE LISTING (24 sequences)

SEQ ID NO: 1 - DNA, 564 bp, Saccharomyces cerevisiae
  misc_feature: APT1 Accession number NM_001182380.1
atgtctatagcaagttatgcccaagagttgaagttggctttacatcaatatccaaacttccctagtgaaggcattctcttcgaagatttcttacccattttcaggaacccaggtcttttccagaagttgatcgatgctttcaaactgcatttagaagaagcttttccagaagttaaaattgattatatcgtcgggttggaatcccgtgggttcttgttcggaccaactttagctttggccctaggtgttggtttcgttccagtcaggaaggcaggtaagctacctggcgaatgttttaaggctacgtacgaaaaggagtacggttctgatctttttgagatacagaaaaacgctattccagcaggttccaacgttatcattgttgatgacattattgccactggtggttctgctgctgcagccggcgaattagttgaacaactcgaagccaaccttttggaatataactttgttatggagttggatttcttgaaaggcaggagtaagttgaatgctccagtgttcactttactgaacgctcaaaaggaagcgttgaaaaaatga

SEQ ID NO: 2 - protein, 187 aa, Saccharomyces cerevisiae
  MISC_FEATURE: Adenine phosphoribosyltransferase, Accession number NP_013690.1
MSIASYAQELKLALHQYPNFPSEGILFEDFLPIFRNPGLFQKLIDAFKLHLEEAFPEVKIDYIVGLESRGFLFGPTLALALGVGFVPVRKAGKLPGECFKATYEKEYGSDLFEIQKNAIPAGSNVIIVDDIIATGGSAAAAGELVEQLEANLLEYNFVMELDFLKGRSKLNAPVFTLLNAQKEALKK

SEQ ID NO: 3 - DNA, 1044 bp, Saccharomyces cerevisiae
  misc_feature: AAH1, Accession number NM_001182979.1
atggtttctgtggagtttttacaggagttaccaaaatgtgagcatcacttgcatttggaaggtactctagaacctgacctattgttcccattagctaaaagaaacgatataattctacctgaaggttttcctaaatcggtcgaggaattaaacgaaaagtataagaagtttcgtgatctgcaggatttcttagattactattatattggtactaatgtcttgattagtgaacaagatttctttgatttggcgtgggcctattttaaaaaagttcacaaacaaggcttggtccatgctgaagtgttttacgaccctcagtcacatacatctaggggcatctccatagaaacagtcactaaaggtttccaaagagcttgtgacaaagccttctctgaatttggtattacatccaagctaattatgtgtctgttaagacacattgaaccagaggaatgtttgaaaactatcgaagaagctaccccatttattaaagatggtactatctctgccttaggattagattctgctgagaaaccatttcccccacatttatttgttgaatgttacggaaaggccgcctcattgaataaagatttaaaactaactgcacacgcaggtgaagaaggccccgctcaattcgtctcggatgctttagacttgttgcaagtaacaagaatcgatcacggtatcaacagtcaatacgacgaggagttattggataggttgtcgcgcgaccagaccatgctaactatttgtcctctctccaacgtgaagctacaagtagtccaatccgtttcagagttaccactacaaaagtttcttgacagagatgttccattttctttaaattctgatgaccccgcctattttggtggttatatcttagatgtctacactcaagtttcgaaagatttcccacactgggaccatgaaacatggggtcgtatcgctaagaacgccattaaaggttcatggtgtgacgataaaagaaagaacggtttgttaagtagagtggacgaagtagtcactaaatattcgcattag

SEQ ID NO: 4 - protein, 347 aa, Saccharomyces cerevisiae
  MISC_FEATURE: Adenine deaminase, Accession number NP_014258.1
MVSVEFLQELPKCEHHLHLEGTLEPDLLFPLAKRNDIILPEGFPKSVEELNEKYKKFRDLQDFLDYYYIGTNVLISEQDFFDLAWAYFKKVHKQGLVHAEVFYDPQSHTSRGISIETVTKGFQRACDKAFSEFGITSKLIMCLLRHIEPEECLKTIEEATPFIKDGTISALGLDSAEKPFPPHLFVECYGKAASLNKDLKLTAHAGEEGPAQFVSDALDLLQVTRIDHGINSQYDEELLDRLSRDQTMLTICPLSNVKLQVVQSVSELPLQKFLDRDVPFSLNSDDPAYFGGYILDVYTQVSKDFPHWDHETWGRIAKNAIKGSWCDDKRKNGLLSRVDEVVTKYSH

SEQ ID NO: 5 - DNA, 1113 bp, Saccharomyces cerevisiae
  misc_feature: ARO3, Accession number NM_001180343.3
atgttcattaaaaacgatcacgccggtgacaggaaacgcttggaagactggagaatcaaaggttatgatccattaacccctccagatctgcttcaacatgaatttccaatttcagccaaaggtgaggaaaacattatcaaggcaagagactccgtctgtgatattttgaatggtaaagatgatcgtttagttatcgtgatcgggccatgttccctacatgaccccaaagccgcttacgattacgctgacagattggctaaaatttcagaaaagttgtcaaaagacttattgattattatgagagcgtatttagaaaaaccaaggactactgttggctggaaagggttgattaacgaccctgatatgaataactcttttcaaatcaataaaggtctacggatttcgagagaaatgttcataaaactggttgaaaaattacccattgctggtgagatgttggataccatttctccgcagtttttgagtgattgtttctccttgggtgccatcggcgccagaactactgaatcccaactgcacagagaattagcatccggtctatctttccctattggatttaagaacggtactgatggtggtttgcaagtcgccatcgacgctatgagagccgctgcacatgaacattacttcctttctgtcacaaagccaggtgtcactgctatcgtgggcactgaaggtaacaaggataccttcctgatcttgagaggtggtaagaacggtactaactttgacaaagaaagtgttcaaaatactaagaaacagttagaaaaggccggtttgactgatgattcccagaaaagaattatgatcgattgttcccacggcaacagtaataaagatttcaagaaccaaccaaaggttgccaaatgtatttatgaccagctgacggagggtgagaatagtctctgtggtgttatgattgagtccaacataaatgaaggtagacaagatattcccaaagaaggtggcagagagggattgaagtatggttgttctgttacggatgcttgtattggctgggagtccaccgaacaggtattggagctattggcagaaggtgttagaaacagaagaaaggccttgaaaaaatag

SEQ ID NO: 6 - protein, 370 aa, Saccharomyces cerevisiae
  MISC_FEATURE: 3-deoxy-7-phosphoheptulonate synthase ARO3, Accession number NP_010320.3
MFIKNDHAGDRKRLEDWRIKGYDPLTPPDLLQHEFPISAKGEENIIKARDSVCDILNGKDDRLVIVIGPCSLHDPKAAYDYADRLAKISEKLSKDLLIIMRAYLEKPRTTVGWKGLINDPDMNNSFQINKGLRISREMFIKLVEKLPIAGEMLDTISPQFLSDCFSLGAIGARTTESQLHRELASGLSFPIGFKNGTDGGLQVAIDAMRAAAHEHYFLSVTKPGVTAIVGTEGNKDTFLILRGGKNGTNFDKESVQNTKKQLEKAGLTDDSQKRIMIDCSHGNSNKDFKNQPKVAKCIYDQLTEGENSLCGVMIESNINEGRQDIPKEGGREGLKYGCSVTDACIGWESTEQVLELLAEGVRNRRKALKK

SEQ ID NO: 7 - DNA, 1113 bp, Saccharomyces cerevisiae
  misc_feature: ARO4, Accession number NM_001178597.1
atgagtgaatctccaatgttcgctgccaacggcatgccaaaggtaaatcaaggtgctgaagaagatgtcagaattttaggttacgacccattagcttctccagctctccttcaagtgcaaatcccagccacaccaacttctttggaaactgccaagagaggtagaagagaagctatagatattattaccggtaaagacgacagagttcttgtcattgtcggtccttgttccatccatgatctagaagccgctcaagaatacgctttgagattaaagaaattgtcagatgaattaaaaggtgatttatccatcattatgagagcatacttggagaagccaagaacaaccgtcggctggaaaggtctaattaatgaccctgatgttaacaacactttcaacatcaacaagggtttgcaatccgctagacaattgtttgtcaacttgacaaatatcggtttgccaattggttctgaaatgcttgataccatttctcctcaatacttggctgatttggtctccttcggtgccattggtgccagaaccaccgaatctcaactgcacagagaattggcctccggtttgtctttcccagttggtttcaagaacggtaccgatggtaccttaaatgttgctgtggatgcttgtcaagccgctgctcattctcaccatttcatgggtgttactaagcatggtgttgctgctatcaccactactaagggtaacgaacactgcttcgttattctaagaggtggtaaaaagggtaccaactacgacgctaagtccgttgcagaagctaaggctcaattgcctgccggttccaacggtctaatgattgactactctcacggtaactccaataaggatttcagaaaccaaccaaaggtcaatgacgttgtttgtgagcaaatcgctaacggtgaaaacgccattaccggtgtcatgattgaatcaaacatcaacgaaggtaaccaaggcatcccagccgaaggtaaagccggcttgaaatatggtgtttccatcactgatgcttgtataggttgggaaactactgaagacgtcttgaggaaattggctgctgctgtcagacaaagaagagaagttaacaagaaatag

SEQ ID NO: 8 - protein, 370 aa, Saccharomyces cerevisiae
  MISC_FEATURE: 3-deoxy-7-phosphoheptulonate synthase ARO4, Accession number NP_009808.1
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 9 - DNA, 1203 bp, Saccharomyces cerevisiae
  misc_feature: GCV1, Accession number NM_001180327.1
atgtctataatcaaaaaaattgtgtttaagagattcaactcaactttgaaaaaaactgctcttcatgaccttcatgtgtcattaggcggtacaatggtaccatacgcaggatattccatgcccgtactatacaagggacagactcatattgaatcacataattggaccaggacaaacgctggtttatttgatgtttctcatatgttacaaagtaagttatctggtcctcattctgttaagtttctgcaacgtgtaacgcccacagatttcaatgccttgcctgttgggtcagggaccttaagtgttctattaaatccacagggaggcgtggtagacgataccatcatcactaaagaaaacgatgacaatgagttctatattgtgaccaatgctggctgtgcggaaagagacaccgagttctttcacgatgagctacaaaatggttctactttagattgtcaatggaaaatcattgaaggtagatcactattggcattgcagggtcctaaagcaaaagatgtgttggaaccacttttatcaaaaacagctcctggaaaggatctaaaagagttattttttggacaaagacatgaatttgcgttaaaggacggttctttggttcaaattgctagaggcgggtacactggtgaagatggatttgagataagcattgcaaatgagaaagcggttgagtttgcggagcaactcttggccaatccagtaatgaagcctatcggactggctgctagagacagtttaaggttggaagctggtatgtgtttgtatggtcatgaattggacgaaagtatcacaccagtagaagctgcgttaaactgggtgatttcaaagtcaagaagggacctagtagatcaaaagtactggttcaatggctatgccaagataatggaccagctaaacaataaaacgtatagcaaggtacgagtcggattcaaatacttgaaaaaggggccagctgccagaaatggtgtcaagatcttcttgcccgatgccgaaacagaggtcggccttgttacatcaggtagtgcatctccaactttaaacaacatcaatattggacaagcttatgtacagaaaggttaccacaagaagggaacgaagttgcttgttcaagtaaggaacaagttctacccgattgaacttgctaaaatgcctcttgtgcccacacattactacaagcagtaa

SEQ ID NO: 10 - protein, 400 aa, Saccharomyces cerevisiae
  MISC_FEATURE: glycine decarboxylase subunit T, Accession number NP_010302.1
MSIIKKIVFKRFNSTLKKTALHDLHVSLGGTMVPYAGYSMPVLYKGQTHIESHNWTRTNAGLFDVSHMLQSKLSGPHSVKFLQRVTPTDFNALPVGSGTLSVLLNPQGGVVDDTIITKENDDNEFYIVTNAGCAERDTEFFHDELQNGSTLDCQWKIIEGRSLLALQGPKAKDVLEPLLSKTAPGKDLKELFFGQRHEFALKDGSLVQIARGGYTGEDGFEISIANEKAVEFAEQLLANPVMKPIGLAARDSLRLEAGMCLYGHELDESITPVEAALNWVISKSRRDLVDQKYWFNGYAKIMDQLNNKTYSKVRVGFKYLKKGPAARNGVKIFLPDAETEVGLVTSGSASPTLNNINIGQAYVQKGYHKKGTKLLVQVRNKFYPIELAKMPLVPTHYYKQ

SEQ ID NO: 11 - DNA, 1524 bp, Saccharomyces cerevisiae
  misc_feature: CYS4, Accession number NM_001181284.3
atgactaaatctgagcagcaagccgattcaagacataacgttatcgacttagttggtaacaccccattgatcgcactgaaaaaattgcctaaggctttgggtatcaaaccacaaatttatgctaagctggaactatacaatccaggtggttccatcaaagacagaattgccaagtctatggtggaagaagctgaagcttccggtagaattcatccttccagatctactctgatcgaacctacttctggtaacaccggtatcggtctagctttaatcggcgccatcaaaggttacagaactatcatcaccttgccggaaaaaatgtctaacgagaaagtttctgtcctaaaggctctgggtgctgaaatcatcagaactccaactgctgctgcctgggattctccagaatcacatattggtgttgctaagaagttggaaaaagagattcctggtgctgttatacttgaccaatataacaatatgatgaacccagaagctcattactttggtactggtcgcgaaatccaaagacagctagaagacttgaatttatttgataatctacgcgctgttgttgctggtgctggtactggtgggactattagcggtatttccaagtacttgaaagaacagaatgataagatccaaatcgttggtgctgacccattcggttcaattttagcccaacctgaaaacttgaataagactgatatcactgactacaaagttgagggtattggttatgattttgttcctcaggttttggacagaaaattaattgatgtttggtataagacagacgacaagccttctttcaaatacgccagacaattgatttctaacgaaggtgtcttggtgggtggttcttccggttctgccttcactgcggttgtgaaatactgtgaagaccaccctgaactgactgaagatgatgtcattgttgccatattcccagattccatcaggtcgtacctaaccaaattcgtcgatgacgaatggttgaaaaagaacaatttgtgggatgatgacgtgttggcccgttttgactcttcaaagctggaggcttcgacgacaaaatacgctgatgtgtttggtaacgctactgtaaaggatcttcacttgaaaccggttgtttccgttaaggaaaccgctaaggtcactgatgttatcaagatattaaaagacaatggctttgaccaattgcctgtgttgactgaagacggcaagttgtctggtttagttactctctctgagcttctaagaaaactatcaatcaataattcaaacaacgacaacactataaagggtaaatacttggacttcaagaaattaaacaatttcaatgatgtttcctcttacaacgaaaataaatccggtaagaagaagtttattaaattcgatgaaaactcaaagctatctgacttgaatcgtttctttgaaaaaaactcatctgccgttatcactgatggcttgaaaccaatccatatcgttactaagatggatttactgagctacttagcataa

SEQ ID NO: 12 - protein, 507 aa, Saccharomyces cerevisiae
  MISC_FEATURE: cystathionine beta-synthase, Accession number NP_011671.3
MTKSEQQADSRHNVIDLVGNTPLIALKKLPKALGIKPQIYAKLELYNPGGSIKDRIAKSMVEEAEASGRIHPSRSTLIEPTSGNTGIGLALIGAIKGYRTIITLPEKMSNEKVSVLKALGAEIIRTPTAAAWDSPESHIGVAKKLEKEIPGAVILDQYNNMMNPEAHYFGTGREIQRQLEDLNLFDNLRAVVAGAGTGGTISGISKYLKEQNDKIQIVGADPFGSILAQPENLNKTDITDYKVEGIGYDFVPQVLDRKLIDVWYKTDDKPSFKYARQLISNEGVLVGGSSGSAFTAVVKYCEDHPELTEDDVIVAIFPDSIRSYLTKFVDDEWLKKNNLWDDDVLARFDSSKLEASTTKYADVFGNATVKDLHLKPVVSVKETAKVTDVIKILKDNGFDQLPVLTEDGKLSGLVTLSELLRKLSINNSNNDNTIKGKYLDFKKLNNFNDVSSYNENKSGKKKFIKFDENSKLSDLNRFFEKNSSAVITDGLKPIHIVTKMDLLSYLA

SEQ ID NO: 13 - DNA, 1434 bp, Caenorhabditis elegans
  misc_feature: FAH-1, Accession number NM_076682.4
cgccgcatgcgtgtacagtttcggagacataacagactgctgtctactcaactccatttttcaaaacttgtattgctttgcaggctaaatcatgaaatccttcgtttctgttcctcaaaactcggatttcccaattcaaaacttaccgtatggtgttttctcaacaaaggctgactcttcccgccacattggagttgcaattggagatcaaatcctcaacttggcagaaattgccaatctttttgacggacctcaattgaaggcacatcaggatgttttcaagcagagcacattgaacgcgttcatggcactgccacgtccagcctggttggaagctcgtgcaagaatccaacaactgctctcagaggattgtgctgttcttagggataacgctcatttgagaagccgggctcttgtcgctcaatccgatgctacaatgcaccttccagctcaaatcggagattacaccgatttctactcctccatccatcacgccaccaacgtcggaatcatgttccgtggaaaggaaaatgctctgatgccaaactggaagtggcttccggtcggataccacggaagagcttcatcaatcgttgtatctggaacagacttgaaaagaccagttggacaaactaaggccccagatgctgaggttccatcgtttggtccatcaaagcttatggactttgagctcgagatggcgttctttgtgggaggtccagaaaatgaattgggaactcgtgttccaatcgagaaggctgaggatcgtatctttggagttgtgctcatgaacgactggagtgctcgcgatatccaagcttgggaatacgttccactcggcccattccttgctaaatcctttgccacaactgtttctccatgggtcgtctcaattgaagctcttcgtccatactttgttgagaatccagtacaagatccagttccaccagcatatctccatcacgatgacccatttacccttgacatcaatttggccgtcagtatccgtccagaaggagatgctgtggaccatattgtctgtaaaactaacttcaagcatttgtattggactctaaaacaacaacttgctcaccacaccgtcaatggatgcaacctccgcgctggagatcttttgggatcaggaactgtatctggaccagaagaaggtgcatatggatccatgctcgagctttcgtggcgcggagccaaagaagttccagttggctcagaaatccgaaagttcttaaaggacggagatgaggtcaacctttctggagtttgtgaaaagaatggagtcagaattggtttcggagagtgccgcggaaaagtcctgccagccgatatctaaaatcatcgtaattaaatcgttttgctttttgtttcattctctcgtttgttagtagtatctcgtcgaataaagttctgaagcccacg

SEQ ID NO: 14 - protein, 418 aa, Caenorhabditis elegans
  MISC_FEATURE: Fumarylacetoacetase, Accession number NP_509083.1
MKSFVSVPQNSDFPIQNLPYGVFSTKADSSRHIGVAIGDQILNLAEIANLFDGPQLKAHQDVFKQSTLNAFMALPRPAWLEARARIQQLLSEDCAVLRDNAHLRSRALVAQSDATMHLPAQIGDYTDFYSSIHHATNVGIMFRGKENALMPNWKWLPVGYHGRASSIVVSGTDLKRPVGQTKAPDAEVPSFGPSKLMDFELEMAFFVGGPENELGTRVPIEKAEDRIFGVVLMNDWSARDIQAWEYVPLGPFLAKSFATTVSPWVVSIEALRPYFVENPVQDPVPPAYLHHDDPFTLDINLAVSIRPEGDAVDHIVCKTNFKHLYWTLKQQLAHHTVNGCNLRAGDLLGSGTVSGPEEGAYGSMLELSWRGAKEVPVGSEIRKFLKDGDEVNLSGVCEKNGVRIGFGECRGKVLPADI

SEQ ID NO: 15 - DNA, 1500 bp, Saccharomyces cerevisiae
  misc_feature: dihydrolipoyl dehydrogenase - LPD1, Accession number NM_001179948.1
atgttaagaatcagatcactcctaaataataagcgtgccttttcgtccacagtcaggacattgaccattaacaagtcacatgatgtagtcatcatcggtggtggccctgctggttacgtggctgctatcaaagctgctcaattgggatttaacactgcatgtgtagaaaaaagaggcaaattaggcggtacctgtcttaacgttggatgtatcccctccaaagcacttctaaataattctcatttattccaccaaatgcatacggaagcgcaaaagagaggtattgacgtcaacggtgatatcaaaattaacgtagcaaacttccaaaaggctaaggatgacgctgttaagcaattaactggaggtattgagcttctgttcaagaaaaataaggtcacctattataaaggtaatggttcattcgaagacgaaacgaagatcagagtaactcccgttgatgggttggaaggcactgtcaaggaagaccacatactagatgttaagaacatcatagtcgccacgggctctgaagttacacccttccccggtattgaaatagatgaggaaaaaattgtctcttcaacaggtgctctttcgttaaaggaaattcccaaaagattaaccatcattggtggaggaatcatcggattggaaatgggttcagtttactctagattaggctccaaggttactgtagtagaatttcaacctcaaattggtgcatctatggacggcgaggttgccaaagccacccaaaagttcttgaaaaagcaaggtttggacttcaaattaagcaccaaagttatttctgcaaagagaaacgacgacaagaacgtcgtcgaaattgttgtagaagatactaaaacgaataagcaagaaaatttggaagctgaagttttgctggttgctgttggtagaagaccttacattgctggcttaggggctgaaaagattggattagaagtagacaaaaggggacgcctagtcattgatgaccaatttaattccaagttcccacacattaaagtggtaggagatgttacatttggtccaatgctggctcacaaagccgaagaggaaggtattgcagctgtcgaaatgttgaaaactggtcacggtcatgtcaactataacaacattccttcggtcatgtattctcacccagaagtagcatgggttggtaaaaccgaagagcaattgaaagaagccggcattgactataaaattggtaagttcccctttgcggccaattcaagagccaagaccaaccaagacactgaaggtttcgtgaagattttgatcgattccaagaccgagcgtattttgggggctcacattatcggtccaaatgccggtgaaatgattgctgaagctggcttagccttagaatatggcgcttccgcagaagatgttgctagggtctgccatgctcatcctactttgtccgaagcatttaaggaagctaacatggctgcctatgataaagctattcattgttga

SEQ ID NO: 16 - protein, 499 aa, Saccharomyces cerevisiae
  MISC_FEATURE: dihydrolipoyl dehydrogenase - LPD1, Accession number NP_116635
MLRIRSLLNNKRAFSSTVRTLTINKSHDVVIIGGGPAGYVAAIKAAQLGFNTACVEKRGKLGGTCLNVGCIPSKALLNNSHLFHQMHTEAQKRGIDVNGDIKINVANFQKAKDDAVKQLTGGIELLFKKNKVTYYKGNGSFEDETKIRVTPVDGLEGTVKEDHILDVKNIIVATGSEVTPFPGIEIDEEKIVSSTGALSLKEIPKRLTIIGGGIIGLEMGSVYSRLGSKVTVVEFQPQIGASMDGEVAKATQKFLKKQGLDFKLSTKVISAKRNDDKNVVEIVVEDTKTNKQENLEAEVLLVAVGRRPYIAGLGAEKIGLEVDKRGRLVIDDQFNSKFPHIKVVGDVTFGPMLAHKAEEEGIAAVEMLKTGHGHVNYNNIPSVMYSHPEVAWVGKTEEQLKEAGIDYKIGKFPFAANSRAKTNQDTEGFVKILIDSKTERILGAHIIGPNAGEMIAEAGLALEYGASAEDVARVCHAHPTLSEAFKEANMAAYDKAIHC

SEQ ID NO: 17 - DNA, 1449 bp, Saccharomyces cerevisiae
  misc_feature: dihydrolipoyllysine-residue acetyltransferase, Accession number NM_001182909.3
atgtctgcctttgtcagggtggttccaagaatatccagaagttcagtactcaccagatcattgagactgcaattgagatgctacgcatcgtacccagagcacaccattattggtatgccggcactgtctcctacgatgacgcaaggtaatcttgctgcttggactaagaaggaaggtgaccaattgtctcccggtgaagttattgccgaaatagaaacagacaaggctcaaatggactttgagttccaagaagatggttacttagccaagattctagttcctgaaggtacaaaggacattcctgtcaacaagcctattgccgtctatgtggaggacaaagctgatgtgccagcttttaaggactttaagctggaggattcaggttctgattcaaagaccagtacgaaggctcagcctgccgaaccacaggcagaaaagaaacaagaagcgccagctgaagagaccaagacttctgcacctgaagctaagaaatctgacgttgctgctcctcaaggtaggatttttgcctctccacttgccaagactatcgccttggaaaagggtatttctttgaaggatgttcacggcactggaccccgcggtagaattaccaaggctgacattgagtcatatctagaaaagtcgtctaagcagtcttctcaaaccagtggtgctgccgccgccactcctgccgccgctacctcaagcactactgctggctctgctccatcgccttcttctacagcatcatatgaggatgttccaatttcaaccatgagaagcatcattggagaacgtttattgcaatctactcaaggcattccatcatacatcgtttcctccaagatatccatctccaaacttttgaaattgagacagtccttgaacgctacagcaaacgacaagtacaaactgtccattaatgacctattagtaaaagccatcactgttgcggctaagagggtgccagatgccaatgcctactggttacctaatgagaacgttatccgtaaattcaagaatgtcgatgtctcagtcgctgttgccacaccaacaggattattgacaccaattgtcaagaattgtgaggccaagggcttgtcgcaaatctctaacgaaatcaaggaactagtcaagcgtgccagaataaacaaattggcaccagaggaattccaaggtgggaccatttgcatatccaatatgggcatgaataatgctgttaacatgtttacttcgattatcaacccaccacagtctacaatcttggccatcgctactgttgaaagggtcgctgtggaagacgccgctgctgagaacggattctcctttgataaccaggttaccataacagggacctttgatcatagaaccattgatggcgccaaaggtgcagaattcatgaaggaattgaaaactgttattgaaaatcctttggaaatgctattgtga

SEQ ID NO: 18 - protein, 482 aa, Saccharomyces cerevisiae
  MISC_FEATURE: dihydrolipoyllysine-residue acetyltransferase, Accession number NP_014328
MSAFVRVVPRISRSSVLTRSLRLQLRCYASYPEHTIIGMPALSPTMTQGNLAAWTKKEGDQLSPGEVIAEIETDKAQMDFEFQEDGYLAKILVPEGTKDIPVNKPIAVYVEDKADVPAFKDFKLEDSGSDSKTSTKAQPAEPQAEKKQEAPAEETKTSAPEAKKSDVAAPQGRIFASPLAKTIALEKGISLKDVHGTGPRGRITKADIESYLEKSSKQSSQTSGAAAATPAAATSSTTAGSAPSPSSTASYEDVPISTMRSIIGERLLQSTQGIPSYIVSSKISISKLLKLRQSLNATANDKYKLSINDLLVKAITVAAKRVPDANAYWLPNENVIRKFKNVDVSVAVATPTGLLTPIVKNCEAKGLSQISNEIKELVKRARINKLAPEEFQGGTICISNMGMNNAVNMFTSIINPPQSTILAIATVERVAVEDAAAENGFSFDNQVTITGTFDHRTIDGAKGAEFMKELKTVIENPLEMLL

SEQ ID NO: 19 - DNA, 1362 bp, Caenorhabditis elegans
  misc_feature: PAH, Accesion number NM_001267255.1
atgaatattgatgaaattagaaagaagtacgccatggaatcgtacgttgctgacgtcaacgccgacattggcaagactactatcgtattcactcttcgtgaaaaggcaggagctctcgctgaaacattgaagctgttccaggcacatgatgtgaatctgtctcacattgaatcaagaccatcaaagactcatgaaggatgctatgaggtgctcgttgaatttgctgaagctgaagaccatcgtaagattgaaggagttattgagcatttccaacaaaaagctgaaaagaaggttcttgttcaagactggaacaccaaaaacaaacaaaacaaggattctgttccatggttcccacagaagatcaacgacatcgatcaatttgccaacagaattctttcttatggagctgagcttgatgccgatcaccctggatttaaggacatgacctaccgcgagcgcagaaagtttttcgccgatattgcattcaacttcaaacacggagacaagatccctactatcacctacactgatgaagaaattgccacgtggcgtacagtctacaacgagctgacagttatgtacccgaaaaacgcttgccaagagttcaactacatattcccactcctccagcagaattgtggttttggacctgaccgcattccacaattgcaggatgtttcagattttttgaaggattgtaccgggtacacgattcgaccagtcgctggtcttctttcttctcgtgatttcttggctggtttggccttccgtgtttttcattccacacaatacattcgccatcattccgctccaaaatacacacctgaaccagatatctgccacgagcttctgggacatgttccactatttgctgatgttgaatttgcacaattctcacaggaaatcggtcttgcttctcttggagctccagatgatgttattgaaaaacttgccacactctactggttcacaatcgaatttggaatctgtcaacaagatggggagaaaaaagcttacggagccggacttttgagttcatttggagagcttcaatatgcgttgagtgataagccggaagttgtagattttgatccagctgtatgttgtgtcaccaaatatccaatcacagaatatcagccaaagtatttcttagctgaatcatttgcaagtgctaagaacaaacttaaatcatgggcagctaccatcaatcgtccattccaaattcgttataatgcttacactcaacgagttgaaattctcgacaaggtagcagcacttcaacgtctcgcaagagacatcagaagtgatatttctactttggaagaagctcttggaaaagtgaacaatctcaagatgaagtga

SEQ ID NO: 20 - protein, 453 aa, Caenorhabditis elegans
  MISC_FEATURE: PAH, Accesion number NP_001254184.1
MNIDEIRKKYAMESYVADVNADIGKTTIVFTLREKAGALAETLKLFQAHDVNLSHIESRPSKTHEGCYEVLVEFAEAEDHRKIEGVIEHFQQKAEKKVLVQDWNTKNKQNKDSVPWFPQKINDIDQFANRILSYGAELDADHPGFKDMTYRERRKFFADIAFNFKHGDKIPTITYTDEEIATWRTVYNELTVMYPKNACQEFNYIFPLLQQNCGFGPDRIPQLQDVSDFLKDCTGYTIRPVAGLLSSRDFLAGLAFRVFHSTQYIRHHSAPKYTPEPDICHELLGHVPLFADVEFAQFSQEIGLASLGAPDDVIEKLATLYWFTIEFGICQQDGEKKAYGAGLLSSFGELQYALSDKPEVVDFDPAVCCVTKYPITEYQPKYFLAESFASAKNKLKSWAATINRPFQIRYNAYTQRVEILDKVAALQRLARDIRSDISTLEEALGKVNNLKMK

SEQ ID NO: 21 - DNA, 1351 bp, Caenorhabditis elegans
  misc_feature: Adenosine deaminase, Accession number NM_068686.5
agaaagcgcacccaagttcaaaatcgacaaaaatgggtggcgtattctcaggagatcgtgtattcaatgactctaaagctatacacaatgtcgataaacaaatgcttcacaatcatgatccgacgttatcagtgagcactccggaagtcaccaccgaaatgcacgagcgtctcaacttcccgaaagtcgaacttcatttgcatcttgatggagccgtccgatttgatacactgatcgatctttctcaacaaaagggaataccactggctggagctaagactgtcgaggaactcaaaaaagttttagtgacccacgagccagcaaatctttcaaaggttctcgaagcgtttgaaatatttttgccggttattcgcggagacttggcagctattgaaagagttgcttatgaactttgtgaggatcagcataacaatggagttgtctattttgaaggaagatattcaccacatttgttgctttgtaatgattatccggaagtaaccgctgctcacgttgtcgccgctgttaagaaaggatttgatcgtggagagaaacagttcggaatcaaagctcgttcaattctttgctgtattcgtggactagacaaaaagttcccacaattgattctcgatttggcaactgacttgaaacaactcggagttgtcgcgattgatgttgctggatcagctcacggagccgatgaacagtacgagccagaagtcgttgctgctttccaagaagcacacaaacgaggaattcatagaactgttcatgctggagagtctggtggtccaaaggaagttattaaagctattgaagatatgtatgcagagagaattggacatggttatagagtaatgagagatgaagaaatgtatttggagcattttgtaaactcaaagaatgttcatttggaagcttgtccatattcatctgtaatgactggtgctgttccattggattggaagaatcatccaattgcacgatgggctaaagatgatgttaatttctctgtatctcgtgatgatccaacatgttttgataattcgatgctcagtgaattgacacttgttcataagcagatcggattggatgtacatcaactgtggaaagcgcaacttaacgctgcccgttcttgtttccttcctgaagacgaaaaagctgaacttgtaaaacgagttgaagctggagagccgccacgtccatgattcatgattaatcactcatttttctaacatttttactcgcccattttcttatctttttctcttgaaacattcctacaaatgaatagacatcttctgatatctcttctgcatccactttcctgaatgaataaacttttgtttcc

SEQ ID NO: 22 - protein, 391 aa, Caenorhabditis elegans
  MISC_FEATURE: Adenosine deaminase, Accession number NP_501087.1
MGGVFSGDRVFNDSKAIHNVDKQMLHNHDPTLSVSTPEVTTEMHERLNFPKVELHLHLDGAVRFDTLIDLSQQKGIPLAGAKTVEELKKVLVTHEPANLSKVLEAFEIFLPVIRGDLAAIERVAYELCEDQHNNGVVYFEGRYSPHLLLCNDYPEVTAAHVVAAVKKGFDRGEKQFGIKARSILCCIRGLDKKFPQLILDLATDLKQLGVVAIDVAGSAHGADEQYEPEVVAAFQEAHKRGIHRTVHAGESGGPKEVIKAIEDMYAERIGHGYRVMRDEEMYLEHFVNSKNVHLEACPYSSVMTGAVPLDWKNHPIARWAKDDVNFSVSRDDPTCFDNSMLSELTLVHKQIGLDVHQLWKAQLNAARSCFLPEDEKAELVKRVEAGEPPRP

SEQ ID NO: 23 - protein, 352 aa, Mus musculus
  MISC_FEATURE: Adenosine deaminase, Accession number NP_001258981.1
MAQTPAFNKPKVELHVHLDGAIKPETILYFGKKRGIALPADTVEELRNIIGMDKPLSLPGFLAKFDYYMPVIAGCREAIKRIAYEFVEMKAKEGVVYVEVRYSPHLLANSKVDPMPWNQTEGDVTPDDVVDLVNQGLQEGEQAFGIKVRSILCCMRHQPSWSLEVLELCKKYNQKTVVAMDLAGDETIEGSSLFPGHVEAYEGAVKNGIHRTVHAGEVGSPEVVREAVDILKTERVGHGYHTIEDEALYNRLLKENMHFEVCPWSSYLTGAWDPKTTHAVVRFKNDKANYSLNTDDPLIFKSTLDTDYQMTKKDMGFTEEEFKRLNINAAKSSFLPEEEKKELLERLYREYQ

SEQ ID NO: 24 - DNA, 1704 bp, Mus musculus
  misc_feature: Adenosine deaminase, Accession number NM_001272052.1
agcgtgggcggggctgtgccggggcagcccggtaaaaaagagcgtggcgggccgcggtctctgagagccatcgggaagcgaccctgccagcgagccaacgcagacccagagagcttcggcggagagaaccgggaacacgctcggaaccatggcccagacacccgcattcaacaaacccaaagtagagttacacgtccacctggatggagccatcaagccagaaaccatcttatactttggcaagaagagaggcatcgccctcccggcagatacagtggaggagctgcgcaacattatcggcatggacaagcccctctcgctcccaggcttcctggccaagtttgactactacatgcctgtgattgcgggctgcagagaggccatcaagaggatcgcctacgagtttgtggagatgaaggcaaaggagggcgtggtctatgtggaagtgcgctatagcccacacctgctggccaattccaaggtggacccaatgccctggaaccagactgaaggggacgtcacccctgatgacgttgtggatcttgtgaaccagggcctgcaggagggagagcaagcatttggcatcaaggtccggtccattctgtgctgcatgcgccaccagcccagctggtcccttgaggtgttggagctgtgtaagaagtacaatcagaagaccgtggtggctatggacttggctggggatgagaccattgaaggaagtagcctcttcccaggccacgtggaagcctatgagggcgcagtaaagaatggcattcatcggaccgtccacgctggcgaggtgggctctcctgaggttgtgcgtgaggctgtggacatcctcaagacagagagggtgggacatggttatcacaccatcgaggatgaagctctctacaacagactactgaaagaaaacatgcactttgaggtctgcccctggtccagctacctcacaggcgcctgggatcccaaaacgacgcatgcggttgttcgcttcaagaatgataaggccaactactcactcaacacagacgaccccctcatcttcaagtccaccctagacactgactaccagatgaccaagaaagacatgggcttcactgaggaggagttcaagcgactgaacatcaacgcagcgaagtcaagcttcctcccagaggaagagaagaaggaacttctggaacggctctacagagaataccaatagccaccacagactgacgcagggcgggtcccctgaagatggcaaggccacttctctgagcctcatcctgtggataaagtctttacaactctgacatattgaccttcattccttccagaccttggagaggccaggtctgtcctctgattggatatcctggctaggtcccaggggacttgacaatcatgcacatgaattgaaaaccttccttctaaagctaaaattatggtgttcaataaagcagctggtgactggtatcttgcagcacatggtgaatatggtctcggggctgctggctaggatgctaagaaaggaggagccctgggccctacgctgagtgtcaggctggggagccagggtctctttcctgcagaagcgattctttcccagaggggctgttggagcagatgctcctgaactctccgcccctttaaccagtcctttggatttatttttattatttttaaatatttaattatgtttatgtatatgggtgtttt

(19) United States
(12) Patent Application Publication
(10) Pub. No.: US 2021/0381022 A1
(43) Pub. Date: Dec. 9, 2021

(54) INTEGRATED DRUG DISCOVERY PLATFORM FOR INBORN ERROR OF METABOLISM DISORDERS

(71) Applicant: RAMOT AT TEL-AVIV UNIVERSITY LTD., Tel Aviv (IL)

(72) Inventors: Ehud GAZIT, Tel Aviv (IL); Dana LAOR, Tel Aviv (IL)

(21) Appl. No.: 17/345,248

(22) Filed: Jun. 11, 2021

Related U.S. Application Data
(63) Continuation-in-part of application No. PCT/IL2019/051366, filed on Dec. 12, 2019.
(60) Provisional application No. 62/779,054, filed on Dec. 13, 2018.

(51) Int. Cl. C12Q 1/02; G01N 33/569; G01N 33/50; A61K 49/00; A61K 31/122; A61K 31/353

(52) U.S. Cl. CPC C12Q 1/025; G01N 33/56961; G01N 33/5014; G01N 33/5038; G01N 2800/7076; G01N 33/5088; A61K 31/122; A61K 31/353; G01N 2800/04; A61K 49/0008

(57) ABSTRACT

The invention relates to in vivo systems and high throughput screening platform for drugs applicable in inborn error of metabolism (IEM) disorders. More specifically, the invention provides yeast screening system of candidate therapeutic compounds applicable in IEM disorders associated with accumulation of at least one metabolite. The systems of the invention comprise yeast cell/s that carry at least one manipulation in at least one yeast metabolic pathway, that leads to accumulation of said metabolite.

### FIELD OF THE INVENTION

The invention relates to drug discovery. More specifically, the present invention provides in vivo systems and high throughput screening platform for drugs applicable in inborn error of metabolism (IEM) disorders.

### BACKGROUND ART

References considered to be relevant as background to the presently disclosed subject matter are listed below:
[1] Chiti, F. & Dobson, C. M. Protein misfolding, amyloid formation, and human disease: a summary of progress over the last decade. Annu. Rev. Biochem. 86, 27-68 (2017).
[2] Adler-Abramovich, L. et al. Phenylalanine assembly into toxic fibrils suggests amyloid etiology in phenylketonuria. Nat. Chem. Biol. 8, 701-706 (2012).
[3] Shaham-Niv, S., Adler-Abramovich, L., Schnaider, L. & E. Gazit. Extension of the generic amyloid hypothesis to nonproteinaceous metabolite assemblies. Sci. Adv. 1, e1500137 16 (2015).
[4] Shaham-Niv, S. et al. Differential Inhibition of Metabolite Amyloid Formation by Generic Fibrillation-Modifying Polyphenols. Comms. Chem. (2018).
[5] Shaham-Niv, S. et al. Formation of apoptosis-inducing amyloid fibrils by tryptophan. Isr. J. Chem. 57, 729-737 (2017).
[6] Gazit, E. Metabolite amyloids; a new paradigm for inborn error of metabolism disorders. J. Inherit. Metab. Dis. 39, 483-488 (2016)
[7] Shaham-Niv, S. et al. Metabolite amyloid-like fibrils interact with model membranes. Chem. Commun. 54, 4561-4564 (2018).
[8] Treusch, S. et al. Functional links between A toxicity, endocytic trafficking, and Alzheimer's disease risk factors in yeast. Science 334, 1241-1245 (2011).
[9] Tardiff, D. F. et al. Yeast reveal a “druggable” Rsp5/Nedd4 network that ameliorates a-synuclein toxicity in neurons. Science 342, 979-983 (2013).
[10] Meriin, A. B. et al. Huntington toxicity in yeast model depends on polyglutamine aggregation mediated by a prion-like protein Rnq1. J. Cell. Biol. 157, 997-1004 (2002).
[11] Sigurdson, C., Polymenidou, M. & Aguzzi, A. Reconstructing prions: fibril assembly from simple yeast to complex mammals. Neurodegenerative Dis. 2, 1-5 (2005).
[12] Aayatekin, C., et al. Translocon Declogger Ste24 Protects against IAPP Oligomer-Induced Proteotoxicity. Cell 173, 62-73 (2018).
[13]. Khurana. V. & Lindquist, S. Modelling neurodegeneration in Saccharomyces cerevisiae: wby cook with baker's yeast? Nat. Rev. Neurosci. 11, 436-449 (2010).
[14] Zaguri, D., Kreiser, T., Shaham-Niv, S., & Gazit, E. Antibodies towards Tyrosine Amyloid-Like Fibrils Allow Toxicity Modulation and Cellular Imaging of the Assemblies. Molecules, 23, 1273 (2018).
[15] Porat, Y., Abramowitz, A. & E. Gazit. Inhibition of amyloid fibril formation by polyphenols: structural similarity and aromatic interactions as a common inhibition mechanism. Chem. Biol. Drug. Des. 67, 27-37 (2006).
[16] Ebrahimi, A. & Schluesener, H. Natural polyphenols against neurodegenerative disorders: potentials and pitfalls. Ageing Res. Rev. 11, 329-345 (2012).
[17] D. A. Kocisko et al. New inhibitors of scrapie-associated prion protein formation in a library of 2000 drugs and natural products. J. Virol. 77, 10288-10294 (2003).
[18] Li, Y., Zhao, J., & Holscher, C. Therapeutic potential of baicalein in alzheimer's disease and Parkinson's disease. CNSdrugs, 31, 639-652 (2017).

Acknowledgement of the above references herein is not to be inferred as meaning that these are in any way relevant to the patentability of the presently disclosed subject matter.
[19] Woidy, M, Ania C. M, and Soren W. G. Inborn errors of metabolism and the human interactome: a systems medicine approach. J. Inherit. Metab. Dis. 41, 285-296 (2018).
[20] WO 2014/145975 A2.
[21] Giaever, G., et al. Functional profiling of the Saccharomyces cerevisiae genome. Nature 418, 387-391 (2002).
[22] Uptain, Susan M., et al. Strains of [PSI+] are distinguished by their efficiencies of prion-mediated conformational conversion. EMBO J. 20(22), 6236-6245 (2001).
[23] Malinovska, L., Kroschwald, S., Munder, M. C., Richter, D. & Alberti, S. Molecular chaperones and stress-inducible protein-sorting factors coordinate the spatiotemporal distribution of protein aggregates. Mol. Biol. Cell 23, 3041-3056 (2012).
[24] Laor, D. et al. Fibril formation and therapeutic targeting of amyloid-like structures in a yeast model of adenine accumulation. Nat. Commun. 10, 62, 1-11 (2019).
[25] Rencus-Lazar, S., DeRowe, Y., Adsi, H., Gazit, E. & Laor, D. Yeast Models for the Study of Amyloid-Associated Disorders and Development of Future Therapy. Front. Mol. Biosci. 6, 15, 1-10 (2019).
[26] Fisher, Alfred L., et al. “The Caenorhabditis elegans K10C2. 4 Gene Encodes a Member of the Fumarylacetoacetate Hydrolase Family A CAENORHABDITIS ELEGANS MODEL OF TYPE I TYROSINEMIA.” Journal of Biological Chemistry 283.14 9127-9135 (2008).

### BACKGROUND OF THE INVENTION

The canonical amyloid hypothesis attributed the formation of nano-scale fibrillar assemblies exclusively to proteins and polypeptides [1]. However, a new paradigm for the pathophysiology of inborn error of metabolism disorders significantly extended the original hypothesis, showing that at millimolar pathological concentrations, the single phenylalanine amino acid can form nanofibrillar structures in aqueous solution and neutral pH in vitro [2]. These nonproteinaceous assemblies exhibit typical apple-green birefringence and clear fluorescence signal upon Congo red staining when examined under cross-polarized light and fluorescent microscopy, intense fluorescence following thioflavin T staining and cell culture cytotoxicity [2,3]. Using electron microscopy, a fibrillar morphology of the phenylalanine assemblies was observed, showing physical properties characteristic of protein amyloids. As opposed to single crystals that show regular geometrical shape consisting of flat faces, amyloids structures have a fibrillar morphology. Based on the similar characteristics to amyloid proteins, these non-proteinaceous assemblies were suggested to display ‘amyloid-like’ properties.

The notable toxicity of the assemblies was suggested to be associated with the neurological damage observed in non-treated patients suffering from the phenylketonuria (PKU) error of metabolism disorder, in which phenylalanine accumulates due to metabolic pathway alteration. Histological post-mortem staining of brain tissues of human PKU patients, as well as of PKU model mice, using specific antibodies raised against phenylalanine fibrils, demonstrated the specificity of the antibodies and the formation of metabolite amyloid-like assemblies in the disease state [2]. Follow-up studies supported the notion that the single phenylalanine amino acid can form amyloid-like nanofibrillar structures, established the mechanism of oligomerization, and determined the ability of the phenylalanine assemblies to interact with phospholipid membranes, similar to protein amyloids [3]. Furthermore, doxycycline, epigallocatechin gallate and tannic acid (TA), known inhibitors of amyloid fibril formation, were shown to counteract both phenylalanine aggregation and cy totoxicity of the assemblies in vitro [4]. Moreover, the amyloid hypothesis was significantly extended by demonstrating that several other metabolites, including additional amino acids and nucleobases, could form such archetypical nanofibrils in vitro, displaying amyloid-like properties [3, 5, 6]. The fact that the alanine amino acid shows none of the above characterizations, as well as no toxic effect when added to cultured cells at high concentrations, demonstrates that the toxic effect is due to the structures formed by certain metabolites [3].

Inborn errors of metabolism, stemming from mutations resulting in enzymatic deficiencies in various metabolic pathways, can lead to the accumulation of substrates. Thus, for example, the required daily allowance (RDA) of phenylalanine for the general population may actually be toxic to individuals with PKU. Therefore, in the absence of strict dietary restrictions, PKU can lead to mental retardation and other developmental abnormalities. The recent extension of the amyloid hypothesis offers new opportunities for both diagnostics as well as therapy of these disorders. Specifically, inborn mutations in genes involved in the adenine salvage pathway in humans can lead to the development of several metabolic disorders as a result of the accumulation of adenine and its derivatives. The inventors previously shown the formation of adenine amyloid-like structures in vitro. These assemblies displayed amyloidogenic properties, including the appearance of typical amyloid fibrils as demonstrated by electron microscopy, positive staining with amyloid-specific dyes, and notable cytotoxicity in cultured cells [3].

Moreover, formation of the adenine structures was shown to be inhibited by amyloid specific inhibitors in vitro and adenine assemblies could interact with a membrane model, similar to their proteinaceous counterparts [4, 7]. Yet, analysis of the formation of amyloid-like assemblies by metabolites has so far been limited to in vitro studies. Thus, there is a genuine need for in vivo models for the formation of such assemblies in order to understand the biological relevance and the consequences of metabolite molecular self-assembly, thereby targeting early stages of formation of metabolite amyloid fibrils. Yeast can assist in revealing the core abnormal processes underlying multiple aspects of biomolecular aggregation [13]. The pioneering work of Susan Lindquist and coworkers, as well as follow-up studies, had established yeast as an excellent model for several amyloid-associated disorders, including Alzheimer's disease [8], Parkinson's disease [9], Huntington's disease [10] and prion disorders [11] and recently also type 2 diabetes [12], supporting the high relevance of this approach for further establishing the extended amyloid hypothesis. WO 2014/145975 [20], by Lindquist et al., discloses yeast based platform for drug discovery for neurodegenerative disorders. However, this platform involves exogenous insertion of human proteins involved in neurodegenerative disorders, that are not naturally expressed in yeast, and as such may not enable targeting early stages of fibril formation. The present invention establishes the first in vivo model for the study of the self-assembly of adenine into amyloid-like structures. The present invention therefore provides a novel methodology for establishing additional yeast models that could most reliably mimic the metabolic state in inborn error of metabolism disorders, as the native metabolic pathways may be manipulated with no artificial introduction of genes.

### SUMMARY OF THE INVENTION

In a first aspect, the invention provides a yeast screening system of candidate therapeutic compounds. In more specific embodiments, the system of the invention comprises. (a) a yeast cell and/or yeast cell line, and/or yeast cell population, and/or any progeny thereof, that carry at least one manipulation in at least one yeast metabolic pathway, that leads to accumulation of at least one metabolite. It should be noted that in some embodiments, the accumulation of such at least one metabolite is associated with at least one inborn error of metabolism (IEM) disorder. In some embodiments, such yeast cell and/or yeast cell line, and/or yeast cell population, and/or any progeny thereof, carry at least one manipulation in a native yeast pathway that may lead to accumulation of such metabolite. In some embodiments, the system of the invention may further optionally comprise (b), at least one reagent or means for determining at least one of, accumulation of the metabolite and at least one phenotype associated with accumulation of said metabolite. In some embodiments, the compound screened by the system disclosed herein is useful in treating, preventing, ameliorating, reducing or delaying the onset of at least one inborn error of metabolism (IEM) disorder associated with accumulation of at least one metabolite.

In another aspect, the invention provides a screening method of candidate therapeutic compounds for treating, preventing, ameliorating, reducing or delaying the onset of at least one IEM disorder associated with accumulation of at least one metabolite. In more specific embodiments, the method of the invention may comprise the steps of. In a first step (a), contacting a manipulated yeast cell and/or yeast cell line, and/or yeast cell population, and/or any progeny thereof with a candidate compound, said yeast cell/cells carry at least one manipulation in at least one yeast metabolic pathway, that leads to accumulation of said metabolite. The second step (b), involves determining and/or measuring in the incubated cells of (a), at least one of, metabolite accumulation and at least one phenotype associated with the accumulation of the metabolite. The next step (c) involves determining that the candidate is a therapeutic compound for said IEM disorder if the level of said phenotype is modulated as compared with the metabolite accumulation and/or phenotype in the absence of the candidate compound.

A further aspect of the invention relates to a method for treating, preventing, ameliorating, reducing or delaying the onset of at least one IEM disorder associated with accumulation of at least one metabolite. In more specific embodiments, the method of the invention may comprise the steps of: First, obtaining a compound that modulates the level of at least one phenotype associated with the accumulation of said metabolite by a screening method comprising:

(i) contacting a yeast cell and/or yeast cell line, and/or yeast cell population, and/or any progeny thereof with a candidate compound, said yeast cell and/or yeast cell line, and/or yeast cell population, and/or any progeny thereof, carry at least one manipulation in at least one yeast metabolic pathway, that leads to accumulation of said metabolite: (ii) measuring and/or determining in the incubated cell/s of (i) the level of accumulation of the metabolite and/or at least one phenotype associated with the accumulation of said metabolite: and (iii) determining that said candidate is a therapeutic compound for said IEM disorder if the accumulated metabolite and/or the phenotype is modulated as compared with the accumulated metabolite and/or the phenotype in the absence of said candidate compound. The next step (b) involves administering a therapeutic effective amount of the compound obtained by step (a) to a subject suffering from IEM disorder associated with accumulation of the metabolite. The invention further provides a therapeutic compound for use in a method for treating, preventing, ameliorating, reducing or delaying the onset of at least one IEM disorder associated with accumulation of at least one metabolite. The therapeutic compound used in said method was identified by the screening method of the invention.

A further aspect of the invention relates to a therapeutic compound for treating, preventing, ameliorating, reducing or delaying the onset of at least one IEM disorder associated with accumulation of at least one metabolite. In some specific embodiments, the compound of the invention may be identified by a method comprising the steps of:

In a first step (a), contacting a manipulated yeast cell and/or yeast cell line, and/or yeast cell population, and/or any progeny thereof, with the cell or cell line that carries at least one manipulation in at least one yeast metabolic pathway, that leads to accumulation of said metabolite. The second step (b), requires measuring in the incubated cells of (a), the accumulated metabolite and/or at least one phenotype associated with the accumulation of the metabolite. The third step (c) involves determining that the candidate is a therapeutic compound for the IEM disorder if the accumulated metabolite and/or the phenotype is modulated as compared with the accumulated metabolite and/or the phenotype in the absence of the candidate compound.

The invention further provides specific compounds identified by the screening methods of the invention. In more specific embodiments, the invention provides at least one small molecule compound of the general formula (II):

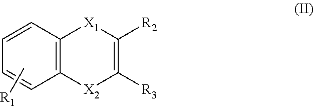

or a pharmaceutically acceptable salt, solvate, hydrate, any stereoisomer thereof or physiologically functional derivative thereof.
wherein each of X₁ and X₂ is independently selected from (CR⁴R⁵), O, S or NR⁴, each R⁴ and R⁵ independently is hydrogen, OH, C₁-C₆ alkyl, C₁-C₆ alkoxy, or CR⁴R⁵ is C═O,
R₁ is selected from hydrogen or —OH

Each of R₂ and R₃ is independently selected from hydrogen, an aryl optionally substituted or R₂ and R₃ form together with additional two carbons atoms, a six-membered ring. In some embodiments, the small molecule compound of the invention is any one of: 1,2,4-Trihydroxyanthraquinone (Purpurin) or any derivatives, analogs, salts and esters thereof, 1,2-Dihydroxvanthracene-9,10-dione (Alizarin) or any derivatives, analogs, salts and esters thereof. 1,2,5,8-tetrahvdroxyanthraquinone (Quinalizarin) or any derivatives, analogs, salts and esters thereof and 5,6,7-trihydroxyflavone (Baicalein) or any derivatives, analogs, salts and esters thereof, or any composition comprising the same. In yet some further embodiments, the invention provides the compound of Formula II, for use in a method for treating, preventing, ameliorating, reducing or delaying the onset of at least one IEM disorder associated with accumulation of at least one metabolite. The invention further provide therapeutic methods for treating, preventing, ameliorating, reducing or delaying the onset of at least one IEM disorder associated with accumulation of at least one metabolite that comprise the step of administering to the subject an effective amount of the compound of Formula II. In some specific embodiments, such compound is any one of 1,2,4-Trihydroxyanthraquinone (Purpurin) or any derivatives, analogs, salts and esters thereof, 1,2-Dihydroxyanthracene-9,10-dione (Alizarin) or any derivatives, analogs, salts and esters thereof, 1,2,5,8-tetrahvdroxyanthraquinone (Quinalizarin) or any derivatives, analogs, salts and esters thereof and 5,6,7-trihydroxyflavone (Baicalein) or any derivatives, analogs, salts and esters thereof. These and other aspects of the invention will become apparent by the hand of the following description and drawings.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1A-1E: Sensitivity of the salvage mutant to adenine feeding

FIG. 1A: Wild-type (WT), aah1Δ, apt1Δ and aah1Δapt1Δ strains were serially diluted and spotted on SD complete medium containing 20 mg/L adenine (SD-com) or on SD medium without adenine (−ADE).

FIG. 1B: Growth curves of WT and aah1Δapt1Δ cells in SD media with or without adenine (ADE). SD-com is denoted as +ADE to emphasize the presence of adenine in the standard yeast growth medium.

FIG. 1C: WT, aah1Δ, apt1Δ and aah1Δapt1Δ strains were serially diluted and spotted on SD medium with various concentrations of adenine, ranging from 40 mg/L (˜300 μM) to 5 μg/L (˜0.0375 μM), or without adenine (−ADE).

FIG. 1D: Cells were grown in the presence of different concentrations of adenine, ranging from 40000 μg/L (˜300 μM) to 0.038 μg/L (˜0.029 nM), in SD medium and the absorbance at 600 nm was measured at the logarithmic phase. The results were fitted using a four-parameter logistic equation (4PL), R2>0.99. Inset shows the absorbance of the WT at 600 nm.

FIG. 1E: Intracellular concentration of adenine determined using GC-MS. WT and aah1Δapt1Δ cells were grown in SD media in the absence (−ADE) or presence (+ADE) of adenine and the metabolites were extracted. *P<0.01 (Student's t-test). Values are the mean±s.d. of three experiments.

FIG. 2: Respiratory competence of the salvage mutant upon adenine feeding using glycerol as a carbon source

WT, aah1Δ, apt1Δ and aah1Δapt1Δ strains were serially diluted and spotted on SG complete medium (2% glycerol) containing 20 mg/L adenine (SG+ADE) or on SG medium without adenine (SG-ADE).

FIG. 3: aah1⁺ and apt1⁺ insertion to aah1Δapt1Δ strain restore the cell growth toxicity observed in the presence of adenine

aah1Δapt1Δ strain transformed with single copy plasmids carrying pRS416-AAH1(pAAH1) and pRS313-APT1(pAPT1) as well as WT and aah1Δapt1Δ strains transformed with vectors only (pRS416 and pRS313) were serially diluted and spotted on SD medium without the relevant markers (uracil and histidine respectively) containing 20 mg/L adenine (SD+ADE) or without adenine (SD-ADE).

FIG. 4: Adenine self-assembly in vitro at different concentrations

Adenine was dissolved to a final concentration of 1 mg/ml to 10 mg/ml, as indicated. Next, 20 μM ThT (final concentration) was added following an overnight incubation and fluorescence emission endpoint measurement at 480 nm (excitation at 450 nm) was carried out.

FIG. 5A-5F: In vivo Raman visualization of adenine accumulation FIG. 5A: Space-resolved Raman spectra of mutant yeast (aah1Δapt1Δ), whole fingerprint region of the Raman spectrum.

FIG. 5B: Space-resolved Raman spectra of mutant yeast (aah1Δapt1Δ), zoom up of nucleic acids and the adenine marker region (800-600 cm-1).

FIG. 5C: Space-resolved Raman spectra of mutant yeast (aah1Δapt1Δ), the corresponding optical image is presented and the measured points are indicated using colored asterisks (gray scale).

FIG. 5D: Raman chemical images of the salvage mutant and wild-type yeast cells in the presence of adenine.

FIG. 5E: Raman chemical images of the salvage mutant and wild-type yeast cells in the absence of adenine.

Raman images of protein (1004 cm⁻¹; magenta), adenine (724 cm⁻¹; blue), nucleic acids (785 cm⁻¹ green) and the relative intensity of adenine and nucleic acids (724/785 cm⁻¹; red) are presented. Corresponding optical images are included for reference. Rel. Int., relative intensity.

FIG. 5F: Calculated average intracellular intensity from yeast cells of 724 cm⁻¹ per pixel, 785 cm-1 per pixel and relative intensity of WT and aah1Δapt1Δ yeast cells. P<0.02 (Student's t-test). Values are the mean±s.d. of three experiments.

FIG. 6A-6B: Raman spectra of adenine

FIG. 6A: Raman band was measured using 5 mg/ml of adenine in 0.5M HCl_{aq} solution.

FIG. 6B: Raman band of adenine in solid from.

FIG. 7. In vivo characterization of distinct amyloid fibers formed by the yeast prion protein Sup35 by ProteoStat staining

10 μl of W303 [psi⁻], weak [PSI⁺] and strong [PSI⁺] were spotted on YPD plate for color distinction following ProteoStat staining and analyzed by flow cytometry, showing a correlation to the level of [PSI⁺]: red, pink and white respectively. *P<0.01 (Student's t-test). Values are the mean±s.d. of three experiments.

FIG. 8A-8F: In vivo formation of amyloid-like structures upon adenine feeding FIG. 8A: Flow cytometry analysis of WT and aah1Δapt1Δ cells under the indicated conditions using ProteoStat staining. *P<0.01 (Student's t-test). Values are the mean f s.d. of three experiments.

FIG. 8B: Flow cytometry analysis of WT and aah1Δapt1Δ cells representative confocal and differential interference contrast (DIC) images.

FIG. 8C: Z-stack followed by 3D reconstruction of aah1Δapt1Δ cells

FIG. 8D: projection of a single section of aah1Δapt1Δ cells using ProteoStat. The analysis was performed using the Imaris software.

FIG. 8E: Representative image of aah1Δapt1Δ cells double-stained with Hoechst and ProteoStat. Cells were visualized using DIC microscopy.

FIG. 8F: Representative confocal and differential interference contrast (DIC) images of WT and aah1Δapt1Δ cells under the indicated conditions. Cells were fixed and subjected to indirect immunofluorescence using a polyclonal antibody raised against adenine amyloid-like assemblies, designated as Anti-ADE_{af(=amyloid fibrils)}.

FIG. 9A-9C: The sensitivity of the salvage mutant to adenine feeding is independent of Hsp104

FIG. 9A: WT, aah1Δ, apt1Δ and aah1Δapt1Δ strains were serially diluted and spotted on SD complete medium containing 20 mg/L adenine (SD+ADE) or on SD medium without adenine (SD-ADE) in the absence (−GdnHCl) or presence (+GdnHCl) of 5 mM guanidine hydrochloride.

FIG. 9B: WT, aah1Δapt1Δ, hsp104Δ and aah1Δapt1Δhsp104Δ strains were serially diluted and spotted on SD complete medium containing 20 mg/L adenine (SD+ADE) or on SD medium without adenine (SD-ADE).

FIG. 9C: Representative confocal and differential interference contrast (DIC) images of WT and Δaah1Δapt1 cells expressing Hsp104-mCherry on SD complete medium containing 20 mg/L adenine at 30° C. and following heat shock (15 minutes at 46° C.). Images in the right panel of each strain/condition are magnified views of the area marked with dashed lines.

FIG. 10A-10D: Addition of TA rescues the toxic effect observed in the adenine salvage mutant

FIG. 10A: WT, aah1Δ, apt1Δ and aah1Δapt1Δ strains were serially diluted and spotted on SD medium without adenine (−ADE) or on SD media containing 2 mg/L adenine with or without various concentrations of TA, as indicated.

FIG. 10B: Dose response curve for aah1Δapt1Δ cells in SD medium containing adenine and TA at different concentrations. The percentage of growth represents the growth with TA compared to the growth without TA.

FIG. 10C: Flow cytometry analysis of WT and aah1Δapt1Δ cells under the indicated conditions following ProteoStat staining. *P<0.01 (Student's t-test). Values are the mean t s.d. of three experiments. TA concentration was 0.5 mM.

FIG. 10D: Representative confocal microcopy images of aah1Δapt1Δ cells under the same conditions as in FIG. 10C. Cells were visualized using DIC microscopy.

FIG. 11A-11D: TA is present inside aah1Δapt1Δ cells together with adenine

FIG. 11A: aah1apt1Δ cells were grown in SD medium containing adenine and TA. LC-MS analysis focused on TA negatively charged molecular peak at 1700 Da, RT around 5.40 minutes.

FIG. 11B: aah1Δapt1Δ cells were grown in SD medium containing adenine and TA. LC-MS analysis focused on adenine positively charged molecular peak at 136.1 Da, RT around 0.70 minutes.

FIG. 11C: aah1Δapt1Δ cells were grown in SD medium containing adenine without TA. LC-MS analysis focused on TA negatively charged molecular peak at 1700 Da. RT around 5.40 minutes.

FIG. 11D: aah1Δapt1Δ cells were grown in SD medium containing adenine without TA. LC-MS analysis focused on adenine positively charged molecular peak at 136.1 Da, RT around 0.70 minutes.

Examined samples were tannic acid and adenine as standards (SND), the growth medium (Media), the washing steps (Wash1-3), the pellet (Pellet) and the supernatant (Sup).

FIG. 12A-12D: Addition of baicalein rescues the toxic effect observed in the adenine salvage mutant

FIG. 12A: Growth curves of WT strains in SD medium containing 2 mg/L adenine (+ADE), with or without the following concentrations of baicalein: 2.5 μM, 5 μM and 10 μM.

FIG. 12B: Growth curves of Δaah1Δapt1 strains in SD medium containing 2 mg/L adenine (+ADE), with or without the following concentrations of baicalein: 2.5 μM, 5 μM and 10 μM.

FIG. 12C: Flow cytometry analysis of Δaah1Δapt1 cells with and without 10 μM baicalein following ProteoStat staining. *P<0.01 (Student's t-test). Values are the mean t s.d. of three experiments.

FIG. 12D: Adenine self-assembly in vitro in the presence of baicalein. ThT fluorescence emission intensity at 480 nm (excitation at 450 nm) in the presence of 8 mg/ml adenine was measured over time in the absence (PBS) and in the presence of baicalein at the following concentrations: 5 μM, 10 μM, 100 μM, 500 μM. Adenine was dissolved and treated as described in the Experimental procedures section.

FIG. 13A-13C: TA rescues salvage model yeast by preventing adenine assembly into toxic amyloid-like structures

FIG. 13A: WT and aah1Δapt1Δ cells diluted to OD₆₀₀ 0.01 were grown in SD media in the presence of adenine. 0.5 mM TA was added to the samples at different OD₆₀₀ values (0.01, 0.05, 0.1 and 0.2). *P<0.01 (Student's t-test). The percentage of growth represents the growth with TA compared to the growth without TA. Values are the mean f s.d. of three experiments. Schematic illustrations of adenine assembly inside the cells at the different OD₆₀₀ values following the addition of TA are shown below the X-axis.

FIG. 13B: Intracellular concentration of adenine determined using GC-MS. WT and aah1Δapt1Δ cells were grown in SD media in the presence of adenine with or without 0.5 mM TA and the metabolites were extracted. ns, not significant (Student's t-test). Values are the mean±s.d. of three experiments.

FIG. 13C: Schematic model of adenine accumulation in WT cells compared to the adenine salvage model in the absence or presence of adenine, and following the addition of the TA inhibitor. The model reflects the relative amounts of the metabolite as determined experimentally. Upon feeding of the salvage mutant with adenine, the metabolite accumulates into ordered assemblies. Administration of the inhibitor prevents the formation of assemblies at the nucleation phase, without any significant effect on the total concentration of the metabolite.

FIG. 14: HTS yeast growth assay

Saccharomyces cerevisiae yeast mutant were grown in presence of adenine and compounds in 384 plates for 20 hours and OD measures were taken every 10 minutes. Potential hits were picked using analyzes of curve slope and area under the curve.

FIG. 15A-15B: Quantification of adenosine aggregates and cell viability in transformed ADA deficient LCLs

FIG. 15A: FACS analyses of adenosine aggregates. LCLs derived from healthy (blue bars) or from ADA deficient (red bars) were incubated for 3 days with or without adenosine and stained for adenosine aggregates with proteostat dye. Fluorescence was measured by FACS.

FIG. 15B: XTT based viability assay. LCLs derived from healthy (blue bars) or from ADA deficient (red bars) were incubated for 3 days with or without adenosine and cell viability was analyzed by XTT based kit.

FIG. 16: Non-linear response to adenosine feeding in LCLs from ADA-deficiency patients compared to healthy LCLs. LCLs derived from healthy or from ADA deficient were grown in the presence of different concentrations of adenosine, ranging from 0.0013 mg/ml to 3.2 mg/ml for 3 days and cell viability was analyzed by XTT based kit.

FIG. 17A-17C: Purpurin rescues adenosine toxicity phenotype in LCLs from ADA-deficiency patients

FIG. 17A: FACS analyses of adenosine aggregates. FACS analyses of LCLs from healthy (1,2,3,4) or ADA deficient (8,11,12) individuals with or without purpurin stained with proteostat dye for detection of adenosine aggregates. Fluorescence of each cell line is relative to the one without adenosine.

FIG. 17B: XTT based viability assay. XTT based cell survival assay with or without purpurin performed on healthy or ADA deficient LCLs. Survival percentage is relative to survival without adenosine.

FIG. 17C: Purpurin showed high potency in rescuing mutant yeast strain from adenine toxicity.

FIG. 18A-18D: Purpurin, Alizarin, Baicalein and Quinalizarin can significantly reduce the aggregation level in patient lymphoblastoids

FIG. 18A: Aggregation level was measured following treatment with Purpurin at different concentrations, using lymphoblasts from ADA-deficiency patients in the presence of adenosine alone (black) and in the presence of adenosine (grey).

FIG. 18B: Aggregation level was measured following treatment with Baicalein at different concentrations, using lymphoblasts from ADA-deficiency patients in the presence of adenosine alone (black) and in the presence of adenosine (grey).

FIG. 18C: Aggregation level was measured following treatment with Alizarin at different concentrations, using lymphoblasts from ADA-deficiency patients in the presence of adenosine alone (black) and in the presence of adenosine (grey).

FIG. 18D: Aggregation level was measured following treatment with Quinalizarin at different concentrations, using lymphoblasts from ADA-deficiency patients in the presence of adenosine alone (black) and in the presence of adenosine (grey).

FIG. 19: Table of compounds identified by the screening method

Chemical structure of Purpurin, Quinalizarin, Alizarin and Baicalein and their calculated IC₅₀ values in human cells

FIG. 20: Inhibition of adenine fibrils formation by different inhibitors

Adenine was dissolved at 90° C. in PBS to a final concentration of 8 mg/ml and mixed with the indicated inhibitor, at the indicated concentrations, or with PBS as a control, followed by addition of ThT in PBS. ThT emission data at 480 nm (excitation at 450 nm) were measured at the end point.

FIG. 21A-21C: Sensitivity of the aro3Δ mutant to tyrosine feeding in a dose-dependent manner.

FIG. 21A: Wild-type (WT) and aro3Δ strains were serially diluted and spotted on 4a.a SD plate and 4a.a SD plates with the indicated tyrosine concentration. Cells were grown at 30° C. for 2 days.

FIG. 21B: Growth curves of WT cells in 4a.a SD media without tyrosine (−Tyr) or with the indicated tyrosine concentration. The absorbance at OD 600 was measured over time. The results represent three biological repeats.

FIG. 21C: Growth curves of aro3Δ cells in 4a.a SD media without tyrosine (−Tyr) or with the indicated tyrosine concentration. The absorbance at OD 600 was measured over time. The results represent three biological repeats.

FIG. 22A-22D: In vivo quantification of the levels of aromatic amino acids in the presence of tyrosine

Intracellular concentrations of the aromatic amino acids were determined using LC-MS. Wild-type (WT) and aro3Δ strains were grown in 4a.a SD media in the absence or the presence of 0.02 mM tyrosine, and the metabolites were extracted. Values are the average±St. dev. of two biological repeats.

FIG. 22A: Intracellular concentrations of tyrosine. **P<0.001 (Student's t-test).

FIG. 22B: Intracellular concentrations of tryptophan.

FIG. 22C: Intracellular concentrations of phenylalanine.

FIG. 22D: Intracellular concentrations of the aromatic amino acids in the presence of tyrosine. *P<0.01 (Student's t-test).

FIG. 23A-23B: In vivo formation of amyloid-like structures upon tyrosine feeding

FIG. 23A: Graphs showing Flow cytometry analysis of wild-type (WT) and aro3Δ mutant under the indicated conditions (tyrosine concentration is 0.02 mM) following ProteoStat® staining.

FIG. 23B: Graph showing the percentage of aggregates, Values are the average±St. dev. of three technical repeats and represent two biological repeats. *P<0.01, **P<0.001 (Student's t-test).

FIG. 24A-24C: Inhibition of tyrosine toxicity by EGCG

FIG. 24A: Wild-type (WT) and aro3Δ strains were serially diluted and spotted on 4a.a SD plate, 4a.a SD plate with 0.03 mM tyrosine (+Tyr) and 4a.a SD with 0.03 mM tyrosine and the indicated concentration of EGCG. Cells were grown at 30° C. for 2 days.

FIG. 24B: Growth curves of WT cells in 4a.a SD media without tyrosine (−Tyr) or with 0.03 mM tyrosine (+Tyr) and with the addition of the indicated concentration of EGCG. The absorbance at OD 600 was measured over time. The results represent three biological repeats.

FIG. 24C: Growth curves of aro3Δ cells in 4a.a SD media without tyrosine (−Tyr) or with 0.03 mM tyrosine (+Tyr) and with the addition of the indicated concentration of EGCG. The absorbance at OD 600 was measured over time. The results represent three biological repeats.

FIG. 25A-25B: Inhibition of tyrosine toxicity by EGCG FIG. 25A: Graphs showing Flow cytometry analysis of wild-type (WT) and aro3Δ mutant under the indicated conditions (Tyrosine concentration is 0.02 mM and EGCG concentration is 5 mM) following ProteoStat® staining.

FIG. 25B: Graph showing the percentage of aggregates, Values are the average f St. dev. of three technical repeats and represent two biological repeats.***P<0.0001 (Student's t-test).

FIG. 26A-26B: Sensitivity of aro4Δ to phenylalanine feeding

FIG. 26A: Wild-type (WT) and aro4Δ strains were serially diluted and spotted on SD medium containing 1 mg/L (0.6 mM) phenylalanine and medium without phenylalanine.

FIG. 26B: Growth curves of WT and aro4Δ cells in SD media with or without phenylalanine (Phe).

FIG. 27A-27E: Yeast model for homocysteine accumulation

FIG. 27A: Wild-type (WT) and cys4Δ strains were serially diluted and spotted on YPD, SD medium containing 13 mg/L cysteine and 100 mg/L homocysteine (SD+cys+Hcy), SD medium without cysteine and homocysteine (SD-cys) or SD medium containing 13 mg/L cysteine without homocysteine (SD+cys).

FIG. 273: Growth curves of WT on SD media with or without homocysteine (Hcy) at different concentrations (20 mg/L, 40 mg/L and 80 mg/L).

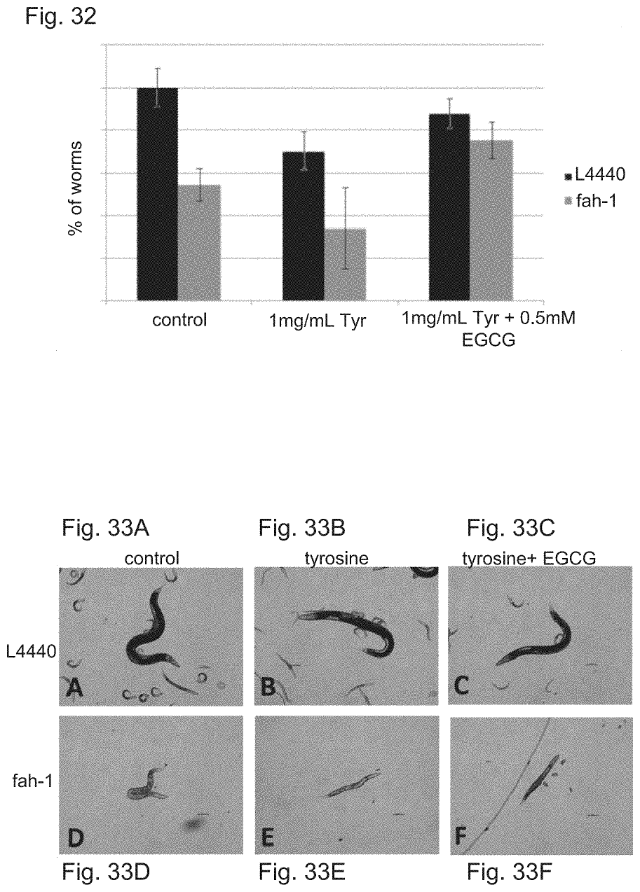

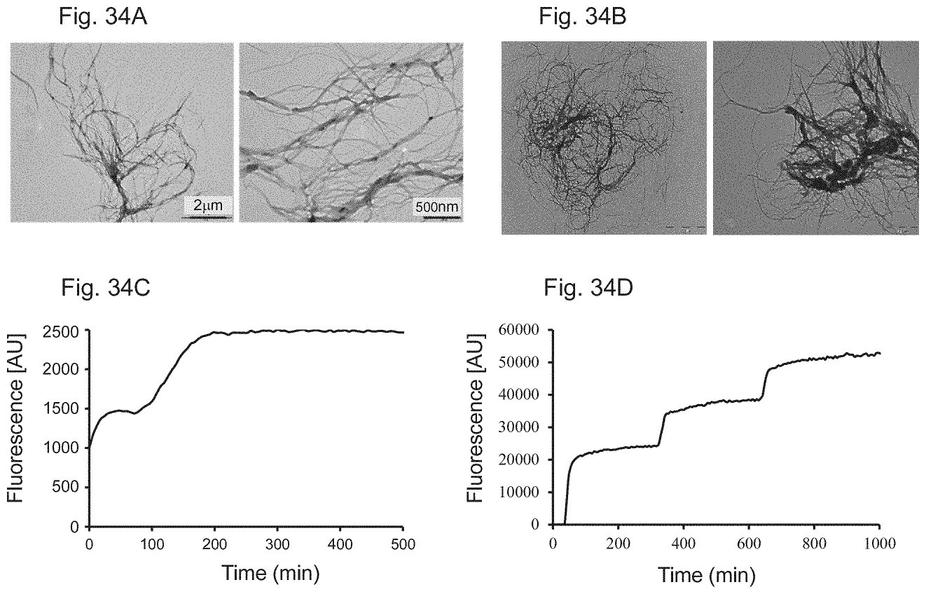

FIG. 27C: Growth curves of cys4Δ on SD media with or without homocysteine (Hcy) at different concentrations (20 mg/L, 40 mg/L and 80 mg/L).

FIG. 27D: Flow cytometry analysis of WT and cys4Δ strains containing 13 mg/L cysteine and 80 mg/L homocysteine under the indicated conditions using ProteoStat staining.

FIG. 27E: WT and cys4Δ strains diluted to OD600 0.01 were grown in SD media containing 13 mg/L cysteine and 80 mg/L homocysteine in the under the indicated conditions. 0.3 mM TA was added to the samples at different OD600. The percentage of growth represents the growth the indicated condition compared to the growth of WT without TA. *P<0.05 (Student's t-test).

FIG. 28A-28B: Sensitivity of gcv1Δ to glycine feeding and amyloid-like staining

FIG. 28A: Growth curves of WT and gcv1Δ cells in SD media with or without 5 mM glycine (Gly).

FIG. 28B: Flow cytometry analysis of WT and gcv1Δ cells under the indicated conditions using ProteoStat staining.

FIG. 29: Yeast model for Isoleucine accumulation and toxicity

Growth curves of WT and lat1Δ on minimal media with or without 150 mg/ml isoleucine. Cells were incubated at 30° C. for 40 h, with continuous shaking.

FIG. 30: Addition of Phenylalanine and Baicalein affects the total number of worms

The number of N2 (WT) worms increased when 100 mM phenylalanine was added to the minimal media, while the number of RB857 (phenylalanine hydroxylase mutant worms) decreased. Addition of Baicalein had a beneficial effect on both strains.

FIG. 31: Addition of Phenylalanine and Baicalein affects the development of worms Addition of 100 mM Phenylalanine to the minimal media of RB857 worms had a devastating effect of their development—none of the worms reached adulthood on day 4, while more WT worms reached adulthood, if compared to the control. Addition of Baicalein had a dramatic beneficial effect on both strains. In this experiment, more than 90% of RB857 worms grown of 100 mM Phenylalanine didn't reach adulthood by day 10.

FIG. 32: Addition of Tyrosine and EGCG affects the total number of worms

Inhibition of fumarylacetoacetate hydrolase (fah-1) by specific RNAi, results decrease in the total number of worms. Addition of tyrosine to the minimal media causes another decrease of the total number of worms, especially in the fah-1 inhibited group. Addition of EGCG caused elevation of the total number of worms.

FIG. 33A-33F: Addition of Tyrosine and EGCG affects the size and fertility of worms

FIG. 33A: Picture showing L440 worm treated with RNAi (control).

FIG. 33B: Picture showing L440 worm treated with RNAi and tyrosine. Addition of tyrosine did not affect the control RNAi treated worms

FIG. 33C: Picture showing L440 worm treated with RNAi and tyrosine+EGCG. Addition of tyrosine+EGCG did not affect the control RNAi treated worms.

FIG. 33D: Picture showing that fah-1 RNAi treated worms were smaller and have decreased fertility, while being able to produce viable offspring.

FIG. 33E: Picture showing that addition of tyrosine to the fah-1 RNAi treated worms causes severe decline in worm fertility.

FIG. 33F: Picture showing that addition of EGCG is able to enhance the fertility of worms.

FIG. 34A-34D. Formation of amyloid-like structures by adenosine self-assembly and guanosine self-assembly

FIG. 34A. TEM images of elongated adenosine fibrils (10 mg/mL). Scale bar is 2 μm and 500 nm.

FIG. 34B. TEM images of elongated guanosine fibrils (1 mg/mL). Scale bar is 2 μm and 500 nm.

FIG. 34C. ThT fluorescence assay of adenosine assemblies.

FIG. 34D. ThT fluorescence assay of guanosine assemblies.

### DETAILED DESCRIPTION OF THE INVENTION

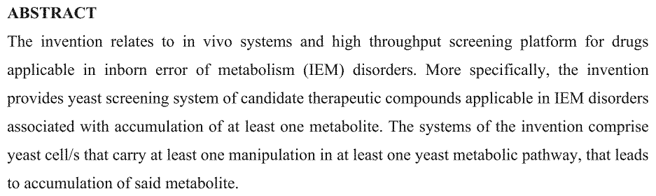

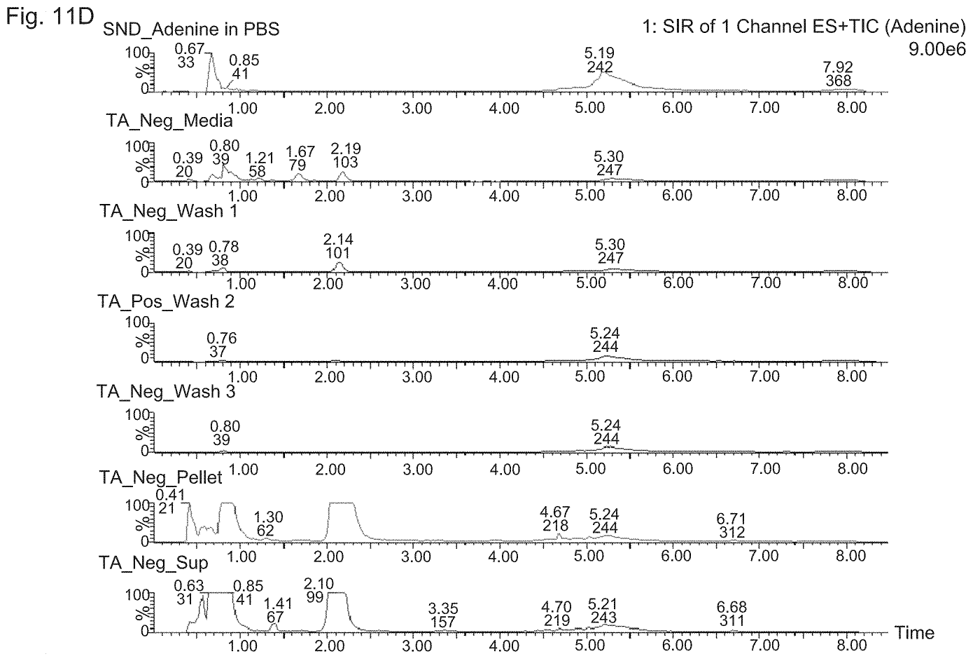

The ability of metabolites to form ordered amyloid-like assemblies in vitro represents a significant extension to the “amyloid hypothesis” and provides a new paradigm for the etiology of inborn error of metabolism disorders. The present invention offers the first in vivo demonstration and key experimental tools for the study of metabolite aggregation phenomena in living systems. One of the most intriguing results is the non-linear dose-dependency of the salvage mutant growth inhibition upon external addition of adenine (FIG. 1D), as well as external addition of tyrosine, phenylalanine, homocysteine and glycine (Example 10, 11, 12 and 13, respectively). This type of cooperative behavior is typical of the well-coordinated self-assembly processes of protein and peptide amyloids. Significant effort was invested in order to understand the organization of amyloids formed by the assembly of various disease-related protein building blocks including the deciphering of primary and secondary nucleation events and the resulting fibril growth. While these highly important studies were performed in vitro, the analysis of such processes in living cells has so far been hampered. The newly established model could provide an invaluable in vivo, yet simple, system to determine amyloid nucleation and assembly in living eukaryotic cells. In the present invention (FIG. 13C), adenine aggregation and the formation of adenine amyloid-like structures inside the cell were demonstrated, as well as the suppression by well-known amyloid inhibitors.

The stereotypical cell-to-cell cerebral spreading of neurodegenerative disorders, such as Alzheimer's disease, Parkinson's disease and amyotrophic lateral sclerosis (ALS) has recently been suggested to be propagated in a prion-like manner. Given the remarkable similarities between protein and metabolite amyloids, prion-like features of metabolite assemblies cannot be ruled out. In fact, such a behavior may provide insights into surprising phenomena, including the maternal error of metabolism disorders, in which the newborn has a normal metabolism but might still suffer from neurodevelopmental aberrations due to maternal effects. Yeast have been successfully used to study cell-to-cell spreading of “yeast prions”. The current system should allow, for the first time, to reveal any possible prion-like effects of nonproteinaceous aggregates. This extends not only the definition of amyloids, but also the definition of metabolite prions-like entities, or metions (metabolite infectious particle).

The yeast model could also serve as the perfect platform for high throughput screening of new therapeutic agents to target metabolite aggregation, as previously demonstrated for yeast models of protein amyloid self-assembly.

Yeast mutations, resulting in the accumulation of given metabolites, as observed in various human metabolic diseases, may be utilized in a simple yet robust way. The ability of TA to reverse the growth inhibition without an effect on metabolite concentration (FIG. 13B) provides a proof of concept for a large-range screen of potential drugs that could target the aggregation process, as well as possible nucleating seeds.

Finally, the current invention provides the first and clear indication that the assembly of metabolites into ordered amyloid-like structures, rather than merely their amount, mediates growth inhibition. Indeed, excessive concentrations of adenine caused a complete growth inhibition. which was rescued with pharmacological intervention. This further raises the question regarding the natural mechanisms that help unicellular and multicellular organisms to avoid aggregation upon temporary or chronic surge in the concentration of a given metabolite. Presumably, similar to the proteostasis machinery, there are cellular mechanisms aimed to avoid metabolite aggregation or clear preformed metabolite nuclei. Such a metabostasis mechanism appears to be crucial, as many of the essential components of life, including aromatic amino acids and purine nucleobases, appear to be highly aggregative. The use of these molecular components is essential to maintain functional biological systems, while safety mechanisms should be available to prevent unwanted association of these aggregation-prone entities.

The present invention provides as a proof of concept effective yeast models for Adenosine deaminase (ADA) deficiency, Tyrosinemia, Phenylketonuria, Homocystinuria and Glycine encephalopathy (non-ketotic hyperglycinemia) that relates to accumulation of glycine. The invention further provides validation models in C. elegance and in rodents to assess the therapeutic compounds identified by the screening methods and systems of the invention.

Thus, in a first aspect. the invention provides a yeast screening system of candidate therapeutic compounds. In more specific embodiments, the system of the invention comprises: (a) a yeast cell and/or yeast cell line and/or yeast cell population, and/or any progeny thereof, that carries at least one manipulation in at least one yeast metabolic pathway, that leads to accumulation of at least one metabolite. In certain embodiments, the metabolite is associated with at least one inborn error of metabolism (IEM) disorder. In some embodiments, such yeast cell and/or yeast cell line, and/or yeast cell population, and/or any progeny thereof carries or comprise or exposed to at least one manipulation in a native yeast pathway that may lead to accumulation of such metabolite. In some embodiments, the system of the invention may further optionally comprise (b), at least one reagent or means for determining at least one of, accumulation of the metabolite and at least one phenotype associated with accumulation of said metabolite. In some embodiments, the therapeutic compounds screened by the system disclosed herein may be useful for treating, preventing, ameliorating, reducing or delaying the onset of at least one inborn error of metabolism (IEM) disorder associated with accumulation of at least one metabolite.

As noted above, the first and essential element or component of the system of the invention is a yeast cell and/or cell line, specifically, a manipulated yeast cell and/or yeast cell line, and/or yeast cell population, and/or any progeny thereof, more specifically. manipulated cell/s, specifically, genetically and/or epigenetically manipulated cells.

Yeasts are eukaryotic, single-celled microorganisms classified as members of the fungus kingdom. Yeasts are unicellular organisms with some species having the ability to develop multicellular characteristics by forming strings of connected budding cells known as pseudohyphae or false hyphae. Yeast sizes vary greatly, depending on species and environment, typically measuring 3-4 μm in diameter, although some yeast strains can grow to 40 μm in size.

Yeasts do not form a single taxonomic or phylogenetic grouping. The term “yeast” is often taken as a synonym for Saccharomyces cerevisiae, but the phylogenetic diversity of yeasts is shown by their placement in two separate phyla: the Ascomycota and the Basidiomycota. The budding yeasts (“true yeasts”) are classified in the order Saccharomycetales. within the phylum Ascomycota.

Still further, Yeast, e.g., the baker's yeast Saccharomyces cerevisiae, has significant advantages as an experimental system. Yeast are straightforward to culture and maintain, have a short generation time, and are highly genetically tractable, meaning that they can be genetically modified, rapidly, predictably, and with high precision using well known and available techniques and reagents, and are amenable to high throughput chemical and genetic screens. Minimal genetic and epigenetic variation within strains contributes to screen reproducibility. Extensive genetic and protein interaction analysis in yeast means that considerable information regarding the yeast interactome, i.e., the set of physical interactions among molecules in a cell and interactions among genes, i.e., genetic interactions, in yeast cells is available. Molecular interactions can occur between molecules belonging to different biochemical families (proteins, nucleic acids, lipids, carbohydrates, etc.) and also within a given family (e.g., nucleotide-nucleotide interactions). While yeast cells lack the complexity of a multicellular organism the highly conserved genome and eukaryotic cellular machinery that they share with human cells affords the possibility of understanding basic cell-autonomous mechanisms and physical and genetic interactions underlying complex disease processes. There are several genus of yeast. More specifically, the yeast may be one that belongs to the genus Saccharomyces, the genus Zygosaccharomyces, the genus Pichia, the genus Kluyveromyces, the genus Candida, the genus Shizosaccharomyces, the genus Issachenkia, the genus Yarrowia, or the genus Hansenula.

The yeast belonging to the genus Saccharomyces may be, for example, S. cerevisiae, S. bayanus, S. boulardii, S. bulderi, S. cariocanus, S cariocus, S. chevaliers, S. dairenensis. S. ellipsoideus, S. eubayanus, S. exiguus. S. florentinus, S. kluyveri, S. martiniae, S. monacensis. S. norbensis. S. paradoxus, S. pastorianus, S. spencerorum, S. turicensis, S. unisporus, S. uvarum, or S. zonatus.

In some specific embodiments, the yeast cell and/or yeast cell line, and/or yeast cell population, and/or any progeny thereof, encompassed by the system of the invention may be Saccharomyces cerevisiae or any strain or isolate thereof. Saccharomyces cerevisiae also known as the Baker's yeast is a species of yeast. It is known to convert by fermentation carbohydrates to carbon dioxide and alcohols-used in baking and for alcoholic beverages. It is also a centrally important model organism in modem cell biology research, and is one of the most thoroughly researched eukaryotic microorganisms. S. cerevisiae cells are round to ovoid, 5-10 μm in diameter. It reproduces by a division process known as budding. Specific examples of Saccharomyces strains that may be used in the invention may include S. boulardii 17 (Sb) (ATCC® MYA796™), S. cerevisiae UFMG A-905 (905), S. cerevisiae Sc47 and S. cerevisiae LII (LII), S. cerevisiae BY4741 (ATCC® Number: 201388™), S. cerevisiae BY4743 (ATCC® 201390™), YPS128 strain, NCYC3290 strain, K11 strain, YB210 strain, CEN.PK strain (ATCC® MYA1108™), PE-2 strain, BG-1 strain (ATCC®, 204700™), and derivatives thereof.

In yet some further specific embodiments, the yeast cells applicable in the systems of the invention may be the BY4741 strain. BY4741 (GenBank: RIS00000000 or GSM1312317, ATCC® Number: 201388™), also referred to as ATCC 4040002, with the deposited Name: Saccharomyces cerevisiae Hansen, having the Genotype: MATa his3delta1 leu2delta0 met15delta0 ura3delta0, is part of a set of deletion strains derived from S288C in which commonly used selectable marker genes were deleted by design in order to minimize or eliminate homology to the corresponding marker genes in commonly used vectors without significantly affecting adjacent gene expression. The yeast strains were all directly descended from FY2, which is itself a direct descendant of S288C. Variation between BY4741 and S288C is miniscule. BY4741 was used as a parent strain for the international systematic Saccharomyces cerevisiae gene disruption project.

It should be understood that the invention provides yeast cell and/or yeast cell line, and/or yeast cell population, and/or any progeny thereof. More specifically. the invention provides yeast cell line that are in some embodiments, cell culture that is derived from one cell or set of cells of the same type (e.g., manipulated in the specific metabolic pathway) and in which under certain conditions the cells proliferate indefinitely. The invention further encompasses any cell population comprising the yeast cells of the invention, or any cell derived therefrom, where at least 10% or more, 25%, 30%, 40%, 50%, 60%, 70%, 80%, 90% or more and preferably. 100%, of the cells in the population are the modulated yeast cells of the invention. It is understood that such cell/s and progeny thereof refer not only to the particular subject cells but to the progeny or potential progeny of such a cell, specifically, any cell derived from such cell. Because certain modification may occur in succeeding generation due to either mutation or environmental influences, such progeny may not, in fact, be identical to the parent cell, but are still included within the scope of the term as used herein.

In yet some further embodiments, the yeast cells of the systems provided by the invention may be a manipulated yeast cell/s that carry at least one manipulation in at least one gene involved with at least one yeast metabolic pathway. In some embodiments, such manipulation is a genetic manipulation, for example, a mutation, deletion, insertion, rearrangement and the like. In some embodiments, these cells carry at least one mutation or any other manipulation in at least one yeast gene involved in the metabolic pathway, leading to accumulation of the metabolite associated with the IEM. It should be understood that the manipulation may be either stable specifically affecting the genome of the cells and passing in any cell divisions and passages, or transient. In transient manipulation it is meant that the modification involved are not stable and therefore are not maintained in the next generation. Such manipulation may be achieved for example by contacting the cells with a modulator that may be a genetic element, a polypeptide or any other modulator (e.g., repressor or activator) that targets or is directed at a nucleic acid sequence encoding a product that is directly or indirectly participating in the metabolic pathway involved with the metabolite accumulated in the IEM disorder. In yet some further embodiments, the yeasts cell/s, cell lines, yeast cell populations or any progeny thereof, may be alternatively or additionally manipulated by at least one epigenetic manipulation.

The yeast cells provided by the systems of the invention contain or carry at least one manipulation in at least one yeast metabolic pathway. Such manipulation leads to accumulation of the specific metabolite associated with the IEM. In some embodiments, the metabolic pathway is associated directly or indirectly with at least one of, synthesis, formation, stability, levels, activity and function of the metabolite. It should be understood that the manipulations in the metabolic pathway in the yeast cell/s, cell lines, yeast cell population, and/or any progeny thereof, may be genetic manipulations, epigenetic manipulations or any combinations thereof. In some embodiments, manipulation as indicated herein refers to genetic and/or epigenetic manipulation. More specifically. manipulation refers to causing at least one of mutation, alteration, abolishment and variation in at least one gene encoding at least one protein that participate in at least one metabolic pathway involved directly or indirectly with at least one of, synthesis, formation, stability, levels, activity and function of the metabolite. In some embodiments, said genetic and/or epigenetic manipulation is performed using gene editing systems. The specific gene encode a protein that participates at least in part of the metabolic pathway. Introduction of such genetic manipulation disturbs the normal function of said protein, thereby leading to accumulation of the specific metabolite. The invention relates to a yeast cell and/or cell line having at least one manipulation, for example, genetic and/or epigenetic manipulation or modification in at least one yeast metabolic pathway. A metabolic pathway, as used herein is a linked series of chemical reactions occurring within a cell. The reactants, products, and intermediates of an enzymatic reaction are known as metabolites, which are modified by a sequence of chemical reactions catalyzed by enzymes. In most cases of a metabolic pathway, the product of one enzyme acts as the substrate for the next. Different metabolic pathways function based on the location within a eukaryotic cell and the significance of the pathway in the given compartment of the cell. For example, the mitochondrial membrane or alternatively, the cytosol. There are two types of metabolic pathways that are characterized by their ability to either synthesize molecules with the utilization of energy (anabolic pathway) or break down of complex molecules by releasing energy in the process (catabolic pathway). In addition to the two distinct metabolic pathways is the amphibolic pathway, which can be either catabolic or anabolic based on the need for or the availability of energy. A manipulation in at least one metabolic pathway in accordance with the invention encompasses a genetic modification that modulates (enhance or inhibit the expression, stability and/or activity) any of the enzymes participating in the pathway, specifically at any stage of the pathway thereby leading to the accumulation of the specific metabolite that is associated with the IEM disorder. The modification according to the invention may be at any metabolic pathway, either catabolic. anabolic or amphibolic pathway as discussed herein.

As indicated herein, the manipulation in at least one metabolic pathway of the yeast leads to accumulation of the specific metabolite associated or linked with the IEM disorder. Accumulation as used herein refers to addition, increase, multiplication, conglomeration, growth by addition, gathering, collecting, agglomeration, accession, intensification, multiplication, enlargement, augmentation in the amount, mass, concentration and/or quantity of the metabolite in the cell over time. The accumulation of the metabolite as referred to herein encompass any increase in about 5%, 10%, 15%, 20%, 25%, 30%, 35%, 40%, 45%, 50%, 55%, 60%, 65%, 70%, 75%, 80%, 85%, 90%, 95%, 100%, as compared to natural concentration, amount or quantity of the metabolite in the cell, under conditions were the metabolic pathway is not manipulated. In some embodiments, the amount, concentration and/or quantity of the accumulated metabolite may be increased by 2, 3, 4, 5, 6, 7, 8, 9, 10, 50, 100, 1000, 10,000, folds or more as compared with the amount, concentration and/or quantity of the metabolite in native condition of non-manipulated pathway. The genetic manipulation discussed herein include any mutation, rearrangement, insertion, deletion, or substitution of one or more nucleotide/s in the coding and/or non-coding region/s of at least one gene that encodes at least one product involved directly or indirectly with at least one of, synthesis, formation, stability. levels. activity and function of the metabolite associated with the IEM disorder.

The term “mutation” as herein defined refers to a change in the nucleotide sequence of the genome of an organism. Mutations may or may not produce observable (phenotypic) changes in the characteristics of an organism. Mutation can result in several different types of change in the DNA sequence: these changes may have no effect, alter the product of a gene, or prevent the gene from functioning properly or completely. There are generally three types of mutations, namely single base substitutions. rearrangement, insertions and deletions and mutations defined as “chromosomal mutations”.

The term “single base substitutions” as herein defined refers to a single nucleotide base which is replaced by another. These single base changes are also called point mutations. There are two types of base substitutions, namely, “transition” and “transversion”. When a purine base (i.e. Adenosine or Thymine) replaces a purine base or a pyrimidine base (Cytosine. Guanine) replaces a pyrimidine base, the base substitution mutation is termed a “transition”. When a purine base replaces a pyrimidine base or vice-versa, the base substitution is called a “transversion”.

Single base substitutions may be further classified according to their effect on the genome, as follows: In missense mutations the new base alters a codon, resulting in a different amino acid being incorporated into the protein chain. In nonsense mutations the new base changes a codon that specified an amino acid into one of the stop codons (taa, tag, tga). This will cause translation of the mRNA to stop prematurely and a truncated protein to be produced. This truncated protein will be unlikely to function correctly.

In silent mutations no change in the final protein product occurs and thus the mutation can only be detected by sequencing the gene. Most amino acids that make up a protein are encoded by several different codons (see genetic code). So, if for example, the third base in the ‘cag’ codon is changed to an ‘a’ to give ‘caa’. a glutamine (Q) would still be incorporated into the protein product, because the mutated codon still codes for the same amino acid. These types of mutations are ‘silent’ and have no detrimental effect. Mutation may also arise from insertions of nucleic acids into the DNA or from duplication or deletions of nucleic acids therefrom. As herein defined, the term “insertions and deletions” refers to extra base pairs that are added or deleted from the DNA of a gene, respectively. The number of bases can range from a few to thousands. More specifically, 1 base or more, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 2000, 3000, 400), 5000, 6000, 7000, 8000, 9000, 10000 and more, 50,000 and more. It should be noted that deletion in the gene in accordance with the invention may be a partial or complete deletion of specific intron/s, exon/s or the entire gene or any homolog thereof, optionally, in both alleles. Insertions and deletions of one or two bases or multiples of one or two bases cause, inter alia, frame shift mutations (i.e. these mutations shift the reading frame of the gene). These can have devastating effects because the mRNA is translated in new groups of three nucleotides and the protein being produced may be useless. Insertions and deletions of three or multiples of three bases may be less substantial because they preserve the open reading frame. It should be understood that the yeast cell and/or yeast cell line and/or yeast cell population, and/or any progeny thereof in accordance with the invention may carry any one of the above mutations or genetic modifications or any combinations thereof, in one (in haploid and diploid yeasts) or in both alleles (in diploid yeasts). Still further, in some embodiments the yeast cell/s of the invention undergo alternatively or additionally at least one epigenetic manipulation in at least one nucleic acid sequence that encodes or regulates the expression of a product involved directly or indirectly in the metabolic pathway. Such epigenetic manipulation (e.g., methylation, gene repression) leads to accumulation of at least one metabolite associated with the IEM disorder. The term “epigenetic modification” refers to a change in genetic information that does not arise from a change in a nucleotide sequence (e.g., a DNA sequence). Typically. epigenetic modifications affect the expression or activity of a target chromatin site (e.g., the expression or activity of a gene), although an epigenetic modification can be any modification of genetic material that does not arise from a nucleotide sequence change but produces a change in a phenotype. Epigenetic modifications typically comprise modifications to a nucleic acid (e.g., DNA) or a protein (e.g., a histone). Such modifications typically comprise methylation, dimethylation, trim ethylation, demethylation, acetylation, deacetylation, citrullination, or a combination thereof. Epigenetic modifications can either decrease or increase the expression or activity of a target site (e.g., gene expression or activity). Epigenetic modifications, and the resulting effects (e.g., changes in gene expression or phenotype). can be either transient or persistent. It should be therefore understood that the manipulated yeast cells of the invention may comprise or exposed to any reagent or means for epigenetic manipulation or modification in at least one metabolic pathway that leads to accumulation of the specific metabolite associated with the IEM disorder. Such manipulation may be performed using any silencing means, for example, specific siRNAs, or other inhibitory nucleic acid molecules, or any gene editing systems that may lead to manipulation (e.g., the CRISPRi or CRISPRa systems) or any chimeras or fusion proteins thereof, for example, dCAS-methyltransferase directed (by specific gRNAs) at regulatory or non-regulatory sequences of a gene encoding a product participating directly or indirectly in a metabolic pathway associated with the metabolite.

It should be also appreciated that the genetic and/or epigenetic manipulation may be transient, stable and/or inducible.

In some specific embodiments, the manipulated yeast cell/s provided by the systems and methods of the invention may carry at least one manipulation that affects a yeast metabolic pathway. In more specific embodiments, such genetic and/or epigenetic manipulation results in reduced function of at least one gene product that participates in the specific target metabolic pathway. Therefore, in some embodiments, the genetic and/or epigenetic manipulation performed in the yeast cells of the systems and methods of the invention leads to loss of function. More specifically, “Loss of function” generally refers to reduction of function or absence of function as compared with a reference level. The reference level may be, e.g., a normal or average level of function possessed by a normal gene product or found in a healthy cell or subject. In certain embodiments the reference level may be the lower limit of a reference range. In certain embodiments the function may be reduced by at least 5%, 10%, 15%, 20%, 25%, 30%, 35%, 40%, 45%, 50%, 55%, 60%, 65%, 70%, 75%, 80%, 85%, 90%, 95%, 99%, or 100% of the reference level.

Still further, a “loss of function mutation” in a gene refers to a mutation that causes loss (reduction or absence) of at least one function normally provided by a gene product of the gene. A loss of function mutation in a gene, for example, a native yeast gene that encodes a product that participates in a native yeast metabolic pathway, may result in a reduced total level of a gene product of the gene in a cell that carry the mutation (e.g., due to reduced expression of the gene, reduced stability of the gene product, or both), reduced or altered activity or function per molecule of the gene product encoded by the mutant gene, or both. The reduction in expression, level, activity per molecule, or total function may be partial or complete. A mutation that confers a complete loss of function, or an allele harboring such a mutation. may be referred to as a null mutation or null allele, respectively. In some embodiments a loss of function mutation in a gene results in a reduction of at least 10%, 20%, 30%, 40%, 50%, 60%, 7(W %, 80%, 90%, 95%, 99%, or 100% in the level or activity of a gene product of the mutant gene, as compared with level or activity of a gene product encoded by a normal allele of the gene. A loss of function mutation may be an insertion, deletion, rearrangement or point mutation. For example, a point mutation may introduce a premature stop codon, resulting in a truncated version of the normal gene product that lacks at least a portion of a domain that contributes to or is essential for activity, such as a catalytic domain or binding domain, or may alter an amino acid that contributes to or is essential for activity, such as a catalytic residue, site of post-translational modification, etc. Alternatively, the genetic manipulation may occur in any coding or non-coding regulatory region that affects the stability, expression and splicing thereof. Still further, as indicated above, the loss of function manipulations may also involve epigenetic manipulations. It should be however noted that in some alternative embodiments, the manipulated yeast cells of the invention may be manipulated to express either endogenously (by enhancing transcription and/or translation or reducing repression of transcription and/or translation) or exogenously (e.g., using an exogenously added nucleic acid sequence, optionally provided in a vector such as a plasmid) metabolic pathway or any parts thereof, that leads to accumulation of the specific metabolite. Such manipulated cells may therefore carry a gain of function mutation or genetic and/or epigenetic manipulation. In more specific embodiments, a “gain of function mutation” in a gene refers to a mutation that causes gain (increase or presence) of at least one function normally not provided. A gain of function mutation in a gene, for example, a native yeast gene that encodes a product that participates in a native yeast metabolic pathway, may result in enhanced total level of a gene product of the gene in a cell that carry the mutation (e.g., due to enhanced expression of the gene, enhanced stability of the gene product, or both), enhanced activity per molecule of the gene product encoded by the mutant gene. or both. The increase in expression, level, activity per molecule, or total function may be partial or complete. Alternatively, the cells may express exogenously added gene that encodes the desired product that lead to accumulation of the metabolite. In some embodiments a gain of function mutation in at least one gene results in an increase of at least 5%, 10%, 20%, 30%, 40%, 50%, 60%, 70%, 80%, 90%, 95%, 99%, or 100% in the level or activity of a gene product of the mutant gene, as compared with level or activity of a gene product encoded by a normal allele of the gene. It should be understood that in some embodiments, the genetic and/or epigenetic manipulation of the yeast cell and/or yeast cell line, and/or yeast cell population, and/or any progeny thereof in accordance with the invention may be performed using any editing system for example, Transcription activator-like effector nucleases (TALEN), Zinc-finger nucleases (ZFNs), clustered regularly interspaced short palindromic repeats (CRISPR-Cas) system, or any fusion proteins thereof. In yet some alternative or additional embodiments, the CRISPR system may be used for epigenetic manipulations, for example using a mutated Cas (dCAS) protein devoid of nucleolytic activity, fused to an effector molecule such as methyl transferase that methylates the target sequences targeted by the gRNAs, or a dCAS protein fused to a repressor, specifically, CRISPR interference (CRISPRi) (e.g., dCAS-repressor) or alternatively, dCAS fused to activator, specifically, CRISPR activation (CRISPRa) (e.g., dCAS-activator). A non-limiting example for repressor useful in the invention as the effector/modifier component, may be the Krüppel associated box (KRAB) domain, which enhances repression of the targets. A non-limiting example for such activator may be the Herpes simplex virus protein vmw65, also known as VP16. As indicated above, in some alternative embodiments, the system of the invention may further comprise as a second element or component, at least one of reagents and/or means for detecting or determining at least one phenotype associated with the accumulation of the metabolite. “Means” as used herein refers to any cellular and non-cellular means, specifically, any natural or artificial cell or cell parts, organs, tissues or any equipment, facility, instrument, machine, program, required for determination, quantitation, recording, computing, visualizing, and evaluating the accumulation of the metabolite involved in the IEM disorder.

In yet some specific embodiments, the system/s provided by the invention may further comprise at least one validation means for the candidate therapeutic compound. Validation means in accordance with the invention is used for checking, verifying, proving the validity or effectivity, establishing documentary evidence demonstrating that the identified compound is suitable for therapy. In more specific embodiments such validation means may be at least one of: (a) at least one unicellular organism that display accumulation of the metabolite: (b) at least one multicellular eukaryotic organism that display accumulation of the metabolite; (c) at least one mammalian cell that display accumulation of the metabolite: and (d) at least one mammalian animal model that display accumulation of the metabolite.

In yet some further embodiments, the system of the invention may include as a means for evaluation, any unicellular organism, specifically, any eukaryotic or prokaryotic cell. In some embodiments, any eukaryotic cells, either of a unicellular or of a multicellular eukaryotic organism or prokaryotic cells (bacteria or archaea) may be used as an evaluation means by the systems of the invention. In some embodiments, eukaryotic cells and/or eukaryotic unicellular or multicellular organisms in accordance with the invention may include any eukaryotic cell or organism, for example, of any organism of the biological kingdom Animalia. In more specific embodiments, the eukaryotic cells of the invention may originate from a mammal, specifically, a human. In yet some further embodiments, such mammal may include any member of the mammalian nineteen orders, specifically, Order Artiodactyla (even-toed hoofed animals), Order Carnivora (meat-eaters), Order Cetacea (whales and purpoises), Order Chiroptera (bats), Order Dermoptera (colugos or flying lemurs), Order Edentata (toothless mammals), Order Hyracoidae (hyraxes, dassies). Order Insectivora (insect-eaters), Order Lagomorpha (pikas, hares, and rabbits), Order Marsupialia (pouched animals), Order Monotremata (egg-laying mammals), Order Perissodactyla (odd-toed hoofed animals), Order Pholidata, Order Pinnipedia (seals and walruses). Order Primates (pnmates). Order Proboscidea (elephants), Order Rodentia (gnawing mammals), Order Sirenia (dugongs and manatees), Order Tubulidentata (aardvarks). In some specific embodiment, such mammal may be at least one of a Cattle, domestic pig (swine, hog), sheep, horse, goat, alpaca, lama and Camels. In yet some further embodiments, the eukaryotic cells of the invention of particular relevance may originate from rodent since it represents the most popular and commonly accepted animal model in research.

In further specific embodiments, additional yeast cell lines may be used for evaluation. In some specific embodiments, Schizosaccharomyces pombe or Candida albicans may be used as a validation means by the systems of the invention.

Thus, in some embodiments. Schizosaccharomyces pombe may be used as a validation means by the systems and methods of the invention. Schizosaccharomyces pombe, also called “fission yeast”, is a species of yeast used in traditional brewing and as a model organism in molecular and cell biology. It is a unicellular eukaryote, whose cells are rod-shaped. Cells typically measure 3 to 4 micrometers in diameter and 7 to 14 micrometers in length. Its genome, which is approximately 14.1 million base pairs, is estimated to contain 4,970 protein-coding genes and at least 450 non-coding RNAs.

These cells maintain their shape by growing exclusively through the cell tips and divide by medial fission to produce two daughter cells of equal size, which makes them a powerful tool in cell cycle research.

In yet some further embodiments, Candida albicans may be used as a validation means by the systems and methods of the invention. Candida albicans is an opportunistic pathogenic yeast that is a common member of the human gut flora. It does not proliferate outside the human body. It is detected in the gastrointestinal tract and mouth in 40-60% of healthy adults. It is usually a commensal organism, but can become pathogenic in immunocompromised individuals under a variety of conditions. It is one of the few species of the genus Candida that causes the human infection candidiasis, which results from an overgrowth of the fungus. C. albicans is the most common fungal species isolated from biofilms either formed on (permanent) implanted medical devices or on human tissue.

It should be appreciated that such additional yeast may be used also by any of the methods of the invention as described herein after.

In yet some further specific embodiments, mammalian cells may be used as an evaluation means by the systems of the invention. More specifically, the mammalian cell may be any cell model, for example, primary cells, including lymphoblast and fibroblast, hematopoietic stem cells (HSCs), Neural stem cells, Mesenchymal stem cells, Muscle stem cells, T cells, embryonic stem cell (ESC)-derived and Induced Pluripotent Stem (iPS) cells derived from a patient suffering from said IEM disorder and cell lines. Specifically, embryonic stem cells, or human embryonic stem cells (hESCs), that were obtained from self-umbilical cord blood just after birth. Embryonic stem cells are pluripotent stem cells derived from the early embryo that are characterized by the ability to proliferate over prolonged periods of culture while remaining undifferentiated and maintaining a stable karyotype, with the potential to differentiate into derivatives of all three germ layers. hESCs may be also derived from the inner cell mass (iCM) of the blastocyst stage (100-200 cells) of embryos generated by in vitro fertilization. However, methods have been developed to derive hESCs from the late morula stage (30-40 cells) and, recently, from arrested embryos (16-24 cells incapable of further development) and single blastomeres isolated from 8-cell embryos.

In further embodiments, the eukaryotic cells according to the invention are totipotent stem cells. Totipotent stem cells are versatile stem cells, and have the potential to give rise to any and all human cells, such as brain, liver, blood or heart cells or to an entire functional organism (e.g. the cell resulting from a fertilized egg). The first few cell divisions in embryonic development produce more totipotent cells. After four days of embryonic cell division, the cells begin to specialize into pluripotent stem cells. Embryonic stem cells may also be referred to as totipotent stem cells.

In further embodiments, the eukaryotic cells according to the invention are pluripotent stem cells. Similar to totipotent stem cells, a pluripotent stem cell refers to a stem cell that has the potential to differentiate into any of the three germ layers: endoderm (interior stomach lining, gastrointestinal tract, the lungs), mesoderm (muscle, bone, blood, urogenital), or ectoderm (epidermal tissues and nervous system). Pluripotent stem cells can give rise to any fetal or adult cell type. However, unlike totipotent stem cells, they cannot give rise to an entire organism. On the fourth day of development, the embryo forms into two layers, an outer layer which will become the placenta, and an inner mass which will form the tissues of the developing human body. These inner cells are referred to as pluripotent cells.

In still further embodiments, the eukaryotic cells that may be applicable for the methods according to the invention, are multipotent progenitor cells. Multipotent progenitor cells have the potential to give rise to a limited number of lineages. As a non-limiting example, a multipotent progenitor stem cell may be a hematopoietic cell, which is a blood stem cell that can develop into several types of blood cells, but cannot into other types of cells. Another example is the mesenchymal stem cell, which can differentiate into osteoblasts, chondrocytes, and adipocytes. Multipotent progenitor cells may be obtained by any method known to a person skilled in the art. More specifically, hematopoietic stem cells (HSCs) originate from the bone marrow. They first differentiate into multipotent progenitor (MPP) cells, and then may differentiate to common lymphoid progenitor (CLP) cells. Still further, in some embodiments, cells suitable in the present application may be immobilized HSC. Hematopoietic stem cells (HSCs) normally reside in the bone marrow but can be forced into the blood, a process termed bone marrow mobilization used to harvest large numbers of HSCs in peripheral blood. One mobilizing agent of choice in accordance with the invention may be granulocyte colony-stimulating factor (G-CSF). The resulting HSCs are further referred as mobilized HSCs.

In some embodiments, pluripotent cells may be used as an evaluating means by the systems and methods of the invention. It should be appreciated that in some embodiments, the target cells may be pluripotent cells, specifically, hematopoietic pluripotent cells. The term “pluripotent” refers to cells with the ability to give rise to progeny that can undergo differentiation, under the appropriate conditions, into cell types that collectively demonstrate characteristics associated with different cell lineages. In yet some further embodiments, such pluripotent cells may be induced pluripotent stem cell. As used herein, an induced pluripotent stem (iPS) cell is a cell that is derived from a somatic cell by reprogramming the cell to a pluripotent state. iPS cells possess certain key features of ES cells including cell morphology, colony morphology, long-term self-renewal. expression of pluripotency-associated markers, similar genome-wide expression profile, ability to form teratomas in immunocompromised mice, and ability to give rise to cells of multiple cell lineages in vitro under appropriate conditions. It will be understood that the term “iPS cell” includes the original derived pluripotent cell and its descendants that retain pluripotent stem cell properties. “Reprogramming”, as used herein, refers to altering the differentiation state or identity of a cell (e.g., its cell type) or a process by which this occurs. In general, reprogramming a first cell generates a second cell with a differentiation state or identity distinct from that which would result from a differentiation program that the first cell or a corresponding cell would normally follow in vivo and results in cells of one or more types distinct from those that the first cell or a corresponding cell would give rise to in vivo. A “corresponding cell” is a cell of the same type or having sufficiently similar features that a person of ordinary skill in the art would reasonably consider it to be of the same or substantially the same cell type. Lymphoblast, immature white blood cell that gives rise to a type of immune cell known as a lymphocyte. Lymphoblast is defined as an enlarged (intermediate or large) lymphocyte that has been activated to divide. It is recognized morphologically by an immature nucleus having fine granular chromatin and often one or more prominent nucleoli. Lymphoblastoid cell lines (LCLs), transformed by Epstein-Barr virus (EBV), are widely used, and are therefore encompassed by the present invention.

In some specific embodiments cells applicable for use as an evaluating means by the systems and methods of the invention may be lymphoblast cells.

In some embodiments, LCL cells obtained from a patient suffering from ADA deficiency, may be used as a validation means. In yet some further embodiments, LCL cells obtained from a patient suffering from Tyrosinemia, may be used as a validation means. In some embodiments, LCL cells obtained from a patient suffering from Phenylketonuria, may be used as a validation means. In some further embodiments, LCL cells obtained from a patient suffering from Homocystinuria, may be used as a validation means. In yet some embodiments, LCL cells obtained from a patient suffering from Glycine encephalopathy may be used as a validation means. In some embodiments, LCL cells obtained from a patient suffering from MSUD, may be used as a validation means.

In yet some further embodiments. multicellular organisms may be used as a validation means by the systems of the invention. It should be understood that the animal models may include any multicellular organism model for any IEM disorder, specifically, and one of ADA deficiency, Tyrosinemia, Phenylketonuria, Homocystinuria, Glycine encephalopathy, MSUD, or any of the IEM disorders disclosed by the invention, for example, by Table 1. In some embodiments, the models may be based on multicellular organisms that were genetically and/or epigenetically modified. Such modification may be performed using any gene editing system [e.g., TALEN, ZFNs, CRISPR-Cas, or any fusion proteins thereof, for example CRISPRi (e.g., dCAS-repressor, or dCas-methyl transferase) or alternatively, CRISPRa (e.g., dCAS-activator)] or alternatively, by any know gene silencing means (e.g., siRNA, anti-sense nucleic acids, miRNA and the like).

In more specific embodiments, multicellular eukaryotic organism useful as an evaluation means in the systems and methods of the invention may be Nematodes models (Caenorhabditis elegans using any gene silencing means, for example, RNAi. mutations or any gene editing system, for example, CRISPR-Cas and the like). Example 14 demonstrates the use of C. elegans as an evaluation means for IEM disorders, for example, Tyrosinemia and Phenylketonuria. In some embodiments, the knock down of the gene encoding a product participating directly or indirectly in the metabolic pathway of tyrosine was achieved using siRNA directed at the fah-1 gene. This silencing resulted in C. elegans having impaired metabolic pathway that leads to accumulation of tyrosine. In some further embodiments, the system of the invention may comprise at least one genetically and/or epigenetic manipulated C. elegans that carries a modification in fumarylacetoacetate hydrolase (FAH-1) gene. More specifically. the fumarylacetoacetate hydrolase (FAH-1) gene as used herein is the fumarylacetoacetate hydrolase (FAH-1) gene in Caenorhabditis elegans (for example strain RB857) having the accession number NM_076682.4 encodes for the enzyme Fumarylacetoacetase or Fumarylacetoacetate hydrolase (having the accession number NP_509083.1). In some embodiments, the gene FAH-1 comprises the nucleic acid sequence as denoted by SEQ ID NO: 13. In some embodiments, the gene FAH-1 encodes for a protein comprising the amino acid sequence as denoted by SEQ ID NO: 14.

In some further embodiments, the system of the invention may comprise at least one genetically manipulated C. elegans that carries a modification in phenylalanine hydroxylase (PAH-1) gene, a homologous for the gene in humans that leads to PKU. In some embodiments, the gene PAH-1 comprises the nucleic acid sequence as denoted by SEQ ID NO: 19. In some embodiments, the gene PAH-1 encodes a protein comprising the amino acid sequence as denoted by SEQ ID NO: 20. Other C. eleganas that are being used by the present invention as validation means include Adenosine deaminase (ADA) deficiency that carries a modification in Adenosine deaminase that leads to accumulation of adenine. In some embodiments, the gene Adenosine deaminase comprises the nucleic acid sequence as denoted by SEQ ID NO: 21. In some embodiments, the gene Adenosine deaminase encodes a protein comprising the amino acid sequence as denoted by SEQ ID NO: 22. In yet some further C. eleganas models that are being used by the present invention as validation means include the C. eleganas Homocystinuria model that carries a modification in the cystathionine β-synthases gene, the C. eleganas Glycine encephalopathy (non-ketotic hyperglycinemia) model that carries a modification in Aminomethyltransferase gene, and the C. eleganas Maple syrup urine disease (MSUD) model that carries a modification in the Lipoamide acyltransferase gene or in any gene/s encoding any other component/s of the BCKDH complex. Thus, in some embodiments, the multicellular organism C. elegans may be used by the systems and methods of the invention as a means for evaluation. More specifically, Caenorhabditis elegans is a free-living, transparent nematode, about 1 mm in length, that lives in temperate soil environments. It is the type species of its genus. It was previously named Rhabditides elegans, and has been placed it in the genus Caenorhabditis, C. elegans is an unsegmented pseudocoelomate and lacks respiratory or circulatory systems. C. elegans is unsegmented, vermiform, and bilaterally symmetrical. It has a cuticle (a tough outer covering, as an exoskeleton), four main epidermal cords, and a fluid-filled pseudocoelom (body cavity). It also has some of the same organ systems as larger animals. About one in a thousand individuals is male and the rest are hermaphrodites. The basic anatomy of C. elegans includes a mouth, pharynx, intestine, gonad, and collagenous cuticle. Like all nematodes, they have neither a circulatory nor a respiratory system.

In yet some further embodiments, Drosophila melanogaster may be used as the multicellular eukaryotic organism applicable as a validation means by the systems and methods of the invention. Drosophila melanogaster is a species of fly (the taxonomic order Diptera) in the family Drosophilidae. The species is known generally as the common fruit fly (though inaccurately) or vinegar fly. The D. melanogaster is a specie widely used as a model organism, for biological research in genetics, physiology, microbial pathogenesis, and life history evolution. Drosophila is typically used in research because it can be readily reared in the laboratory, has only four pairs of chromosomes, breeds quickly, and lays many eggs.

It should be understood that the invention encompasses the use of any in vivo animal model of any eukaryotic organism, specifically, any mammalian organism, and more specifically any rodent model. Rodents are mammals of the order Rodentia, which are characterized by a single pair of continuously growing incisors in each of the upper and lower jaws. Rodents are the largest group of mammals. Non-limiting examples for such rodents that are applicable in the present invention. appear in the following list of rodents, arranged alphabetically by suborder and family. Suborder Anomaluromorpha includes the anomalure family (Anomaluridae) [anomalure (genera Anomalurus, Idiurus, and Zenkerella)], the spring hare family (Pedetidae) [spring hare (Pedetes capensis)]. The suborder Castorimorpha includes the beaver family (Castoridae) [beaver (genus Castor), giant beaver (genus Castoroides; extinct)], the kangaroo mice and rats (family Heteromyidae) [kangaroo mouse (genus Microdipodops), kangaroo rat (genus Dipodomys), pocket mouse (several genera)], the pocket gopher family (Geomyidae) [pocket gopher (multiple genera)]. Suborder Hystricomorpha, includes the agouti family (Dasy proctidae), acouchy (genus Myoprocta) [agouti (genus Dasyprocta)], the American spiny rat family (Echimyidae), the American spiny rat (multiple genera), the blesmol family (Bathyergidae) [blesmol (multiple genera)], the cane rat family (Thryonomyidae) [cane rat (genus Thryonomys)], the cavy family (Caviidae) [capybara (Hydrochoerus hydrochaeris), guinea pig (Cavia porcellus) mara (genus Dolichotis)], the chinchilla family (Chinchillidae) [chinchilla (genus Chinchilla), viscacha (genera Lagidium and Lagostomus)], the chinchilla rat family (Abrocomidae) [chinchilla rat (genera Cuscomys and Abrocoma)], the dassie rat family (Petromuridae) [dassie rat (Petromus typicus)], the degu family (Octodontidae) [degu (genus Octodon)], the diatomyid family (Diatomyidae), the giant hutia family (Heptaxodontidae), the gundi family (Ctenodactylidae) [gundi (multiple genera)], the hutia family (Capromyidae) [hutia (multiple genera)], the New World porcupine family (Erethizontidae) [New World porcupine (multiple genera)], the nutria family (Myocastoridae) [nutria (Myocastor coypus)], the Old World porcupine family (Hystricidae) [Old World porcupine (genera Atherurus, Hystrix, and Trichys)], the paca family (Cuniculidae) [paca (genus Cuniculus)], the pacarana family (Dinomyidae) [pacarana (Dinomys branickii)], the tuco-tuco family (Ctenomyidae) [tuco-tuco (genus Ctenomys)]. The suborder Myomorpha that includes the cricetid family (Cricetidae) [American harvest mouse (genus Reithrodontomys), cotton rat (genus Sigmodon), deer mouse (genus Peromyscus), grasshopper mouse (genus Onychomys), hamster (various genera), golden hamster (Mesocricetus auratus), lemming (various genera) maned rat (Lophiomys imhausi), muskrat (genera Neofiber and Ondatra), rice rat (genus Oryzomys), vole (various genera), meadow vole (genus Microtus), woodland vole (Microtus pinetorum), water rat (various genera), woodrat (genus Neotoma), dipodid family (Dipodidae), birch mouse (genus Sicista), jerboa (various genera), jumping mouse (genera Eozapus, Napaeozapus, and Zapus)], the mouselike hamster family (Calomyscidae), the murid family (Muridae) [African spiny mouse (genus Acomys), bandicoot rat (genera Bandicota and Nesokia), cloud rat (genera Phloeomys and Crateromys), gerbil (multiple genera), sand rat (genus Psammomys), mouse (genus Mus), house mouse (Mus musculus), Old World harvest mouse (genus Micromys), Old World rat (genus Rattus), shrew rat (various genera), water rat (genera Hydromys, Crossomys, and Colomys), wood mouse (genus Apodemus)], thenesomyid family (Nesomyidae), African pouched rat (genera Beamys, Cricetomys, and Saccostomus)], the Oriental dormouse family (Platacanthomyidae)[Asian tree mouse (genera Platacanthomys and Typhlomys)], the spalacid family (Spalacidae)[bamboo rat (genera Rhizomys and Cannomys), blind mole rat (genera Nannospalax and Spalax), zokor (genus Myospalax), suborder Sciuromorpha], the dormouse family (Gliridae) [dormouse (various genera), desert dormouse (Selevinia betpakdalaensis)], the mountain beaver family (Aplodontiidae) [mountain beaver (Aplodontia rufa)], the squirrel family (Sciuridae) [chipmunk (genus Tamias), flying squirrel (multiple genera), ground squirrel (multiple genera), suslik (genus Spermophilus), marmot (genus Marmota), groundhog (Marmota monax), prairie dog (genus Cynomys), tree squirrel (multiple genera)].

In yet some further embodiments, the rodent model of the invention may be a mouse model. A mouse, plural mice, is a small rodent characteristically having a pointed snout, small rounded ears, a body-length scaly tail and a high breeding rate. The best known mouse species is the common house mouse (Mus musculus). Species of mice are mostly found in Rodentia, and are present throughout the order. Typical mice are found in the genus Mus.

In some specific embodiments any murine models of IEM may be used as an evaluation means by the methods and systems of the invention. In more specific embodiments, such murine models may be prepared using gene editing tools, for example, site-specific endonucleases, such as zinc-finger nucleases and the CRISPR/Cas system, in combination with delivery vectors engineered to target disease tissue. In yet some further embodiments, mammalian animal model may be any murine model for each specific IEM disorder. Non-limiting examples for such models include a model for Ada^{-/-} mouse model having a mutation in Adenosine deaminase, the Fah^{-/-} mouse model having a knock-out of the fumarylacetoacetate hydrolase (FAH), the last enzyme in the tyrosine catabolic pathway, for tyrosinemia type I (HT-I), the spf^{ash} OTC mouse model Ornithine transcarbamylase (OTC) deficiency, for Phenylketonuria the Pah^{-/-} mouse model having a mutation in Phenylalanine

hydroxylase, for Argininemia the Arg^{-/-} mouse model having a mutation in Arginase-1, for Asparagine synthetase deficiency the Asns^{-/-} mouse model having a mutation in Asparagine synthetase, for Homocystinuria the CBS^{-/-} mouse model having a mutation in Cystathionine Beta-Synthase. for Cystinosis the Ctns^{-/-} mouse model having a mutation in Cystinosin, for Glycine encephalopathy the Gldc^{-/-} mouse model having a mutation in Glycine decarboxylaseand the like. As indicated herein above, the systems of the invention may comprise in some embodiments, means for detecting and determining the accumulation of the metabolite, as well as any phenotype associated with such accumulation. In some further embodiments, phenotype associated with accumulation of the metabolite as determined by the system of the invention may be at least one of cell toxicity and formation of metabolite aggregates.

A phenotype, as used herein, is the composite of the observable characteristics or traits, of a cell that display accumulation of the specific metabolite. It includes morphological or physical and structural properties, as well as biochemical and physiological properties. As used herein, “phenotypes associated with accumulation of a specific metabolite” may be in some embodiments, cellular phenotypes that are associated with the IEM disease, that are detectable in cells of a subject suffering from said IEM disorder. A cellular phenotype may be any detectable characteristic or property of a cell. In the context of the present disclosure, a cellular “phenotype” associated with an IEM disease may be any detectable deviation from a characteristic or property displayed by a cell that distinguishes the cell from a normal cell or cell derived from a subject who does not have the TEM disease and is not at increased risk of developing the IEM disease relative to the general population.

Thus, in some embodiments, a phenotype associated with accumulation of a specific metabolite may be cell toxicity. More specifically, toxicity or cell toxicity as used herein may be reflected by viability of the cells, shape, cell growth, cell function and cell death.

In yet some further embodiments, cell toxicity, may be reflected by induction of any one of oxidative stressors, nitrosative stressors, proteasome inhibitors, inhibitors of mitochondrial function, ionophores, inhibitors of vacuolar ATPases, inducers of endoplasmic reticulum (ER) stress, and inhibitors of endoplasmic reticulum associated degradation (ERAD). Thus, according to some embodiments, the system of the invention may further comprise at least one reagent and/or means for measuring and/or detecting cell toxicity.

In some specific embodiments, toxicity, or cell toxicity, may be determined by the systems and/or methods of the invention by any means for quantification or measuring at least one of cell viability, cell proliferation, cell apoptosis, and any toxic phenotype on the organism or cell.

Thus, according to some embodiments, the system of the invention may further comprise at least one reagent and/or means for measuring at least one of cell viability, cell proliferation and cell apoptosis.

In some specific embodiments, cell viability may be determined by 2,3-bis-(2-methhoxy-4-nitro-5-sulphophenyl)-2H-tetrazolium-5-carboxanilide (XTT) viability assay (not useful for cysteine accumulation), Methylene Blue, PrestoBlue viability reagent, the fluorescent intercalator 7-aminoactinomycin D (7-AAD), LIVE/DEAD Viability Kits, cell growth by turbidity, for example at OD600, or by any means for cell counting. Thus, in some embodiments, the systems of the invention may comprise at least one reagent required for performing any cell viability and proliferation assay, specifically, any of the assays disclosed above.

In yet some further embodiments, toxicity may be evaluated by measuring apoptosis of the cells. In some embodiments, apoptosis may be determined by at least one of DNA fragmentation (TUNNEL, terminal deoxynucleotidyl transferase dUTP nick end labeling), caspase and/or PARP1 phosphorylation, annexin V and propidium iodide (PI) assay. Thus, in some embodiments, the systems of the invention may comprise at least one reagent required for performing any apoptosis or any other cell death assay, specifically, any of the assays disclosed above.

It should be noted that in some embodiments, other toxic phenotype that may be determined on the organism/cell may include, activation of cell stress pathways (e.g., heat shock response), poor fertility, destruction of organs and tissue damages, DNA mutagenesis, ER stress, cell energy status and ATP content, oxidative stress, mitochondrial dysfunction, mitochondrial damage. activation of autophagy and activation of necrosis.

Thus, in some embodiments, the systems of the invention may comprise at least one reagent required for performing any of the cell toxicity assay disclosed above.

In yet some further embodiments, a phenotype associated with the accumulation of a specific metabolite may be the formation of metabolite aggregates. The term “metabolite aggregates” as used herein relates to accumulation of the specific metabolite in the cell in a specific fibrillar structures. More specifically, as used herein, Metabolite aggregates or metabolite structures refer to well-ordered assembly or elongated nanoscale fibrillary structures of non-protein entities such as metabolites. These supramolecular fibrillar structures are formed via self-association of entities that accumulate in a cell. The formation of these fibrils, may resemble to amyloid-like fibrils and is typically described by a nucleation-dependent polymerization mechanism, which comprises nucleation and elongation, and is often considered as a kind of crystallization. The time period showing the mass increment of metabolite fibrils is referred to as the elongation phase, and the induction period before the elongation is called the lag phase. It should be noted that in some embodiments, metabolite aggregates may be also referred to herein as amyloid-like structures. Thus, according to some embodiments, the system of the invention may further comprise at least one reagent and/or means for measuring formation and existence of metabolite aggregates. It should be however noted that in some embodiments, the system of the invention may comprise means and/or reagents for measuring and evaluating the level of the specific metabolite in the cell, specifically, reagents and means for detecting and quantifying metabolite accumulation in the cell. In yet some further embodiments, accumulation of the metabolite and/or formation of metabolite aggregates may be determined by the system of the invention using at least one of metabolic profiling, microscopy, light diffraction, absorption or scattering assay, spectrometric assay, immunological assay, Nuclear magnetic resonance (NMR), Liquid Chromatography, flow cytometry, and stereoscopy.

In yet some further embodiments, metabolite aggregation may be measured using at least one of Dye-binding specificity (for example, using thioflavin T (ThT) and congo red, or staining with Proteostat) microscopy, X-ray fiber diffraction, X-ray crystallography, X-ray powder diffraction.

X-ray single crystal diffraction, mass spectrometry (including Ion-mobility spectrometry, mass spectrometry (IMS-MS)), immunological assay (e.g. using a specific antibody that specifically recognize fibrillary assemblies), flow cytometry, circular dichroism (CD) spectrometry, vibrational CD, Raman Spectroscopy, density functional theory (DFT) quantum mechanics methods, Fourier-transformed infrared spectroscopy dynamic light scattering (DLS), liquid chromatography and NMR. Microscopy, such as TEM (transmission electron microscope). confocal fluorescence microscopy, confocal Raman microscopy, indirect immunofluorescence. It should be understood that the systems and methods of the invention further encompass any reagent or means, for example, cellular or non-cellular, natural or artificial means, specifically, any equipment, machine, instrument, database, computer program, for performing any of the procedures and processes indicated above for detecting, determining measuring and/or visualizing the formation of metabolite aggregates or metabolite accumulation. It should be appreciated that any means involved in the detection methods disclosed herein may be comprised within the system of the invention and used by any of the methods of the invention as a means for determining the accumulation and/or aggregation of the metabolite.

More specifically, Transmission electron microscopy (TEM, also sometimes conventional transmission electron microscopy or CTEM) is a microscopy technique in which a beam of electrons is transmitted through a specimen to form an image. The specimen is most often an ultrathin section less than 100 nm thick or a suspension on a grid. An image is formed from the interaction of the electrons with the sample as the beam is transmitted through the specimen. The image is then magnified and focused onto an imaging device, such as a fluorescent screen, a layer of photographic film, or a sensor such as a charge-coupled device. Transmission electron microscopes are capable of imaging at a significantly higher resolution than light microscopes, owing to the smaller de Broglie wavelength of electrons. This enables the instrument to capture fine detail, as small as a single column of atoms, which is thousands of times smaller than a resolvable object seen in a light microscope.

A scanning electron microscope (SEM) is a type of electron microscope that produces images of a sample by scanning the surface with a focused beam of electrons. The electrons interact with atoms in the sample, producing various signals that contain information about the surface topography and composition of the sample. The electron beam is scanned in a raster scan pattern, and the position of the beam is combined with the detected signal to produce an image. SEM can achieve resolution better than 1 nanometer.

Still further, Environmental SEM (ESEM) allowed samples to be observed in low-pressure gaseous environments (e.g. 1-50 Torr or 0.1-6.7 kPa) and high relative humidity (up to 100%). This was made possible by the development of a secondary-electron detector capable of operating in the presence of water vapor and by the use of pressure-limiting apertures with differential pumping in the path of the electron beam to separate the vacuum region (around the gun and lenses) from the sample chamber. ESEM is especially useful for non-metallic and biological materials because coating with carbon or gold is unnecessary.

X-ray crystallography is a technique used for determining the atomic and molecular structure of a crystal, in which the crystalline structure causes a beam of incident X-rays to diffract into many specific directions. By measuring the angles and intensities of these diffracted beams, a crystallographer can produce a three-dimensional picture of the density of electrons within the crystal. From this electron density, the mean positions of the atoms in the crystal can be determined, as well as their chemical bonds, their crystallographic disorder, and various other information.

Circular dichroism (CD) is dichroism involving circularly polarized light, i.e., the differential absorption of left- and right-handed light. Left-hand circular (LHC) and right-hand circular (RHC) polarized light represent two possible spin angular momentum states for a photon, and so circular dichroism is also referred to as dichroism for spin angular momentum. It is exhibited in the absorption bands of optically active chiral molecules. CD spectroscopy has a wide range of applications in many different fields. Most notably, UV CD is used to investigate the secondary structure of proteins.

Density functional theory (DFT) is a computational quantum mechanical modelling method used in physics, chemistry and materials science to investigate the electronic structure (principally the ground state) of many-body systems, in particular atoms, molecules, and the condensed phases. Using this theory, the properties of a many-electron system can be determined by using functionals, i.e. functions of another function. which in this case is the spatially dependent electron density. DFT is among the most popular and versatile methods available in condensed-matter physics, computational physics, and computational chemistry.

Dynamic light scattering (DLS) is a technique used to determine the size distribution profile of small particles in suspension or polymers in solution. In the scope of DLS, temporal fluctuations are usually analyzed by means of the intensity or photon auto-correlation function (also known as photon correlation spectroscopy or quasi-elastic light scattering). In the time domain analysis, the autocorrelation function (ACF) usually decays starting from zero delay time, and faster dynamics due to smaller particles lead to faster decorrelation of scattered intensity trace.

Ion-mobility spectrometry-mass spectrometry (IMS-MS), also known as ion-mobility separation-mass spectrometry, is an analytical chemistry method that separates gas phase ions on a millisecond timescale using ion-mobility spectrometry and uses mass spectrometry on a microsecond timescale to identify components in a sample. It should be noted that this method may be used for evaluating and measuring the levels of the metabolite and thereby for determining metabolite accumulation.

Metabolic Profiling. Metabolic profiling is a study of chemical processes that are associated to and involve metabolites. It is a study of chemical fingerprints that are very unique and that any specific physiological processes in a cell always leave behind. Metabolic profiling can also be defined as the use of analytical methods in measurement and interpretation of various endogenous low molecular weight and intermediates from their samples. This study makes use of metabolome and it provides a critical view of the physiological characteristic of a cell, tissue or the whole organism as compared to proteomic analysis and mRNA analysis. Metabonomics and metabolomics are other terms used in description of this study.

It should be understood that in some embodiments, the effect of the examined candidate on metabolite accumulation may be determined by additional parameters served herein as phenotype, specifically, when the candidate is evaluated using mammalian cells and specifically where a multicellular organism or a mammalian animal are used by the systems and methods of the invention as evaluation means. In yet some specific embodiments, such measured parameters may include morphology. motility. fertility, lethality, development, maturation, puberty, or any other behavioral or physiological parameters or phenotypes specifically associated with the particular IEM disease. It should be understood that any method and means described herein in connection with the systems of the invention may be also applicable for any of the methods of the invention and for any aspect disclosed herein.

As indicated above, the system of the invention may be suitable for screening of candidates for treating disorders associated with accumulation of at least one metabolite. Metabolite, as used herein, is an organic compound which is an intermediate end product of metabolism or a metabolic process or pathway. A primary metabolite is directly involved in normal growth, development. and reproduction such as amino acid, nucleotide, carboxylic acid, alcohols, antioxidants, or vitamins. A secondary metabolite is not directly involved in those processes, but usually has an important ecological function such as pigments or antibiotics. In some specific embodiments, such metabolite may be any one of a nucleobase, an amino acid residue, carbohydrate, fatty acid and ketone, sterols, porphyrin and haem. lipid and lipoprotein, neurotransmitters, vitamins and (non-protein) cofactors, trace elements, metals, metabolites associated with energy metabolism, metabolites associated with peroxisome functions, or any intermediate product, derivative or metabolite thereof.

In more specific embodiments such metabolite may at least one nucleobase, any derivative, any intermediate product thereof, or any combination or mixture thereof.

Nucleobases, also known as nitrogenous bases or often simply bases, are nitrogen-containing biological compounds that form nucleosides, which in turn are components of nucleotides, with all of these monomers constituting the basic building blocks of nucleic acids. The ability of nucleobases to form base pairs and to stack one upon another leads directly to long-chain helical structures such as ribonucleic acid (RNA) and deoxyribonucleic acid (DNA).

Five nucleobases, adenine (A), cytosine (C), guanine (G). thymine (T), and uracil (U), are called primary or canonical. They function as the fundamental units of the genetic code, with the bases A, G, C, and T being found in DNA while A. G, C, and U are found in RNA. Thymine and uracil are identical excepting that T includes a methyl group that U lacks.

Adenine and guanine have a fused-ring skeletal structure derived of purine, hence they are called purine bases. Similarly, the simple-ring structure of cytosine, uracil, and thymine is derived of pyrimidine, so those three bases are called the pyrimidine bases. Each of the base pairs in a typical double-helix DNA comprises a purine and a pyrimidine: either an A paired with a T or a C paired with a G. These purine-pyrimidine pairs, which are called base complements, connect the two strands of the helix and are often compared to the rungs of a ladder. The pairing of purines and pyrimidines may result, in part, from dimensional constraints, as this combination enables a geometry of constant width for the DNA spiral helix. The A-T and C-G pairings function to form double or triple hydrogen bonds between the amine and carbonyl groups on the complementary bases.

It should be understood that the invention further encompasses any modified nucleobases, for example, modified adenosine or guanosine such as Hypoxanthine, anthine, Inosine, Xanthosine, 7-Methylguanosine (m⁷G), 7-Methylguanosine (m⁷G), or modified cytosine. thymine or uridine such as Dihydrouracil, 5-Methylcytosine. 5-Hydroxymethylcytosine, Dihydrouridine, 5-Methylcytidine.

Still further, Nucleosides are glycosylamines that can be thought of as nucleotides without a phosphate group. A nucleoside consists simply of a nucleobase (also termed a nitrogenous base) and a five-carbon sugar (either ribose or deoxyribose), whereas a nucleotide is composed of a nucleobase, a five-carbon sugar, and one or more phosphate groups. In a nucleoside, the base is bound to either ribose or deoxyribose via a beta-glycosidic linkage. Examples of nucleosides include cytidine, uridine, adenosine, guanosine, thymidine and inosine. Nucleotides, are organic molecules that serve as the monomer units for forming the nucleic acid polymers deoxyribonucleic acid (DNA) and ribonucleic acid (RNA), both of which are essential biomolecules within all life-forms on Earth. Nucleotides are the building blocks of nucleic acids; they are composed of three subunit molecules: a nitrogenous base, a five-carbon sugar (ribose or deoxyribose), and at least one phosphate group. A nucleoside is a nitrogenous base and a 5-carbon sugar. Thus a nucleoside plus a phosphate group yields a nucleotide.

In more specific embodiments such nucleobase may be at least one of purine nucleobases, any derivative or any intermediate product thereof. Purine is a heterocyclic aromatic organic compound that consists of a pyrimidine ring fused to an imidazole ring. There are many naturally occurring purines. They include the nucleobases adenine and guanine. Other notable purines are hypoxanthine, xanthine, theobromine, caffeine, uric acid and isoguanine. Aside from the crucial roles of purines (adenine and guanine) in DNA and RNA, purines are also significant components in a number of other important biomolecules, such as ATP, GTP, cyclic AMP, NADH, and coenzyme A.

In yet some further specific embodiments, such purine nucleobase may be at least one of adenine, and/or any derivative and intermediate thereof.

Adenine, is a nucleobase (a purine derivative). It is one of the four nucleobases in the nucleic acid of DNA that are represented by the letters G-C-A-T. The three others are guanine, cytosine and thymine. Its derivatives have a variety of roles in biochemistry including cellular respiration, in the form of both the energy-rich adenosine triphosphate (ATP) and the cofactors nicotinamide adenine dinucleotide (NAD) and flavin adenine dinucleotide (FAD). It also has functions in protein synthesis and as a chemical component of DNA and RNA. The shape of adenine is complementary to either thymine in DNA or uracil in RNA.

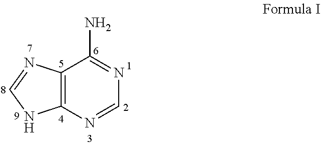

Adenine structure, with standard numbering is presented herein by Formula I.

Adenine forms adenosine, a nucleoside, when attached to ribose, and deoxyadenosine when attached to deoxyribose. It forms adenosine triphosphate (ATP), a nucleoside triphosphate, when three phosphate groups are added to adenosine. Adenosine triphosphate is used in cellular metabolism as one of the basic methods of transferring chemical energy between chemical reactions.

In more specific embodiments, the system of the invention may screen for candidates for the treatment of IEM disorder associated with accumulation of at least one of adenine and any derivatives thereof. In more specific embodiments, such disorder may be adenosine deaminase (ADA) deficiency. In more specific embodiments such disorder may be associated with accumulation of at least one of adenosine and 2′-deoxyadenosine.

Adenosine deaminase deficiency (also called ADA deficiency or ADA-SCID) is an autosomal recessive metabolic disorder that causes immunodeficiency. It accounts for about 15% of all cases of severe combined immunodeficiency (SCID). Additional symptoms of ADA deficiency are pneumonia, chronic diarrhea, and widespread skin rashes. Adenosine deaminase (ADA) is an enzyme which controls adenosine levels in the body by converting adenosine to inosine. It has two active forms. ADA1 is the monomeric intracellular form. ADA1 is integral in functioning of lymphoblasts, and otherwise can be primarily found in airway epithelial cells. ADA2 functions extracellularly as a homodimer which exerts its function in both autocrine and paracrine fashions on monocytes and macrophages.

ADA deficiency is due to a lack of the enzyme adenosine deaminase (encoded by a gene on chromosome 20). This deficiency results in an accumulation of adenosine and deoxyadenosine. It is believed that adenosine and deoxyadenosine accumulation can, lead to: a buildup of dATP in all cells, which inhibits ribonucleotide reductase and prevents DNA synthesis, so cells are unable to divide (since developing T cells and B cells are some of the most mitotically active cells, they are highly susceptible to this condition); an increase in S-adenosylhomocysteine since the enzyme adenosine deaminase is important in the purine salvage pathway (both substances are toxic to immature lymphocytes, which thus fail to mature). Because T cells undergo proliferation and development in the thymus, affected individuals typically have a small, underdeveloped thymus. As a result, the immune system is severely compromised or completely lacking.

In yet some further embodiments, such disorder may be adenine phosphoribosyltransferase (APRT) deficiency. In yet some further embodiments, such disorder may be associated with accumulation of at least one of adenine and 2,8-dihydroxyadenine. Adenine phosphoribosyltransferase deficiency (also called APRT deficiency or 2,8 dihydroxyadenine urolithiasis) is an autosomal recessive metabolic disorder associated with a mutation in the enzyme adenine phosphoribosyltransferase. The disorder results in accumulation of the insoluble purine 2,8-dihydroxyadenine. It can result in nephrolithiasis (kidney stones), acute renal failure and permanent kidney damage.

In embodiments that relate to systems that are specifically adapted for screening for IEMs such as ADA, the system of the invention may comprise specific yeast cell and/or yeast cell line, and/or yeast cell population, and/or any progeny thereof, that display, at least in part, phenotype associated with ADA.

Thus, in some further embodiments, the system of the invention may comprise at least one genetically manipulated yeast cell and/or yeast cell line, and/or yeast cell population, and/or any progeny thereof, that carry a modification in at least one of Adenine phosphoribosyltransferase 1 (APT1) and Adenine deaminase (AAH1) yeast genes.

More specifically, the Adenine phosphoribosyltransferase 1 (APT1) yeast gene as used herein is the Adenine phosphoribosyltransferase 1 (APT1) gene in Saccharomyces cerevisiae (strain ATCC 204508/S288c or Baker's yeast) having the accession number NM_001182380.1 encodes for the enzyme Adenine phosphoribosyltransferase 1 (having the accession number NP_013690.1), which catalyzes a salvage reaction resulting in the formation of AMP, that is energetically less costly than de novo synthesis.

In some embodiments, the gene APT1 comprises the nucleic acid sequence as denoted by SEQ ID NO: 1. In some embodiments, the gene APT1 encodes for a protein comprising the amino acid sequence as denoted by SEQ ID NO: 2.

In yet some further embodiments, the Adenine deaminase (AAH1) yeast gene as used herein is the Adenine Amino Hydrolase 1 (AAH1) gene in Saccharomyces cerevisiae (strain ATCC 204508/S288c or Baker's yeast) having the accession number NM_001182979.1 encodes for the enzyme Adenine deaminase (having the accession number NP_014258.1) which catalyzes the hydrolytic deamination of adenine to hypoxanthine and plays an important role in the purine salvage pathway and in nitrogen catabolism.

In some embodiments, the gene AAH1 comprises the nucleic acid sequence as denoted by SEQ ID NO: 3. In some embodiments, the gene AAH1 encodes for a protein comprising the amino acid sequence as denoted by SEQ ID NO: 4.

In yet some further specific embodiments, such genetically manipulated yeast cell and/or cell line display reduced or no expression of APT1 and AAH1 genes. In some embodiments, such mutated yeast cell line display accumulation of at least one of adenine and any derivative thereof.

In some specific embodiments, the genetically and/or epigenetically manipulated yeast cell and/or cell line is a knockout mutated cell and/or cell line of APT1 and AAH1 genes. In some specific embodiments, the APT1 and AAH1 knock out yeast cell and/or yeast cell line, and/or yeast cell population, and/or any progeny thereof, has been prepared by removal of the AAH1 and/or APT1 open reading frames from the start codon to the stop codon. In more specific embodiments the AAH1 Location: Chromosome XIV 359596 . . . 360639 and APT1 Location: Chromosome XIII 228937 . . . 229500. In more specific embodiments the yeast cell/s of the invention, any cell line thereof, cell population or any progeny thereof, has deletion of the AAH1 gene open reading frame in Chromosome XIV from position 359596 to position 360639 and deletion of the APT1 in Chromosome XIII from position 228937 to position 229500. In yet some further embodiments. the yeast cell of the invention and/or any cell line, cell population and progeny thereof comprise selection markers that replace the open reading frames from the start codon to the stop codon. It should be understood that the specific manipulated yeast cell of the invention or any population, cell line or progeny thereof are provided by the invention and used herein by the systems and methods described herein only as non-limiting embodiments. It must be understood that any genetic and/or epigenetic manipulation, for example, mutation, deletion or insertion may be performed at any part or portion of the APT1 and/or AAH1 genes. For example, only part of the open reading frame (10%, 20%, 30%, 405<50%, 60%, 70%, 80%, 90% and more 95% and more) can be deleted, mutated or otherwise modified (including epigenetic modifications such as methylation and the like), provided that such manipulation/s lead to accumulation of adenosine. In yet some further embodiments, the manipulation may be either transient, and/or stable and/or inducible. In yet some further embodiments, the manipulation may be epigenetic and may involve. methylation and/or repression of transcription or translation.

In yet some further embodiments, the systems of the invention may be applicable for disorders associated with accumulation of purines. In some embodiments, such disorders may result from deficiency of Purine nucleoside phosphorylase, leading to a disorder indicated PNP-deficiency. Purine nucleoside phosphorylase deficiency, often called PNP-deficiency, is a rare autosomal recessive metabolic disorder which results in immunodeficiency. The disorder is caused by a mutation of the purine nucleoside phosphorylase (PNP) gene, located at chromosome 14. PNP is a key enzyme in the purine catabolic pathway, and is required for purine degradation. Specifically. it catalyzes the conversion of inosine to hypoxanthine and guanosine to guanine. A deficiency of it leads to buildup of elevated deoxy-GTP (dGTP) levels resulting in T-cell toxicity and deficiency. In some further embodiments, the system of the invention may be designed for screening candidate compounds for treating IEM disorders associated with accumulation of a metabolite such as uric acid. According to such embodiments, the system may be useful for screening for candidate therapeutic compounds for treating IEM disorder associated with uric acid accumulation, such disorder may be for example, Gout disease.

Gout is a form of inflammatory arthritis characterized by recurrent attacks of a red, tender, hot, and swollen joint. It may also result in tophi, kidney stones, or urate nephropathy. Gout is due to persistently elevated levels of uric acid in the blood. This occurs due to a combination of diet and genetic factors. Gout is partly genetic. The SLC2A9, SLC22A12, and ABCG2 genes have been found to be commonly associated with gout and variations in them can approximately double the risk. The rare genetic disorders familial juvenile hyperuricemic nephropathy. medullary cystic kidney disease, phosphoribosylpyrophosphate synthetase super activity and hypoxanthine-guanine phosphoribosyltransferase deficiency as seen in Lesch-Nyhan syndrome, are complicated by gout. Gout is a disorder of purine metabolism, and occurs when its final metabolite, uric acid, crystallizes in the form of monosodium urate, precipitating and forming deposits (tophi) in joints. on tendons, and in the surrounding tissues.

In yet some further embodiments, the systems of the invention may be applicable for disorders associated with accumulation of pyrimidines.

In yet some further embodiments, the nucleobase may be a pyrimidine nucleobase.

Pyridine is a basic heterocyclic organic compound with the chemical formula C₅H₅N. It is structurally related to benzene, with one methine group (═CH—) replaced by a nitrogen atom. The pyridine ring occurs in many important compounds, including azines and the vitamins niacin and pyridoxine. Pyrimidine is an aromatic heterocyclic organic compound similar to pyridine. One of the three diazines (six-membered heterocyclics with two nitrogen atoms in the ring) has the nitrogen atoms at positions 1 and 3 in the ring. The other diazines are pyrazine (nitrogen atoms at the 1 and 4 positions) and pyridazine (nitrogen atoms at the 1 and 2 positions). In nucleic acids, three types of nucleobases are pyrimidine derivatives: cytosine (C), thymine (T), and uracil (U). The pyrimidine ring system has wide occurrence in nature as substituted and ring fused compounds and derivatives, include the nucleotides cytosine. thymine and uracil, thiamine (vitamin B1) and alloxan.

In yet some further embodiments, any intermediate product of such nucleobases are also encompassed by the invention. Non-limiting examples for such intermediates may include Orotic acid, that is an intermediate product in pyrimidine synthesis.

In yet some further specific embodiments, the IEM targeted by the system of the invention may be a disorder associated or caused by accumulation of thymidine nucleosides, due to loss of Thymidine phosphorylase activity. The associated IEM disorder may be therefore Mitochondrial neurogastrointestinal encephalopathy syndrome (MNGIE).

Mitochondrial neurogastrointestinal encephalopathy syndrome (MNGIE) is a rare autosomal recessive mitochondrial disease. Like other mitochondrial diseases, MNGIE is a multisystem disorder. MNGIE is caused by mutations in the TYMP gene, which encodes the enzyme thymidine phosphorylase. Mutations in this gene result in a loss of thymidine phosphorylase activity. Thymidine phosphorylase is responsible for breaking down thymidine nucleosides into thymine and 2-deoxyribose 1-phosphate. Without normal thymidine phosphorylase activity, thymidine nucleosides begin to build up in cells. High nucleoside levels are toxic to mitochondrial DNA.

In some further embodiments, the system of the invention may be designed for screening candidate compounds for treating IEM disorders associated with accumulation of a metabolite such as at least one amino acid residue, any derivative, or any intermediate product or metabolite thereof.

Amino acid is an organic compound containing amine (—NH2) and carboxyl (—COOH) functional groups, along with a side chain (R group) specific to each amino acid. As used herein, amino acid refers to a naturally occurring or synthetic amino acid, an amino acid analog, or an amino acid mimetic that functions in a manner similar to a naturally occurring amino acid.

Naturally occurring amino acids are those encoded by the genetic code, as well as those amino acids that are later modified, e.g., hydroxyproline, γ-carboxyglutamate, and O-phosphoserine.

In some embodiments, an amino acid or amino acid residue may refer to Arginine (denoted by Arg or R), Lysine (denoted by Lys or K), Aspartic acid (denoted by Asp or D), Glutamic acid (denoted by Glu or E), Glutamine (denoted by Gln or Q), Asparagine (denoted by Asn or N), Histidine (denoted by His or H), Serine (denoted by Ser or S), Threonine (denoted by Thr or T), Tyrosine (denoted by Tyr or Y), Cysteine (denoted by Cys or C), Tryptophan (denoted by Trp or W), Alanine (denoted by Ala or A), Isoleucine (denoted by Ile or I, Leucine (denoted by Leu or L), Methionine (denoted by Met or M), Phenylalanine (denoted by Phe or F), Valine (denoted by Val or V), Proline (denoted by Pro or P), Glycine (denoted by Gly or G.

The amino acids are characterized on the basis of their polarity, charge. solubility. hydrophobicity, hydrophilicity, and/or the amphiphathic nature. Nonpolar “hydrophobic” amino acids are such as valine, isoleucine, leucine, methionine, phenylalanine, tryptophan, cysteine, alanine, tyrosine, histidine, threonine, serine, proline, glycine, arginine and lysine; “polar” amino acids are such as arginine, lysine, aspartic acid. glutamic acid, asparagine, glutamine; “positively charged” amino acids are such as arginine, lysine and histidine; “acidic” amino acids are such as aspartic acid, asparagine, glutamic acid and glutamine; “aromatic” amino acids include tryptophan, tyrosine, naphthylalanine, and phenylalanine. About 500 naturally occurring amino acids are known (though only 20 appear in the genetic code) and can be classified in many ways. They can be classified according to the core structural functional groups' locations as alpha- (α-), beta- (β-), gamma- (γ-) or delta- (δ-) amino acids: other categories relate to polarity. pH level, and side chain group type (aliphatic, acyclic, aromatic, containing hydroxyl or sulfur, etc.).

Amino acid analogs are compounds that have the same fundamental chemical structure as naturally occurring amino acids, i.e., alpha carbon that is bound to a hydrogen, a carboxyl group, an amino group, and an R group. e.g., homoserine. norleucine, methionine sulfoxide, methionine methyl sulfonium. Such analogs have modified R groups or modified peptide backbones, but retain the same basic chemical structure as a naturally occurring amino acid. Amino acid mimetics are chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

Amino acid analogs are such as homo-amino acids, N-alkyl amino acids, dehydroamino acids, aromatic amino acids and α,α-disubstituted amino acids, e.g., cystine, 5-hydroxylysine, 4-hydroxyproline, a-aminoadipic acid, a-amino-n-butyric acid, 3,4-dihydroxyphenylalanine, homoserine, α-methylserine, omithine, pipecolic acid, ortho, meta or para-aminobenzoic acid, citrulline, canavanine, norleucine, d-glutamic acid, aminobutyric acid, L-fluorenylalanine, L-3-benzothienylalanine and thyroxine.

In yet some further specific embodiments such amino acid residue or any intermediate product or metabolite thereof may be at least one of: Phenylalanine, Tyrosine, Glycine, Homocysteine, Arginine, Cysteine, Isoleucine, Leucine, Lysine, Methionine, Proline, Tryptophane, Valine, N-acetylaspartate (NAA), Homogentisic acid, any branched-chain amino acid and any derivatives thereof. A branched-chain amino acid (BCAA) is an amino acid having an aliphatic side-chain with a branch (a central carbon atom bound to three or more carbon atoms). Among the proteinogenic amino acids, there are three BCAAs: leucine, isoleucine, and valine. Non-proteinogenic BCAAs include 2-aminoisobutyric acid.

It should be noted that Homogentisic acid, also known as melanic acid, is an intermediate in the breakdown or catabolism of tyrosine and phenylalanine.

Still further, N-Acetylaspartic acid, or N-acetylaspartate (NAA), is a derivative of aspartic acid. In yet some further specific embodiments, the system of the invention may be designed for screening for therapeutic compounds useful for treating IEM disorder associated with accumulation of at least one of Phenylalanine and any derivatives thereof. In more specific embodiments, such IEM disorder may be Phenylketonuria.

Phenylketonuria (PKU) is an inborn error of metabolism that results in decreased metabolism of the amino acid phenylalanine. It is due to mutations in the PAH gene, which results in low levels of the enzyme phenylalanine hydroxylase. This enzyme is necessary to metabolize the amino acid phenylalanine (Phe) to the amino acid tyrosine (Tyr). When PAH activity is reduced. phenylalanine accumulates and is converted into phenylpyruvate (also known as phenylketone), which can be detected in the urine. It is autosomal recessive and the PAH gene is located on chromosome 12. There are two main types, classic PKU and variant PKU, depending on if any enzyme function remains. PAH deficiency causes a spectrum of disorders, including classic phenylketonuria (PKU) and mild hyperphenylalaninemia (also known as “hyperphe” or “mild HPA”), a less severe accumulation of phenylalanine. Patients with “hyperphe” may have more functional PAH enzyme and be able to tolerate larger amounts of phenylalanine in their diets.

Phenylalanine is a large, neutral amino acid (LNAA). LNAAs compete for transport across the blood-brain barrier (BBB) via the large neutral amino acid transporter (LNAAT). If phenylalanine is in excess in the blood, it will saturate the transporter. Excessive levels of phenylalanine tend to decrease the levels of other LNAAs in the brain. As these amino acids are necessary for protein and neurotransmitter synthesis, Phe buildup hinders the development of the brain, causing intellectual disability. In accordance with such embodiments, the screening systems of the invention may comprise a yeast cell and/or yeast cell line, and/or yeast cell population, and/or any progeny thereof having manipulated aro4 gene. This genetic manipulation leads to accumulation of phenylalanine. In some further embodiments, the system of the invention may comprise at least one genetically manipulated yeast cell line that carry a modification in ARO4 yeast gene. More specifically, the AROmatic amino acid requiring 4 (ARO4) yeast gene as used herein is the AROmatic amino acid requiring 4 (ARO4) in Saccharomyces cerevisiae (strain ATCC 204508/S288c or Baker's yeast) having the accession number NM_001178597.1 encodes for the enzyme 3-deoxy-D-arabino-heptulosonate 7-phosphate synthase or Phospho-2-dehydro-3-deoxyheptonate aldolase, tyrosine-inhibited or DAHP synthase or Phospho-2-keto-3-deoxyheptonate aldolase (having the accession number NP_009808.1). In some embodiments, the gene ARO4 comprises the nucleic acid sequence as denoted by SEQ ID NO: 7. In some embodiments, the gene ARO4 encodes for a protein comprising the amino acid sequence as denoted by SEQ ID NO: 8. In some specific embodiments, the ARO4 knock out yeast cell and/or yeast cell line, and/or yeast cell population, and/or any progeny thereof, has been prepared by removal of the ARO4 open reading frames from the start codon to the stop codon. In more specific embodiments the ARO4 location is Chromosome II 716882 . . . 717994.

In more specific embodiments the yeast cell/s of the invention, any cell line thereof, cell population or any progeny thereof, has deletion of the ARO4 gene open reading frame in Chromosome II from position 716882 to position 717994. In yet some further embodiments, the yeast cell of the invention and/or any cell line, cell population and progeny thereof comprise selection markers that replace the open reading frames from the start codon to the stop codon. It should be understood that the specific manipulated yeast cell of the invention or any population, cell line or progeny thereof are provided by the invention and used herein by the systems and methods described herein only as non-limiting embodiments. It must be understood that any genetic and/or epigenetic manipulation, for example, mutation, deletion or insertion to any part or portion of ARO4 gene may be performed. For example, only part of the open reading frame (10%, 20%, 30%, 405<50%, 60%, 70%, 80%, 90% and more 95% and more) can be deleted, mutated or otherwise modified (including epigenetic modifications such as methylation and the like), provided that such manipulation/s lead to accumulation of phenylalanine. In yet some further embodiments, the manipulation may be either transient, and/or stable and/or inducible. In yet some further embodiments, the manipulation may be epigenetic and may involve, methylation and/or repression of transcription or translation.

In yet some further specific embodiments, the system of the invention may be designed for screening for therapeutic compounds useful for treating TEM disorder associated with accumulation of at least one of Tyrosine and any derivatives thereof. In more specific embodiments, such IEM disorder may be is Tyrosinemia.

Tyrosinemia or tyrosinaemia, is an error of metabolism in which the body cannot effectively break down the amino acid tyrosine. There are several types of tyrosinemias that result from dysfunction of various genes in the phenylalanine and tyrosine catabolic pathway, and are inherited in an autosomal-recessive pattern.

Type I tyrosinemia results from a mutation in the FAH gene, which encodes the enzyme fumarylacetoacetase. As a result of FAH deficiency, the substrate fumarylacetoacetate can accumulate in proximal renal tubular cells and hepatocytes, resulting in damage to the kidney and liver, respectively. Type I tyrosinemia can be detected via blood tests for the presence of a fumarylacetoacetate metabolite, succinylacetone, which is considered a pathognomonic indicator for the disease.

Type II tyrosinemia results from a mutation in the TAT gene, which encodes the enzyme tyrosine aminotransferase. As a result of TAT deficiency, the substrate tyrosine accumulates, causing ophthalmologic and dermatologic abnormalities. Type II tyrosinemia can be detected via the presence of significantly elevated plasma tyrosine levels, and the diagnosis can be confirmed by detection of a mutation in TAT in cultured fibroblasts.

Type III tyrosinemia results from a mutation in the HPD gene, which encodes the enzyme 4-hydroxyphenylpyruvate dioxygenase. Type III tyrosinemia is the rarest of the three conditions, with only a few cases ever reported. Most of those cases have included intellectual disability and neurologic dysfunction. Type III tyrosinemia can be diagnosed by detection of a mutation in HPD in cultured fibroblasts. In accordance with such embodiments, the screening systems of the invention may comprise a yeast cell and/or yeast cell line, and/or yeast cell population, and/or any progeny thereof having manipulated Aro3 gene. This genetic manipulation leads to accumulation of tyrosine. In some further embodiments, the system of the invention may comprise at least one genetically manipulated yeast cell and/or yeast cell line, and/or yeast cell population, and/or any progeny thereof that carry a modification in ARO3 yeast genes. More specifically, the AROmatic amino acid requiring 3 (ARO3) yeast gene as used herein is the AROmatic amino acid requiring 3 (ARO3) in Saccharomyces cerevisiae (strain ATCC 204508/S288c or Baker's yeast) having the accession number NM_001180343.3 encodes for the enzyme 3-deoxy-7-phosphoheptulonate synthase also named Phospho-2-dehydro-3-deoxyheptonate aldolase, phenylalanine inhibited or 3-deoxy-D-arabino-heptulosonate 7-phosphate synthase or DAHP synthase or Phospho-2-keto-3-deoxyheptonate aldolase (having the accession number NP_010320.3).

In some embodiments, the gene ARO3 comprises the nucleic acid sequence as denoted by SEQ ID NO: 5. In some embodiments, the gene ARO3 encodes for a protein comprising the amino acid sequence as denoted by SEQ ID NO: 6. In some specific embodiments, the ARO3 knock out yeast cell and/or yeast cell line, and/or yeast cell population, and/or any progeny thereof, has been prepared by removal of the ARO3 open reading frames from the start codon to the stop codon. In more specific embodiments the ARO3 location is Chromosome IV 521816 . . . 522928. In more specific embodiments the yeast cell/s of the invention, any cell line thereof, cell population or any progeny thereof, has deletion of the ARO3 gene open reading frame in Chromosome IV from position 521816 to position 522928. In yet some further embodiments, the yeast cell of the invention and/or any cell line, cell population and progeny thereof comprise selection markers that replace the open reading frames from the start codon to the stop codon. It should be understood that the specific manipulated yeast cell of the invention or any population, cell line or progeny thereof are provided by the invention and used herein by the systems and methods described herein only as non-limiting embodiments. It must be understood that any genetic and/or epigenetic manipulation, for example, mutation, deletion or insertion to any part or portion of the ARO3 gene may be performed. For example, only part of the open reading frame (10%, 20%, 30%, 405<50%, 60%, 70%, 80%, 90% and more 95% and more) can be deleted, mutated or otherwise modified (including epigenetic modifications such as methylation and the like), provided that such manipulation/s lead to accumulation of tyrosine. In yet some further embodiments, the manipulation may be either transient, and/or stable and/or inducible. In yet some further embodiments, the manipulation may be epigenetic and may involve, methylation and/or repression of transcription or translation.

In some further specific embodiments, the system of the invention may be designed for screening for therapeutic compounds useful for treating IEM disorder associated with accumulation of at least one of Glycine and any derivatives thereof. In more specific embodiments, such IEM disorder may be Glycine encephalopathy.

Glycine encephalopathy (also known as non-ketotic hyperglycinemia or NKH), is an autosomal recessive disorder of glycine metabolism. After phenylketonuria, glycine encephalopathy is the second most common disorder of amino acid metabolism.

It is caused by a defect in the glycine cleavage system (GCS), which is made up of four protein subunits. Each of these four subunits is encoded by a separate gene. Defects in three of these four genes have been linked to glycine encephalopathy i.e. the GLDC gene which encodes the “glycine dehydrogenase” subunit, also called “glycine decarboxylase”, the GCST gene (or also called or AMT gene) which encodes the “aminomethyltransferase” subunit and the GCSH gene which encodes the subunit “glycine cleavage system protein H”. There is a fourth unit in the complex, dihydrolipoamide dehydrogenase or GCSL (no mutations in GCSL found to be associated with glycine encephalopathy).

All forms of glycine encephalopathy show elevated levels of glycine in the plasma, as well as in cerebral spinal fluid (CSF). Glycine which is the simplest amino acid, can act as a neurotransmitter in the brain, or as an inhibitor in the spinal cord and brain stem, while having excitatory effects in the cortex of the brain.

Patients with glycine encephalopathy present neurological symptoms, including intellectual disability (IQ scores below 20 are common), hypotonia, apneic seizures, and brain malformations.

In accordance with such embodiments, the screening systems of the invention may comprise a yeast cell and/or yeast cell line, and/or yeast cell population, and/or any progeny thereof having manipulated GTCV1 gene. This genetic manipulation leads to accumulation of glycine. In some further embodiments, the system of the invention may comprise at least one genetically manipulated yeast cell line that carry a modification in GCV1 yeast gene. More specifically, the Glycine cleavage system T protein (GCV1) yeast gene as sued herein is the Glycine cleavage system T protein (GCV1) in Saccharomyces cerevisiae (strain ATCC 204508/S288c or Baker's yeast) having the accession number NM_001180327.1 encodes for the enzyme Glycine cleavage system T protein or glycine decarboxylase subunit T or Aminomethyltransferase, mitochondrial (having the accession number NP_010302.1). In some embodiments, the gene GCV1 comprises the nucleic acid sequence as denoted by SEQ ID NO: 9. In some embodiments, the gene GCV1 encodes for a protein comprising the amino acid sequence as denoted by SEQ ID NO. 10. In some specific embodiments, the GCV1 knock out yeast cell and/or yeast cell line, and/or yeast cell population, and/or any progeny thereof, has been prepared by removal of the GCV1 open reading frame from the start codon to the stop codon. In more specific embodiments the GCV1 Location is: Chromosome IV 484163 . . . 485365. In more specific embodiments the yeast cell/s of the invention, any cell line thereof, cell population or any progeny thereof, has deletion of the GCV1 gene open reading frame in Chromosome IV from position484163 to position 485365. In yet some further embodiments, the yeast cell of the invention and/or any cell line, cell population and progeny thereof comprise selection markers that replace the open reading frames from the start codon to the stop codon. It should be understood that the specific manipulated yeast cell of the invention or any population, cell line or progeny thereof are provided by the invention and used herein by the systems and methods described herein only as non-limiting embodiments. It must be understood that any genetic and/or epigenetic manipulation, for example, mutation, deletion or insertion to any part or portion of the GCV1 gene may be performed. For example, only part of the open reading frame (10%, 20%, 30%, 405<50%, 60%, 70%, 80%, 90% and more 95% and more) can be deleted, mutated or otherwise modified (including epigenetic modifications such as methylation and the like), provided that such manipulation/s lead to accumulation of glycine. In yet some further embodiments, the manipulation may be either transient, and/or stable and/or inducible. In yet some further embodiments, the manipulation may be epigenetic and may involve. methylation and/or repression of transcription or translation.

In yet some further specific embodiments, the system of the invention may be designed for screening for therapeutic compounds useful for treating IEM disorder associated with accumulation of at least one of Homocysteine and any derivatives thereof. In some embodiments, such IEM disorder may be Homocystinuria.

Classical homocystinuria, also known as cystathionine beta synthase deficiency or CBS deficiency, is an inherited disorder of the metabolism of the amino acid methionine due to a deficiency of cystathionine beta synthase. It is an inherited autosomal recessive disorder. It is characterized by an accumulation of the amino acid homocysteine in the serum and an increased excretion of homocysteine in the urine.

Mutations in the CBS, MTHFR, MTR, MTRR, and MMADHC genes cause homocystinuria.

The CBS gene encodes an enzyme called cystathionine beta-synthase. This enzyme acts in a chemical pathway and is responsible for converting the amino acid homocysteine to a molecule called cystathionine. As a result of this pathway, other amino acids, including methionine, are produced. Mutations in the CBS gene disrupt the function of cystathionine beta-synthase, preventing homocysteine from being used properly. As a result, this amino acid and toxic byproducts substances build up in the blood. Some of the excess homocysteine is excreted in urine. Rarely, homocystinuria can be caused by mutations in several other genes. The enzymes made by the MTHFR, MTR, MTRR, and MMADHC genes play roles in converting homocysteine to methionine. Mutations in any of these genes lead to a buildup of homocysteine in the blood.

Still further, the system of the invention may be particularly adapted for IEM disorders involved with metabolites associated with amino acid metabolism. In accordance with such embodiments, the screening systems of the invention may comprise a yeast cell and/or yeast cell line, and/or yeast cell population, and/or any progeny thereof having manipulated CYS4 gene. This genetic manipulation leads to accumulation of homocysteine. In some further embodiments, the system of the invention may comprise at least one genetically manipulated yeast cell line that carry a modification in CYS4 yeast gene.

More specifically, the Cystathionine beta-synthase 4 (CYS4) yeast gene as sued herein is the Cystathionine beta-synthase 4 (CYS4) in Saccharomyces cerevisiae (strain ATCC 204508/S288c or Baker's yeast) having the accession number NM_001181284.3 encodes for the enzyme Cystathionine beta-synthase or Beta-thionase or Serine sulfhydrase or Sulfur transfer protein 4 (having the accession number NP_011671.3).

In some embodiments, the gene CYS4 comprises the nucleic acid sequence as denoted by SEQ ID NO: 11. In some embodiments, the gene CYS4 encodes for a protein comprising the amino acid sequence as denoted by SEQ ID NO: 12. In some specific embodiments, the CYS4 knock out yeast cell and/or yeast cell line, and/or yeast cell population. and/or any progeny thereof, has been prepared by removal of the CYS4 open reading frame from the start codon to the stop codon. In more specific embodiments the CYS4 Location is: Chromosome VII 798543 . . . 800066. In more specific embodiments the yeast cell/s of the invention, any cell line thereof, cell population or any progeny thereof, has deletion of the CYS4 gene open reading frame in Chromosome VII from position 798543 to position 800066. In yet some further embodiments, the yeast cell of the invention and/or any cell line, cell population and progeny thereof comprise selection markers that replace the open reading frames from the start codon to the stop codon. It should be understood that the specific manipulated yeast cell of the invention or any population, cell line or progeny thereof are provided by the invention and used herein by the systems and methods described herein only as non-limiting embodiments. It must be understood that any genetic and/or epigenetic manipulation, for example, mutation, deletion or insertion to any part or portion of the gene may be performed. For example, only part of the open reading frame (10%, 20%, 30%, 405<50%, 60%, 70%, 80%, 90% and more 95% and more) can be deleted, mutated or otherwise modified (including epigenetic modifications such as methylation and the like), provided that such manipulation/s lead to accumulation of homocysteine. In yet some further embodiments, the manipulation may be either transient, and/or stable and/or inducible. In yet some further embodiments, the manipulation may be epigenetic and may involve, methylation and/or repression of transcription or translation. In some embodiments, the system of the invention may be applicable for Argininemia. Argininemia also called arginase deficiency, is an autosomal recessive urea cycle disorder where a deficiency of the enzyme arginase causes a buildup of arginine and ammonia in the blood. The nervous system is especially sensitive to the effects of excess ammonia. This disease is caused by mutations in the ARG gene. This gene encodes the enzyme arginase that controls the last steps of the urea cycle, which produces urea by extracting nitrogen from arginine. Thus in patients with arginase deficiency, arginine is not broken down properly and accumulates.

In yet some embodiments, the system of the invention may be applicable for MSUD. Maple syrup urine disease (MSUD) is an autosomal recessive metabolic disorder affecting branched-chain amino acids. The disease is named for the presence of sweet-smelling urine of patients, an odor similar to that of maple syrup.

Mutations in the following genes cause maple syrup urine disease: the BCKDHA gene encoding for the Branched-Chain Alpha-Keto Acid Dehydrogenase subunit alpha, the BCKDHB gene encoding for the Branched-Chain Alpha-Keto Acid Dehydrogenase subunit beta, the DBT gene encoding the Lipoamide acyltransferase component of the branched-chain alpha-keto acid dehydrogenase complex, the DLD gene encoding the enzyme Dihydrolipoamide dehydrogenase. These four genes produce proteins that work together as the branched-chain alpha-keto acid dehydrogenase complex. The complex is essential for breaking down the amino acids leucine, isoleucine, and valine.

Mutations in any of these genes reduces or eliminates the function of the enzyme complex, preventing the normal breakdown of isoleucine, leucine, and valine. As a result, these amino acids and their by-products build up in the body. High levels of these substances are particularly toxic to the brain and but also to other organs. In accordance with such embodiments, the screening systems of the invention may comprise a yeast cell and/or yeast cell line, and/or yeast cell population. and/or any progeny thereof having manipulated the BCKAD complex including the LAT1 gene (encoding for dihydrolipoyllysine-residue acetyltransferase) and LPD1 gene (encoding for dihydrolipoyl dehydrogenase). This genetic manipulation leads to accumulation of leucine, isoleucine, and valine. In some further embodiments, the system of the invention may comprise at least one genetically manipulated yeast cell line that carry a modification the BCKAD complex including the LAT1 gene and LPD1 gene.

In some embodiments, the gene LPD1 comprises the nucleic acid sequence as denoted by SEQ ID NO: 15. In some embodiments, the gene LPD1 encodes for a protein comprising the amino acid sequence as denoted by SEQ ID NO: 16.

In some embodiments, the gene LAT1 comprises the nucleic acid sequence as denoted by SEQ ID NO: 17. In some embodiments, the gene LAT1 encodes for a protein comprising the amino acid sequence as denoted by SEQ ID NO: 18.

In some embodiments, the system of the invention may be applicable for ASD. Asparagine synthetase deficiency (ASD) is a neurometabolic disorder characterized by severe congenital microcephaly, severe global developmental delay, intractable seizure disorder, and spastic quadriplegia. It is caused by a homozygous or compound heterozygous mutation in the ASNS gene on chromosome 7. This gene encodes for the enzyme Asparagine synthetase (or aspartate-ammonia ligase) which is a chiefly cytoplasmic enzyme that generates asparagine from aspartate.

In some embodiments, the systems and methods of the invention may be applicable for Hypertryptophanemia. Hypertryptophanemia, also called familial hypertryptophanemia, is a rare autosomal recessive metabolic disorder that results in a massive buildup of the amino acid tryptophan in the blood. Congenital abnormalities in tryptophan metabolism appear to be responsible for the tryptophanemia and tryptophanuria. The underlying genetic cause of hypertryptophanemia is currently unknown.

In some embodiments, the systems and methods of the invention may be applicable for Cystinuria. Cystinuria is an inherited autosomal recessive disease that is characterized by high concentrations of the amino acid cysteine in the urine, leading to the formation of cystine stones in the kidneys, ureter, and bladder. Cystinuria is caused by mutations in the SLC3A1 and SLC7A9 genes. These defects prevent proper reabsorption of basic, or positively charged, amino acids: Cysteine, lysine, omithine, arginine.

In some further specific embodiments, the systems and methods of the invention may be designed for screening for therapeutic compounds useful for treating IEM disorder associated with accumulation of at least one purines, pyrimidines and nucleotides.

In yet some further specific embodiments, the systems and methods of the invention may be designed for screening for therapeutic compounds useful for treating IEM disorder associated with accumulation of at least one of Uric-acid any derivatives thereof. In more specific embodiments, such IEM disorder may be Gout.

In yet some further specific embodiments, the systems and methods of the invention may be designed for screening for therapeutic compounds useful for treating TEM disorder associated with accumulation of at least one of Thymidine and any derivatives thereof. In more specific embodiments, such IEM disorder may be Mitochondrial neurogastrointestinal encephalomyopathy.

In yet some further specific embodiments, the systems and methods of the invention may be designed for screening for therapeutic compounds useful for treating TEM disorder associated with accumulation of at least one of Guanosine and any derivatives thereof. In more specific embodiments, such IEM disorder may be Purine nucleoside phosphorylase deficiency.

In yet some further specific embodiments, the systems and methods of the invention may be designed for screening for therapeutic compounds useful for treating IEM disorder associated with accumulation of at least one of Xanthine and any derivatives thereof. In more specific embodiments, such IEM disorder may be Xanthinuria.

In yet some further specific embodiments, the systems and methods of the invention may be designed for screening for therapeutic compounds useful for treating IEM disorder associated with accumulation of at least one of SAICAr or S-Ado and any derivatives thereof. In more specific embodiments, such TEM disorder may be Adenylosuccinase lyase deficiency.

In yet some further specific embodiments, the systems and methods of the invention may be designed for screening for therapeutic compounds useful for treating IEM disorder associated with accumulation of at least one of Orotic acid and any derivatives thereof. In more specific embodiments, such IEM disorder may be Orotic aciduria.

In yet some further specific embodiments, the systems and methods of the invention may be designed for screening for therapeutic compounds useful for treating IEM disorder associated with accumulation of at least one of Uracil and any derivatives thereof. In more specific embodiments, such IEM disorder may be Ornithine transcarbamylase deficiency.

In yet some further specific embodiments, the systems and methods of the invention may be designed for screening for therapeutic compounds useful for treating IEM disorder associated with accumulation of at least one of Uracil and any derivatives thereof. In more specific embodiments, such IEM disorder may be Dihydropyrimidine dehydrogenase deficiency.

In yet some further specific embodiments, the systems and methods of the invention may be designed for screening for therapeutic compounds useful for treating IEM disorder associated with accumulation of at least one of N-carbamyl-β-alanine and any derivatives thereof. In more specific embodiments, such IEM disorder may be β-Ureidopropionase deficiency.

In yet some further specific embodiments, the systems and methods of the invention may be designed for screening for therapeutic compounds useful for treating TEM disorder associated with accumulation of at least one of AICA-ribotide and any derivatives thereof. In more specific embodiments, such IEM disorder may be AICA-Ribosiduria.

In some further specific embodiments, the systems and methods of the invention may be designed for screening for therapeutic compounds useful for treating TEM disorder associated with accumulation of at least one amino acid residue.

In yet some further specific embodiments. the systems and methods of the invention may be designed for screening for therapeutic compounds useful for treating IEM disorder associated with accumulation of at least one of Arginine and any derivatives thereof. In more specific embodiments, such IEM disorder may be Argininemia.

In yet some further specific embodiments, the systems and methods of the invention may be designed for screening for therapeutic compounds useful for treating TEM disorder associated with accumulation of at least one of Isoleucine or Leucine or Valine and any derivatives thereof. In more specific embodiments, such IEM disorder may be Maple syrup urine.

In yet some further specific embodiments, the systems and methods of the invention may be designed for screening for therapeutic compounds useful for treating IEM disorder associated with accumulation of at least one of Aspartate or Glutamate and any derivatives thereof. In more specific embodiments, such TEM disorder may be Asparagine synthetase deficiency.

In yet some further specific embodiments, the systems and methods of the invention may be designed for screening for therapeutic compounds useful for treating IEM disorder associated with accumulation of at least one of Homocysteine and any derivatives thereof. In more specific embodiments, such TEM disorder may be Homocystinuria.

In yet some further specific embodiments, the systems and methods of the invention may be designed for screening for therapeutic compounds useful for treating IEM disorder associated with accumulation of at least one of Cystine and any derivatives thereof. In more specific embodiments. such IEM disorder may be Cystinuria.

In yet some further specific embodiments, the systems and methods of the invention may be designed for screening for therapeutic compounds useful for treating IEM disorder associated with accumulation of at least one of Glycine and any derivatives thereof. In more specific embodiments, such IEM disorder may be Glycine encephalopathy.

In yet some further specific embodiments, the systems and methods of the invention may be designed for screening for therapeutic compounds useful for treating TEM disorder associated with accumulation of at least one of Methionine and any derivatives thereof. In more specific embodiments, such IEM disorder may be Mud's disease.

In yet some further specific embodiments. the systems and methods of the invention may be designed for screening for therapeutic compounds useful for treating IEM disorder associated with accumulation of at least one of Histidine and any derivatives thereof. In more specific embodiments, such IEM disorder may be Histidinemia.

In yet some further specific embodiments, the systems and methods of the invention may be designed for screening for therapeutic compounds useful for treating IEM disorder associated with accumulation of at least one of D-glyceric acid and any derivatives thereof. In more specific embodiments, such IEM disorder may be Glycerate kinase deficiency.

In yet some further specific embodiments, the systems and methods of the invention may be designed for screening for therapeutic compounds useful for treating TEM disorder associated with accumulation of at least one of Homogentisic acid and any derivatives thereof. In more specific embodiments, such IEM disorder may be Alkaptonuria.

In yet some further specific embodiments. the systems and methods of the invention may be designed for screening for therapeutic compounds useful for treating IEM disorder associated with accumulation of at least one of Proline and any derivatives thereof. In more specific embodiments, such IEM disorder may be Hyperprolinemia.

In yet some further specific embodiments, the systems and methods of the invention may be designed for screening for therapeutic compounds useful for treating TEM disorder associated with accumulation of at least one of Ammonia and any derivatives thereof. In more specific embodiments, such IEM disorder may be Citrullinemia type 1.

In yet some further specific embodiments, the systems and methods of the invention may be designed for screening for therapeutic compounds useful for treating IEM disorder associated with accumulation of at least one of Ammonia and any derivatives thereof. In more specific embodiments, such IEM disorder may be Ornithine transcarbamylase deficiency.

In yet some further specific embodiments, the systems and methods of the invention may be designed for screening for therapeutic compounds useful for treating IEM disorder associated with accumulation of at least one of Cystathionine and any derivatives thereof. In more specific embodiments, such IEM disorder may be Cystathioninuria.

In yet some further specific embodiments, the systems and methods of the invention may be designed for screening for therapeutic compounds useful for treating IEM disorder associated with accumulation of at least one of Methylmalonic acid and any derivatives thereof. In more specific embodiments, such IEM disorder may be Methylmalonic aciduria.

In yet some further specific embodiments, the systems and methods of the invention may be designed for screening for therapeutic compounds useful for treating IEM disorder associated with accumulation of at least one of Sulfites and any derivatives thereof. In more specific embodiments, such IEM disorder may be Sulfite oxidase deficiency.

In yet some further specific embodiments, the systems and methods of the invention may be designed for screening for therapeutic compounds useful for treating TEM disorder associated with accumulation of at least one of Formiminoglutamate or folates and any derivatives thereof. In more specific embodiments, such IEM disorder may be Glutamate formiminotransferase deficiency.

In yet some further specific embodiments, the systems and methods of the invention may be designed for screening for therapeutic compounds useful for treating IEM disorder associated with accumulation of at least one of Lysine or hydroxylysine or tryptophan and any derivatives thereof. In more specific embodiments, such IEM disorder may be Glutaric acidemia type I.

In yet some further specific embodiments, the systems and methods of the invention may be designed for screening for therapeutic compounds useful for treating IEM disorder associated with accumulation of at least one of Isovaleric acid and any derivatives thereof. In more specific embodiments, such IEM disorder may be Isovaleric acidemia.

In yet some further specific embodiments, the systems and methods of the invention may be designed for screening for therapeutic compounds useful for treating TEM disorder associated with accumulation of at least one of Valine and any derivatives thereof. In more specific embodiments, such IEM disorder may be Isobutyryl-CoA dehydrogenase deficiency.

In yet some further specific embodiments. the systems and methods of the invention may be designed for screening for therapeutic compounds useful for treating IEM disorder associated with accumulation of at least one of Sarcosine and any derivatives thereof. In more specific embodiments, such IEM disorder may be Sarcosinemia.

In yet some further specific embodiments, the systems and methods of the invention may be designed for screening for therapeutic compounds useful for treating TEM disorder associated with accumulation of at least one of Tyrosine and any derivatives thereof. In more specific embodiments, such IEM disorder may be Hawkinsinuria.

In yet some further specific embodiments, the systems and methods of the invention may be designed for screening for therapeutic compounds useful for treating IEM disorder associated with accumulation of at least one of Methionine and any derivatives thereof. In more specific embodiments, such IEM disorder may be Glycine N-methyltransferase deficiency.

In yet some further specific embodiments, the systems and methods of the invention may be designed for screening for therapeutic compounds useful for treating IEM disorder associated with accumulation of at least one of N-acetyl-L-aspartic acid and any derivatives thereof. In more specific embodiments, such IEM disorder may be Canavan disease.

In yet some further specific embodiments, the systems and methods of the invention may be designed for screening for therapeutic compounds useful for treating TEM disorder associated with accumulation of at least one of Lysine and any derivatives thereof. In more specific embodiments, such IEM disorder may be Hyperlysinemia.

In yet some further specific embodiments, the systems and methods of the invention may be designed for screening for therapeutic compounds useful for treating IEM disorder associated with accumulation of at least one of Methionine and any derivatives thereof. In more specific embodiments, such IEM disorder may be Hypermethioninemia.

In yet some further specific embodiments, the systems and methods of the invention may be designed for screening for therapeutic compounds useful for treating IEM disorder associated with accumulation of at least one of Methionine and any derivatives thereof. In more specific embodiments, such IEM disorder may be Hypermethioninemia.

In some further specific embodiments, the systems and methods of the invention may be designed for screening for therapeutic compounds useful for treating IEM disorder associated with accumulation of at least one carbohydrate metabolite. A carbohydrate is a biomolecule consisting of carbon (C), hydrogen (H) and oxygen (O) atoms, usually with a hydrogen-oxygen atom ratio of 2:1 (as in water) and thus with the empirical formula Cm(H2O)n (where m may be different from n). This formula holds true for monosaccharides. Some exceptions exist; for example, deoxyribose, a sugar component of DNA, has the empirical formula C5H10O4. The term is most common in biochemistry, where it is a synonym of saccharide, a group that includes sugars, starch, and cellulose. The saccharides are divided into four chemical groups: monosaccharides, disaccharides, oligosaccharides, and polysaccharides. Monosaccharides and disaccharides, the smallest (lower molecular weight) carbohydrates, are commonly referred to as sugars. While the scientific nomenclature of carbohydrates is complex, the names of the monosaccharides and disaccharides very often end in the suffix-ose, as in the monosaccharides fructose (fruit sugar) and glucose (starch sugar) and the disaccharides sucrose (cane or beet sugar) and lactose (milk sugar). In scientific literature, the term “carbohydrate” has many synonyms, like “sugar” (in the broad sense), “saccharide”, “ose”, “glucide”, “hydrate of carbon” or “polyhydroxy compounds with aldehyde or ketone”.

In yet some further specific embodiments, the systems and methods of the invention may be designed for screening for therapeutic compounds useful for treating IEM disorder associated with accumulation of at least one of Glycogen and any derivatives thereof. In more specific embodiments, such IEM disorder may be Pompe Disease.

In yet some further specific embodiments, the systems and methods of the invention may be designed for screening for therapeutic compounds useful for treating TEM disorder associated with accumulation of at least one of Galactose or Galactitol and any derivatives thereof. In more specific embodiments, such IEM disorder may be Galactokinase deficiency.

In yet some further specific embodiments, the systems and methods of the invention may be designed for screening for therapeutic compounds useful for treating IEM disorder associated with accumulation of at least one of Lactic acid or Ammonia and any derivatives thereof. In more specific embodiments, such TEM disorder may be Pyruvate carboxylase deficiency.

In yet some further specific embodiments, the systems and methods of the invention may be designed for screening for therapeutic compounds useful for treating IEM disorder associated with accumulation of at least one of Oxalate and any derivatives thereof. In more specific embodiments, such IEM disorder may be Primary hyperoxaluria type II.

In some further specific embodiments, the systems and methods of the invention may be designed for screening for therapeutic compounds useful for treating IEM disorder associated with accumulation of at least one lipid or lipoprotein metabolite. A lipid is a biomolecule that is soluble in nonpolar solvents. Non-polar solvents are typically hydrocarbons used to dissolve other naturally occurring hydrocarbon lipid molecules that do not (or do not easily) dissolve in water, including fatty acids, waxes, sterols, fat-soluble vitamins (such as vitamins A, D, E, and K), monoglycerides, diglycerides, triglycerides, and phospholipids. Lipids are also defined as hydrophobic or amphiphilic small molecules; the amphiphilic nature of some lipids allows them to form structures such as vesicles, multilamellar/unilamellar liposomes. or membranes in an aqueous environment. Biological lipids originate entirely or in part from two distinct types of biochemical subunits or “building-blocks”: ketoacyl and isoprene groups. Using this approach, lipids may be divided into eight categories: fatty acids, glycerolipids, glycerophospholipids, sphingolipids, saccharolipids, and polyketides (derived from condensation of ketoacyl subunits); and sterol lipids and prenol lipids (derived from condensation of isoprene subunits).

Although the term “lipid” is sometimes used as a synonym for fats, fats are a subgroup of lipids called triglycerides. Lipids also encompass molecules such as fatty acids and their derivatives (including tri-, di-, monoglycerides, and phospholipids), as well as other sterol-containing metabolites such as cholesterol. A lipoprotein is a biochemical assembly of a lipid and a protein whose primary purpose is to transport hydrophobic lipid (also known as fat) molecules in water, as in blood plasma or other extracellular fluids. They have a single-layer phospholipid and cholesterol outer shell, with the hydrophilic portions oriented outward toward the surrounding water and lipophilic portions of each molecule oriented inwards toward the lipids molecules within the particles. Thus, the complex serves to emulsify the fats in extracellular fluids. A special kind of proteins, called apolipoproteins, are embedded in the outer shell, both stabilizing the complex and giving it a functional identity which determines its fate. Many enzymes, transporters, structural proteins, antigens, adhesins, and toxins are lipoproteins. Examples include plasma lipoprotein particles (HDL, LDL, IDL, VLDL and chylomicrons), which enable fats to be carried in all extracellular water, including the blood stream. In yet some further specific embodiments, the systems and methods of the invention may be designed for screening for therapeutic compounds useful for treating IEM disorder associated with accumulation of at least one of Mevalonic acid and any derivatives thereof. In more specific embodiments, such TEM disorder may be Mevalonate kinase deficiency. Other lipid or lipoprotein IEM conditions encompassed by the invention include Lipoprotein lipase Deficiency and LCAT deficiency. In some further specific embodiments, the systems and methods of the invention may be designed for screening for therapeutic compounds useful for treating IEM disorder associated with accumulation of at least one sterol metabolite. Sterols, also known as steroid alcohols, are a subgroup of the steroids and an important class of organic molecules. The steroid core structure is typically composed of seventeen carbon atoms, bonded in four “fused” rings: three six-member cyclohexane rings and one five-member cyclopentane ring. Sterols are forms of steroids with a hydroxy group at position three and a skeleton derived from cholestane. They are a type of lipid. The most familiar type of animal sterol is cholesterol, which is vital to cell membrane structure, and functions as a precursor to fat-soluble vitamins and steroid hormones. In yet some further specific embodiments, the systems and methods of the invention may be designed for screening for therapeutic compounds useful for treating IEM disorder associated with accumulation of at least one of Cholesterol precursors such as for example 7-Dehydrocholesterol (7-DHC) and any derivatives thereof. In more specific embodiments, such IEM disorder may be Smith-Lemli-Opitz syndrome.

In some further specific embodiments, the systems and methods of the invention may be designed for screening for therapeutic compounds useful for treating IEM disorder associated with accumulation of at least one metabolite of the energy metabolism. Energy metabolism is the process of generating energy (ATP) from nutrients. Metabolism comprises a series of interconnected pathways that can function in the presence or absence of oxygen. Aerobic metabolism converts one glucose molecule into 30-32 ATP molecules. Fermentation or anaerobic metabolism is less efficient than aerobic metabolism. A peroxisome is a membrane-bound organelle (formerly known as a microbody), found in the cytoplasm of most eukaryotic cells.

Peroxisomes are oxidative organelles. Frequently, molecular oxygen serves as a co-substrate, from which hydrogen peroxide (H₂O₂) is then formed. Peroxisomes owe their name to hydrogen peroxide generating and scavenging activities. They perform key roles in lipid metabolism and the conversion of reactive oxygen species. Peroxisomes are involved in the catabolism of very long chain fatty acids, branched chain fatty acids, bile acid intermediates, D-amino acids, and polyamines, the reduction of reactive oxygen species—specifically hydrogen peroxide—and the biosynthesis of plasmalogens, i.e., ether phospholipids critical for the normal function of mammalian brains and lungs.

In yet some further specific embodiments, the systems and methods of the invention may be designed for screening for therapeutic compounds useful for treating TEM disorder associated with accumulation of at least one of Ethylmalonic acid and any derivatives thereof. In more specific embodiments, such IEM disorder may be Ethyhmalonic encephalopathy.

In yet some further specific embodiments. the systems and methods of the invention may be designed for screening for therapeutic compounds useful for treating IEM disorder associated with accumulation of at least one of Pyruvate and any derivatives thereof. In more specific embodiments, such IEM disorder may be Pyruvate dehydrogenase phosphatase deficiency.

In some further specific embodiments, the systems and methods of the invention may be designed for screening for therapeutic compounds useful for treating TEM disorder associated with accumulation of at least one metabolite of porphyrin and haem metabolism. Porphyrins are a group of heterocyclic macrocycle organic compounds, composed of four modified pyrrole subunits interconnected at their a carbon atoms via methine bridges (═CH—). With a total of 26 n-electrons, of which 18 n-electrons form a planar, continuous cycle, the porphyrin ring structure is often described as aromatic. In animals. the committed step for porphyrin biosynthesis is the formation of 6-aminolevulinic acid (6-ALA, 5-ALA or dALA) by the reaction of the amino acid glycine with succinyl-CoA from the citric acid cycle. Heme or haem is a coordination complex consisting of an iron ion coordinated to a porphyrin acting as a tetradentate ligand, and to one or two axial ligands. Many porphyrin-containing metalloproteins have heme as their prosthetic group; these are known as hemoproteins. Hemes are most commonly recognized as components of hemoglobin, the red pigment in blood, but are also found in a number of other biologically important hemoproteins such as myoglobin, cytochromes, catalases, heme peroxidase, and endothelial nitric oxide synthase.

In yet some further specific embodiments, the systems and methods of the invention may be designed for screening for therapeutic compounds useful for treating IEM disorder associated with accumulation of at least one of Iron and any derivatives thereof. In more specific embodiments, such TEM disorder may be hereditary hemochromatosis.

In yet some further specific embodiments, the systems and methods of the invention may be designed for screening for therapeutic compounds useful for treating TEM disorder associated with accumulation of at least one of Delta-aminolevulinic acid and any derivatives thereof. In more specific embodiments, such IEM disorder may be Aminolevulinate dehydratase deficiency.

Additional IEM disorders associated with Metabolite Of Porphyrin And Haem Metabolism, include X-linked protoporphyria. In some further specific embodiments, the systems and methods of the invention may be designed for screening for therapeutic compounds useful for treating IEM disorder associated with accumulation of at least one metabolite of neurotransmitter metabolism. Neurotransmitters are endogenous chemicals that enable neurotransmission. It is a type of chemical messenger which transmits signals across a chemical synapse, such as a neuromuscular junction, from one neuron (nerve cell) to another “target” neuron, muscle cell, or gland cell. Neurotransmitters are released from synaptic vesicles in synapses into the synaptic cleft, where they are received by neurotransmitter receptors on the target cells. Their exact numbers are unknown, but more than 200 chemical messengers have been uniquely identified.

In yet some further specific embodiments, the systems and methods of the invention may be designed for screening for therapeutic compounds useful for treating TEM disorder associated with accumulation of at least one of gamma-amino butyric acid (GABA) and any derivatives thereof.

In more specific embodiments, such IEM disorder may be Succinic semialdehyde dehydrogenase deficiency. In some additional embodiments, TEM disorders associated with Neurotransmitters, includes Aromatic L-amino acid decarboxylase (A ADC) deficiency.

In some further specific embodiments, the systems and methods of the invention may be designed for screening for therapeutic compounds useful for treating IEM disorder associated with accumulation of at least one metabolite of lipid or glycoprotein metabolism.

In some more specific embodiments, such IEM disorder may be a lysosomal disorder.

In yet some further specific embodiments. the systems and methods of the invention may be designed for screening for therapeutic compounds useful for treating IEM disorder associated with accumulation of at least one of Globotrinosylceramide and any derivatives thereof. In more specific embodiments, such IEM disorder may be Fabry disease.

In yet some further specific embodiments, the systems and methods of the invention may be designed for screening for therapeutic compounds useful for treating TEM disorder associated with accumulation of at least one of Glycoproteins and any derivatives thereof. In more specific embodiments, such IEM disorder may be Aspartylglucosaminuria.

In some further specific embodiments, the systems and methods of the invention may be designed for screening for therapeutic compounds useful for treating IEM disorder associated with accumulation of at least one metabolite of fatty acid or ketone body metabolism. A fatty acid is a carboxylic acid with a long aliphatic chain, which is either saturated or unsaturated. Most naturally occurring fatty acids have an unbranched chain of an even number of carbon atoms, from 4 to 28. Fatty acids differ by length, often categorized as short to very long. Short-chain fatty acids (SCFA) are fatty acids with aliphatic tails of five or fewer carbons (e.g. butyric acid). Medium-chain fatty acids (MCFA) are fatty acids with aliphatic tails of 6 to 12 carbons, which can form medium-chain triglycerides. Long-chain fatty acids (LCFA) are fatty acids with aliphatic tails of 13 to 21 carbons. Very long chain fatty acids (VLCFA) are fatty acids with aliphatic tails of 22 or more carbons. Saturated fatty acids have no C═C double bonds. They have the same formula CH₃(CH₂)ₙCOOH, with variations in “n”. Unsaturated fatty acids have one or more C═C double bonds. The C═C double bonds can give either cis or trans isomers. A ketone is a functional group with the structure RC(═O)R′, where R and R′ can be a variety of carbon-containing substituents. Ketones and aldehydes are simple compounds that contain a carbonyl group (a carbon-oxygen double bond). Many sugars are ketones, known collectively as ketoses. Fatty acid synthesis proceeds via ketones. For example, acetoacetate is an intermediate in the Krebs cycle which releases energy from sugars and carbohydrates.

In yet some further specific embodiments, the systems and methods of the invention may be designed for screening for therapeutic compounds useful for treating IEM disorder associated with accumulation of at least one of fatty acids and any derivatives thereof. In more specific embodiments, such IEM disorder may be Malonyl-CoA decarboxylase deficiency.

In some further specific embodiments, the systems and methods of the invention may be designed for screening for therapeutic compounds useful for treating IEM disorder such as Peroxisomal disorders. In yet some further specific embodiments, the systems and methods of the invention may be designed for screening for therapeutic compounds useful for treating IEM disorder associated with accumulation of at least one of Oxalate and any derivatives thereof. In more specific embodiments, such IEM disorder may be Primary hyperoxaluria type 1.

In some further specific embodiments, the systems and methods of the invention may be designed for screening for therapeutic compounds useful for treating IEM disorder associated with accumulation of at least one metabolite of the metabolism of vitamins or (non-protein) cofactors. A vitamin is an organic molecule (or related set of molecules) that is an essential micronutrient that an organism needs in small quantities for the proper functioning of its metabolism. Essential nutrients cannot be synthesized in the organism, either at all or not in sufficient quantities, and therefore must be obtained through the diet. Vitamin C can be synthesized by some species but not by others; it is not a vitamin in the first instance but is in the second. The term vitamin does not include the three other groups of essential nutrients: minerals, essential fatty acids, and essential amino acids. Most vitamins are not single molecules, but groups of related molecules called vitamers. For example, vitamin E consists of four tocopherols and four tocotrienols. The thirteen vitamins required by human metabolism are: vitamin A (as all-trans-retinol, all-trans-retinyl-esters, as well as all-trans-beta-carotene and other provitamin A carotenoids), vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid or folate), vitamin B12 (cobalamins), vitamin C (ascorbic acid), vitamin D (calciferols), vitamin E (tocopherols and tocotrienols), and vitamin K (quinones). A cofactor is a non-protein chemical compound or metallic ion that is required for an enzyme's activity as a catalyst, a substance that increases the rate of a chemical reaction. Cofactors can be considered “helper molecules” that assist in biochemical transformations. The rates at which these happen are characterized by in an area of study called enzyme kinetics. Cofactors can be divided into two types, either inorganic ions, or complex organic molecules called coenzymes. Coenzymes are mostly derived from vitamins and other organic essential nutrients in small amounts.

In yet some further specific embodiments, the systems and methods of the invention may be designed for screening for therapeutic compounds useful for treating IEM disorder associated with accumulation of at least one of Homocysteine and any derivatives thereof. In more specific embodiments, such IEM disorder may be Methylenetetrahydrofolate reductase deficiency.

In yet some further specific embodiments, the systems and methods of the invention may be designed for screening for therapeutic compounds useful for treating IEM disorder associated with accumulation of at least one of Phenylalanine and any derivatives thereof. In more specific embodiments, such IEM disorder may be Hyperphenylalaninemia (PKU type 2).

In yet some further specific embodiments, the systems and methods of the invention may be designed for screening for therapeutic compounds useful for treating IEM disorder associated with accumulation of at least one of Pterins and any derivatives thereof. Pterin, as used herein is a heterocyclic compound composed of a pteridine ring system, with a “keto group” (a lactam) and an amino group on positions 4 and 2 respectively. It is structurally related to the parent bicyclic heterocycle called pteridine. Pterins, as a group, are compounds related to pterin with additional substituents. The biosynthesis of pterins begins with guanosine triphosphate (GTP), which is the substrate for GTP cyclohydrolase I. In more specific embodiments, such IEM disorder may be Sepiapterin Reductase Deficiency.

In some further specific embodiments, the systems and methods of the invention may be designed for screening for therapeutic compounds useful for treating IEM disorder such as disorders concerning the metabolism of trace elements and metals. An essential trace element is a dietary element that is needed in very minute quantities for the proper growth, development, and physiology of the organism. The dietary elements or essential trace elements are those that are required to perform vital metabolic activities in organisms. Examples of essential trace elements in animals include Fe (hemoglobin), Cu (respiratory pigments), Co (Vitamin B12), Mn and Zn (enzymes). Some examples within the human body are cobalt, copper, fluorine, iodine, iron, manganese and zinc. Metals can be categorized according to their physical or chemical properties: including ferrous and non-ferrous metals; brittle metals and refractory metals; heavy and light metals; and base, noble, and precious metals. They may be categorized on the basis of their chemical properties into alkali and alkaline earth metals; transition and post-transition metals; and lanthanides and actinides. Some metals are either essential nutrients (typically iron, cobalt, and zinc), or relatively harmless (such as ruthenium, silver, and indium), but can be toxic in larger amounts or certain forms.

In yet some further specific embodiments, the systems and methods of the invention may be designed for screening for therapeutic compounds useful for treating TEM disorder associated with accumulation of at least one of Copper and any derivatives thereof. In more specific embodiments, such IEM disorder may be Wilson's disease.

In yet some further specific embodiments. the systems and methods of the invention may be designed for screening for therapeutic compounds useful for treating IEM disorder associated with accumulation of sphingolipid. Sphingolipids are a class of lipids containing a backbone of sphingoid bases, a set of aliphatic amino alcohols that includes sphingosine. A sphingolipid with an R group consisting of a hydrogen atom only is a ceramide. Other common R groups include phosphocholine, yielding a sphingomyelin, and various sugar monomers or dimers, yielding cerebrosides and globosides, respectively. Cerebrosides and globosides are collectively known as glycosphingolipids. Sphingoid bases are the fundamental building blocks of all sphingolipids. The main mammalian sphingoid bases are dihydrosphingosine and sphingosine, while dihydrosphingosine and phytosphingosine are the principle sphingoid bases in yeast. Sphingosine, dihydrosphingosine, and phytosphingosine may be phosphorylated. Ceramides, as a general class, are N-acylated sphingoid bases lacking additional head groups. Dihydroceramide is produced by N-acylation of dihydrosphingosine. Phytoceramide is produced in yeast by hydroxylation of dihydroceramide at C-4. Complex sphingolipids may be formed by addition of head groups to ceramide or phytoceramide: Sphingomyelins have a phosphocholine or phosphoethanolamine molecule with an ester linkage to the 1-hydroxy group of a ceramide. Glycosphingolipids are ceramides with one or more sugar residues joined in a β-glycosidic linkage. Cerebrosides have a single glucose or galactose at the 1-hydroxy position. Sulfatides are sulfated cerebrosides. Gangliosides have at least three sugars, one of which must be sialic acid. In some embodiments, disorders associated with accumulation of Sphingolipids include Krabbe disease and Nephrotic syndrome type 14 (SGPL1). In yet some further specific embodiments, the systems and methods of the invention may be designed for screening for therapeutic compounds useful for treating IEM disorder associated with accumulation of phospholipids. More specifically, Phospholipids are a class of lipids that are a major component of all cell membranes. They can form lipid bilayers because of their amphiphilic characteristic. The structure of the phospholipid molecule generally consists of two hydrophobic fatty acid “tails” and a hydrophilic “head” consisting of a phosphate group. The two components are usually joined together by a glycerol molecule. The phosphate groups can be modified with simple organic molecules such as choline, ethanolamine or serine. Examples of phospholipids are such as Phosphatidic acid (phosphatidate) (PA), Phosphatidylethanolamine (cephalin) (PE), Phosphatidylcholine (lecithin) (PC), Phosphatidylserine (PS), Phosphoinositides, Phosphatidylinositol (PI), Phosphatidylinositol phosphate (PIP), Phosphatidylinositol bisphosphate (PIP2) and Phosphatidylinositol trisphosphate (PIP3). In some embodiments, disorders associated with accumulation of Phospholipids include Sengers syndrome.

It should be noted IEM disorders relevant for the systems and methods of the invention are indicated in the following Table 1, that further specify the relevant mutated gene involved.

TABLE 1
IEM disorders

| | | | |
|---|---|---|---|
| | | Gene | Accession |
| Disorders | Metabolites | Homo sapiens | number |

Disorders in the metabolism of purines, pyrimidines and nucleotides

| | | | |
|---|---|---|---|
| Adenosine deaminase 1 | Adenosine | Adenosine | NM_000022 |
| deficiency/ | | deaminase 1 (ADA1) | |
| ADA-SCID | | | |
| Adenosine deaminase 2 | Adenosine | Adenosine | NM_001282225 |
| | | deaminase 2 (ADA2) | |
| Adenine | Adenine, 2,8- | Adenine | NM_000485 |
| phosphoribosyl- | dihydroxyadenin | phosphoribosyltrans- | |
| transferase deficiency | | ferase (APRT) | |
| Gout | Uric-acid | Solute Carrier | NM_001001290 |
| | | Family 2 Member 9 | and NM_004827 |
| | | (SLC2A9) and | |
| | | Adenosine | |
| | | triphosphate Binding | |
| | | Cassette Subfamily | |
| | | G Member 2 | |
| | | (ABCG2) | |
| Mitochondrial | Thymidine | Thymidine | NM_001953 |
| neurogastrointestinal | | Phosphorylase | |
| encephalomyopathy | | (TYMP) | |
| Purine nucleoside | Guanosine | Purine Nucleoside | NM_000270.2 |
| phosphorylase | | Phosphorylase (PNP) | |
| deficiency | | | |
| Lesch-Nyhan syndrome/ | Uric-acid | Hypoxanthine | NM_000194 |
| Hypoxanthine | | Phosphoribosyl- | |
| phosphoribosyl- | | transferase 1 | |
| transferase 1 | | (HPRT1) | |
| Xanthinuria | Xanthine | Xanthine | NM_000379 |
| | | Dehydrogenase | |
| | | (XDH) | |
| Adenylosuccinase lyase | SAICAr and S-Ado | Adenylosuccinase | NM_000026 |
| deficiency | | lyase (ADSL) | |
| Orotic aciduria | Orotic acid | Uridine | NM_000373 |
| | | Monophosphate | |
| | | Synthetase (UMPS) | |
| Ornithine | Uracil | Ornithine | NM_000531 |
| transcarbamylase | | transcarbamylase | |
| deficiency | | (OTC) | |
| Dihydropyrimidine | Uracil | Dihydropyrimidine | NM_000110 |
| dehydrogenase | | dehydrogenase | |
| deficiency | | (DPYD) | |
| Inosine triphosphatase | Inosine triphosphate | Inosine triphosphate | NM_033453 |
| deficiency | | pyrophosphohydro- | |
| | | lase (ITPA) | |
| β-Ureidopropionase | N-carbamyl-β-alanine | Ureidopropionase | NM_016327 |
| deficiency | | beta 1 (UPBI) | |
| AICA-Ribosiduria | AICA-ribotide | Aicar | NM_004044 |
| | | Transformylase/Imp | |
| | | Cyclohydrolase | |
| | | (ATIC) | |

Disorders of amino acid and peptide metabolism

| | | | |
|---|---|---|---|
| Argininemia | Arginine | Arginase 1 (ARGI) | NM_000045 |
| Maple syrup urine | Isoleucine/Leucine/ | Branched chain keto | NM_000709, |
| | Valine | acid dehydrogenase | NM_000056, |
| | | E1 alpha | NM_001918 |
| | | (BCKDHA), | |
| | | Branched chain keto | |
| | | acid dehydrogenase | |
| | | E1 beta (BCKDHB), | |
| | | Dihydrolipoamide | |
| | | Branched chain | |
| | | Transacylase (DBT) | |
| Asparagine synthetase | Aspartate, Glutamate | Asparagine | NM_001673 |
| deficiency | | synthetase (ASNS) | |
| Phenylketonuria | Phenylalanine | Phenylalanine | NM_000277 |
| | | Hydroxylase (PAH) | |
| Hypertryptophanemia | Tryptophan | | |
| Tyrosinemia type I | Tyrosine | Fumarylacetoacetate | NM_000137 |
| | | Hydrolase (FAH) | |
| Homocystinuria | Homocysteine | Cystathionine-Beta- | NM_000071 |
| | | Synthase (CBS) | |
| Cystinuria | Cystine | solute carrier family | NM_000341 and |
| | | 3 member 1 | NM_001126335 |
| | | (SLC3A1), solute | |
| | | carrier family 7 | |
| | | member 9 (SLC7A9) | |
| Cystinosis | Cystine | Cystinosin (CTNS) | |
| Glycine encephalopathy | Glycine | aminomethyltransfer- | NM_000481, |
| | | ase (AMT), glycine | NM_000170, |
| | | decarboxylase | NM_004483 |
| | | (GLDC), glycine | |
| | | cleavage system | |
| | | protein H (GCSH) | |
| Mudd's disease | Methionine | methionine | NM_000429 |
| | | adenosyltransferase | |
| | | 1A (MAT) | |
| Histidinemia | Histidine | histidine ammonia- | NM_001258333 |
| | | lyase (HAL) | |
| Glycerate kinase | D-glyceric acid | glycerate kinase | NM_145262 |
| deficiency | | (GLYCTK) | |
| Alkaptonuria | Homogentisic acid | homogentisate 1,2- | NM_000187 |
| | | dioxygenase (HGD) | |
| Hyperprolinemia | Proline | aldehyde | NM_001161504 |
| | | dehydrogenase 4 | and NM_016335 |
| | | family member A1 | |
| | | (ALDH4A1) and | |
| | | proline | |
| | | dehydrogenase 1 | |
| | | (PRODH) | |
| Citrullinemia type 1 | Ammonia | argininosuccinate | NM_000050 |
| | | synthase 1 (ASS1) | |
| Ornithine | Ammonia | ornithine | NM_000531 |
| transcarbamylase (OTC) | | carbamoyltransferase | |
| deficiency | | (OTC) | |
| Cystathioninuria | Cystathionine | cystathionine | NM_001902 |
| | | gamma-lyase (CTH) | |
| Methylmalonic aciduria | Methylmalonic acid | methylmalonyl-CoA | NM_000255, |
| | | mutase (MMUT), | NM_172250, |
| | | metabolism of | NM_052845, |
| | | cobalamin associated | NM_015702, |
| | | A (MMAA), | NM_032601 |
| | | metabolism of | |
| | | cobalamin associated | |
| | | B (MMAB), | |
| | | metabolism of | |
| | | cobalamin associated | |
| | | D (MMADHC), and | |
| | | methylmalonyl-CoA | |
| | | epimerase (MCEE) | |
| Sulfite oxidase | Sulfites | sulfite oxidase | NM_000456 |
| deficiency | | (SUOX) | |
| Glutamate | Formiminoglutamate | formimidoyltransfer- | NM_006657 |
| formiminotransferase | and folates | ase cyclodeaminase | |
| deficiency | | (FTCD) | |
| Glutaric acidemia type I | Lysine, hydroxylysine, | glutaryl-CoA | NM_000159 |
| | and tryptophan | dehydrogenase | |
| | | (GCDH) | |
| Isovaleric acidemia | Isovaleric acid | isovaleryl-CoA | NM_001159508 |
| | | dehydrogenase | |
| | | (IVD) | |
| Isobutyryl-CoA | Valine | acyl-CoA | NM_014384 |
| dehydrogenase | | dehydrogenase | |
| deficiency | | family member 8 | |
| | | (ACAD8) | |
| Sarcosinemia | Sarcosine | sarcosine | NM_001134707 |
| | | dehydrogenase | |
| | | (SARDH) | |
| Hawkinsinuria | Tyrosine | 4- | NM_002150 |
| | | hydroxyphenylpyru- | |
| | | vate dioxygenase | |
| | | (HPD) | |
| Glycine N- | Methionine | Glycine N- | NM_018960 |
| methyltransferase | | methyltransferase | |
| deficiency | | (GNMT) | |
| Tyrosinemia type II | Tyrosine | tyrosine | NM_000353 |
| | | aminotransferase | |
| | | (TAT) | |
| Tyrosinemia type III | Tyrosine | 4- | NM_002150 |
| | | hydroxyphenylpyru- | |
| | | vate dioxygenase | |
| | | (HPD) | |
| Cystathioninuria | Cystathionine | cystathionine | NM_001902 |
| | | gamma-lyase(CTH) | |
| Canavan disease | N-acetyl-L-aspartic acid | aspartoacylase | NM_000049 |
| | | (ASPA) | |
| Hyperlysinemia | Lysine | aminoadipate- | NM_005763 |
| | | semialdehyde | |
| | | synthase (AASS) | |
| Hypermethioninemia | Methionine | adenosylhomocystein- | NM_000687, |
| | | ase (AHCY), | NM_018960 and |
| | | glycine N- | NM_000429 |
| | | methyltransferase | |
| | | (GNMT), and | |
| | | methionine | |
| | | adenosyltransferase | |
| | | 1A (MAT1A) | |
| Methylmalonic | Methylmalonic acid | methylmalonyl-CoA | NM_000255, |
| acidemia | | mutase (MMUT), | NM_172250, |
| | | metabolism of | NM_052845, |
| | | cobalamin associated | NM_015702, |
| | | A (MMAA), | NM_032601 |
| | | metabolism of | |
| | | cobalamin associated | |
| | | B (MMAB), | |
| | | metabolism of | |
| | | cobalamin associated | |
| | | D (MMADHC), and | |
| | | methylmalonyl-CoA | |
| | | epimerase (MC EE) | |

Disorders of carbohydrate metabolism

| | | | |
|---|---|---|---|
| Pompe Disease | Glycogen | glucosidase alpha, | NM_000152 |
| | | acid (GAA) | |
| Galactokinase | Galactose, Galactitol | galactokinase | NM_000154 |
| deficiency | | (GALK) | |
| Pyruvate carboxylase | Lactic acid, Ammonia | Pyruvate carboxylase | NM_001040716 |
| deficiency | | (PC) | |
| Primary hyperoxaluria | Oxalate | glyoxylate and | NM_012203 |
| type II | | hydroxypyruvate | |
| | | reductase (GRHPR) | |

Disorders of lipid and lipoprotein metabolism

| | | | |
|---|---|---|---|
| Mevalonate kinase | Mevalonic acid | Mevalonate kinase | NM_000431 |
| deficiency | | (MVK) | |
| Lipoprotein lipase | triglycerides | lipoprotein lipase | |
| Deficiency | | | |
| LCAT deficiency | cholesterol | lecithin-cholesterol | |
| | | acyltransferase | |

Disorders of the metabolism of sterols

| | | | |
|---|---|---|---|
| Smith-Lemli-Opitz | Cholesterol precursors, | 7-dehydrocholesterol | NM_001360 |
| syndrome | including 7DHC | reductase (DHCR7) | |

Disorders of energy metabolism

| | | | |
|---|---|---|---|
| Ethylmalonic | Ethylmalonic acid | ethylmalonic | NM_014297 |
| encephalopathy | | encephalopathy 1 | |
| | | (ETHE1) | |
| Pyruvate dehydrogenase | Pyruvate | Pyruvate | NM_018444 |
| phosphatase deficiency | | dehydrogenase | |
| | | phosphatase (PDP1) | |

Disorders of porphyrin and haem metabolism

| | | | |
|---|---|---|---|
| Hereditary | Iron | homeostatic iron | NM_000410 |
| hemochromatosis | | regulator (HEE) | |
| Aminolevulinate | Delta-aminolevulinic | Aminolevulinate | NM_001003945 |
| dehydratase deficiency | acid | dehydratase (ALAD) | |
| X-linked protoporphyria | Porphyrins | 5′-aminolevulinate | |
| | | synthase 2 (ALAS2) | |

Disorders of neurotransmitter metabolism

| | | | |
|---|---|---|---|
| Succinic semialdehyde | gamma-amino butyric | aldehyde | NM_001080 |
| dehydrogenase | acid (GABA) | dehydrogenase 5 | |
| deficiency | | family member A1 | |
| | | (ALDH5A1) | |
| Aromatic L-amino acid | L-DOPA | dopa decarboxylase | |
| decarboxylase (AADC) | | | |
| deficiency | | | |

Lysosomal disorders

| | | | |
|---|---|---|---|
| Fabry disease | Globotriaosylceramide | galactosidase alpha | NM_000169 |
| | | (GLA) | |
| Aspartylglucosaminuria | Glycoproteins | aspartylglucosamini- | NM_000027 |
| | | dase (AGA) | |
| Gaucher's disease | Glucocerebroside | glucosylceramidase | NM_000157 |
| | | beta (GBA) | |
| Batten's disease | Lipofuscins | Ceroid lipofucinosis | |
| | | (1-10) | |
| Niemann-Pick disease | Cholesterol and other | NPC1 gene (NPC | |
| type C | lipids | type 1C) or the NPC2 | |
| | | gene (NPC type 2C) | |

Disorders of fatty acid and ketone body metabolism

| | | | |
|---|---|---|---|
| Malonyl-CoA | fatty acids | malonyl-CoA | NM__ 012213 |
| decarboxylase | | decarboxylase | |
| deficiency | | (MLYCD) | |

Peroxisomal disorders

| | | | |
|---|---|---|---|
| Primary hyperoxaluria | Oxalate | alanine-glyoxylate | NM_000030 |
| type 1 | | and serine-pyruvate | |
| | | aminotransferase | |
| | | (AGXT) | |

Disorders in the metabolism of vitamins and (non-protein) cofactors

| | | | |
|---|---|---|---|
| Methylenetetrahydro- | Homocysteine | methylenetetrahydro | NM_005957 |
| folate reductase | | folate reductase | |
| deficiency | | (MTHFR) | |
| Hyperphenylalaninemia | Phenylalanine | quinoid | NM_000320 |
| (PKU type 2) | | dihydropteridine | |
| | | reductase (QDPR) | |
| Sepiapterin Reductase | Pterins | Sepiapterin | NM_003124 |
| Deficiency | | Reductase (SPR) | |

Disorders in the metabolism of trace elements and metals

| | | | |
|---|---|---|---|
| Wilson's disease | Copper | ATPase copper | NM_000053 |
| | | transporting beta | |
| | | (ATP7B) | |

Sphingolipids

| | | |
|---|---|---|
| Krabbe disease | galactosylceramide and | galactosylceramidase |
| | galactosylsphingosine | (galactocerebrosidase). |
| | | Gatactosylceramiciase |
| Nephrotic syndrome | glycosphingolipids and | sphingosine-1- |
| type 14 (SGPL1) | phosphosphingolipids | phosphate lyase 1 |

Phospholipids

| | | |
|---|---|---|
| Sengers syndrome | lipid and glycogen | acylglycerol kinase |

As shown herein, the present invention provides systems and methods for screening for candidate compounds that may be used for treating IM disorders associated with accumulation of metabolites. In some embodiments. any metabolites are applicable in the present invention provided that said metabolite is not a peptide. In yet some further embodiments, IEM disorders associated with any metabolite, may include disorders associated with accumulation of peptides with the proviso that said peptides are not beta amyloid peptides, synuclein, tau or any amyloid-like peptides.

In yet some further embodiments the systems and methods of the invention may be applicable for any disorders associated with accumulation and/or aggregation of metabolites with the proviso that said disorder is not a neurodegenerative disorder, specifically, any one of Alzheimer's disease, Parkinson's disease, synucleopathies tauopathies and the like, ALS, Huntington disease and any prion disease. In yet some further embodiments, the systems and methods of the invention may be applicable for any disorders associated with accumulation and/or aggregation of metabolites with the proviso that said disorder is not a Gaucher disease.

In some specific embodiments thereof, the invention provides a yeast screening system of candidate therapeutic compounds for treating, preventing, ameliorating, reducing or delaying the onset of adenosine deaminase (ADA) deficiency associated with accumulation of at least one of adenine and any derivative thereof. In some specific embodiments, such system comprises: (a) a yeast cell and/or yeast cell line, and/or yeast cell population. and/or any progeny thereof that carry at least one manipulation in at least one yeast metabolic pathway, that leads to accumulation of at least one of adenine and any derivative thereof. In some embodiments, the system of the invention may optionally further comprise (b), at least one reagent or means for determining at least one of accumulation of said metabolite and at least one phenotype associated with accumulation of said adenine and any derivative thereof.

It must be understood that any means disclosed above in connection with any of the other aspects of the invention is also applicable for this aspect.

In yet some further embodiments, the system of the invention may further comprise at least one validation means for the candidate therapeutic compound. More specifically, such validation means may be at least one of: (a) at least one unicellular organism that display accumulation of at least one of adenine and any derivative thereof; (b) at least one multicellular eukaryotic organism that display accumulation of at least one of adenine and any derivative thereof; (c) at least one mammalian cell that display accumulation of at least one of adenine and any derivative thereof; and (d) at least one mammalian animal model that display accumulation of at least one of adenine and any derivative thereof. In some embodiments, the ADA system of the invention may use primary LCL cells obtained from a patient suffering from ADA-deficiency, as a validation means for evaluating any compound that modulate, and specifically reduce the adenosine accumulation in the cells. In yet some further specific and non-limiting embodiments, the ADA-SCID murine model may be used as a validation means in the ADA-system of the invention.

Adenosine Deaminase Severe Combined Immunodeficiency (ADA-SCID) is unique among the different genetic causes of human SCID in that it is not lymphocyte-specific, but rather a systemic purine metabolic disorder causing SCID. The enzyme adenosine deaminase (ADA) is important for the degradation and salvage of purine metabolites. Elevated levels of these metabolites result in impaired lymphoid development and a severe combined immunodeficiency disease (SCID) as well as several non-immune abnormalities including skeletal alterations, lung alterations, hepatic and renal disease. Furthermore, neurological manifestations arise from the accumulation of adenosine and its derivatives and their effect on the nervous system are also described. The pleotrophic effect owing to ADA-SCID contributed to its definition as a ‘systemic’ metabolic disorder. Despite many years of research, a satisfactory explanation for the comprehensive pathology of ADA-SCID is not well explained. In some embodiments, the ADA-SCID mouse comprise at least one modification in the sequence encoding the murine adenosine deaminase. In some specific embodiments the nucleic acid sequence encoding the murine adenosine deaminase may comprise the nucleic acid sequence as denoted by SEQ ID NO. 24. In yet some further embodiments, such nucleic acid sequence encodes the adenosine deaminase that comprise the amino acid sequence as denoted by SEQ ID NO. 23.

In yet some specific embodiments, the system of the invention may comprise genetically manipulated yeast cell and/or yeast cell line, and/or yeast cell population, and/or any progeny thereof that display reduced or no expression of APT1 and AAH1 genes. In more specific embodiments such genetically and/or epigenetically manipulated cell and/or cell line display accumulation of at least one of adenine and any derivative thereof. It should be appreciated that the specific yeast cell/s of the invention as described above are applicable in the present aspect as well. It must be understood that any means disclosed above in connection with any of the other aspects of the invention is also applicable for this aspect.

In some specific embodiments thereof, the invention provides a yeast screening system of candidate therapeutic compounds for treating, preventing, ameliorating, reducing or delaying the onset of Tyrosinemia associated with accumulation of at least one of tyrosine and any derivative thereof. In some specific embodiments, such system comprises: (a) a yeast cell and/or yeast cell line, and/or yeast cell population, and/or any progeny thereof that carry at least one manipulation in at least one yeast metabolic pathway that leads to accumulation of at least one of tyrosine and any derivative thereof. In some embodiments, the system of the invention may optionally further comprise (b), at least one reagent or means for determining at least one of accumulation of said tyrosine and at least one phenotype associated with accumulation of said tyrosine and any derivative thereof. In yet some further embodiments, the system of the invention may further comprise at least one validation means for the candidate therapeutic compound. More specifically, such validation means may be at least one of: (a) at least one unicellular organism that display accumulation of at least one of tyrosine and any derivative thereof: (b) at least one multicellular eukaryotic organism that display accumulation of at least one of tyrosine and any derivative thereof; (c) at least one mammalian cell that display accumulation of at least one of tyrosine and any derivative thereof; and (d) at least one mammalian animal model that display accumulation of at least one of tyrosine and any derivative thereof. In some embodiments, the Tyrosinemia system of the invention may use primary LCL cells obtained from a patient suffering from Tyrosinemia, as a validation means for evaluating any compound that modulate, and specifically reduce the tyrosine accumulation in the cells. In some embodiments, as an evaluation means, a C. elegans model for type I Tyrosinemia may be used. As indicated by Example 14, an RNAi that specifically silences the C. elegans fumarylacetoacetate hydrolase (fah-1) gene was used. In some embodiments, the C. elegans FAH-1 gene comprises the nucleic acid sequence as denoted by SEQ ID NO: 13. In some embodiments, the gene FAH-1 encodes for a protein comprising the amino acid sequence as denoted by SEQ ID NO: 14. In yet some further specific and non-limiting embodiments, the Fah^{-/-} mouse model having a knock-out of the fumarylacetoacetate hydrolase (FAH), may be used as a validation means in the Tyrosinemia-system of the invention. In yet some specific embodiments, the system of the invention may comprise genetically and/or epigenetically manipulated yeast cell and/or yeast cell line, and/or yeast cell population, and/or any progeny thereof that display reduced or no expression of the aro3 gene. Specifically, the yeast cell and/or yeast cell line, and/or yeast cell population. and/or any progeny thereof is the aro3Δ mutant that have a deletion of the aro3 gene. In more specific embodiments such mutated cell line display accumulation of at least one of tyrosine and any derivative thereof. It must be understood that any means disclosed above in connection with any of the other aspects of the invention is also applicable for this aspect.

In some specific embodiments thereof, the invention provides a yeast screening system of candidate therapeutic compounds for treating, preventing, ameliorating, reducing or delaying the onset of Phenylketonuria associated with accumulation of at least one of phenylalanine and any derivative thereof. In some specific embodiments, such system comprises: (a) a yeast cell and/or yeast cell line, and/or yeast cell population, and/or any progeny thereof that carry at least one manipulation in at least one yeast metabolic pathway that leads to accumulation of at least one of phenylalanine and any derivative thereof. In some embodiments, the system of the invention may optionally further comprise (b), at least one reagent or means for determining at least one of accumulation of said phenylalanine and at least one phenotype associated with accumulation of said phenylalanine an any derivative thereof.

In yet some further embodiments, the system of the invention may further comprise at least one validation means for the candidate therapeutic compound. More specifically, such validation means may be at least one of: (a) at least one unicellular organism that display accumulation of at least one of phenylalanine and any derivative thereof; (b) at least one multicellular eukaryotic organism that display accumulation of at least one of phenylalanine and any derivative thereof; (c) at least one mammalian cell that display accumulation of at least one of phenylalanine and any derivative thereof: and (d) at least one mammalian animal model that display accumulation of at least one of phenylalanine and any derivative thereof. In some embodiments, the Phenylketonuria-system of the invention may use primary LCL cells obtained from a patient suffering from Phenylketonuria, as a validation means for evaluating any compound that modulate, and specifically reduce the phenylalanine accumulation in the cells. In some embodiments, as an evaluation means, a C. elegans model for type I Phenylketonuria may be used. As indicated by Example 14, a C. elegans strain with mutation in phenylalanine hydroxylase (RB857) is used. In some embodiments, the gene PAH-1 comprises the nucleic acid sequence as denoted by SEQ ID NO: 19. In some embodiments, the gene PAH-1 encodes for a protein comprising the amino acid sequence as denoted by SEQ ID NO: 20. In yet some further specific and non-limiting embodiments, the murine model deficient in phenylalanine hydroxylase (PAH) activity may be used as a validation means in the Phenylketonuria-system of the invention.

In yet some specific embodiments, the system of the invention may comprise genetically and/or epigenetically manipulated yeast cell and/or yeast cell line, and/or yeast cell population, and/or any progeny thereof that display reduced or no expression of aro4 gene. In more specific embodiments such mutated cell and/or yeast cell line, and/or yeast cell population, and/or any progeny thereof display accumulation of at least one of phenylalanine and any derivative thereof. It must be understood that any means disclosed above in connection with any of the other aspects of the invention is also applicable for this aspect.

In some specific embodiments thereof, the invention provides a yeast screening system of candidate therapeutic compounds for treating. preventing, ameliorating. reducing or delaying the onset of Glycine encephalopathy associated with accumulation of at least one of glycine and any derivative thereof. In some specific embodiments, such system comprises: (a) a yeast cell and/or yeast cell line, and/or yeast cell population, and/or any progeny thereof that carry at least one manipulation in at least one yeast metabolic pathway that leads to accumulation of at least one of glycine and any derivative thereof. In some embodiments, the system of the invention may optionally further comprise (b). at least one reagent or means for determining at least one of accumulation of said glycine and at least one phenotype associated with accumulation of said glycine and any derivative thereof. In yet some further embodiments, the system of the invention may further comprise at least one validation means for the candidate therapeutic compound. More specifically, such validation means may be at least one of: (a) at least one unicellular organism that display accumulation of at least one of glycine and any derivative thereof; (b) at least one multicellular eukaryotic organism that display accumulation of at least one of glycine and any derivative thereof: (c) at least one mammalian cell that display accumulation of at least one of glycine and any derivative thereof: and (d) at least one mammalian animal model that display accumulation of at least one of glycine and any derivative thereof. In some embodiments, the Glycine encephalopathy-system of the invention may use primary LCL cells obtained from a patient suffering from Glycine encephalopathy, as a validation means for evaluating any compound that modulate, and specifically reduce the glycine accumulation in the cells. In some embodiments, as an evaluation means, a C. elegans model for Glycine encephalopathy may be used. Glycine encephalopathy (non-ketotic hyperglycinemia) that carries a modification in gest-1 gene as described above. In yet some further embodiments, for Glycine encephalopathy the Gldc^{-/-} mouse model having a mutation in Glycine decarboxylase gene.

In yet some specific embodiments, the system of the invention may comprise genetically and/or epigenetically manipulated yeast cell and/or yeast cell line, and/or yeast cell population, and/or any progeny thereof that display reduced or no expression of the GCV1 gene. Specifically, the yeast cell and/or yeast cell line, and/or yeast cell population, and/or any progeny thereof is the GCV1 mutant that have a deletion of the GCV1 gene. In more specific embodiments such mutated cell and/or yeast cell line, and/or yeast cell population, and/or any progeny thereof display accumulation of at least one of glycine and any derivative thereof. It must be understood that any means disclosed above in connection with any of the other aspects of the invention is also applicable for this aspect.

In some specific embodiments thereof, the invention provides a yeast screening system of candidate therapeutic compounds for treating, preventing, ameliorating, reducing or delaying the onset of Homocystinuria associated with accumulation of at least one of homocysteine and any derivative thereof. In some specific embodiments, such system comprises: (a) a yeast cell and/or yeast cell line, and/or yeast cell population, and/or any progeny thereof that carry at least one manipulation in at least one yeast metabolic pathway that leads to accumulation of at least one of homocysteine and any derivative thereof. In some embodiments, the system of the invention may optionally further comprise (b), at least one reagent or means for determining at least one of accumulation of said homocysteine and at least one phenotype associated with accumulation of said homocysteine and any derivative thereof. In yet some further embodiments, the system of the invention may further comprise at least one validation means for the candidate therapeutic compound. More specifically, such validation means may be at least one of; (a) at least one unicellular organism that display accumulation of at least one of homocysteine and any derivative thereof; (b) at least one multicellular eukaryotic organism that display accumulation of at least one of homocysteine and any derivative thereof: (c) at least one mammalian cell that display accumulation of at least one of homocysteine and any derivative thereof; and (d) at least one mammalian animal model that display accumulation of at least one of homocysteine and any derivative thereof. In some embodiments, the Homocystinuria-system of the invention may use primary LCL cells obtained from a patient suffering from Homocystinuria, as a validation means for evaluating any compound that modulate, and specifically reduce the homocysteine accumulation in the cells. In some embodiments, as an evaluation means, a C. elegans model for Homocystinuria may be used, specifically, with mutation in (cystathionine β-synthases, CBS). In yet some further specific and non-limiting embodiments, a murine model for Homocystinuria, specifically, with mutation in (cystathionine β-synthases, CBS), the CBS-F mouse model, may be used as a validation means in the Homocystinuria-system of the invention.

In yet some specific embodiments, the system of the invention may comprise genetically and/or epigenetically manipulated yeast cell and/or yeast cell line, and/or yeast cell population, and/or any progeny thereof that display reduced or no expression of the CYS4 gene. Specifically, the yeast cell and/or yeast cell line, and/or yeast cell population, and/or any progeny thereof is the CYS4 mutant that have a deletion of the CYS4 gene. In more specific embodiments such mutated cell line display accumulation of at least one of homocystein and any derivative thereof.

In some specific embodiments, the phenotype associated with accumulation of said adenine and any derivative thereof may be at least one of cell toxicity and formation of metabolite aggregates. According to such embodiments, where the system of the invention further comprise at least one reagent or means for determining at least one of, accumulation of adenine or any derivatives thereof and at least one phenotype associated with the accumulation of adenine and any derivatives thereof.

Thus, in more specific embodiments (a) cell toxicity may be determined by measuring at least one of cell viability, cell proliferation. cell apoptosis and any toxic phenotype on the organism or cell.

In yet some further embodiments (b), the accumulation of adenine and any derivatives thereof and/or the formation of metabolite aggregates may be determined by at least one of metabolic profiling assay, microscopy, light diffraction, absorption or scattering assay. spectrometric assay, immunological assay, flow cytometry, Liquid Chromatography, NMR and stereoscopy.

In yet some further specific embodiments, metabolite accumulation and/or metabolite aggregates may be determined by at least one of Dye-binding specificity, microscopy, X-ray fiber diffraction, X-ray powder diffraction. X-ray single crystal diffraction, mass spectrometry (including ion mobility), immunological assay (e.g. using a specific antibody), flow cytometry, circular dichroism (CD) spectrometry, vibrational CD. Raman Spectroscopy, Fourier-transformed infrared spectroscopy dynamic light scattering (DLS), Nuclear magnetic resonance (NMR) and Liquid Chromatography (HPLC and UPLC). It should be further noted that in certain embodiments, the system provided by the invention may include or comprise at least one means or reagents required for performance of at least one of these assays for determining the level of the metabolite, specifically, the accumulation of the metabolite and/or for metabolite aggregate detection and/or quantification.

It should be appreciated that the invention further encompasses in a further aspect thereof the manipulated yeast cell, cell line or yeast cell populations or any progenies thereof as described above. In some specific embodiments, the invention provides yeast cell/s and/or cell lines having deletion of the entire open reading frame of the APT1 and AAH1 genes, as described above. This cell/s is applicable for adenosine accumulation.

In yet some further embodiments, the yeast cell and/or yeast cell line, and/or yeast cell population. and/or any progeny thereof provided by the invention is the aro3Δ mutant that have a deletion of the entire open reading frame of the aro3 gene. This cell/s is applicable for tyrosine accumulation. In some further embodiments, the yeast cell and/or yeast cell line, and/or yeast cell population, and/or any progeny thereof provided by the invention is the aro4Δ mutant that have a deletion of the entire open reading frame of the aro4 gene. This cell/s is applicable for phenylalanine accumulation.

Still further in some embodiments, the yeast cell and/or yeast cell line, and/or yeast cell population, and/or any progeny thereof provided by the invention is the gcv1Δ mutant that have a deletion of the entire open reading frame of the gcv1 gene. This cell/s is applicable for glycine accumulation. In yet some further embodiments, the yeast cell and/or yeast cell line, and/or yeast cell population, and/or any progeny thereof provided by the invention is the cys4Δ mutant that have a deletion of the entire open reading frame of the cys43 gene. This cell/s is applicable for homocystein accumulation.

In another aspect thereof, the invention provides a screening method of candidate therapeutic compounds for treating. preventing, ameliorating, reducing or delaying the onset of at least one IEM disorder associated with accumulation of at least one metabolite. In more specific embodiments, the method of the invention may comprise the steps of: in a first step (a), contacting and/or incubating a manipulated yeast cell and/or yeast cell line, and/or yeast cell population, and/or any progeny thereof with or in the presence of a candidate compound. It should be noted that the yeast cell and/or cell line carry at least one manipulation in at least one yeast metabolic pathway, that leads to accumulation of said metabolite. The second step (b), involves determining or measuring in the incubated cells of (a), at least one of (or the level of), metabolite accumulation and at least one phenotype associated with the accumulation of the metabolite.

The third step (c) involves determining that the candidate is a therapeutic compound for said IEM disorder if the level of said phenotype is modulated as compared with the level of the phenotype in the absence of the candidate compound.

In more specific embodiments, the method of the invention may further comprise the step of validating a candidate compound that display a modulated level of the phenotype as obtained in step (c). In more specific embodiments such additional validation may comprise the steps of: First (I), applying and or contacting the candidate compound obtained by the method of the invention on at least one of (i) at least one unicellular organism that display accumulation of the metabolite: (ii) at least one multicellular eukaryotic organism that display accumulation of the metabolite; (iii) at least one mammalian cell that display accumulation of the metabolite; and (iv) at least one mammalian animal model that display accumulation of the metabolite. The next step (II), involves measuring in the cells, unicellular organism, multicellular organism or mammal of (I), the level of metabolite accumulation and/or at least one phenotype associated with the accumulation of the metabolite.

In the next step (III), determining that the evaluated candidate is a therapeutic compound for the IEM disorder if the level of the metabolite accumulation and/or phenotype is modulated as compared with the level of the metabolite accumulation and/or phenotype in the absence of the candidate compound.

The methods of the invention involve the step of contacting the cells of the invention with the candidate compound. The term “contacting” as used herein, means to bring, put, incubate, apply or mix together. More specifically, in the context of the present invention, the term “contacting” includes all measures or steps, which allow the candidate therapeutic compound of the invention such that they are in direct or indirect contact with the yeast cell, cell line, cell population or any progeny thereof. Still further, a “candidate compound” as used herein, is any substance with a potential to reduce, alleviate, prevent, or reverse the accumulation/aggregation of the metabolite/s associated with the IEM disorder in the cell or tissue of patient. Various type of candidate substances may be screened by the methods and systems of the invention. Genetic agents can be screened by contacting the yeast cell with a nucleic acid construct encoding for a gene. For example, one may screen cDNA libraries expressing a variety of genes. In other examples one may contact the yeast cell with other proteins or polypeptides which may confer the therapeutic effect. Thus, candidate compound that may be screened according to the methods of the invention include those encoding for any proteins or polypeptides, receptors, enzymes, ligands, regulatory factors, and structural proteins. Candidate substances also include nuclear proteins, cytoplasmic proteins, mitochondrial proteins, secreted proteins, plasmalemma-associated proteins, serum proteins, viral antigens, bacterial antigens, protozoal antigens and parasitic antigens. Candidate compounds additionally comprise proteins, lipoproteins, glycoproteins, phosphoproteins and nucleic acids (for example, RNAs such as ribozymes or antisense nucleic acids). Proteins or polypeptides which can be screened using the methods of the present invention include chaperone proteins, hormones, growth factors, neurotransmitters, enzymes, clotting factors, apolipoproteins, receptors, drugs, oncogenes, tumor antigens, tumor suppressors, structural proteins, viral antigens, parasitic antigens and bacterial antigens. In addition, numerous methods are currently used for random and/or directed synthesis of peptide, and nucleic acid based compounds. The nucleic acid or protein sequences include the delivery of DNA expression constructs that encode them. In yet some further embodiments, the candidate compound may be a small molecule, as describe herein after in connection with other aspects of the invention.

In some embodiments, the methods of the invention, using the systems provided herein, comprise steps that involve assessment and measurement of an end point product, specifically the accumulation of the metabolite and/or at least one phenotype associated with the accumulation of the metabolite. The terms “assessing”, “determining”, “evaluating”, “measuring”, “assaying” are used interchangeably herein to refer to any form of detection or measurement, and include determining whether a substance, signal, phenotype, etc., is present or not. The result of an assessment may be expressed in qualitative and/or quantitative terms. Assessing may be relative or absolute. “Assessing the level of” includes determining the amount of something that is present or determining whether it is present or absent. In some embodiments, assessing the level of the accumulated metabolite and/or the presence of a toxic phenotype and/or of a metabolite aggregate. In some embodiments, the methods of the invention screen for candidate compound/s that modulate the level of the accumulation of the metabolite and/or the level of a phenotype, specifically toxic phenotype, associated with accumulation of the metabolite. “Modulate” as used herein means to decrease (e.g., inhibit, reduce, suppress) or alternatively, increase (e.g., stimulate, activate, enhance) a level, response, property, activity, pathway. or process. A “modulator”, as used herein when referred to the candidate compound tested or evaluated by the systems and methods of the invention, is compound capable of modulating a level of the accumulation of the metabolite and/or the level of a phenotype associated with said accumulation. A modulator may be an inhibitor, antagonist, activator, or agonist. In some embodiments modulation may refer to an alteration, e.g., inhibition or increase, of the of the accumulation of the metabolite and/or the level of a phenotype associated with said accumulation by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100%. Specifically, as compared with the level of the accumulation of the metabolite and/or the level of a phenotype associated with said accumulation in the absence of such modulator.

In some embodiments, modulated refers to either reduced or elevated. In yet some further specific embodiments, the candidate a therapeutic compound for the IEM disorder if the level of said phenotype is reduced as compared with the level of the phenotype in the absence of said candidate compound.

In more specific embodiments, a decrease or inhibition of growth or viability indicates toxicity of the accumulated metabolite in the yeast cell or in the cell, unicellular organism, multicellular organism or mammal of the validation means described herein. Toxicity of the accumulated metabolite in yeast correlates with human and/or other mammalian IEM state associated with abnormal accumulation, and/or aggregation. of the metabolite. If such a yeast cell is exposed to a candidate compound, one can test the ability of the compound to modulate, e.g., inhibit, toxicity in the cell by measuring growth or viability of the cell and comparing the growth or viability with the growth or viability of a yeast cell cultured in the absence of the compound. For example, a screen may be performed that comprises culturing yeast cells that carry at least one manipulation in at least one pathway involved in the metabolism of said metabolite in the presence of a compound, measuring cell growth or viability in the presence of the compound, and comparing cell growth or viability measured in the presence of the compound to cell growth or viability in the absence of the candidate compound. If cell growth or viability is increased or decreased in the presence of the compound as compared to cell growth or viability in the absence of the compound, the compound is identified as a compound that modulates toxicity induced by the accumulated metabolite. In some embodiments a method of screening for a compound that decreases toxicity associated with accumulation of a specific metabolite may comprise: contacting a yeast cell that carry at least one manipulation in at least one pathway involved in the metabolism of said metabolite with a test compound, and evaluating the yeast cell for viability, wherein an increase in viability of the yeast cell as compared to viability of the yeast cell in the absence of the compound indicates that the compound decreases toxicity associated with accumulation of the specific metabolite in a specific IEM disorder. In some embodiments, compound that inhibit toxicity associated with the accumulated metabolite are candidate therapeutic compounds for treating an IEM disorder characterized by accumulation of the metabolite. In certain embodiments the metabolite accumulates and in further embodiments, forms detectable aggregates in the yeast cell. Compounds can be tested for their ability to modulate, e.g., inhibit, formation or persistence of metabolite aggregates. or alternatively, inhibit the accumulation of the metabolite, or even reduce, at least in part the level of the metabolite.

In yet some further specific embodiments, a screen may be performed that comprises culturing yeast cells that carry at least one manipulation in at least one pathway involved in the metabolism of said metabolite in the presence of a compound, measuring metabolite accumulation and/or metabolite aggregation in the presence of the compound, and comparing metabolite accumulation and/or metabolite aggregation measured in the presence of the compound to metabolite accumulation and/or metabolite aggregation in the absence of the candidate compound. If metabolite accumulation and/or metabolite aggregation is increased or decreased in the presence of the compound as compared to metabolite accumulation and/or metabolite aggregation in the absence of the compound, the compound is identified as a compound that modulates metabolite accumulation (level) and/or metabolite aggregation of the accumulated metabolite. In some embodiments a method of screening for a compound that decreases accumulation of a specific metabolite or formation of metabolite aggregate may comprise: contacting a yeast cell that carry at least one manipulation in at least one pathway involved in the metabolism of the metabolite with a compound; and evaluating the yeast cell for metabolite level and/or metabolite aggregation, wherein a decrease in metabolite accumulation and/or metabolite aggregation of the yeast cell as compared to metabolite accumulation or level and/or metabolite aggregation of the yeast cell in the absence of the compound indicates that the compound decreases toxicity associated with accumulation of the specific metabolite in a specific IEM disorder. In some embodiments, compound that inhibit toxicity and/or metabolite aggregation associated with the accumulated metabolite by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or about 100%, are candidate therapeutic compounds that may be suitable for treating an IEM disorder characterized by accumulation of the metabolite.

It should be understood that the yeast cells are indicated herein for simplicity, since this step is mandatory. However, when the methods of the invention further comprise validation step, similar comparison of the effect of the tested candidate on accumulation of the metabolite and/or the level of the phenotype associated with said accumulation, also in the cell, unicellular organism, multicellular organism or mammal of the validation means described herein, is to be compared to the effect in the absence of the candidate compound.

In some specific embodiments, the phenotype associated with accumulation of the metabolite determined or evaluated by the methods of the invention may be at least one of cell toxicity and formation of metabolite aggregate.

In yet some further embodiments of the methods of the invention, cell toxicity is determined by the method of the invention by measuring at least one of cell viability. cell proliferation, cell apoptosis and any toxic phenotype on the organism or cell.

As mentioned herein in connection with other aspects of the invention, in some specific embodiments, cell viability may be determined by 2,3-bis-(2-methhoxy-4-nitro-5-sulphophenyl)-2H-tetrazolium-5-carboxanilide (XTT) viability assay (not useful for cysteine accumulation), Methylene Blue, PrestoBlue viability reagent, the fluorescent intercalator 7-aminoactinomycin D (7-AAD), LIVE/DEAD Viability Kits, cell growth by turbidity, for example at OD600, or by any means for cell counting.

Thus, in some embodiments, the methods of the invention may comprise at least one step required for performing any cell viability and proliferation assay, specifically. any of the assays disclosed above.

In yet some further embodiments, toxicity may be evaluated by measuring apoptosis of the cells. In some embodiments, apoptosis may be determined by at least one of DNA fragmentation (TUNNEL, terminal deoxynucleotidyl transferase dUTP nick end labeling), caspase and or parp1 phosphorylation, annexin V and propidium iodide (PI) assay. Thus, in some embodiments, the methods of the invention may comprise at least one step required for performing any apoptosis or any other cell death assay, specifically, any of the assays disclosed above.

It should be noted that in some embodiments, other toxic phenotype that may be determined on the organism/cell may include, activation of cell stress pathways (e.g., heat shock response), poor fertility, destruction of organs and tissue damages, DNA mutagenesis. ER stress, cell energy status and ATP content, oxidative stress, mitochondrial dysfunction, mitochondrial damage, activation of autophagy and activation of necrosis.

Thus, in some embodiments, the methods of the invention may further comprise at least one step required for performing any of the cell toxicity assay disclosed above.

In yet some further embodiments, metabolite accumulation and/or formation of metabolite aggregation may be determined by the method of the invention using at least one of microscopy, light diffraction, absorption, or scattering assay, spectrometric assay, immunological assay, Liquid Chromatography, NMR. flow cytometry, and stereoscopy.

In yet some further embodiments, metabolite accumulation and/or metabolite aggregation may be measured using at least one of Dye-binding specificity (for example, using thioflavin T (ThT) and congo red, microscopy, X-ray fiber diffraction, X-ray powder diffraction. X-ray single crystal diffraction, mass spectrometry (including ion mobility), immunological assay (e.g. using a specific antibody), flow cytometry, circular dichroism (CD) spectrometry, vibrational CD, Raman Spectroscopy, Fourier-transformed infrared spectroscopy dynamic light scattering (DLS), Liquid Chromatography (HPLC and UPLC) and NMR. Microscopy, metabolic profiling, such as TEM (transmission electron microscope), confocal fluorescence microscopy, confocal Raman microscopy, indirect immunofluorescence.

As indicated above, the method of the invention is intended for screening for candidate compounds that may be useful in treating IEM disorders associated with aggregation of at least one metabolite. In more specific embodiments, such metabolite may be any one of a nucleobase, an amino acid residue, carbohydrate, fatty acid and ketone, sterols, porphyrin and haem, lipid and lipoprotein, neurotransmitters, vitamins and (non-protein) cofactors, trace elements, metals, metabolites associated with energy metabolism. metabolites associated with peroxisome functions, or any intermediate product, derivative or metabolite thereof.

It should be appreciated that metabolite as used in the present aspect may be any of the metabolites as described herein before in connection with the systems of the invention.

In some specific embodiments of the methods of the invention, such metabolite may be at least one nucleobase, any derivative, any intermediate product thereof or any combinations or mixtures thereof.

In more specific embodiments, the method of the invention may be directed for screening for therapeutic compounds applicable in IEM disorders associated with aggregation of at least one nucleobase. In some specific embodiments, such nucleobase may be at least one purine nucleobases, or any derivative, or any intermediate product thereof.

In yet some further specific embodiments of the methods of the invention, such purine nucleobase may be at least one of adenine and any derivative thereof.

Thus, in some specific embodiments the methods of the invention may be applicable for IEM disorder associated with accumulation of at least one of adenine and any derivatives thereof. In more specific embodiments, such IEM disorder may be ADA deficiency or APRT deficiency.

In yet some further embodiments, screening methods applicable for ADA deficiency or APRT deficiency, may provide genetically and/or epigenetically manipulated yeast cell and/or yeast cell line, and/or yeast cell population, and/or any progeny thereof that carry a modification in at least one of APT7 and AAH1 yeast genes.

In more specific embodiments of the methods of the invention, such genetically and/or epigenetically manipulated yeast cell and/or yeast cell line, and/or yeast cell population, and/or any progeny thereof display reduced or no expression of APT1 and AAH1 genes. In more specific embodiments, such mutated cell and/or yeast cell line, and/or yeast cell population, and/or any progeny thereof display accumulation of at least one of adenine and any derivative thereof.

In some specific embodiments, the genetically and/or epigenetically manipulated yeast cell and/or yeast cell line, and/or yeast cell population, and/or any progeny thereof used by the methods of the invention is a knockout mutated cell line of APT1 and AAH1 genes. This cell line is particularly applicable for ADA.

It should be further appreciated that the invention further encompasses any of the yeast cell and/or yeast cell line, and/or yeast cell population, and/or any progeny thereof discussed herein, specifically the manipulated yeast cell lines that display accumulation of a specific metabolite as discussed above. In some embodiments, the present invention further provides a knockout mutated yeast cell and/or cell line that do not express the APT) and AAH1 genes.

In yet some further embodiments, the methods of the invention may be designed for IEM disorders associated with accumulation of a metabolite such as uric acid. Accordingly, such screening method may be applicable for IEM disorder associated with uric acid accumulation, such as Gout disease.

Still further, in some embodiments, the methods of the invention may be designed for IEM disorders associated with accumulation of at least one amino acid residue, or any intermediate product or metabolite thereof.

In more specific embodiments of the methods of the invention, such amino acid residue or any intermediate product or metabolite thereof may be at least one of: Phenylalanine, Tyrosine, Homocysteine, Glycine, Arginine, Cysteine, Isoleucine, Leucine, Lysine, Methionine, Proline, Tryptophane, Valine. N-acetylaspartate (NAA), Homogentisic acid and any derivatives thereof. Thus, in some specific embodiments, the methods of the invention may be designed for screening of therapeutic compounds suitable for IEM disorder associated with accumulation of at least one of Phenylalanine and any derivatives thereof. In more specific embodiments, such IEM may be Phenylketonuria. In some specific embodiments, the genetically and/or epigenetically manipulated yeast cell and/or yeast cell line, and/or yeast cell population, and/or any progeny thereof used by the methods of the invention is a knockout mutated cell line of ARO4 gene. This cell line is particularly applicable for Phenylketonuria.

In yet some further embodiments, the methods of the invention may be designed for screening of therapeutic compounds suitable for IEM disorder associated with accumulation of at least one of Tyrosine and any derivatives thereof. In more specific embodiments, such IEM disorder may be Tyrosinemia. In some specific embodiments, the genetically and/or epigenetically manipulated yeast cell and/or yeast cell line, and/or yeast cell population, and/or any progeny thereof used by the methods of the invention is a knockout mutated cell line of ARO3 gene. This cell line is particularly applicable for Tyrosinemia.

In yet some further embodiments, the methods of the invention may be designed for screening of therapeutic compounds suitable for IEM disorder associated with accumulation of at least one of Glycine and any derivatives thereof. In more specific embodiments, such IEM disorder may be Glycine encephalopathy. In some specific embodiments, the genetically and/or epigenetically manipulated yeast cell and/or yeast cell line, and/or yeast cell population, and/or any progeny thereof used by the methods of the invention is a knockout mutated cell line of GCV1 genes. This cell and/or yeast cell line, and/or yeast cell population, and/or any progeny thereof is particularly applicable for Glycine encephalopathy.

In yet some further specific embodiments, the methods of the invention may be designed for screening of therapeutic compounds suitable for IEM disorder associated with accumulation of at least one of Homocysteine and any derivatives thereof. In more specific embodiments, such IEM disorder may be Homocystinuria. In some specific embodiments, the genetically and/or epigenetically manipulated yeast cell and/or yeast cell line, and/or yeast cell population, and/or any progeny thereof used by the methods of the invention is a knockout mutated cell line of CYS4 gene. This cell line is particularly applicable for Homocystinuria.

In yet some further specific embodiments, the methods of the invention may be designed for screening of therapeutic compounds suitable for IEM disorder associated with accumulation of at least one of leucine, isoleucine, and valine and any derivatives thereof. In more specific embodiments, such IEM disorder may be Maple syrup urine disease (MSUD). In some specific embodiments, the genetically and/or epigenetically manipulated yeast cell and/or yeast cell line. and/or yeast cell population, and/or any progeny thereof used by the methods of the invention is a knockout mutated cell line of the LAT1 gene and LPD1 gene. This cell and/or yeast cell line, and/or yeast cell population. and/or any progeny thereof is particularly applicable for MSUD.

In yet some further embodiments, the methods provided by the invention may further comprise the step of administering to a subject suffering from the specific IEM, an effective amount of said therapeutic compound.

In some aspect thereof, the invention further provides a screening method for a modulator of metabolite self-assembly and/or formation of metabolite aggregation in eukaryotic cells. In some embodiments, the method comprising the steps of:

First (a), incubating a yeast cell and/or yeast cell line, and/or yeast cell population, and/or any progeny thereof in the presence of a candidate compound. The yeast cell and/or cell line carry at least one manipulation in at least one yeast metabolic pathway that leads to accumulation of said metabolite. In the next step (b), measuring in the incubated cells of (a) at least one of the level of the metabolite, specifically metabolite accumulation and the level of at least one phenotype associated with the accumulation of the metabolite. In the next step (c), determining that the candidate modulates formation of metabolite aggregates if the level of said phenotype is modulated as compared with the level of the phenotype in the absence of said candidate compound.

In some embodiments, modulated refers to either reduced or elevated. In yet some further specific embodiments, the candidate reduce formation of metabolite aggregates if the level of said phenotype is reduced as compared with the level of the phenotype in the absence of said candidate compound.

It should be appreciated that in some embodiments, screening methods in accordance with the invention may be a high throughput screen (HTS). High throughput screens often involve testing large numbers of compounds with high efficiency, e.g., in parallel. For example, tens or hundreds of thousands of compounds can be routinely screened in short periods of time, e.g., hours to days. Often such screening is performed in multi-well plates containing, e.g., e.g., 96, 384, 1536, 3456, or more wells (sometimes referred to as micro-well or microtiter plates or dishes) or other vessels in which multiple physically separated cavities or depressions or areas are present in or on a substrate. High throughput screens can involve use of automation, e.g., for liquid handling, imaging, data acquisition and processing, etc. Certain general principles and techniques that may be applied in embodiments of a HTS are known in the art, for example, as described in Macarron R & Hertzberg R P. Design and implementation of high-throughput screening assays. Methods Mol Biol., 565: 1-32, 2009.

In another aspect thereof, the invention provides a screening method for candidate therapeutic compounds for treating. preventing, ameliorating, reducing or delaying the onset of ADA deficiency associated with accumulation of adenine and any derivative thereof. In more specific embodiments, the method comprising the steps of: First in step (a), incubating and/or contacting a genetically and/or epigenctically manipulated yeast cell and/or yeast cell line, and/or yeast cell population, and/or any progeny thereof in the presence of, or with a candidate compound. The yeast cell/s carry at least one manipulation in at least one yeast metabolic pathway that leads to accumulation of the at least one of adenine and any derivative thereof. In the next step (b), measuring in the incubated cells of (a), the level of accumulated metabolite and/or the level of at least one phenotype associated with the accumulation of said at least one of adenine and any derivative thereof. In the third step (c), determining that the candidate is a therapeutic compound for the ADA deficiency if the level of said phenotype is modulated as compared with the level of the accumulated metabolite and/or the phenotype in the absence of said candidate compound.

In some embodiments. the level of the phenotype may be modulated, specifically, either reduced or alternatively elevated as compared with the absence of the candidate compound. In yet some further embodiments, the candidate is a therapeutic compound for the ADA deficiency if the level of the accumulated metabolite and/or the at least one phenotype is reduced as compared with the level of the phenotype in the absence of said candidate compound.

In some specific embodiments, the methods of the invention may further comprise the step of validating a candidate compound displaying modulated level of said phenotype as obtained in step (c), by applying said candidate compound on at least one of: (a) at least one unicellular organism that display accumulation of the at least one of adenine and any derivative thereof; (b) at least one multicellular eukaryotic organism that display accumulation of said at least one of adenine and any derivative thereof; (c) at least one mammalian cell that display accumulation of said at least one of adenine and any derivative thereof and (d) at least one mammalian animal model that display accumulation of said at least one of adenine and any derivative thereof. It should be understood that any of the validation means described in connection with the systems of the invention are applicable for this aspect as well (e.g., cells and animal models).

In more specific embodiments, the genetically and/or epigenetically manipulated yeast cell and/or yeast cell line, and/or yeast cell population, and/or any progeny thereof provided by the methods of the invention may display reduced or no expression of APT1 and AAH1 genes. In more specific embodiments, the mutated cell and/or cell line display accumulation of adenine and any derivative thereof.

In more specific embodiments, the phenotype associated with accumulation of the adenine and any derivative thereof measured and/or evaluated by the methods of the invention may be at least one of cell toxicity and formation of metabolite aggregates.

Still further, the invention provides a screening method for candidate therapeutic compounds for treating, preventing, ameliorating, reducing or delaying the onset of Tyrosinemia associated with accumulation of tyrosine and any derivative thereof. In more specific embodiments, the method comprising the steps of:

First in step (a), contacting and/or incubating a genetically and/or epigenetically manipulated yeast cell and/or yeast cell line, and/or yeast cell population, and/or any progeny thereof with, or in the presence of a candidate compound. The yeast cell/s carry at least one manipulation in at least one yeast metabolic pathway that leads to accumulation of the at least one of tyrosine and any derivative thereof. In the next step (b), measuring in the incubated cells of (a), the level of accumulated metabolite and/or the level of at least one phenotype associated with the accumulation of said at least one of tyrosine and any derivative thereof. In the third step (c), determining that the candidate is a therapeutic compound for the tyrosinemia if the level of said phenotype is modulated as compared with the level of the accumulated metabolite and/or the phenotype in the absence of said candidate compound. In some embodiments, the level of the phenotype may be modulated, specifically, either reduced or alternatively elevated as compared with the absence of the candidate compound. In yet some further embodiments, the candidate is a therapeutic compound for the tyrosinemia if the level of the accumulated tyrosine and/or the at least one phenotype is reduced as compared with the level of the phenotype in the absence of said candidate compound. In some specific embodiments, the methods of the invention may further comprise the step of validating a candidate compound displaying modulated level of said phenotype as obtained in step (c), by applying said candidate compound on at least one of: (a) at least one unicellular organism that display accumulation of at least one of tyrosine and any derivative thereof; (b) at least one multicellular eukaryotic organism that display accumulation of at least one of tyrosine and any derivative thereof; (c) at least one mammalian cell that display accumulation of at least one of tyrosine and any derivative thereof; and (d) at least one mammalian animal model that display accumulation of at least one of tyrosine and any derivative thereof. It should be understood that any of the validation means described in connection with the systems of the invention are applicable for this aspect as well (e.g., cells and animal models). In more specific embodiments, the genetically and/or epigenetically manipulated yeast cell and/or yeast cell line, and/or yeast cell population, and/or any progeny thereof provided by the methods of the invention may display reduced or no expression of ARO3 gene. In more specific embodiments, the mutated cell line display accumulation of tyrosine and any derivative thereof. In more specific embodiments, the phenotype associated with accumulation of the tyrosine and any derivative thereof measured and/or evaluated by the methods of the invention may be at least one of cell toxicity and formation of metabolite aggregates.

Still further, the invention provides a screening method for candidate therapeutic compounds for treating, preventing, ameliorating, reducing or delaying the onset of Phenylketonuria associated with accumulation of phenylalanine and any derivative thereof. In more specific embodiments, the method comprising the steps of: First in step (a), contacting and/or incubating a genetically and/or epigenetically manipulated yeast cell and/or yeast cell line, and/or yeast cell population, and/or any progeny thereof with, or in the presence of a candidate compound. The yeast cell/s carry at least one manipulation in at least one yeast metabolic pathway that leads to accumulation of the at least one of phenylalanine and any derivative thereof. In the next step (b), measuring or determining in the incubated cells of (a), the level of accumulated metabolite and/or the level of at least one phenotype associated with the accumulation of said at least one of phenylalanine and any derivative thereof. In the third step (c), determining that the candidate is a therapeutic compound for the Phenylketonuria if the level of said phenotype is modulated as compared with the level of the accumulated metabolite and/or the phenotype in the absence of said candidate compound. In some embodiments, the level of the phenotype may be modulated, specifically, either reduced or alternatively elevated as compared with the absence of the candidate compound. In yet some further embodiments, the candidate is a therapeutic compound for the Phenylketonuria if the level of the accumulated tyrosine and/or the at least one phenotype is reduced as compared with the level of the phenotype in the absence of said candidate compound. In some specific embodiments. the methods of the invention may further comprise the step of validating a candidate compound displaying modulated level of said phenotype as obtained in step (c), by applying said candidate compound on at least one of: (a) at least one unicellular organism that display accumulation of at least one of phenylalanine and any derivative thereof; (b) at least one multicellular eukaryotic organism that display accumulation of at least one of phenylalanine and any derivative thereof; (c) at least one mammalian cell that display accumulation of at least one of phenylalanine and any derivative thereof; and (d) at least one mammalian animal model that display accumulation of at least one of phenylalanine and any derivative thereof. It should be understood that any of the validation means described in connection with the systems of the invention are applicable for this aspect as well (e.g., cells and animal models). In more specific embodiments, the genetically and/or epigenetically manipulated yeast cell and/or yeast cell line, and/or yeast cell population, and/or any progeny thereof provided by the methods of the invention may display reduced or no expression of ARO4 gene. In more specific embodiments, the mutated cell line display accumulation of phenylalanine and any derivative thereof. In more specific embodiments, the phenotype associated with accumulation of the phenylalanine and any derivative thereof measured and/or evaluated by the methods of the invention may be at least one of cell toxicity and formation of metabolite aggregates.

The invention further provides a screening method for candidate therapeutic compounds for treating, preventing, ameliorating, reducing or delaying the onset of Glycine encephalopathy associated with accumulation of glycine and any derivative thereof. In more specific embodiments, the method comprising the steps of: First in step (a), incubating a genetically and/or epigenetically manipulated yeast cell and/or yeast cell line, and/or yeast cell population, and/or any progeny thereof in the presence of a candidate compound. The yeast cell/s carry at least one manipulation in at least one yeast metabolic pathway that leads to accumulation of the at least one of glycine and any derivative thereof. In the next step (b), measuring in the incubated cells of (a), the level of accumulated glycine and/or the level of at least one phenotype associated with the accumulation of said at least one of glycine and any derivative thereof. In the third step (c), determining that the candidate is a therapeutic compound for the Glycine encephalopathy if the level of said phenotype is modulated as compared with the level of the accumulated glycine and/or the phenotype in the absence of said candidate compound. In some embodiments, the level of the phenotype may be modulated, specifically, either reduced or alternatively elevated as compared with the absence of the candidate compound. In yet some further embodiments, the candidate is a therapeutic compound for the Glycine encephalopathy if the level of the accumulated glycine and/or the at least one phenotype is reduced as compared with the level of the phenotype in the absence of said candidate compound. In some specific embodiments, the methods of the invention may further comprise the step of validating a candidate compound displaying modulated level of said phenotype as obtained in step (c), by applying said candidate compound on at least one of: (a) at least one unicellular organism that display accumulation of at least one of glycine and any derivative thereof; (b) at least one multicellular eukaryotic organism that display accumulation of at least one of glycine and any derivative thereof, (c) at least one mammalian cell that display accumulation of at least one of glycine and any derivative thereof; and (d) at least one mammalian animal model that display accumulation of at least one of glycine and any derivative thereof. It should be understood that any of the validation means described in connection with the systems of the invention are applicable for this aspect as well (e.g., cells and animal models). In more specific embodiments, the genetically and/or epigenetically manipulated yeast cell and/or yeast cell line, and/or yeast cell population, and/or any progeny thereof provided by the methods of the invention may display reduced or no expression of GCV1 gene. In more specific embodiments, the mutated cell line display accumulation of glycine and any derivative thereof. In more specific embodiments, the phenotype associated with accumulation of the glycine and any derivative thereof measured and/or evaluated by the methods of the invention may be at least one of cell toxicity and formation of metabolite aggregates. The invention further provides a screening method for candidate therapeutic compounds for treating, preventing, ameliorating, reducing or delaying the onset of Homocystinuria associated with accumulation of homocysteine and any derivative thereof. In more specific embodiments, the method comprising the steps of: First in step (a), incubating a genetically and/or epigenetically manipulated yeast cell and/or yeast cell line. and/or yeast cell population, and/or any progeny thereof in the presence of a candidate compound. The yeast cell/s carry at least one manipulation in at least one yeast metabolic pathway that leads to accumulation of the at least one of homocysteine and any derivative thereof. In the next step (b), measuring in the incubated cells of (a). the level of accumulated homocysteine and/or the level of at least one phenotype associated with the accumulation of said at least one of homocysteine and any derivative thereof. In the third step (c), determining that the candidate is a therapeutic compound for the Homocystinuria if the level of said phenotype is modulated as compared with the level of the accumulated homocysteine and/or the phenotype in the absence of said candidate compound. In some embodiments, the level of the phenotype may be modulated, specifically, either reduced or alternatively elevated as compared with the absence of the candidate compound. In yet some further embodiments. the candidate is a therapeutic compound for the Homocystinuria if the level of the accumulated homocysteine and/or the at least one phenotype is reduced as compared with the level of the phenotype in the absence of said candidate compound. In some specific embodiments, the methods of the invention may further comprise the step of validating a candidate compound displaying modulated level of said phenotype as obtained in step (c), by applying said candidate compound on at least one of (a) at least one unicellular organism that display accumulation of at least one of homocysteine and any derivative thereof: (b) at least one multicellular eukaryotic organism that display accumulation of at least one of homocysteine and any derivative thereof; (c) at least one mammalian cell that display accumulation of at least one of homocysteine and any derivative thereof; and (d) at least one mammalian animal model that display accumulation of at least one of homocysteine and any derivative thereof. It should be understood that any of the validation means described in connection with the systems of the invention are applicable for this aspect as well (e.g., cells and animal models). In more specific embodiments, the genetically and/or epigenetically manipulated yeast cell and/or yeast cell line, and/or yeast cell population, and/or any progeny thereof provided by the methods of the invention may display reduced or no expression of CYS4 gene. In more specific embodiments, the mutated cell line display accumulation of homocysteine and any derivative thereof. In more specific embodiments, the phenotype associated with accumulation of the homocysteine and any derivative thereof measured and/or evaluated by the methods of the invention may be at least one of cell toxicity and formation of metabolite aggregates.

Still further, in some embodiments, cell toxicity is determined by measuring at least one of cell viability, cell proliferation, cell apoptosis and any toxic phenotype on the organism or cell. In yet some further additional or alternative embodiments, the accumulation of the metabolite and/or the formation of metabolite aggregates may be determined by at least one of microscopy, light diffraction, absorption or scattering assay, metabolic profiling, spectrometric assay, immunological assay, liquid chromatography, NMR, flow cytometry, and stereoscopy.

A further aspect of the invention relates to a method for treating, preventing, ameliorating, reducing or delaying the onset of at least one IEM disorder associated with accumulation of at least one metabolite. In more specific embodiments, the method of the invention may comprise the steps of:

First, obtaining a compound that modulates the level of at least one phenotype associated with the accumulation of said metabolite by a screening method comprising:

(i) contacting and/or incubating a yeast cell and/or yeast cell line, and/or yeast cell population, and/or any progeny thereof with, or in the presence of a candidate compound. The yeast cell/s carry at least one manipulation in at least one yeast metabolic pathway that leads to accumulation of said metabolite: (ii) measuring or determining in the incubated cells of (i) the level of accumulation of the metabolite and/or at least one phenotype associated with the accumulation of said metabolite; and (iii) determining that said candidate is a therapeutic compound for said IEM disorder if the level of said the accumulated metabolite and/or the phenotype is modulated as compared with the level of the phenotype in the absence of said candidate compound. The next step (b) involves administering a therapeutic effective amount of the compound obtained by step (a) to a subject suffering from IEM disorder associated with accumulation of the metabolite.

It should be further understood that the invention further encompasses a therapeutic effective amount of at least one a compound that modulates the level of at least one phenotype associated with the accumulation of a metabolite, for use in a method for treating, preventing, ameliorating, reducing or delaying the onset of at least one IEM disorder associated with accumulation of said least one metabolite. The compound is obtained by the screening methods of the invention as described herein above.

As noted by this aspect, the invention provides therapeutic methods for treating IEM disorders. Inborn error of metabolism disorders (IEM) or inherited metabolic disorders are heritable (genetic) disorders in which the lack of enzymes specific to each disease result in the accumulation of downstream products. The pathology in metabolic disorders involve accumulation of metabolites that can be toxic or interfere with normal function. More than 1000 metabolic disorders have been reported and described so far.

A hierarchical classification of IEM provided by the Society for the Study of Inborn Errors of Metabolism (SSIEM) listed the disorders into 15 disease groups as follows: disorders of amino acid metabolism, disorders of metabolism of purines, disorders of pyrimidines and nucleotides (nucleobases), disorders of peptide metabolism, disorders of carbohydrate metabolism, disorders of fatty acid and ketone body metabolism, disorders of energy metabolism, disorders of sterol metabolism, disorders of porphyrin and haem metabolism, disorders of lipid and lipoprotein metabolism, congenital disorders of glycosylation or of other protein modification, lysosomal disorders, peroxisomal disorders, disorders in the metabolism of vitamins and (non-protein) co-factors, disorders in the metabolism of trace elements and metals, disorders and variants in the metabolism of xenobiotics [19].

Examples of IEM disorders applicable in the present invention may include but are not limited to: Adenine phosphoribosyltransferase deficiency or APRT (Adenine). Adenosine Deaminase Severe Combined Immunodeficiency or ADA-SCID (adenosine), Phenylketonuria (phenyalanine accumulation), Tyrosinemia (tyrosine accumulation), Glycine encephalopathy (glycine accumulation), Gout (uric acid accumulation), Mitochondrial neurogastrointestinal encephalomyopathy or MNGIE (Thymidine phosphorylase deficiency), Purine nucleoside phosphorylase deficiency or PNP-deficiency (guanosine), Asparagine synthetase deficiency (Aspartate, Glutamate), Homocystinuria (Homocysteine), Argininemia or Arginase deficiency (arginine), Maple syrup urine disease (Isoleucine or Leucine or valine), Cystinuria, cystinosis (cysteine), Non-ketotic hyperglycinemia, D-glyceric aciduria, iminoglycinuria (Glycine), Alkaptonuria (Homogentisic acid), Saccharopinuria (Lysine), Hypermethioninemia (Methionine), Canavan disease (N-Acetyl aspartate), Ornithine transcarbamylase deficiency (Orotic acid), Hyperprolinemia, iminoglycinuria (Proline), Hypertryptophanemia, Hartnup disease (Tryptophan), Omithine transcarbamylase deficiency, dihydropyrimidine dehydrogenase deficiency (Uracil), Lesch-Nyhan syndrome (Uric acid), or Isobutyryl-CoA dehydrogenase deficiency (Valine).

It should be noted that the methods of the invention may be applicable for any IEM disorder, specifically any IEM disorder disclosed by the invention herein before in connection with other aspects of the invention. In some embodiments, the IEM disorders disclosed by Table 1 herein before, are also applicable for the present aspect.

In some embodiments, the therapeutic compound administered by the methods of the invention may be obtained by any screening methods as defined by the invention and disclosed herein above. As indicated herein, the invention provides therapeutic methods and compounds for treating IEM disorders in a subject.

As used herein, “disease”, “disorder”, “condition” and the like, as they relate to a subject's health, are used interchangeably and have meanings ascribed to each and all of such terms. It should be appreciated that the invention provides therapeutic methods applicable for any of the disorders disclosed above, as well as to any condition or disease associated therewith. It is understood that the interchangeably used terms “associated”, “linked” and “related”, when referring to pathologies herein, and in some embodiments refers to the metabolite associated with the IEM disorder, mean diseases, disorders, conditions, or any pathologies which at least one of: share causalities. co-exist at a higher than coincidental frequency, or where at least one accumulated metabolite share causalities, co-exist at a higher than coincidental frequency with at least one disorder, specifically, IEM disorder, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms.

The terms “treat, treating, treatment” as used herein and in the claims mean ameliorating one or more clinical indicia of disease activity by administering a pharmaceutical composition of the invention in a patient having a pathologic disorder.

The term “treatment” as used herein refers to the administering of a therapeutic amount of the compounds obtained by the screening systems and methods provided by the invention, or any composition thereof which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease form occurring or a combination of two or more of the above.

The term “prevention” as used herein, includes the prevention or postponement of development of the disease, prevention or postponement of development of symptoms and/or a reduction in the severity of such symptoms that will or are expected to develop, preventing the occurrence or reoccurrence of the acute disease attacks. These further include ameliorating existing symptoms, preventing-additional symptoms and ameliorating or preventing the underlying metabolic causes of symptoms.

The term “amelioration” as referred to herein, relates to a decrease in the symptoms, and improvement in a subject's condition brought about by the compositions and methods according to the invention, wherein said improvement may be manifested in the forms of inhibition of pathologic processes associated with the IEM disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described below.

The terms “delay”, “delaying the onset”, “retard” and all variations thereof are intended to encompass the slowing of the progress and/or exacerbation of a pathologic disorder or an infectious disease and their symptoms slowing their progress. further exacerbation or development, so as to appear later than in the absence of the treatment according to the invention. More specifically, treatment or prevention include the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%. about 30% to 35%, about 35% to 40%, about 40% to 45%, about 45% to 50%, about 50% to 55%, about 55% to 600/o, about 60% to 65%, about 65% to 70%, about 75% to 80%. about 80% to 85% about 85% to 90%, about 90% to 95%, about 95% to 99%, or about 99% to 99.9%.

With regards to the above, it is to be understood that, where provided, percentage values such as, for example, 10%, 5(0%, 120%, 500%, etc., are interchangeable with “fold change” values, i.e., 0.1, 0.5, 1.2, 5, etc., respectively.

The present invention relates to the treatment of subjects, or patients, in need thereof. By “patient” or “subject in need” it is meant any organism to whom the preventive and prophylactic combinations. composition/s. kit/s. and methods herein described is desired, including humans and domestic mammals. In some specific embodiments, the treated subject may be a human subject. The subject may be male or female, a child or an adult. In exemplary embodiments, the subject is an adult (e.g., at least 18 years old). The present invention relates to the treatment of subjects, or patients, in need thereof. It should be further noted that particularly in case of human subject, administering of the compositions of the invention to the patient includes both self-administration and administration to the patient by another person.

The terms “effective amount” or “sufficient amount” mean an amount necessary to achieve a selected result. The “effective treatment amount” is determined by the severity of the disease in conjunction with the preventive or therapeutic objectives, the route of administration and the patient's general condition (age, sex, weight and other considerations known to the attending physician).

A further aspect of the invention relates to a therapeutic compound for treating, preventing, ameliorating, reducing or delaying the onset of at least one IEM disorder associated with accumulation of at least one metabolite. In some specific embodiments, the compound of the invention may be identified by a method comprising the steps of:

In a first step (a), providing a manipulated yeast cell and/or yeast cell line, and/or yeast cell population, and/or any progeny thereof that carry at least one manipulation in at least one yeast metabolic pathway, that leads to accumulation of said metabolite. The second step (b), involves incubating of the yeast cells in the presence of a candidate compound. The third step (c), requires measuring in the incubated cells of (b), the level of the accumulated metabolite and/or the level of at least one phenotype associated with the accumulation of the metabolite. The next step (d) involves determining that the candidate is a therapeutic compound for the IEM disorder if the level of the accumulated metabolite and/or the phenotype is modulated as compared with the level of the accumulated metabolite and/or the phenotype in the absence of the candidate compound.

In some embodiments, the therapeutic compound of the invention may be identified by any of the methods of the invention as defined herein.

In some embodiments, the compounds of the invention may be at least one of a small molecule, aptamer, a peptide, a nucleic acid molecule and an immunological agent, and any combinations thereof.

More specifically, the present invention provides therapeutic compounds based on screening of candidate compounds using the systems and methods provided by the invention.

A “Compound” is used herein to refer to any substance, agent (e.g., molecule), supramolecular complex, material, or combination or mixture thereof. A compound may be any agent that can be represented by a chemical formula, chemical structure, or sequence. Example of compounds applicable for the present invention, include, e.g., small molecules, polypeptides, nucleic acids (e.g., RNAi agents, antisense oligonucleotide, aptamers), lipids, polysaccharides, etc. In general, compounds may be obtained using any suitable method known in the art. The ordinary skilled artisan will select an appropriate method based, e.g., on the nature of the compound. A compound may be at least partly purified. In some embodiments a compound may be provided as part of a composition, which may contain, e.g., a counter-ion, aqueous or non-aqueous diluent or carrier, buffer, preservative, or other ingredient, in addition to the compound, in various embodiments. In some embodiments a compound may be provided as a salt, ester, hydrate, or solvate. In some embodiments a compound is cell-permeable, e.g., within the range of typical compounds that are taken up by cells and acts intracellularly, e.g., within mammalian cells, to produce a biological effect. Certain compounds may exist in particular geometric or stereoisomeric forms. Such compounds, including cis- and trans-isomers, E- and Z-isomers, R- and S-enantiomers, diastereomers, (D)-isomers, (L)-isomers, (−)—and (+)-isomers, racemic mixtures thereof, and other mixtures thereof are encompassed by this disclosure in various embodiments unless otherwise indicated. Certain compounds may exist in a variety or protonation states, may have a variety of configurations, may exist as solvates (e.g., with water (i.e. hydrates) or common solvents) and/or may have different crystalline forms (e.g., polymorphs) or different tautomeric forms. Embodiments exhibiting such alternative protonation states, configurations. solvates, and forms are encompassed by the present disclosure where applicable. Still further, in certain embodiments, candidate compounds can be screened from large libraries of synthetic or natural compounds. A compound to be tested may be referred to as a test compound or a candidate compound. Any compound may be used as a test compound in various embodiments. In some embodiments a library of FDA approved compounds that can be used by humans may be used. Compound libraries are commercially available from a number of companies including but not limited to Maybridge Chemical Co. (Trevillet, Cornwall, UK), Comgenex (Princeton, N.J.), Microsource (New Milford, Conn.), Aldrich (Milwaukee, Wis.), AKos Consulting and Solutions GmbH (Basel, Switzerland), Ambinter (Paris, France), Asinex (Moscow, Russia), Aurora (Graz, Austria), BioFocus DPI, Switzerland, Bionet (Camelford, UK), ChemBridge, (San Diego, Calif.), ChemDiv, (San Diego, Calif.), Chemical Block Lt, (Moscow, Russia), ChemStar (Moscow, Russia), Exclusive Chemistry, Ltd (Obninsk, Russia), Enamine (Kiev, Ukraine), Evotec (Hamburg, Germany), Indofine (Hillsborough, N.J.), Interbio screen (Moscow, Russia), Interchim (Montlucon, France), Life Chemicals, Inc. (Orange, Conn.), Microchemistry Ltd. (Moscow, Russia), Otava, (Toronto, ON), PharmEx Ltd. (Moscow, Russia), Princeton Biomolecular (Monmouth Junction, N.J.), Scientific Exchange (Center Ossipee, N.H.), Specs (Delft, Netherlands), TimTec (Newark, Del.), Toronto Research Corp. (North York ON), UkrOrgSynthesis (Kiev, Ukraine), Vitas-M, (Moscow, Russia). Zelinsky Institute, (Moscow, Russia), and Bicoll (Shanghai, China). Combinatorial libraries are available and can be prepared. Libraries of natural compounds in the form of bacterial, fungal, plant and animal extracts are commercially available or can be readily prepared by methods well known in the art. Compounds isolated from natural sources, such as animals, bacteria, fungi, plant sources, and marine samples may be tested for the presence of potentially useful pharmaceutical compounds. It will be understood that the agents to be screened could also be derived or synthesized from chemical compositions or man-made compounds. In some embodiments a library useful in the present invention may comprise at least 10,000 compounds, at least 50,000 compounds, at least 100,000 compounds, at least 250,000 compounds, or more.

In some specific embodiments, the compound of the invention may be a small molecule. A “small molecule” as used herein, is an organic molecule that is less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding). e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups. It should be understood that the candidate compound used in the systems and methods of the invention may be any of the compounds as described herein above.

In some embodiments, the compound identified by the screening method of the invention and may be used for the IEM disorders may be a compound of the general Formula (II):

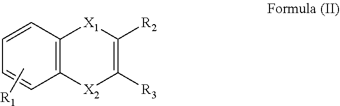

or a pharmaceutically acceptable salt, solvate, hydrate, any stereoisomer thereof or physiologically functional derivative thereof,

wherein each of X₁ and X₂ is independently selected from (CR⁴R⁵), O, S or NR⁴; each R⁴ and R⁵ independently is hydrogen, OH, C₁-C₆ alkyl, C₁-C₆ alkoxy, or CR⁴R⁵ is C═O, R₁ is selected from hydrogen or —OH

Each of R₂ and R₃ is independently selected from hydrogen, an aryl optionally substituted or R₂ and R₃ form together with additional two carbons atoms, a six-membered ring.

In yet some further embodiments, such compound may be any compound of the general formula (III):

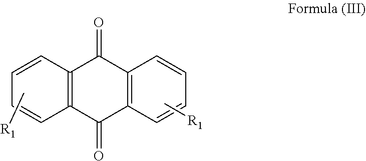

or a pharmaceutically acceptable salt, solvate, hydrate, any stereoisomer thereof or physiologically functional derivative thereof.

each of R₁ is independently selected from hydrogen or —OH.

In yet some further embodiments, the compound of the invention may be the compound of the general formula (IV):

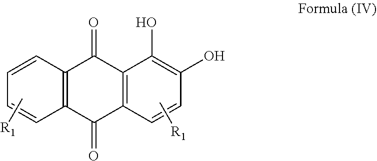

or a pharmaceutically acceptable salt, solvate, hydrate, any stereoisomer thereof or physiologically functional derivative thereof.

each of R₁ and R₅ is independently selected from hydrogen and —OH.

In more specific embodiments, the compound of any one of any one of formulas II, III and IV, may be 1,2,4-Trihydroxyanthraquinone (Purpurin) or any derivatives, analogs, salts and esters thereof.

Purpurin or 1,2,4-Trihydroxyanthraquinone is an anthraquinone. It is a naturally occurring red/yellow dye. It is formally derived from 9,10-anthraquinone by replacement of three hydrogen atoms by hydroxyl (OH) groups.

Purpurin is also called verantin, smoke Brown G, hydroxylizaric acid, and C.I. 58205. Purpurin occurs in the roots of the madder plant (Rubia tinctorum), together with alizarin (1,2-dihydroxyanthraquinone). The root actually contains colorless glycosides of the dyes. Purpurin is a crystalline solid, that forms orange needles melting at 259° C. (498° F.), but becomes red when dissolved in ethanol, and yellow when dissolved with alkalis in boiling water. It is insoluble in hexane but soluble in chloroform, and can be obtained from chloroform as reddish needles. Unlike alizarin, purpurin is dissolved by boiling in a solution of aluminum sulfate, from which it can be precipitated by acid. This procedure can be used to separate the two dyes.

Purpurin has the following chemical structure, as denoted by Formula V:

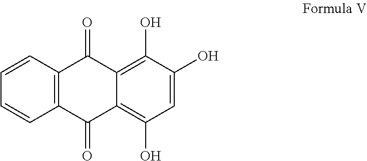 [SMILES: O=C1c2ccccc2C(=O)c2c(O)c(O)cc(O)c21]

The systematic (IUPAC) name of Purpurin is 1,2,4-trihydroxyanthracene-9,10-dione (C₁₄H₈O₅; CAS number: 81-54-9). The molecular weight of purpurin is 256.21 gram/mol.

“Purpurin” further encompass pharmaceutically acceptable salts of purpurin and amorphous and crystalline states of purpurin or of a salt thereof, including any polymorph thereof. Purpurin preparations are formulated for oral, buccal, injectable and transdermal administration.

It should be therefore understood that the term “purpurin” as used by the invention encompasses any form, brand, derivative, enantiomer, metabolite of the compound of Formula V, or any mixture thereof. Purpurin displays a Xanthin Oxidase inhibitor activity. Xanthine oxidase is a form of xanthine oxidoreductase, a type of enzyme that generates reactive oxygen species. These enzymes catalyze the oxidation of hypoxanthine to xanthine and can further catalyze the oxidation of xanthine to uric acid. These enzymes play an important role in the catabolism of purines.

In other specific embodiments, the compound of any one of any one of formulas II, III and IV, may be 1,2-dihydroxyanthraquinone (alizarin) or any derivatives, analogs, salts and esters thereof. Alizarin (also known as 1,2-dihydroxyanthraquinone, Mordant Red 11, C.I. 58000, and Turkey Red) is an organic compound used as a prominent red dye, principally for dyeing textile fabrics. Alizarin is the main ingredient for the manufacture of the madder lake pigments known to painters as Rose madder and Alizarin crimson. It may be derived from the roots of plants of the madder genus. Alizarin is one of ten dihydroxyanthraquinone isomers. Its molecular structure can be viewed as being derived from anthraquinone by replacement of two neighboring hydrogen atoms (H) by hydroxyl groups (—OH). It is soluble in hexane and chloroform, and can be obtained from the latter as red-purple crystals, melting point 277-278° C. Alizarin changes color depending on the pH of the solution it is in, thereby making it a pH indicator.

Alizarin has the following chemical structure, as denoted by Formula VI:

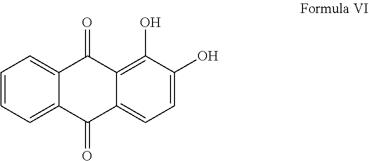 [SMILES: O=C1c2ccccc2C(=O)c2c1ccc(O)c2O]

The systematic (IUPAC) name of Alizarin is 1,2-Dihydroxyanthracene-9,10-dione (C₁₄H₈O₄; CAS number: 72-48-0). The molecular weight of Alizarin is 240.214 g/mol.

“Alizarin” further encompass pharmaceutically acceptable salts of alizarin and amorphous and crystalline states of alizarin or of a salt thereof, including any polymorph thereof. Alizarin preparations are formulated for oral, buccal, injectable and transdermal administration.

It should be therefore understood that the term “alizarin” as used by the invention encompasses any form, brand, derivative, enantiomer, metabolite of the compound of Formula VI, or any mixture thereof.

In yet some further specific embodiments, the compound of any one of any one of formulas II, III and IV, may be 1,2,5,8-tetrahydroxyanthraquinone (Quinalizarin) or any derivatives, analogs, salts and esters thereof. Quinalizarin or 1,2,5,8-tetrahydroxyanthraquinone is an organic compound. It is one of many tetrahydroxyanthraquinone isomers, formally derived from anthraquinone by replacement of four hydrogen atoms by hydroxyl (OH) groups. Quinalizarin is an inhibitor of the enzyme protein kinase CK2. It is more potent and selective than emodin.

Quinalizarin has the following chemical structure, as denoted by Formula VII:

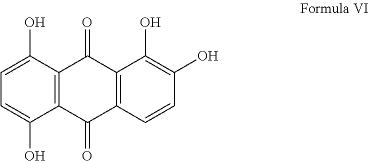 [SMILES: O=C1c2ccc(O)c(O)c2C(=O)c2c(O)ccc(O)c21]

The systematic (IUPAC) name of Quinalizarin is 1,2,5,8-Tetrahydroxyanthracene-9,10-dione (C₁₄H₈O₆; CAS number: 81-61-8). The molecular weight of Quinalizarin is 272.212 gram/mol. “Quinalizarin” further encompass pharmaceutically acceptable salts of Quinalizarin and amorphous and crystalline states of Quinalizarin or of a salt thereof, including any polymorph thereof. Quinalizarin preparations are formulated for oral, buccal. injectable and transdermal administration.

It should be therefore understood that the term “Quinalizarin” as used by the invention encompasses any form, brand, derivative, enantiomer, metabolite of the compound of Formula VII, or any mixture thereof.

In more specific embodiments, the small molecule compound of formula II, may be 5,6,7-trihydroxyflavone (baicalein) or any derivatives, analogs, salts and esters thereof.

Baicalein (5,6,7-trihydroxyflavone) is a flavone, a type of flavonoid, originally isolated from the roots of Scutellaria baicalensis and Scutellaria lateriflora. It is also reported in Oroxylum indicum (Indian trumpetflower) and Thyme. It is the aglycone of baicalin.

Baicalein, along with its analogue baicalin, is a positive allosteric modulator of the benzodiazepine site and/or a non-benzodiazepine site of the GABA_{A} receptor. Baicalein is also an antagonist of the estrogen receptor, or an antiestrogen. It has been shown to inhibit also certain types of lipoxygenases and act as an anti-inflammatory agent. Baicalein is an inhibitor of CYP2C9, an enzyme of the cytochrome P450 system that metabolizes drugs in the body.

Baicalein has the following chemical structure, as denoted by Formula VIII:

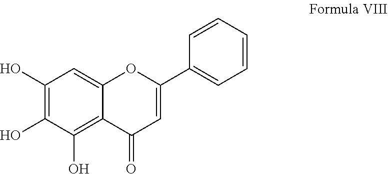 [SMILES: O=c1cc(-c2ccccc2)oc2cc(O)c(O)c(O)c12]

The systematic (IUPAC) name of Baicalein is 5,6,7-Trihydroxy-2-phenyl-chromen-4-one (C₁₅H₁₀O₅; CAS number: 491-67-8). The molecular weight of Baicalein is 270.240 gram/mol. “Baicalein” further encompass pharmaceutically acceptable salts of baicalein and amorphous and crystalline states of baicalein or of a salt thereof, including any polymorph thereof. Baicalein preparations are formulated for oral, buccal, injectable and transdermal administration.

It should be therefore understood that the term “baicalein” as used by the invention encompasses any form, brand, derivative, enantiomer, metabolite of the compound of Formula VIII, or any mixture thereof.

A further aspect of the invention relates to a method for treating, preventing, ameliorating, reducing or delaying the onset of ADA deficiency in a mammalian subject. In more specific embodiments the method of the invention may comprise the step of administering to said subject a therapeutic effective amount of 1,2,4-Trihydroxyanthraquinone (Purpurin) or any derivatives, analogs, salts and esters thereof, or any composition comprising the same.

Still further, the invention further provides the 1,2,4-Trihydroxyanthraquinone (Purpurin) or any derivatives, analogs, salts and esters thereof for use in a method of treating, preventing, ameliorating, reducing or delaying the onset of ADA deficiency in a mammalian subject.

In yet some further embodiments, the invention relates to a method for treating, preventing, ameliorating, reducing or delaying the onset of ADA deficiency in a mammalian subject. In more specific embodiments the method of the invention may comprise the step of administering to said subject a therapeutic effective amount of 1,2-dihydroxyanthraquinone (alizarin) or any derivatives, analogs, salts and esters thereof, or any composition comprising the same.

Still further, the invention further provides the 1,2-dihydroxyanthraquinone (alizarin) or any derivatives, analogs, salts and esters thereof for use in a method of treating, preventing, ameliorating, reducing or delaying the onset of ADA deficiency in a mammalian subject.

Still further, the invention relates to a method for treating, preventing, ameliorating, reducing or delaying the onset of ADA deficiency in a mammalian subject. In more specific embodiments the method of the invention may comprise the step of administering to said subject a therapeutic effective amount of 1,2,5,8-tetrahydroxyanthraquinone (Quinalizarin) or any derivatives, analogs, salts and esters thereof, or any composition comprising the same.

Still further, the invention further provides the 1,2,5,8-tetrahydroxyanthraquinone (Quinalizarin) or any derivatives, analogs, salts and esters thereof for use in a method of treating, preventing, ameliorating, reducing or delaying the onset of ADA deficiency in a mammalian subject.

The invention further provides a method for treating, preventing, ameliorating, reducing or delaying the onset of ADA deficiency in a mammalian subject. In more specific embodiments the method of the invention may comprise the step of administering to said subject a therapeutic effective amount of 5,6,7-trihydroxyflavone (baicalein) or any derivatives, analogs, salts and esters thereof, or any composition comprising the same.

Still further, the invention further provides the 5,6,7-trihydroxyflavone (baicalein) or any derivatives, analogs, salts and esters thereof for use in a method of treating, preventing, ameliorating, reducing or delaying the onset of ADA deficiency in a mammalian subject.

A further aspect of the invention relates to a method for treating, preventing, ameliorating. reducing or delaying the onset of Phenylketonuria (PKU) in a mammalian subject. In more specific embodiments the method of the invention may comprise the step of administering to said subject a therapeutic effective amount of 5,6,7-trihydroxyflavone (baicalein) or any derivatives, analogs, salts and esters thereof. or any composition comprising the same.

Still further, the invention further provides the 5,6,7-trihydroxyflavone (baicalein) or any derivatives, analogs, salts and esters thereof for use in a method of treating, preventing, ameliorating, reducing or delaying the onset of Phenylketonuria (PKU) in a mammalian subject. As indicated herein, the therapeutic compounds identified using the systems and methods of the invention, for example, purpurin, alizarin, Quinalizarin and baicalein, or any combinations thereof may be formulated in a pharmaceutical composition. Suitable preparations, e.g., substantially pure preparations. of an active compound. e.g., an active compound identified as described herein, may be combined with one or more pharmaceutically acceptable carriers or excipients, etc., to produce an appropriate pharmaceutical composition. The term “pharmaceutically acceptable carrier or excipient” refers to a carrier (which term encompasses carriers, media, diluents, solvents, vehicles. etc.) or excipient which does not significantly interfere with the biological activity or effectiveness of the active ingredient(s) of a composition and which is not excessively toxic to the host at the concentrations at which it is used or administered. Other pharmaceutically acceptable ingredients can be present in the composition as well. Suitable substances and their use for the formulation of pharmaceutically active compounds is well-known in the art (see, for example, “Remington's Pharmaceutical Sciences”, E. W. Martin, 19th Ed., 1995, Mack Publishing Co.: Easton, Pa., and more recent editions or versions thereof, such as Remington: The Science and Practice of Pharmacy. 21st Edition. Philadelphia, Pa. Lippincott Williams & Wilkins, 2005, for additional discussion of pharmaceutically acceptable substances and methods of preparing pharmaceutical compositions of various types).

A pharmaceutical composition is typically formulated to be compatible with its intended route of administration. For example, preparations for parenteral administration include sterile aqueous or non-aqueous solutions, suspensions, and emulsions. Aqueous carriers include water, alcoholic/aqueous solutions, emulsions or suspensions, including saline and buffered media, e.g., sodium chloride solution, Ringer's dextrose, dextrose and sodium chloride, lactated Ringer's. Examples of non-aqueous solvents are propylene glycol, polyethylene glycol, vegetable oils such as olive oil, and injectable organic esters such as ethyl oleate. fixed oils, polyethylene glycols, glycerine, propylene glycol or other synthetic solvents; preservatives. e.g., antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. Such parenteral preparations can be enclosed in ampoules, disposable syringes or multiple dose vials made of glass or plastic. Pharmaceutical compositions and agents for use in such compositions may be manufactured under conditions that meet standards or criteria prescribed by a regulatory agency such as the US FDA (or similar agency in another jurisdiction) having authority over the manufacturing, sale, and/or use of therapeutic compounds. For example, such compositions and compounds may be manufactured according to Good Manufacturing Practices (GMP) and/or subjected to quality control procedures appropriate for pharmaceutical agents to be administered to humans.

For oral administration, compounds can be formulated by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Suitable excipients for oral dosage forms are, e.g., fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl cellulose, sodium carboxymethylcellulose, and/or polyvinylpyrrolidone (PVP). If desired, disintegrating agents may be added, such as the cross linked polyvinyl pyrrolidone, agar, or alginic acid or a salt thereof such as sodium alginate. Optionally the oral formulations may also be formulated in saline or buffers for neutralizing internal acid conditions or may be administered without any carriers. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push fit capsules made of gelatin. as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art.

Formulations for oral delivery may incorporate agents to improve stability in the gastrointestinal tract and/or to enhance absorption.

For administration by inhalation, pharmaceutical compositions may be delivered in the form of an aerosol spray from a pressured container or dispenser which contains a suitable propellant, e.g., a gas such as carbon dioxide, a fluorocarbon, or a nebulizer. Liquid or dry aerosol (e.g., dry powders, large porous particles, etc.) can be used. The disclosure contemplates delivery of compositions using a nasal spray or other forms of nasal administration. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers. For topical applications, pharmaceutical compositions may be formulated in a suitable ointment, lotion, gel, or cream containing the active components suspended or dissolved in one or more pharmaceutically acceptable carriers suitable for use in such composition.

For local delivery to the eye, pharmaceutical compositions may be formulated as solutions or micronized suspensions in isotonic, pH adjusted sterile saline, e.g., for use in eye drops, or in an ointment. In some embodiments intraocular administration is used. Routes of intraocular administration include, e.g., intravitreal injection. retrobulbar injection, peribulbar injection, subretinal, sub-Tenon injection, and subconjunctival injection. Pharmaceutical compositions may be formulated for transmucosal or transdermal delivery. For transmucosal or transdermal administration, penetrants appropriate to the barrier to be permeated may be used in the formulation. Such penetrants are generally known in the art. Pharmaceutical compositions may be formulated as suppositories (e.g., with conventional suppository bases such as cocoa butter and other glycerides) or as retention enemas for rectal delivery. In some embodiments, a pharmaceutical composition includes one or more compounds intended to protect the active agent(s) against rapid elimination from the body, such as a controlled release formulation, implants (e.g., macroscopic implants such as discs, wafers, etc.), microencapsulated delivery system, etc.

Compounds may be encapsulated or incorporated into particles, e.g., microparticles or nanoparticles. Biocompatible polymers, e.g., biodegradable biocompatible polymers, can be used, e.g., in the controlled release formulations, implants, or particles. A polymer may be a naturally occurring or artificial polymer. Depending on the particular polymer, it may be synthesized or obtained from naturally occurring sources. A compound may be released from a polymer by diffusion, degradation or erosion of the polymer matrix, or combinations thereof. A polymer or combination of polymers, or delivery format (e.g., particles, macroscopic implant) may be selected based at least in part on the time period over which release of an agent is desired. A time period may range, e.g., from a few hours (e.g., 3-6 hours) to a year or more. In some embodiments a time period ranges from 1-2 weeks up to 3-6 months, or between 6-12 months. After such time period release of the agent may be undetectable or may be below therapeutically useful or desired levels. A polymer may be a homopolymer, copolymer (including block copolymers). straight, branched-chain, or cross-linked. Various polymers of use in drug delivery are described in Jones, D., Pharmaceutical Applications of Polymers for Drug Delivery, ISBN 1-85957479-3, ChemTec Publishing, 2004. Useful polymers include, but are not limited to, poly-lactic acid (PLA), poly-glycolic acid (PGA), poly-lactide-co-glycolide (PLGA), poly(phosphazine), poly (phosphate ester), polycaprolactones, polyanhydrides, ethylene vinyl acetate, polyorthoesters, polyethers, and poly (beta amino esters). Other polymers useful in various embodiments include polyamides, polyalkylenes, polyalkylene glycols, polyalkylene oxides. polyalkylene terepthalates, polyvinyl alcohols, polyvinyl ethers, polyvinyl esters. poly-vinyl halides, polyvinylpyrrolidone, polyglycolides, polysiloxanes, polyurethanes and co-polymers thereof, poly(methyl methacrylate). poly(ethyl methacrylate), poly(butylmethacrylate). poly(isobutyl methacrylate). poly(hexylmethacrylate), poly(isodecyl methacrylate), poly(lauryl methacrylate), poly(phenyl methacrylatepoly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene, polyvinylpyrrolidone, poly(butyric acid), poly(valeric acid), and poly(lactide-cocaprolactone). Peptides, polypeptides, proteins such as collagen or albumin, polysaccharides such as sucrose, chitosan, dextran, alginate, hyaluronic acid (or derivatives of any of these) and dendrimers are of use in certain embodiments. Methods for preparation of such will be apparent to those skilled in the art. Additional polymers include cellulose derivatives such as, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxymethylcellulose. carboxylethyl cellulose, cellulose triacetate, cellulose sulphate sodium salt, polycarbamates or polyureas, cross-linked poly(vinyl acetate) and the like, ethylene-vinyl ester copolymers such as ethylene-vinyl acetate (EVA) copolymer, ethylene-vinyl hexanoate copolymer, ethylene-vinyl propionate copolymer, ethylene-vinyl butyrate copolymer, ethylene-vinyl pentantoate copolymer, ethylene-vinyl trimethyl acetate copolymer, ethylene-vinyl diethyl acetate copolymer, ethylene-vinyl 3-methyl butanoate copolymer, ethylene-vinyl 3-3-dimethyl butanoate copolymer, and ethylene-vinyl benzoate copolymer, or mixtures thereof. Chemical derivatives of the afore-mentioned polymers, e.g., substitutions, additions of chemical groups, for example, alkyl, alkylene, hydroxylations, oxidations, and other modifications routinely made by those skilled in the art can be used. A particle, implant, or formulation may be composed of a single polymer or multiple polymers. A particle or implant may be homogeneous or non-homogeneous in composition. In some embodiments a particle comprises a core and at least one shell or coating layer, wherein, in some embodiments, the composition of the core differs from that of the shell or coating layer. A therapeutic compound may be physically associated with a particle, formulation. or implant in a variety of different ways. For example, compounds may be encapsulated. attached to a surface, dispersed homogeneously or nonhomogeneously in a matrix, etc. Methods for preparation of such formulations, implants, or particles will be apparent to those skilled in the art. Liposomes or other lipid-containing particles can be used as pharmaceutically acceptable carriers in certain embodiments. In some embodiments a controlled release formulation, implant, or particles may be introduced or positioned within a specific tissue, near said tissue or its blood supply. etc. Microparticles and nanoparticles can have a range of dimensions. In some embodiments a microparticle has a diameter between 100 nm and 100 μm. In some embodiments a microparticle has a diameter between 100 nm and 1 μm, between 1 μm and 20 μm, or between 1 μm and 10 μm. In some embodiments a microparticle has a diameter between 100 nm and 250 nm, between 250 nm and 500 nm. between 500 nm and 750 nm. or between 750 nm and 1 μm. In some embodiments a nanoparticle has a diameter between 10 nm and 100 nm, e.g., between 10 nm and 20 nm. between 20 nm and 50 nm, or between 50 nm and 100 nm. In some embodiments particles are substantially uniform in size or shape. In some embodiments particles are substantially spherical. In some embodiments a particle population has an average diameter falling within any of the afore-mentioned size ranges. In some embodiments a particle population consists of between about 20% and about 100% particles falling within any of the afore-mentioned size ranges or a subrange thereof. e.g. about 40%, 40%, 50%, 60%, 70%, 80%, 90%, 95%, etc. In the case of non-spherical particles, the longest straight dimension between two points on the surface of the particle rather than the diameter may be used as a measure of particle size. Such dimension may have any of the length ranges mentioned above. In some embodiments a particle comprises a detectable label or detection reagent or has a detectable label or detection reagent attached thereto. In some embodiments a particle is magnetic, e.g., to facilitate removal or separation of the particle from a composition that comprises the particle and one or more additional components.

Forms of polymeric matrix that may contain and/or be used to deliver a therapeutic compound include films, coatings, gels (e.g., hydrogels), which may be implanted or applied to an implant or indwelling device such as a stent or catheter. In general, the size, shape, and/or composition of a polymeric material, matrix, or formulation may be appropriately selected to result in release in therapeutically useful amounts over a useful time period, in the tissue into the polymeric material, matrix, or formulation is implanted or administered.

In some embodiments, a pharmaceutically acceptable salt, ester, salt of such ester, active metabolite, prodrug, or any adduct or derivative of a compound or an analog thereof which upon administration to a subject in need thereof is capable of providing the compound, directly or indirectly, may be used. The term “pharmaceutically acceptable salt” refers to those salts which are, within the scope of sound medical judgment, suitable for use in contact with the tissues of humans and/or lower animals without undue toxicity, irritation, allergic response and the like, and which are commensurate with a reasonable benefit/risk ratio. A wide variety of appropriate pharmaceutically acceptable salts are well known in the art.

A therapeutically effective dose of an active compound in a pharmaceutical composition may be within a range of about 1 μg/kg to about 500 mg/kg body weight, about 0.001 mg/kg to about 100 mg/kg, about 0.001 mg/kg to about 10 mg/kg, about 0.01 mg/kg to about 25 mg/kg, about 0.1 mg/kg to about 20 mg/kg body weight, about 1 mg/kg to about 10 mg/kg, about 1 mg/kg to about 3 mg/kg, about 3 mg/kg to about 5 mg/kg, about 5 mg/kg to about 10 mg/kg. In some embodiments doses of compounds may range, e.g., from about 10 μg to about 10,000 mg, e.g., from about 100 μg to about 5,000 mg, e.g., from about 0.1 mg to about 1000 mg once or more per day, week, month, or other time interval, in various embodiments. In some embodiments a single dose is administered while in other embodiments multiple doses are administered. Those of ordinary skill in the art will appreciate that appropriate doses in any particular circumstance depend upon the potency of the compound(s) utilized, and may optionally be tailored to the particular recipient. The specific dose level for a subject may depend upon a variety of factors including the activity of the specific compound (s) employed, severity of the disease or disorder, the age. body weight, general health of the subject, etc.

In certain embodiments a therapeutic compound may be used at the maximum tolerated dose or a sub-therapeutic dose or any dose there between, e.g., the lowest dose effective to achieve a therapeutic effect. Maximum tolerated dose (MTD) refers to the highest dose of a pharmacological or radiological treatment that can be administered without unacceptable toxicity, that is, the highest dose that has an acceptable risk/benefit ratio, according to sound medical judgment. In general, the ordinarily skilled practitioner can select a dose that has a reasonable risk/benefit ratio according to sound medical judgment. A MTD may, for example, be established in a population of subjects in a clinical trial. In certain embodiments a compound is administered in an amount that is lower than the MTD, e.g., the compound is administered in an amount that is about 10%, 20%, 30%, 40%, 50%, 60%, 70%, 80%, or 90% of the MTD.

It may be desirable to formulate pharmaceutical compositions, particularly those for oral or parenteral compositions, in unit dosage form for ease of administration and uniformity of dosage. Unit dosage form, as that term is used herein, refers to physically discrete units suited as unitary dosages for the subject to be treated; each unit containing a predetermined quantity of active compound(s) calculated to produce the desired therapeutic effect in association with an appropriate pharmaceutically acceptable carrier.

It will be understood that a therapeutic regimen may include administration of multiple unit dosage forms over a period of time. In some embodiments, a subject is treated for between 1-7 days. In some embodiments a subject is treated for between 7-14 days. In some embodiments a subject is treated for between 14-28 days. In other embodiments, a longer course of therapy is administered, e.g., over between about 4 and about 10 weeks. In some embodiments multiple courses of therapy are administered. In some embodiments, treatment may be continued indefinitely. For example, a subject suffering from an IEM disorder may continue to be treated indefinitely: a subject at risk of developing a IEM disease may be treated for any period during which such risk exists, e.g., indefinitely. A subject may receive one or more doses a day, or may receive doses every other day or less frequently, within a treatment period. Treatment courses may be intermittent.

In some embodiments, a compound is provided in a pharmaceutical pack or kit comprising one or more containers (e.g., vials, ampoules, bottles) containing the active compound and, optionally, one or more other pharmaceutically acceptable ingredients. Optionally associated with such container(s) can be a notice in the form prescribed by a governmental agency regulating the manufacture, use or sale of pharmaceutical products, which notice reflects approval by the agency of manufacture, use or sale for human administration. The notice may describe, e.g., doses, routes and/or methods of administration, approved indications (e.g., IEM disorder that the compound or pharmaceutical composition has been approved for use in treating), mechanism of action, or other information of use to a medical practitioner and/or patient. Different ingredients may be supplied in solid (e.g., lyophilized) or liquid form. Each ingredient will generally be suitable as aliquoted in its respective container or provided in a concentrated form. Kits may also include media for the reconstitution of lyophilized ingredients. The individual containers of the kit are preferably maintained in close confinement for commercial sale. One of ordinary skill in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

All scientific and technical terms used herein have meanings commonly used in the art unless otherwise specified. The definitions provided herein are to facilitate understanding of certain terms used frequently herein and are not meant to limit the scope of the present disclosure.

Before specific aspects and embodiments of the invention are described in detail, it is to be understood that this invention is not limited to particular methods. and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, references to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. More specifically, the terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method.

The term “about” as used herein indicates values that may deviate up to 1%, more specifically 5%. more specifically 10%, more specifically 15%, and in some cases up to 20% higher or lower than the value referred to, the deviation range including integer values, and, if applicable, non-integer values as well, constituting a continuous range. As used herein the term “about” refers to f 10%. It should be noted that various embodiments of this invention may be presented in a range format. It should be understood that the description in range format is merely for convenience and brevity and should not be construed as an inflexible limitation on the scope of the invention. Accordingly. the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

The examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments. unless the embodiment is inoperative without those elements.

Various embodiments and aspects of the present invention as delineated hereinabove and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims. the singular forms “a”, “an” and “the” include plural referents unless the content clearly dictates otherwise.

### EXAMPLES

### Experimental Procedures

### Strains and Culture:

Yeast strains and plasmids used in this study are listed in Table 2 and 3 respectively. Strains were cultured in synthetic defined (SD) media consisting a defined mixture of amino acids and nucleobases. Adenine (adenine hemisulfate salt, Sigma-Aldrich) was supplemented in the indicated concentrations.

TABLE 2
list of strains

| Yeast Strains | Genetic background | Source |
|---|---|---|
| Wild-type, BY4741 | MATa, his3-Δ1, leu2Δ-0, met15- | [21] |
| | Δ0, ura3-Δ0 | |
| apt1Δ | MATa, his3-Δ1, leu2Δ-0, met15- | [21] |
| | Δ0, ura3-Δ0, apt1::hphMX | |
| aah1Δ | MATa, his3-Δ1, leu2Δ-0, met15- | [21] |
| | Δ0, ura3-Δ0, aah1::KanMX | |
| aah1Δapt1Δ | MATa, his3-Δ1, leu2Δ-0, met15- | This |
| | Δ0, ura3-Δ0, aah1:: KanMX, | application |
| | apt1::hphMX | |
| W303 | MATa, ade2-1, trp1-1, can1-100, | [21] |
| | leu2-3 his3-11,15, ura3-1 | |
| 74-D694, strong | ade 1-14 (UGA), trp1-289 (UAG), | [22] |
| [PSI⁺] [pin−] | leu2-3,112, his3Δ −200, ura3-52, | |
| | strong [PSI⁺] [pin⁻] variant | |
| 74-D694, weak | ade 1-14 (UGA), trp1-289 (UAG), | |
| [PSI⁺] [pin−] | leu2-3,112 , his3Δ −200, ura3-52, | |
| | weak [PSI⁺] [pin⁻] variant | |
| hsp104Δ | MATa, his3-Δ1, leu2Δ-0, met15- | This |
| | Δ0, ura3-Δ0, hsp104:: NAT | application |
| aah1Δapt1Δhsp104Δ | MATa, his3-Δ1 , leu2Δ-0, met15- | This |
| | Δ0, ura3-Δ0, hsp104:: NAT, | application |
| | aah1:: KanMX, apt1::hphMX | |
| Wild-type, pRS313 | MATa, his3-Δ1, leu2Δ-0, met15- | This |
| pRS416 | Δ0, ura3-Δ0, pRS313, pRS416 | application |
| aah1Δapt1Δ, | MATa, his3-Δ1, leu2Δ-0, met15- | This |
| pRS313 | Δ0, ura3-Δ0, aah1:: KanMX, | application |
| pRS416 | apt1::hphMX, pRS313, pRS416 | This |
| aah1Δapt1Δ, | MATa, his3-Δ1, leu2Δ-0, met15- | application |
| pRS313 | Δ0, ura3-Δ0, aah1:: KanMX, | |
| pRS416aah1 | apt1::hphMX, pAPT1, pAAH1 | |
| Wild-type, | MATa, his3-Δ1, leu2Δ-0, met15- | This |
| Hsp104-mCherry | Δ0, ura3-Δ0, pAG415-Hsp104- | application |
| | mCherry | |
| aah1Δapt1Δ4 | MATa, his3-Δ1, leu2Δ-0, met15- | This |
| Hsp104-mCherry | Δ0, ura3-Δ0, aah1:: KanMX, | application |
| | apt1::hphMX, pAG415-Hsp104- | |
| | mCherry | |

TABLE 3
list of plasmids

| Plasmid name | Description | Source |
|---|---|---|
| pUC57-AAH1 | pUC57 derivative containing a | GenScript |
| | denovo synthesis of AAH1 gene | |
| pUC57-APT1 | pUC57 derivative containing a de | GenScript |
| | novo synthesis of APT1 gene | |
| pRS313 | Yeast centromere vector with a | [21] |
| | HIS3 marker | |
| pAPT1 | [pRS313] APT1 | This application |
| pRS416 | Yeast centromere vector with a | [21] |
| | URA3 marker | |
| pAAH1 | [pRS416] AAH1 | This application |
| pRS415 | Yeast centromere vector with a | [21] |
| | LEU2 marker | |
| pAG415-Hsp104- | pAG415GPD-Hsp104-mCherry | [23] |
| mCherry | | |

### Construction of Aah1ΔApt1Δ Strain:

The kanMX6 cassette in the apt1::kanMX6 strain was replaced by a PCR product of the HygromycinR cassette using U2/D2 oligonucleotides that contain a homology sequence for homologous recombination. Cells were grown in rich medium (YPD) for 24 hours before plating on YPD plates containing Hygromycin (200 mg/L). The double mutant strain apt1::hphMX6 aah1::kanMX6 was generated by transforming a PCR product of the KanMX cassette with 5′ and 3′ flanking sequences of AAH1 into the apt1::hphMX6 strain. Cells were grown in YPD for ˜24 hours before plating on YPD plates containing G418 (200 mg/L). Genetic disruptions were confirmed by PCR using an oligonucleotide upstream of the deletion and a reverse oligonucleotide within the HygR or the KanMX gene.

### Yeast Growth Assays:

Strains were grown over night at 30° C. in SD medium without adenine. For spotting assays, strains were diluted to 6.25*10⁷ cells/mL and were then 5-fold serially diluted and spotted on SD media with the indicated concentrations of adenine or TA (Sigma-Aldrich). Plates were incubated at 30° C. for 2 days. The results displayed are representative of three biological experiments. For OD₆₀₀ measurements, strains were diluted to OD₆₀₀ 0.01, 200 μL of cells were platted on 96 wells plates and incubated at 30° C. for 25 hours with continuous shaking. OD₆₀₀ was measured using Tecan™ SPARK 10M plate reader. The results displayed are representative of three biological experiments performed in triplicate.

For the dose-response curve of WT and aah1Δapt1Δ cell growth (FIG. 1D), strains were cultured in SD media containing different concentrations of adenine (from 0.03 μg/L to 40 mg/L) and OD₆₀₀ measurements were performed when the cells reached log phase. The results displayed are representative of three biological experiments. Results were fitted to a standard logistic four-parameter equation using the OriginLab software. The equation used was.

$$y = d + \frac{a - d}{( 1 + ( \frac{X}{{c}^{b}} )}$$

where y is the OD₆₀₀ value, d is the maximum OD₆₀₀ value, a is the minimum OD₆₀₀ value, x is adenine concentration (μg), c is the point of inflection and b is the slope factor of the curve.

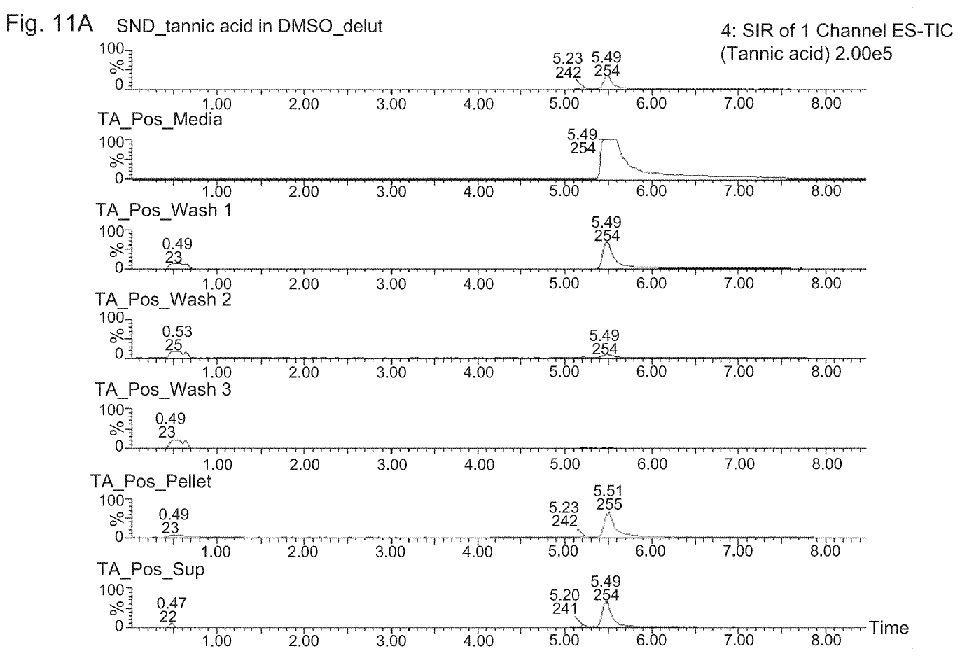

For the percentage of growth analysis (FIG. 10B and FIG. 13A), OD₆₀₀ was measured when cells reached log phase. The percentage of growth represents the growth with TA compared to the growth without TA. The results displayed are representative of three biological experiments performed in triplicate.

### GC-MS:

Metabolites were extracted as previously described (Tu, B. P. et al. Proc. Natl. Acad. Sci. U.S.A. 104, 16886-16891 (2007)). Briefly, 4 mL of 60% methanol/10 mM Tricine (pH 7.4) that was maintained at −20° C. were added to 2 mL of logarithmic cells. The number of cells was counted using a hemocytometer. Cells were incubated for 5 min at −20° C., centrifuged at 1,000 g for 3 min at 4° C., washed with 1 mL of the same buffer and resuspended in 1 mL of 75% ethanol/0.5 mM Tricine (pH 7.4). Intracellular metabolites were extracted by incubating at 80° C. for 3 min and at 4° for 5 min. Samples were centrifuged at 20,000 g for 1 min, and 0.9 mL of the supernatant was transferred to a new tube, centrifuged again for 10 min and 0.8 mL was transferred to a new tube. Samples were stored at −80° C. until analysis. For each strain and/or condition, 7 samples were extracted: 3 samples for measurement and 4 samples for spiking adenine standard. Matrix-matched standard curves for each cell pool were constructed. Stock solution of adenine (1 mg/mL in 1 M HCl) was sequentially diluted 10-, 100-, and 1000-fold into solutions that were spiked to 4 samples of the same group of extracts plus one blank in amounts providing final exogenous adenine concentrations of 1, 5, 10 and 50 μg/mL after derivatization. All samples were evaporated in N2-flow and then lyophilized for 2 hrs. Each sample was derivatized by adding 30 μL of MSTFA (Restek, #35600) and shaking at 60′C for 1 hr. The mixtures were transferred to 2 mL autosampler glass vials with 100 μL glass inserts. Acquisition was performed in GC-MS system comprising an Agilent 7890A gas chromatograph and LECO Pegasus HT Time-of-Flight Mass Spectrometer (MassBank Record: OUF00096) using a Rxi-5Sil MS (Restek) column. Samples were analyzed using the splitless mode; the temperature of injector was set to 230° C. and the transfer line to 250° C. The injected analytes were separated using the following chromatographic conditions: 1 mL/min of helium as a carrier gas was held at 8(PC for 2 min, ramped to 33(PC at 15° C./min and then held at this temperature for another 6 min. For mass-spectrometry, electron impact mode with ionization voltage was 70 eV, mass range 85-500 m/z and acquisition rate of 20 spectra per second. The ion source chamber was set to 20(° C., and the detector voltage was 1650V. LECO ChromaTOF software was used for acquisition control and data processing with 264 m/z at 722-sec peak for quantitation, with the limit of quantitation being 0.01 μg/mL. Adenine concentration was normalized to the number of cells per each sample and divided by 108 cells. The results displayed are representative of three biological experiments performed in triplicate. Values are the mean±s.d. of three independent experiments.

### Flow Cytometry:

An amount of 1 mL of logarithmic cells were washed with PBS buffer and sonicated using 15 s pulses at 20% power. For each sample, 2*10⁶ cells were resuspended with ProteoStat dye (Enzo Life Sciences) diluted 1:3000 in ProteoStat assay buffer. Cells were incubated for 15 min at room temperature protected from light. Flow cytometry was preformed using Stratedigm S1000EXi and the CellCapTure software (Stratedigm, San Jose, Calif.). Live cells were gated (P1) by forward scatter and side scatter. Fluorescence channels for FITC (530/30) and PE-Cy5 (676/29) were used utilizing a 488 nm laser source. A total of 50,000 events were acquired for each sample. Analyses were performed using FlowJo software (TreeStar, version 10). The results displayed are representative of three biological experiments performed in triplicate.

### Confocal Microscopy:

An amount of 1 mL of logarithmic cells were washed with PBS buffer, sonicated using 15 s pulses at 20% power and resuspended in 50 μL of ProtesoStat dye diluted 1:250 in ProteoStat assay buffer. Cells were incubated for 15 min at room temperature protected from light. 10 μL of each sample was deposited on poly-lysine coated glass slides (Sigma-Aldrich). Cells were imaged using Leica TCS SP8 laser confocal microscope with 63×1.4 NA or 100*1.4 NA oil objectives. To visualize the nuclei, Hoechst (33342; ImmunoChimestry) was used. An argon laser with 488 excitation line was used for ProteoStat (emission wavelength, 500 to 600 nm), and a 405 UV laser was used to excite Hoechst fluorescence (emission wavelength, 415 to 450 nm). The results displayed are representative of three biological experiments.

### Confocal Raman Microscopy:

All Raman spectroscopic and imaging experiments were performed with a laboratory-made confocal Raman microscope, as previously reported (Noothalapati, H, et al. Sci. Rep. 6, 27789 (2016)). A He-Ne laser with an output wavelength of 632.8 nm was used for excitation. Laser power of 5 mW was used at the sample point with an exposure of 0.5 s per point. For Raman imaging experiments, a step size of 0.6 μm was used. The results displayed are representative of three biological experiments.

### Antibody Formation:

Adenine assemblies were formed as previously described for antibody formation of tyrosine fibrils (Zaguri, D., et al. Molecules, 23, 1273 (2018)). Briefly, adenine at a final concentration of 8 mg/ml was dissolved in PBS at 90° C., followed by overnight gradual cooling. These structures served as antigens in a series of seven rabbit immunization cycles followed by purification using protein G column.

### Indirect Immunofluorescence:

Indirect immunofluorescence was performed as previously described (Schmelzle, T., et al. Mol. Cell. Biol. 24, 338-351 (2004)). An amount of 8 ml of yeast cells at the logarithmic phase were fixed for 2 h with formaldehyde (3.7% final) and potassium phosphate buffer (100 mM final, pH 6.5), washed and resuspended in sorbitol buffer (1.2 M sorbitol and 100 mM potassium phosphate, pH 6.5). Sorbitol buffer supplemented with DTT (10 mM) and Zymolyase 20T (12.5 mg/ml) was added for 1.5 hrs at 37° C. to digest the cell wall. The cells were then fixed on poly-L-lysine-coated glass slides and permeabilized with PBT (PBS with 1% BSA and 0.1% Triton X-100). Immunofluorescence directed against adenine amyloid-like structures was performed using the anti-ADE antibody described above at a dilution of 1:200 for 2 h followed by three washings and incubation with Cy3-conjugated anti-rabbit diluted 1:200 for an additional 1.5 hrs. DNA was stained with 4′,6-diamidino-2-phenylindole (DAPI). Cells were visualized with an Evos microscope (60× objective) and imaged using Leica TCS SP8 laser confocal microscope with 63×1.4 NA or 100×1.4 NA oil objectives.

### ThT Fluorescence Endpoint Measurements:

Adenine was dissolved at various concentrations. ranging from 1 mg/ml to 10 mg/ml at 90° C. in PBS and plated on a 96-well black plate together with 20 μm ThT in PBS (final concentration). Following an overnight incubation at room temperature. ThT emission signal at 480 nm (excitation at 450 nm) was measured using a Tecan™ Infinite® 200 PRO plate reader. The results displayed are representative of three biological experiments performed in triplicate.

### ThT Kinetic Assay of Adenine Self-Assembly Inhibition:

Adenine was dissolved to a final concentration of 8 mg/ml at 90° C. in PBS. The solution was plated on a 96-well black plate and mixed with the inhibitor baicalein at the stated concentration, and with 20 μm ThT (final concentration). Fluorescence (excitation at 450 nm, emission at 480 nm) was recorded over time using a Tecan™ Infinite® 200 PRO plate reader. Data processing was performed using the OriginLab software. The results displayed are representative of three biological experiments performed in triplicate.

### LC-MS:

100 ml of cells at the logarithmic phase were centrifuged and washed three times with PBS. 1 ml of the growth medium and of each wash were collected for LC-MS analysis. After the third wash, the pellet was resuspended in PBS together with acid-washed glass beads. Tubes were vortexed for 45 minutes in 4° C. using disruptor-genie. Cell debris and soluble material were separated by centrifugation. Both fractions as well as the medium and washes were analyzed by MS. The pellet was resuspended in 200 μl DMSO, heated for 5 minutes at 85° C. and sonicated for 5 minutes at 40° C. The lysis mixture was centrifuged at 6000 rpm for 10 minutes. 150 μl of the lysate were transferred to a Waters 96 well ACQUITY collection plate for LC-MS analysis. 20 μl of each sample were injected to Waters Autopurification system analytical module equipped with SQD2 MS detector at the following conditions: a. LC: Waters XSelect Peptide CSH C18 column (5 μm, 4.6 mm×100 mm) using a 10 minute gradient from 95:5 Water:acetonitrile (both with 0.1% formic acid) to acetonitrile (0.1% formic acid); b. MS: acquisition parameters were optimized in order to select the proper ion for selected ion monitoring (SIR) experiments. The protonated molecule of adenine [M+H]⁺ at m/z 136.1 and deprotonated molecule of tannic acid [M−H]⁻ at m/z 1700.1 were obtained at a cone voltage of 80 V. The results displayed are representative of three biological experiments.

### Plasmids Cloning:

The gap-repair cloning system was used as previously described (Ma, H., et al. Gene 58, 201-216 (1987)). to clone the pUC57-AAH1 and pUC57-APT1 vectors into pRS416 and pRS313 plasmids respectively. Candidates were confirmed by PCR using oligonucleotides upstream and downstream to the insertion site.

### High Throughput Screen Assay in Yeast:

Yeast strains were grown over night at 30° C. in SD medium without adenine and were diluted to OD₆₀₀ 0.005. 80 μL of cells were platted on 384 wells plates and incubated at 30° C. for 22 hours with continuous shaking. OD₆₀₀ was measured using Tecan™ SPARK 10M plate reader.

### IC50 Assay:

Yeast strains were diluted to OD₆₀₀ 0.005. 80 μL of cells were platted on 384 wells plates. Compounds were added with Tecan™ D300 printer at relevant concentrations and plates were incubated at 30° C. for 22 hours with continuous shaking. OD₆₀₀ was measured using Tecan™ SPARK 10M plate reader. IC50 calculations were performed with CDD software.

### IC50 Assay in Cell Lines:

24 hours after seeding in full medium cells were washed with medium without serum and placed in medium without serum. Following 72 hours of incubation with or without Adenosine (A9251 SIGMA) (200 ug/ml), cells were washed with PBS and stained for adenosine aggregates with Proteostat dye (ENZ-51023-KP002). Proteostat concentration was 1:3000 of diluted stock. Following 15 minutes' incubation, cells were washed with PBS and mean fluorescence was measured with FACS.

Cells were treated with adenosine alone or with adenosine with compounds. Compound activity was tested in 19 concentrations in range from 50 uM to 0.003 uM. While samples with adenosine alone showed high signal because of strong staining of adenosine aggregates, addition of candidate compounds inhibited the formation of these aggregates thus reducing staining.

Readouts: Proteostat Fluorescence intensity was measured with FACS at 2 excitation wavelengths: 488 nm and 550 nm. Emission wavelength was 600 nm.

TABLE 4
Plate layout (example):

| | blank | | | | blank | | | | blank | | | |
|---|---|---|---|---|---|---|---|---|---|---|---|---|
| compound | 1 | 2 | 3 | 4 | 5 | 6 | 7 | 8 | 9 | 10 | 11 | 12 |
| A | cells | cells | cells | cells | cells + | cells + | cells + | cells + | cells + | cells + | cells + | cells + |
| | | | | | A | A | A | A | 10 μM | 10 μM | 10 μM | 10 μM |
| | | | | | | | | | compound | compound | compound | compound |
| B | 50 | 50 | 50 | 50 | 41.7 | 41.7 | 41.7 | 41.7 | 34.72 | 34.72 | 34.72 | 34.72 |
| C | 28.5 | 28.5 | 28.5 | 28.5 | 24.11 | 24.11 | 24.11 | 24.11 | 20.09 | 20.09 | 20.09 | 20.09 |
| D | 16.7 | 16.7 | 16.7 | 16.7 | cells + | cells + | cells + | cells + | 11.63 | 11.63 | 11.63 | 11.63 |
| | | | | | A | A | A | A | | | | |
| E | 9.69 | 9.69 | 9.69 | 9.69 | 8.08 | 8.08 | 8.08 | 8.08 | 6.5 | 6.5 | 6.5 | 6.5 |
| F | 3.25 | 3.25 | 3.25 | 3.25 | 1.67 | 1.67 | 1.67 | 1.67 | 0.8 | 0.8 | 0.8 | 0.8 |
| G | 0.32 | 0.32 | 0.32 | 0.32 | cells + | cells + | cells + | cells + | 0.05 | 0.05 | 0.05 | 0.05 |
| | | | | | A | A | A | A | | | | |
| H | 0.02 | 0.02 | 0.02 | 0.02 | 0.008 | 0.008 | 0.008 | 0.008 | 0.003 | 0.003 | 0.003 | 0.003 |

### Aggregation Assay on Primary Lymphoblasts (LCLs):

24 hours after seeding in 96 round bottom plates in full medium cells were washed once with medium without serum and placed in medium without serum. Following 72 hours of incubation with or without Adenosine (200 ug/ml) and with or without purpurin (2 uM), cells were washed once with PBS and stained for adenosine aggregates with Proteostat dye (Enzo Life Sciences). Proteostat concentration was 1:4000 of diluted stock. Following 15 minutes incubation, cells were washed with PBS and mean flow cytometry was preformed using Stratedigm S1000EXi and the CellCapTure software (Stratedigm, San Jose, Calif.). Analyses were performed using FlowJo software (TreeStar, version 10).

### LCLs Survival Assay:

24 hours after seeding in 96 round bottom plates in full medium cells were washed once with medium without serum and placed in medium without serum. Following 72 hours of incubation with or without Adenosine (200 ug/ml) and with or without purpurin (2 uM), XTT based cell viability assay was performed. Absorbance was measured with Tecan™ SPARK 10M plate reader.

### Cell Lines and Media:

LCLs derived from B lymphocytes by transformation with EBV were obtained from Coriell Institute. Cells were grown in RPMI medium supplemented with 15% FCS, PSN antibiotics mixture and non-essential amino acids.

C. elegans Growth and Media

Wild-type N2, RB857 and NL2099 worms were obtained from the Caenorhabditis Genetics Center. RNAi clones was obtained from the Ahringer RNAi library as previously described [Fisher A L, et al., J. Biol. Chem. 283: 9127-9135(2008)]. Worms were grown on NGM media without peptone and the metabolites were added into a boiling media. Baicalein and EGCG were added to a media after cooled down to 45 degrees.

### Example 1

### In Vivo Model for Adenine Accumulation and Toxicity

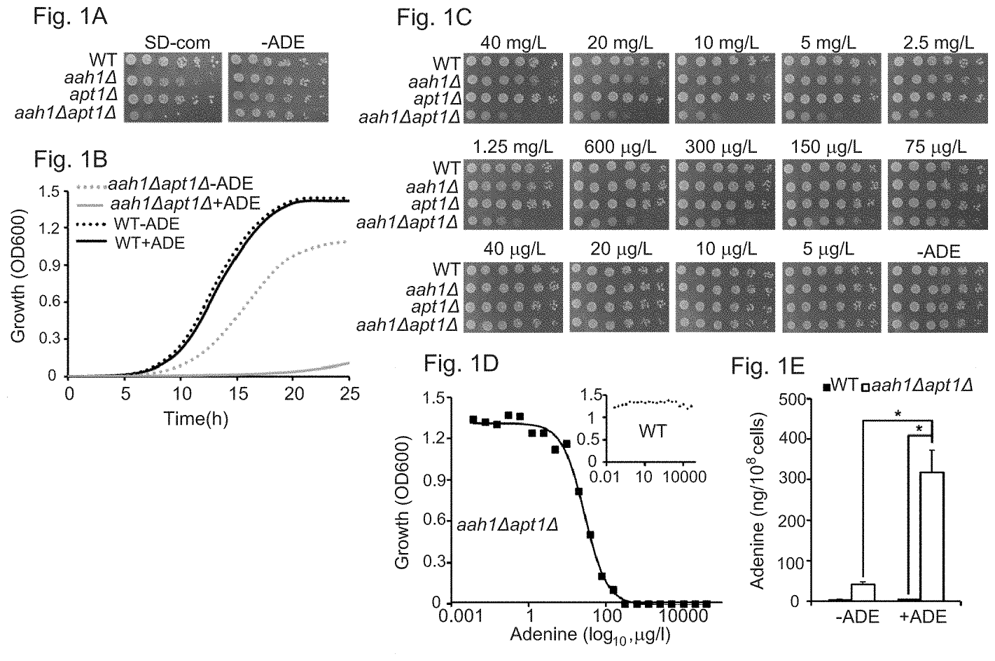

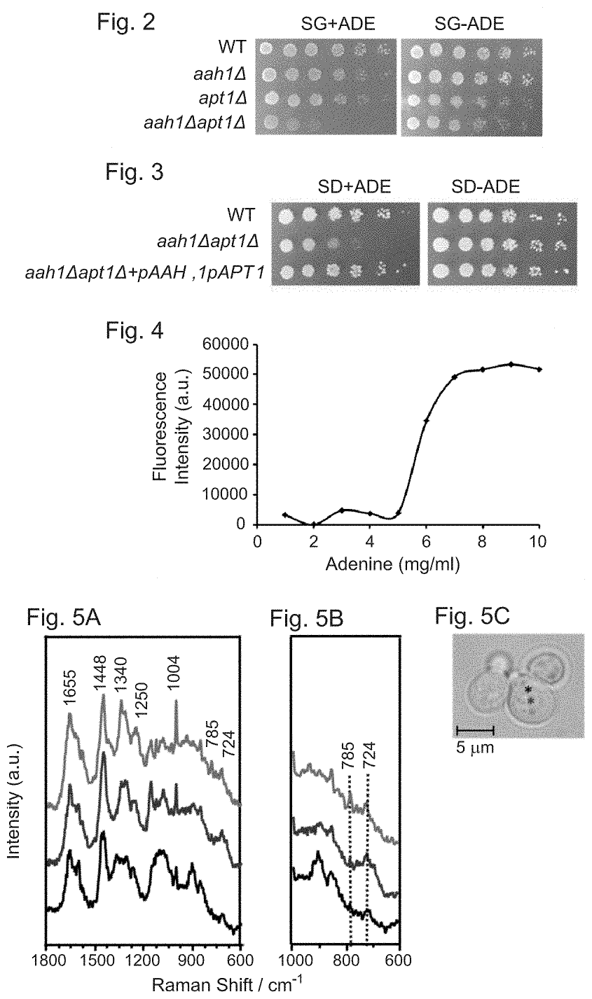

Purine biosynthesis pathways are crucial for the normal function of cells and are conserved between yeast and humans. Both the adenine phosphoribosyltransferase (APRT) and adenosine deaminase (ADA) enzymes take part in adenine salvage in humans and mutations in their encoding genes can lead to the accumulation of adenine and its derivatives. Mutation in APRT leads to the adenine phosphoribosyltransferase deficiency and mutation in ADA leads to adenosine deaminase deficiency (Valaperta, R. et al. BMC Nephrol. 15:102 (2014)). In budding yeast. the APRT and ADA orthologs (APT) and AAH1, respectively) are similarly involved in adenine salvage. Apt1 catalyzes the formation of AMP from adenine and Aah1 converts adenine to hypoxanthine. To reveal the importance of these two enzymes for cell growth, an adenine salvage mutant was generated by disruption of both APT) and AAH1 genes as described above by deleting the AAH1 gene open reading frame in Chromosome XIV from position 359596 to position 360639 and deleting of the APT1 in Chromosome XIII from position 228937. to position 229500, and replacing the open reading frames with selectable markers. The double mutant showed a slow growth phenotype on synthetic defined (SD) medium containing a specific mixture of amino acids and nucleobases (SD complete), compared to the growth of the wild-type and both single mutants (FIG. 1A-1B). Interestingly, removal of adenine from the medium dramatically improved cell growth of the metabolite salvage mutant and had no significant effect on any of the other strains (FIG. 1A-1B). The double mutant showed a slow growth phenotype also when using a minimal medium containing glycerol as a non-fermentable carbon source (FIG. 2). Thus, the reduced growth of the salvage mutant depended only on the presence of adenine, regardless of the carbon source, ruling out the possibility that the salvage mutant had lost respiratory competence due to mitochondrial mutations. To verify that the toxic effect observed for the salvage mutant is indeed due to disruption of the AAH1 and APT1 genes, single copy plasmids carrying both genes were introduced to the mutant cells by transformation. As shown in FIG. 3, the restoration of the genes indeed rescued the growth phenotype. Altogether, the presence of adenine at the normal concentration used for wild-type yeast growth leads to a significant cell growth decrease in a strain that is defective in the biosynthesis downstream to adenine. This is indeed an unusual phenomenon as in most cases (excluding mutations in transport systems) the absence of a given metabolite. rather than its presence in the physiological concentration needed for normal yeast growth. serves as a limiting factor of wildtype yeast growth. This is analogous to the toxicity of normal metabolite concentrations observed in some inborn error of metabolism disorders, such as PKU and tyrosinemia, where the RDA for the general population is actually toxic to the affected individuals, due to accumulation of the metabolites in the absence of salvage. The effect indeed appears to reflect toxicity, as increasing adenine levels in the medium led to decreased cell growth in a dose-dependent manner (FIG. 1C-1D), similar to the toxic effect previously observed in in vitro cell culture studies [3]. It should be noted that the toxic effect on cell growth due to adenine accumulation is very similar to the outcome of protein amyloid expression in yeast cells [8, 9, 10].

### Example 2

### Non-Linear Response to Adenine Feeding

The dose-response curve of cell growth as a function of adenine concentration was fitted using a four-parameter logistic equation (4PL), producing a typical sigmoidal shaped-curve, with no effect at lower concentrations and a sharp increase in the inhibitory effect upon reaching a critical concentration threshold (FIG. 1D). Thus, cell growth appears to be affected by adenine levels in a non-linear cooperative manner. This is consistent with the mechanism of nucleation-growth as observed in micelle formation (assembly above a critical micelle concentration, CMC) or the formation of amyloids by the assembly of protein monomers.

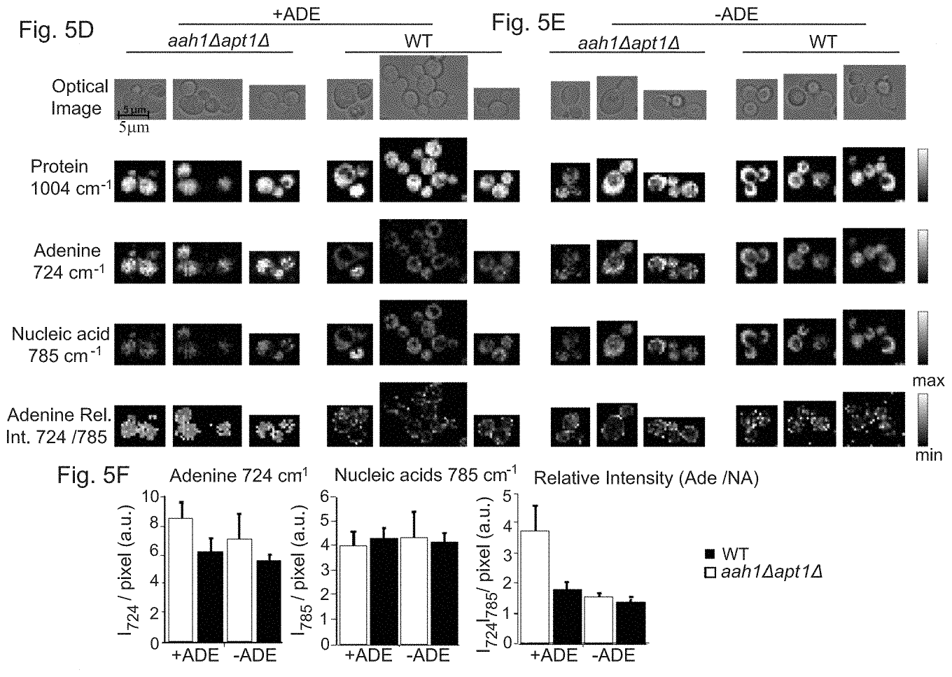

Indeed, in vitro studies also showed non-linear self-assembly of adenine at different concentrations (FIG. 4). Thus, both in vitro as well as in vivo data are presented regarding adenine accumulation at different concentrations and, for the first time, the possible correlation between the assembly mechanism of fibril formation and cellular growth. These results imply that above a critical concentration, the favorable energetic state dictates the formation of the toxic assemblies, while at lower concentrations the metabolites are most likely at the normal physiological state.

To quantify the intracellular concentrations of adenine, gas chromatography mass-spectrometry (GC-MS) was used. Analysis of the cellular adenine concentrations under different conditions indicated that on SD media in the presence of the metabolite, adenine levels were indeed significantly higher in the salvage mutant as compared to the wild-type strain (×45 fold, FIG. 1E), suggesting a clear correlation between the feeding amounts of adenine and growth inhibition. Similarly, when not following a very strict diet, inborn error of metabolism patients have 1-2 orders of magnitude higher concentrations of metabolites, as compared to the general population.

### Example 3

### In Vivo Visualization of Adenine Accumulation by Raman Imaging

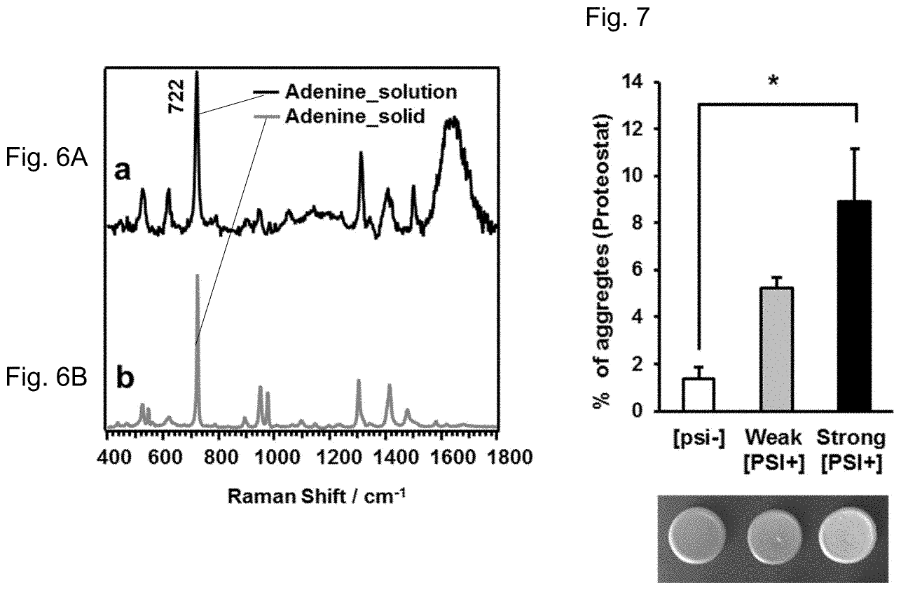

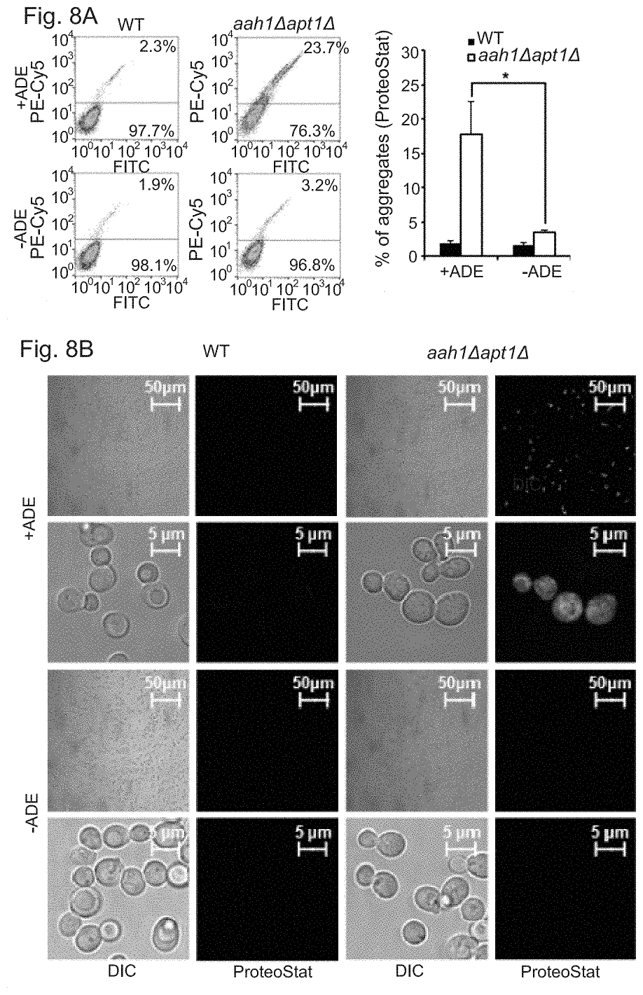

To detect the accumulation of adenine in vivo, Raman microspectroscopy was performed. Raman spectroscopy coupled with microscopy has recently emerged as a promising tool to trace intracellular processes in vivo and was successfully used in yeast to follow glucose assimilation into intracellular components (Noothalapati Venkata, H. N. & Shigeto, S. Chem. Biol. 19, 1373-1380 (2012)). Raman spectrum provides rich and highly specific chemical information, and molecular distribution within single cells can thus be visualized at a sub-μm spatial resolution. Moreover, being a vibrational spectroscopic technique, label-free imaging could be performed, as it requires no exogenous dye probe. Space-resolved Raman spectra obtained at three different points in the cytoplasm of salvage mutant single cells (aah1Δapt1Δ) in the presence of adenine showed Raman bands characteristic to proteins (FIG. 5A-5C). Major features included 1004 cm⁻¹ [phenylalanine ring breathing], 1250 cm⁻¹ [amide III], 1340 cm⁻¹ & 1448 cm¹ [C—H bend] and 1655 cm⁻¹ [amide I]. By carefully examining the region between 800-700 cm⁻¹, it was possible to observe a band at 785 cm⁻¹ corresponding to ring breathing modes of nucleobases such as uracil, cytosine, thymine and nucleic acid backbone vibration (FIG. 5B). Additionally, a band was also observed at 724 cm 1 corresponding to adenine ring breathing modes (Raman band of adenine in solid and solution forms are shown in FIGS. 6A, 6B). According to these observations, the 785 cm⁻¹ band was chosen as a marker of nucleic acids in general while 724 cm⁻¹ served as an adenine marker. As expected, the intensities of these two bands varied depending on the location in the cell. As shown in FIG. 5B, while the red and black spectra showed intense adenine marker with very low nucleic acid band (indicating high prevalence of adenine), respectively, the spectrum in green showed comparable intensities of these two bands, which is typical of nucleic acids. Thus, these bands can be used to study adenine accumulation.

In order to further investigate adenine accumulation and its distribution in living yeast cells, Raman imaging experiments were performed on both mutant and wild-type yeast in the presence (FIG. 5D) or absence (FIG. 5E) of adenine. The cytoplasm was first imaged, which usually contains high concentrations of dissolved macromolecules such as proteins, using phenylalanine ring breathing mode at 1004 cm⁻¹. No significant difference was observed, indicating that proteins were similarly distributed under all conditions. In the presence of externally added adenine, images constructed using 724 cm⁻¹ showed high intracellular abundance of adenine in the double mutants, while its distribution was very low in wild-type yeast. In the absence of externally added adenine, the intensity of intracellular adenine was comparable in both mutant and wild-type cells. To exclude the contribution of adenine from nucleic acids, the relative intensity of 724 cm⁻¹ images and nucleic acids 785 cm⁻¹ images (724/785 cm-1) were calculated, as shown in red. Relative intensity images showed adenine accumulation and the appearance of subcellular regions of high adenine concentration only in double mutant yeast cells in the presence of adenine. Average intracellular intensity of 724 cm 1 per pixel, 785 cm 1 per pixel and the relative intensity per pixel of WT and aah1Δapt1Δ cells were calculated (FIG. 5F). The results reinforce the imaging results, showing significant adenine levels only in the mutant cells. These results preclude the possibility that the adenine is dispersed inside the cell and strongly suggest the formation of adenine aggregates in the salvage mutant in the presence of adenine.

### Example 4

### In Vivo Formation of Amyloid-Like Assemblies Upon Adenine Accumulation

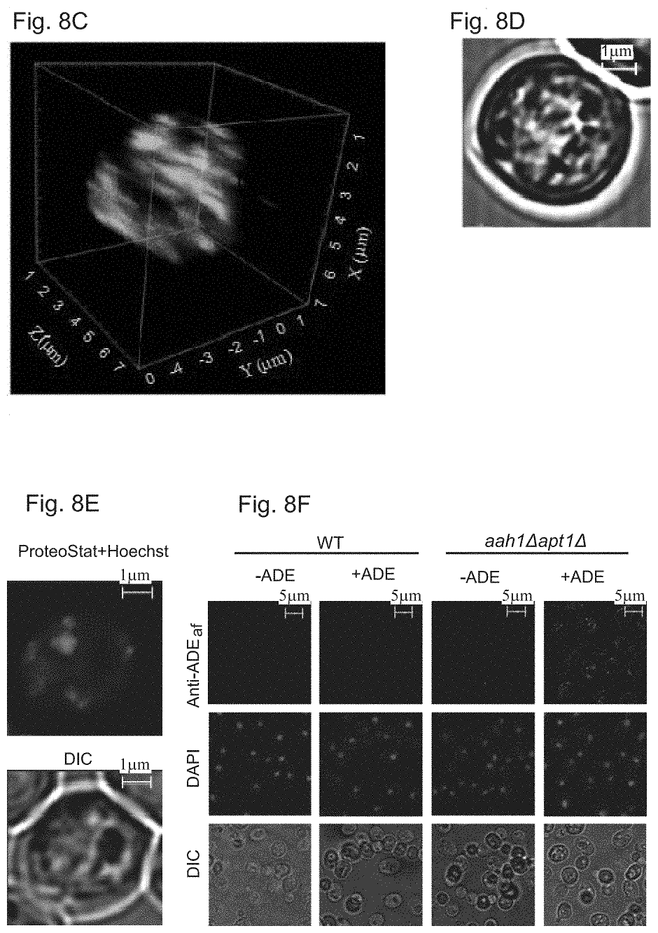

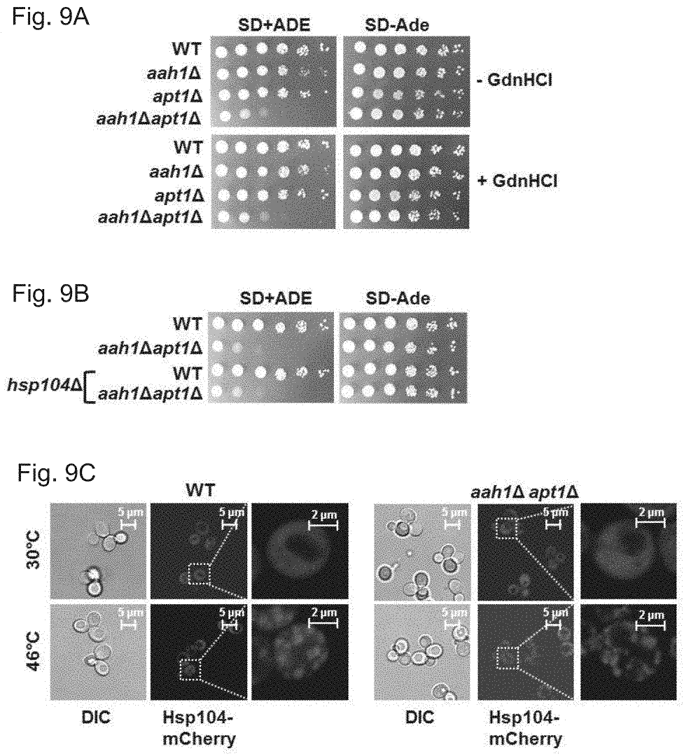

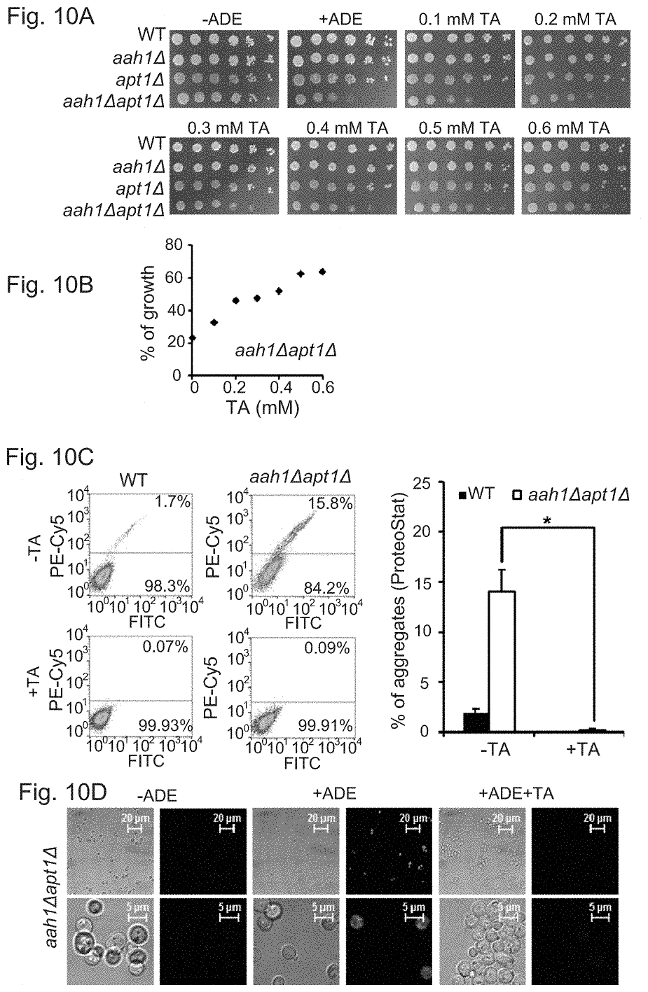

To examine whether the observed non-linear behavior of the dose-dependent toxicity and the intracellular aggregation of adenine detected by Raman microspectroscopy is indeed associated with self-organization of the metabolites into amyloid-like assemblies in vivo. the cells were stained with ProteoStat, an amyloid-specific fluorescent dye. This reagent was previously shown to facilitate specific and sensitive detection of amyloid aggregates in living cells (Navarro. S. & Ventura, S. Biotechnol. J. 9, 1259-1266 (2014)). To validate the possible identification of amyloid fibrils by ProteoStat staining in yeast, detection of the prion protein Sup35 was examined (FIG. 7), showing significant and gradual increase in the staining of [psi−] compared to two types of [PSI⁺] aggregates, weak [PSI⁺] and strong [PSI⁺]. Flow cytometry and confocal microscopy were then employed to detect the presence of intracellular amyloid-like adenine aggregates. Both techniques clearly indicated the presence of amyloid-like structures in the adenine salvage mutant. Flow cytometry showed a higher degree of aggregation in the mutant compared to wild-type cells in the presence of the metabolite (FIG. 8A). Moreover, consistent with the indicated adenine sensitivity (FIG. 1A), removal of adenine from the medium reduced the level of aggregation in the mutant cells (FIG. 8A). Confocal microscopy further allowed the identification of the intracellular localization of the adenine aggregates, showing clearly stained dots only in the salvage mutant and specifically following the addition of adenine (FIG. 8B). Z-stack followed by 3D reconstruction (FIG. 8C), as well as projection of a single section of the Z-stack (FIG. 8D), showed that the stained dots were localized inside the cell, excluding the possibility of their attachment to the outer membrane or cell wall. Furthermore, Hoechst staining showed no localization, ruling out the possibility that the stained dots were inside the nucleus (FIG. 8E). The Hsp104 chaperon was previously shown in yeast to play a pivotal role in the formation of numerous aggregates by structurally-unrelated proteins and its deletion partially restored the viability of cells expressing Aβ and polyQ in yeast models for Alzheimer's disease and Huntington's disease, respectively [10]. To test whether the toxicity following adenine accumulation in the salvage mutant is mediated by Hsp104, cell growth upon adenine addition was examined in the presence of guanidine hydrochloride that was repeatedly shown to inhibit Hsp104 activity, as well as in a hsp104Δ mutant background (FIG. 9A-9B). No Hsp104 dependency was observed, suggesting that the toxicity induced by adenine accumulation involves a different mechanism than the Hsp104-associated one. To further validate this observation, the chaperon response was examined using a Hsp104-mCherry strain [23] (FIG. 9C). While Hsp104 recruitment and accumulation was observed under heat shock stress and in the presence of adenine both in wild-type and in the salvage mutant cells, no such dots appeared under optimal growth temperature, suggesting a different mechanism than the Hsp104-associated that was reported.

### In Vivo Formation of Adenine Amyloid-Like Assemblies

To confirm that the staining with the amyloid-specific ProteoStat dye specifically identifies adenine amyloid-like structures, polyclonal antibodies against adenine fibrils structures were generated, as previously described by the inventors for tyrosine fibrils [14]. After staining with the anti-adenine fibrils antibody, stained dots could be detected only in the salvage mutant in the presence of adenine (FIG. 8F). Furthermore, the dots appeared inside the cells but outside of the nucleus, in accordance with the ProteoStat staining (FIG. 8B). This result suggests that the observed cellular toxicity is directly linked to self-assembled amyloid-like adenine structures.

### Example 5

### Inhibition of Adenine Toxicity by TA

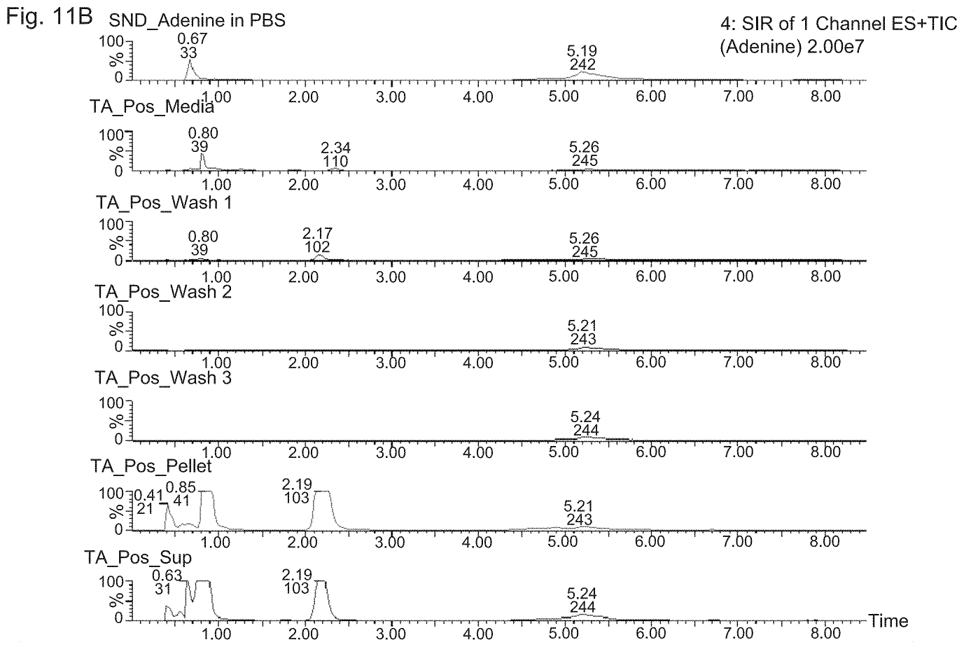

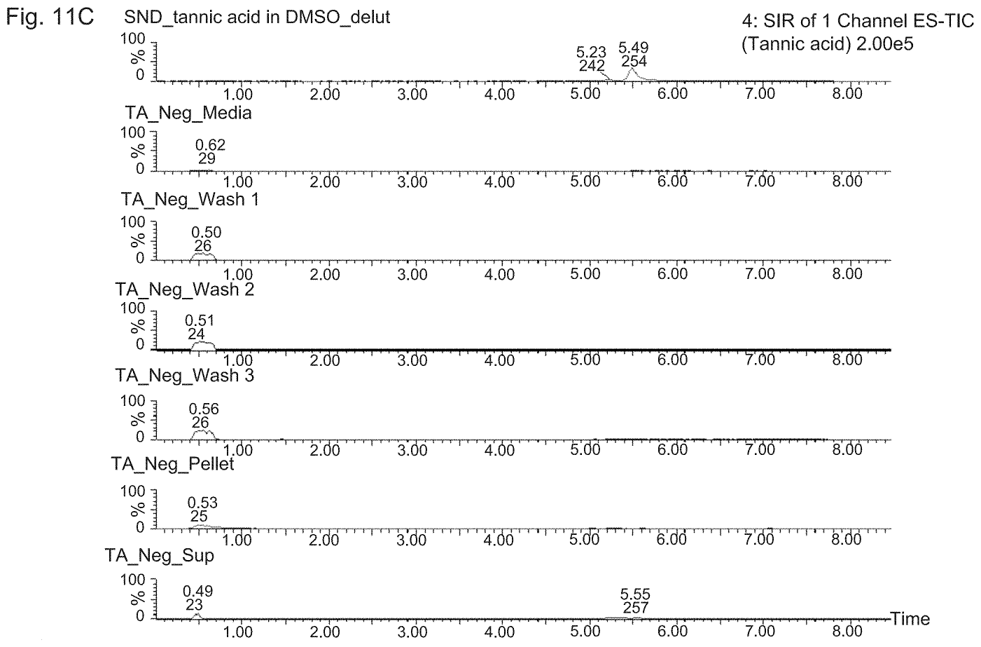

The next aim was to manipulate the formation of the intracellular adenine amyloid-like structures formed at high cellular concentrations of adenine upon feeding. Polyphenols comprise a large group of natural and synthetic small molecules, which were repeatedly shown to inhibit the formation of protein amyloid fibrils [15] including the formation of aggregates that is associated with neurodegenerative diseases [16]. The effect of TA, a widely studied polyphenol which was suggested as a potent inhibitor of β-amyloid fibrillation and of the assembly of the PrPsc prion protein [17]. Moreover, the inventors have previously demonstrated the inhibition of adenine self-assembly in vitro by this polyphenol compound [4]. It was found that in the presence of adenine, the addition of Tannic acid (TA) to the yeast media clearly improved cell growth of the salvage mutant in a dose-dependent manner (FIG. 10A-10B). Staining of the cells with the ProteoStat amyloid-specific dye followed by flow cytometry allowed the detection of a decrease in metabolite aggregation in the presence of the inhibitor, despite the external addition of adenine to the media (FIG. 10C). Furthermore, the absence of the stained dots following the addition of TA was demonstrated by confocal microscopy (FIG. 10D). Based on mass-spectroscopy analysis (FIG. 11A-11B), TA and adenine were detected inside the cell suggesting that TA can enter the cells. To verify the molecular identity of the TA peak, the same analysis was performed on samples that contained adenine but not TA, showing only the peak corresponding to adenine (FIG. 11C-11D). In order to ascertain that the inhibition by TA represents a general phenomenon applicable for other known amyloid inhibitors, baicalein, an additional polyphenolic inhibitor of protein amyloid formation, that was recently shown to bear therapeutic potential for Alzheimer's and Parkinson's disease was examined [18]. Indeed, the use of this inhibitor resulted in a significant effect on yeast cell growth (FIG. 12A-12B) and on adenine aggregation (FIG. 12C), as well as in a dose-dependent inhibition of adenine self-assembly in vitro (FIG. 12D).

### Example 6

### TA Mechanism of Action

In order to examine the in vivo mechanism underlying the inhibition of adenine amyloid-like formation by TA, the inhibitor was added at different time points of yeast growth. TA was found to hinder the formation of adenine amyloid-like assemblies when added at earlier time points of yeast growth, while it had no effect on their growth when added at later stages, suggesting that TA is most effective at the nucleation and early oligomerization stage of the metabolite self-assembly (FIG. 13A). These results further support a correlation between adenine aggregation into ordered structures and growth inhibition, suggesting that the toxic effect is induced by adenine accumulation into amyloid-like species. This effect can be rescued by either removal of adenine from the medium or addition of the amyloid inhibitor, thereby modulating the assembly process. Finally, it was examined whether the dramatic effect of the inhibitor on the viability of the mutant cells actually reflects a change in the concentration of adenine in the metabolite salvage model, in addition to the inhibition of amyloid-like structure formation. For this purpose, the intracellular concentrations of adenine with and without the inhibitor were compared using mass spectrometry. Evidently, while the addition of the inhibitor drastically improved cell growth, the intracellular levels of adenine remained constant (FIG. 13B). Thus, the change in cell growth appears to occur specifically due to the inhibition of adenine aggregate formation, and not as a result of a decrease in adenine levels, further indicating that the assemblies, rather than free metabolite molecules, mediate the cell toxicity (as modeled in FIG. 13C).

### Example 7

### HTS Yeast Growth Assay

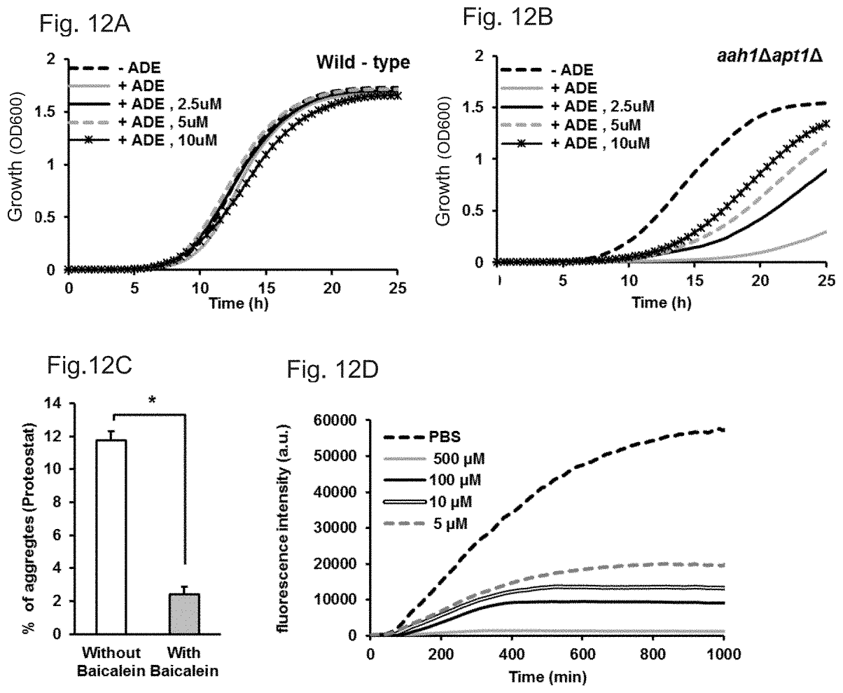

The high-throughput screening was calibrated for 384 plate format using phenotypic assay based on yeast growth rate (measured by OD). The goal was to find hit compounds that will dramatically improve the growth rate as compared to the salvage mutant in the presence of adenine. All relevant controls were used including: mutant yeast grown without adenine addition, WT yeast, and mutant yeast in the presence of adenine with Prazosin as a positive control compound. Potential hits were selected after calculations of growth curve slopes and area under the curve during the logarithmic phase. After hit validation and dose response experiments, hits displaying low IC50 rates were selected implying on a good potential for some of the compounds (FIG. 14).

### Example 8

Primary Lymphoblasts (LCLs) from ADA Patients Assay: Aggregation and Toxicity

In order to verify the efficiency of the resulting hits (as defined above) to dissolve adenosine aggregates and consequently improve cell viability, hits validation assays were established on transformed LCLs generated from ADA deficient patients and healthy individuals.

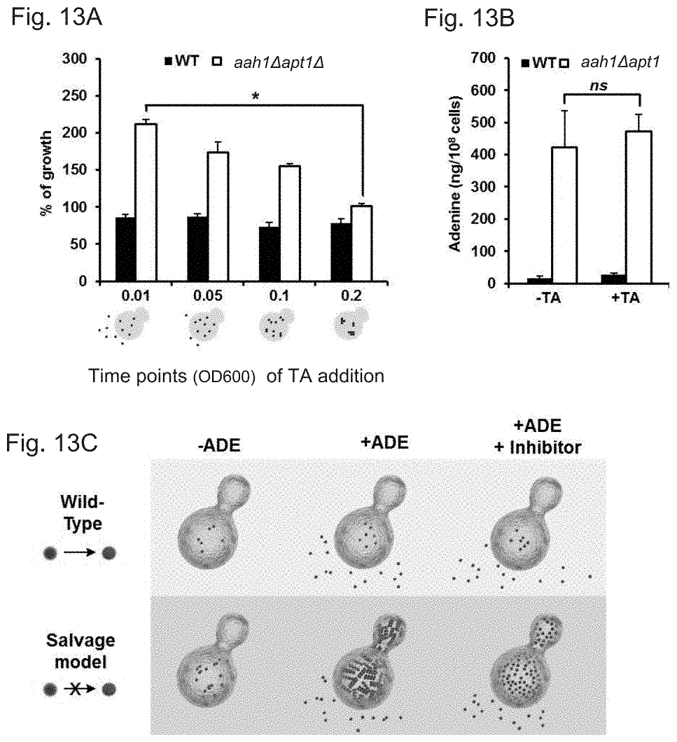

For quantification of intracellular adenosine aggregates by FACS analyses, 100,000 cells were seeded in 96 well plates in full medium. After 24 hours, cells were centrifuged, washed once and added with serum free medium with or without Adenosine (200 μg/ml). After incubation of 72 hours cells were washed with PBS and stained for adenosine aggregates with Proteostat dye. Following 15 minutes incubation, cells were washed with PBS and mean fluorescence was measured with FACS. Mean cell fluorescence of every cell line was normalized to its signal without adenosine and data is presented as percentage of aggregates. The results demonstrate that ADA deficient LCLs (FIG. 15A cell lines 8,9,11,12) show 2-3 fold higher staining as compared to healthy LCLs (FIG. 15A cell lines 1,2,3,4). The unaffected ADA (+/−) cell line (FIG. 15A cell line 6) shows staining levels similar to healthy cells.

In order to assess the toxic effect of adenosine aggregates, XTT based cell viability assays were performed on healthy and ADA deficient LCLs. Cells went through similar procedure as described above except adenosine was at 300 μg/ml. Survival percentage was calculated as absorbance with adenosine divided by absorbance without adenosine. The results show that in the presence of adenosine, healthy LCLs survival is above 70% (FIG. 15B cell lines 1,2,3,4) while ADA deficient LCLs survival is below 40%. Moreover. 4 out of 5 ADA deficient cell lines display less than 20% survival (FIG. 15B cell lines 7,8,9,11,12). The unaffected ADA (+/−) cell line (FIG. 15A cell line 6) shows survival levels similar to healthy cells.

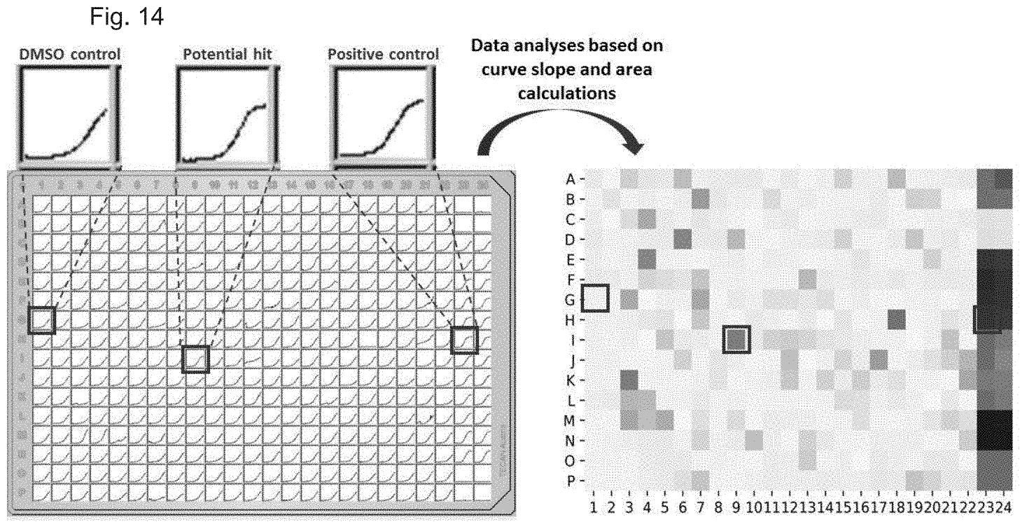

In addition, cell growth appears to be affected by adenosine levels in a non-linear cooperative manner, similar to in vitro studies and in vivo studies in the adenine salvage mutant (FIG. 16). Above a critical concentration, the favorable energetic state dictates the formation of the toxic assemblies, while at lower concentrations the metabolites are most likely at the normal physiological state.

### Example 9

### Hit Validation on ADA-SCID Patients

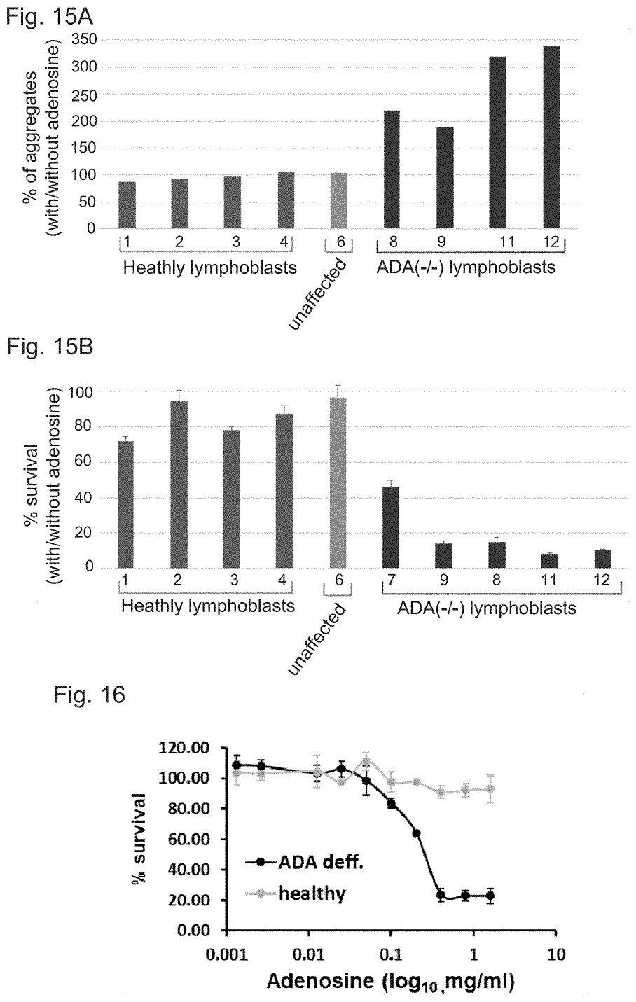

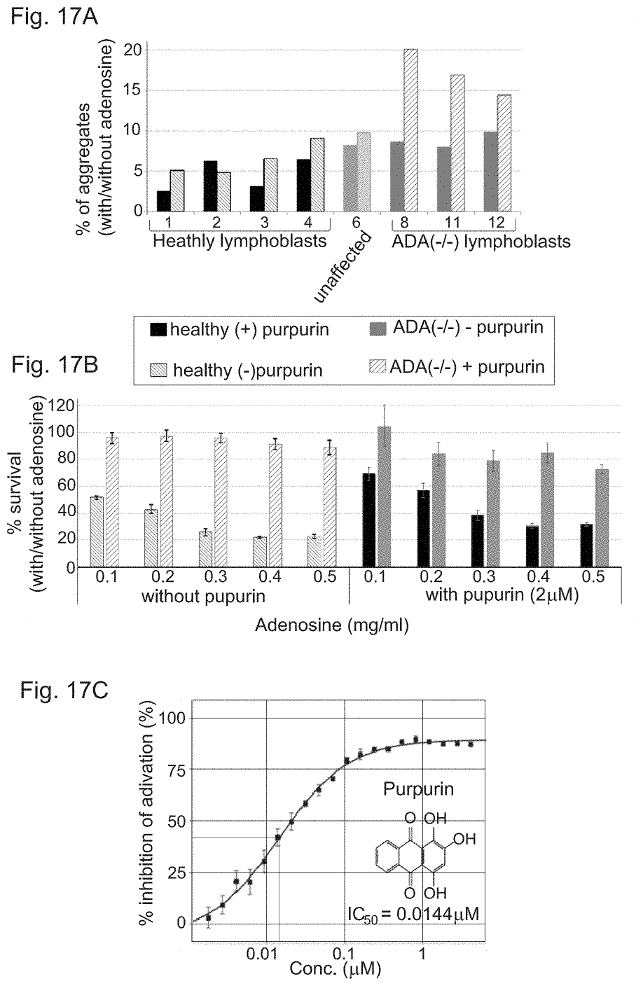

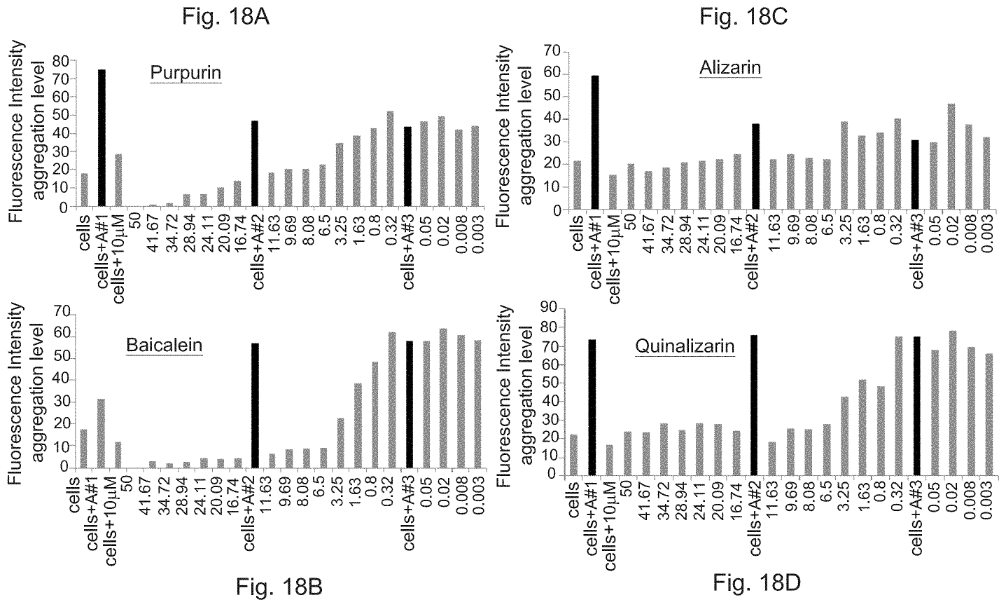

Next, hit compounds that were identified using the HTS yeast system were tested for their activity in the LCLs system. For proof of concept. Purpurin was chosen and showed high potency in rescuing mutant yeast strain from adenine toxicity with IC50 of 0.0144 uM (FIG. 17C). LCLs went through same procedure as described above in the LCLs assay development section and in addition were added with 2 μM of Purpurin or with relevant concentration of DMSO as control. FACS analyses showed, that upon Purpurin addition, strong Proteostat staining of adenosine aggregates observed with ADA deficient LCLs reduced to significantly lower levels, similar to those observed with healthy LCLs (FIG. 17A). Moreover, ADA deficient LCLs treated with Purpurin showed substantial improvement in cell viability as compared to control LCLs (FIG. 17B). Taken together these results show reproducibility of activity of hit compounds selected in the HTS yeast system. An example of that is Purpurin that is active both in the yeast system, and in ADA deficient LCLs. It was thus demonstrated that assays developed in ADA-deficient LCLs reinforce the data obtained in yeast HTS system and could be further used for hits validation. Next, additional hits were further validated using patient derived lymphoblastoids (FIG. 18A-18D). Hits validation was done for four compounds—purpurin, alizarin, baicalein and quinalizarin. Cells were treated with adenosine alone or with adenosine with the compounds. Compound activity was tested in 19 concentrations in range from 50 μM to 0.003 μM (FIG. 18A-18D). While samples with adenosine alone showed high signal because of strong staining of adenosine aggregates, addition of candidate compounds inhibited the formation of these aggregates thus reducing staining. According to the results, IC50 values for each compounds were calculated (FIG. 19).

To further examine if the compounds specifically target the adenosine amyloid-like structures. the compounds were tested using adenine in-vitro aggregation assay. Adenine was self-assembled with and without the inhibitors and stained with the amyloids dye, Thioflavin T (ThT). FIG. 34 demonstrates formation of amyloid-like structures by adenosine self-assembly and guanosine self-assembly. More specifically. Adenosine (30 mg/mL) and guanosine (3 mg/ml) were dissolved in PBS at 90° C., followed by the addition of ThT at final concentration of 20 μM. ThT emission data at 480 nm (excitation at 450 nm) was measured over time. TEM images show elongated adenosine fibrils (10 mg/mL) and guanosine fibrils (1 mg/mL) (FIGS. 34A and 34B, respectively). ThT fluorescence assay of adenosine and guanosine assemblies, are shown by FIGS. 34C, and 34D respectively.

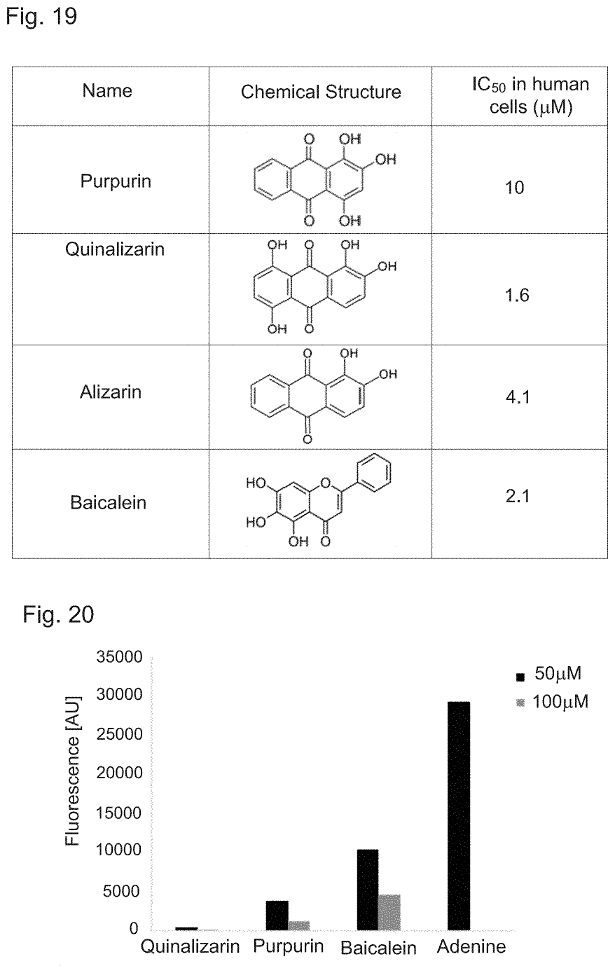

Next, three compounds (Purpurin, Baicalein and Quinalizarin) significantly reduced the amyloids staining, suggesting a direct effect on the adenine structures (FIG. 20).

### Example 10

### A Model for Tyrosine Self-Assembly in Yeast

### Dose-Dependent Sensitivity of the Aro3Δ Mutant to Tyrosine

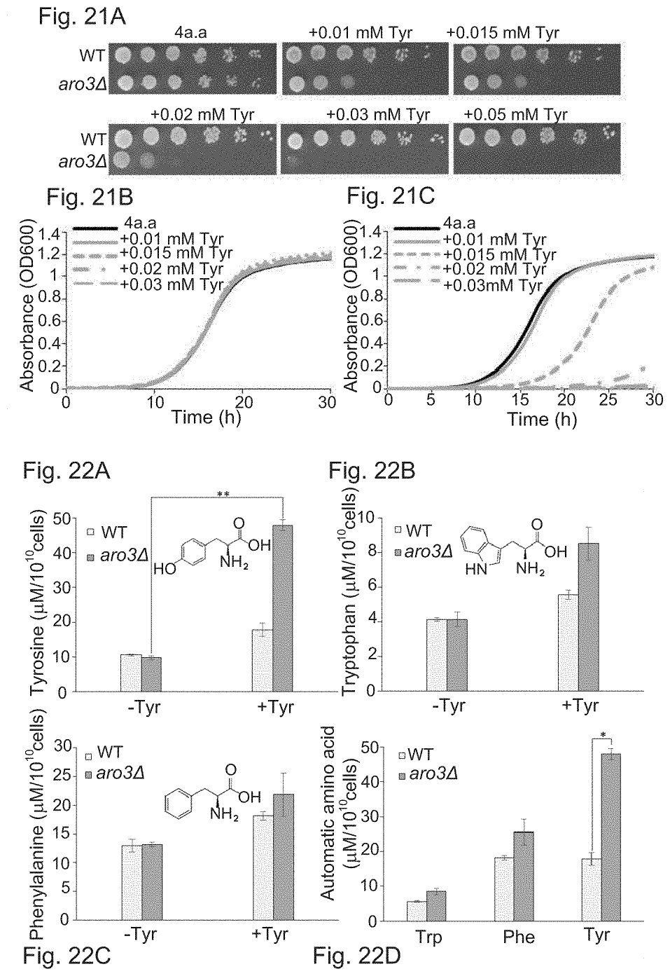

The focus was put on a strain with a deletion of the ARO3 gene in which a significant decrease in cellular growth was observed upon tyrosine feeding (see FIG. 21). The inventors aimed to determine whether the toxic effect was influenced by different levels of tyrosine. Thus, the cellular growth of wild-type and the aro3Δ mutant was examined by serial dilutions on selective plates that were supplemented with different concentrations of tyrosine (FIG. 21A). In addition, cellular growth was also examined by measuring the turbidity of the medium upon addition of the indicated concentration of tyrosine (FIG. 21B). These assays revealed that increasing tyrosine concentration in the medium results in an increase of growth inhibition. Thus, cell growth is affected by tyrosine levels in a dose-dependent manner which further supports the notion that the toxicity in the aro3Δ mutant results from the presence of tyrosine.

### Intracellular Concentrations of the Aromatic Amino Acids

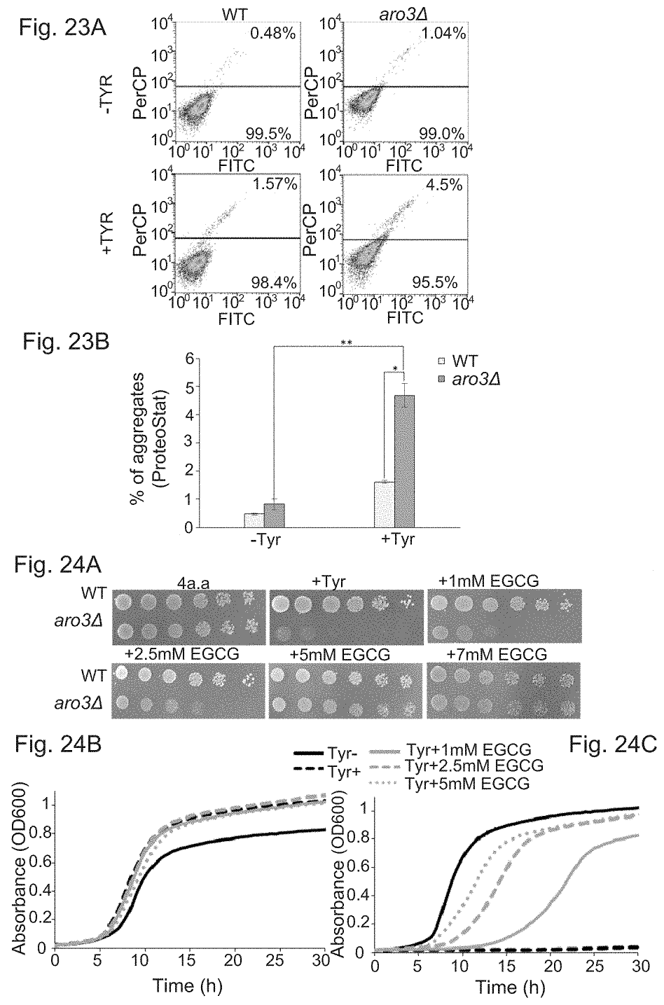

The determination of the actual intracellular concentration of the metabolites under the experimental conditions plays a major role in the understanding of the molecular basis and mechanisms underlying the formation of the fibrillar amyloid-like assemblies in vivo. Liquid Chromatography-Mass Spectrometry (LC-MS), a well-established method that was previously shown to be effectual and reliable in the determination of the intracellular concentration of numerous metabolites in yeast cells, was used for this purpose. It was demonstrated that growth of the aro3Δ mutant on 4a.a SD media containing tyrosine results in significantly higher levels of intracellular tyrosine compared to the aro3Δ mutant cells in the absence of tyrosine in the medium as well as to wild-type cells in the presence or absence of tyrosine (FIG. 22A). These results support the findings demonstrating a clear correlation between the mutation in the ARO3 gene and the accumulation of tyrosine. Since the tyrosine biosynthesis pathway is associated with the biosynthesis of the other aromatic amino acids, phenylalanine and tryptophan, the intracellular concentrations were quantified as well. A less significant change in the levels of these amino acids was observed (FIG. 22B-22D). These results suggest a strong correlation between the growth inhibition observed upon the addition of tyrosine to the medium and the accumulation of intracellular toxic levels of tyrosine.

### Amyloid-Like Assemblies' Formation Upon Tyrosine Accumulation

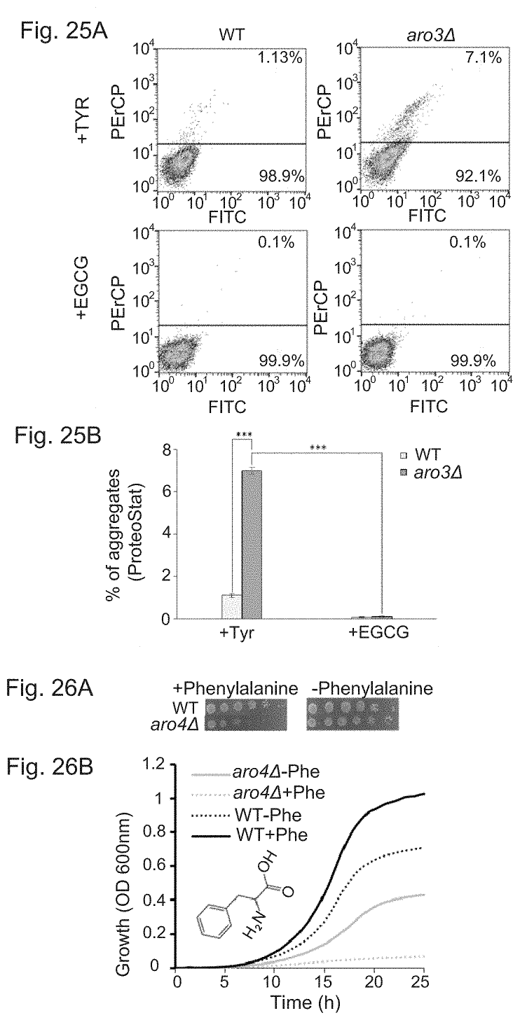

In order to examine whether the inhibition in cell growth in a non-linear cooperative manner upon tyrosine feeding (FIG. 23A, 23B) is associated with in vivo formation of amyloid-like assemblies, cells were stained with ProteoStat®, a well-established amyloid-specific fluorescent dye that was previously shown to detect the presence of intracellular protein amyloid-like aggregates in living bacterial cells with high specificity, even when the target proteins are expressed at low levels. Furthermore, this reagent was used for the first time by the inventors in yeast cells for demonstrating intracellular assembly of metabolite amyloid-like structures [24-25] This study indicates that this reagent can efficiently penetrates the cell wall in contrast to the commonly used amyloid-specific dye, ThT, in which a membrane perforation is required.

Flow cytometry (FACS) analysis revealed that growth of aro3Δ mutant on 4a.aSD media containing tyrosine results in a significantly higher percentage of aggregates compared to the aro3Δ mutant cells in the absence of tyrosine as well as to wild-type cells in the presence or absence of tyrosine which showed a significantly lower percentage of aggregates (FIG. 23A, B). These results suggest that the slow growth phenotype observed in the aro3Δ mutant cells might accrue due to higher levels of toxic aggregates.

### Inhibition of Tyrosine Toxicity in Yeast by the Polyphenol EGCG

The next aim was to manipulate the formation of the intracellular tyrosine amyloid-like structures formed at high cellular concentrations of tyrosine upon feeding. One extensively explored group of inhibitors, which have been shown to alter the formation of amyloid structures and efficiently reduce amyloid-related cell death, regardless of the identity of the amyloidogenic building block and its amino acids sequence, are the polyphenol molecules. Herein, the effect of EGCG was examined, an aromatic polyphenolic compound, known to effectively inhibit protein amyloid assemblies and in vitro inhibition of metabolite amyloid-like fibrils including that of tyrosine.

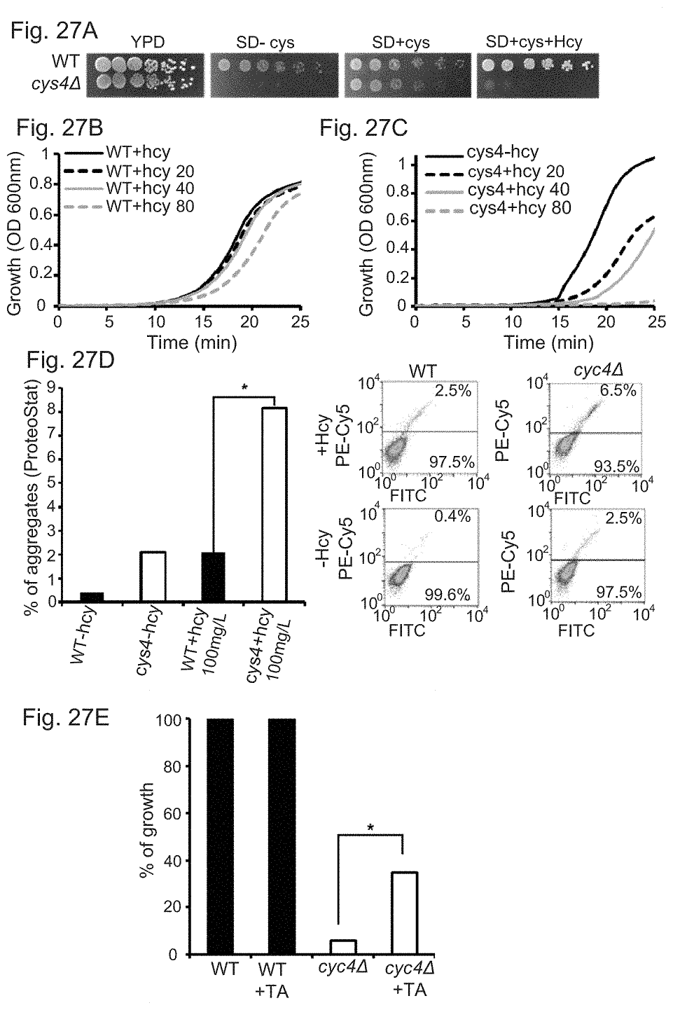

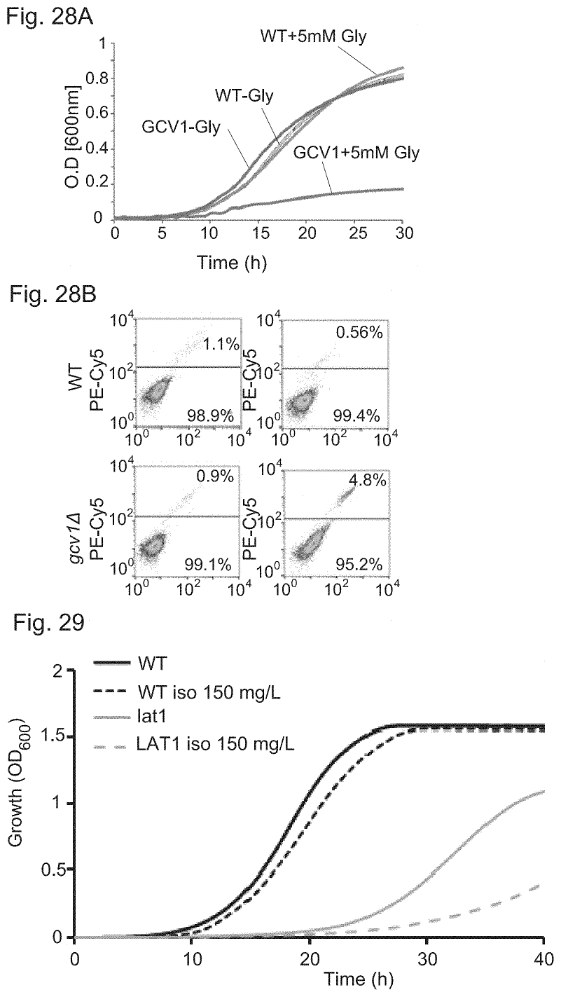

In order to examine the effect of EGCG on cell growth, the aro3Δ mutant was spotted on 4a.a SD plates that contained tyrosine alone or tyrosine with different concentrations of EGCG. While in the absence of EGCG a remarkable inhibition of cellular growth was observed, an addition of EGCG leads to a significant improvement in growth in a dose-dependent manner (FIG. 24A, 24C). Furthermore, it was shown by monitoring the cell growth kinetics that the lag phase of the aro3Δ mutant was notably shorter following EGCG treatment in comparison to untreated cells in a dose-dependent manner, wherein at the highest concentration a nearly complete recovery could be observed. Thus, the lag phase in the presence of EGCG was nearly the same as cells unexposed to tyrosine, indicating the potent ability of EGCG to eliminate the tyrosine sensitivity of the aro3Δ mutant. While a slight improvement in the growth of wild-type cells was observed upon the addition of tyrosine, no significant changes were shown upon the addition of EGCG (FIG. 24B). Next, the inventors aimed to gain additional insights into the effect of EGCG on the amyloid-like fibrils formed by the accumulation of tyrosine in the aro3Δ mutant cells. For that purpose, cells were stained with the amyloid-specific dye ProteoStat®, and FACS was applied to detect tyrosine aggregates. While a significantly higher percentage of aggregates was measured in the aro3Δ mutant in the presence of tyrosine compared to the wild-type cells, the addition of EGCG resulted in a dramatic reduction in the tyrosine aggregates percentage in the mutant cells. (FIG. 25). These results imply that EGCG possesses the ability to diminish the formation of the toxic assembly of tyrosine.

### Example 11

### Yeast Model for Phenylalanine Accumulation

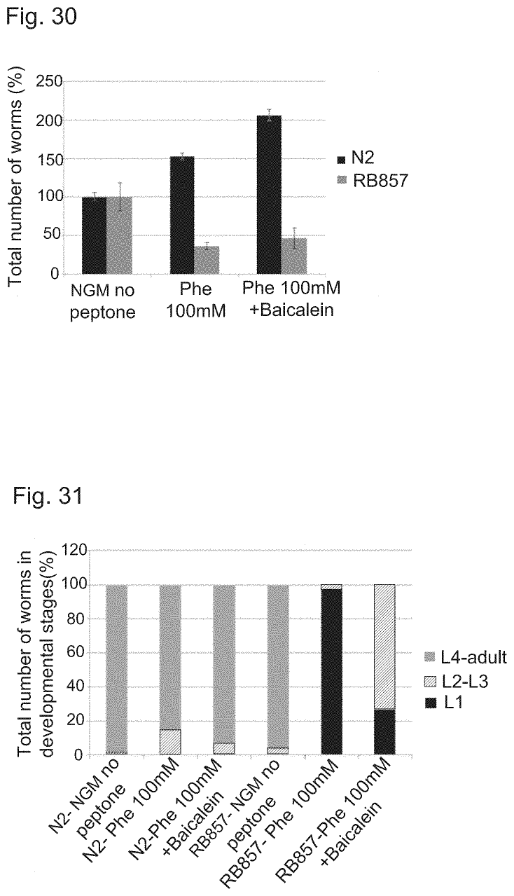

An in vivo yeast model system was established for the study of phenylketonuria. The focus was put on a strain with a deletion of the ARO4 gene. The inventors aimed to determine whether the toxic effect is influenced by phenylalanine feeding. Thus, the cellular growth of wild-type and the aro4Δ mutant was examined by serial dilutions on selective plates that were supplemented with phenylalanine. In addition, cellular growth was also examined by measuring the turbidity of the medium upon addition of phenylalanine (FIG. 26A, 26B). These assays revealed that the presence of phenylalanine in the medium results in growth inhibition.

### Example 12

### A Model for Homocysteine Self-Assembly in Yeast

Homocystinuria results from cystathionine beta synthase (CBS) deficiency, leading to excess of Hcy. A knockout mutation in CYS4, the highly-conserved functional yeast ortholog of the human cystathionine beta synthase (CBS) known to be essential for Hcy salvage, was therefore generated. Due to the contribution of this enzyme to cysteine biosynthesis, it was first shown that its absence confers cysteine dependence (FIG. 27A). Indeed, sensitivity to Hcy supplied in the growth medium was observed in the CYS4 mutant, compared to the wild-type strain, in a dose depended manner (FIG. 27A-C). To examine whether the observed toxicity was associated with the in vivo formation of amyloid-like assemblies, the cells were stained with the amyloid-specific fluorescent dye ProteoStat which allows to detect intracellular amyloid fibrils. The results showed a significantly higher level of aggregation in the salvage mutant compared to wild-type cells in the presence of Hcy, as well as compared to the mutant strain in the absence of the metabolite, indicating the presence of amyloid-like structures in the Hcy salvage mutant (FIG. 27D). Next, to further establish the amyloid-like nature of the aggregates, the fibrillation-modifying polyphenolic compound tannic acid (TA) was used (FIG. 27E). The results indicate that while no effect was observed in the wild-type strain, the addition of TA significantly ameliorated the growth inhibition of the salvage mutant strain in the presence of Hcy.

### Example 13

### A Model for Glycine Self-Assembly in Yeast

The IEM Non Ketotic Hyperglycinemia (NKH) affects mainly newborns and infants and is lethal in many of the cases or otherwise can cause severe damage to the nervous system, impeding normal motor and intellectual development. In this study. an in vivo yeast model system was established for the study of NKH whereby addition of glycine to the growth medium resulted in dose-dependent growth inhibition (FIG. 28A). In addition, upon glycine feeding, a fluorescent signal was detected using the amyloid dye ProteoStat, which is known to effectively detect the presence of intracellular amyloid-like deposits (FIG. 28B). This model opens up exciting prospects for understanding the mechanism of the disease and for drug screening in order to identify potential treatment for NKH.

### Example 14

### A Model for Isoleucine Self-Assembly in Yeast

To establish an in vivo model in yeast for the study of BCAA accumulation and toxicity, the LAT1 gene was disrupted. LAT1 encodes the dihydrolipoamide acetyltransferase component (E2) of the yeast pyruvate dehydrogenase complex. LAT1 catalyzes the oxidative decarboxylation of pyruvate to acetyl-CoA and mutations in DBT1, the human homolog, can lead to Maple Syrup Urine Disease (MSUD). Indeed, as demonstrated by FIG. 29, sensitivity to the branched-chain amino acid (BCAA) isoleucine (BCAAs may include leucine, isoleucine and valine) supplied in the growth medium was clearly observed.

### Example 15

Using the Nematode Worm Caenorhabditis elegans Models for IEM's as a Multicellular Organism for Hits Validation
A Model for PKU in C. elegans

Studies in C. elegans showed that it could serve as an excellent model to study neurodegenerative disorders. To study PKU in C. elegans model, a strain was used with mutation in the PAH-1 gene (RB857)]. Addition of phenylalanine significantly reduced the number of worms (FIG. 30). Next, the effect of one of the previously identified hits i.e. baicalein was examined on the development of worms. The life cycle of C. elegans is comprised of the embryonic stage, four larval stages (L1-L4) and adulthood. The end of each larval stage is marked with a molt, during which a new, stage-specific cuticle is synthesized and the old one is shed. Addition of 100 mM phenylalanine to the minimal media of RB857 worms had a devastating effect of their development—none of the worms reached adulthood on day 4, while more WT worms reached adulthood, if compared to the control (FIG. 31). In addition, adding Baicalein had a dramatic beneficial effect on both strains (FIG. 31).

A model for Tyrosinemia in C. elegans.

Mutations in different enzymes that are involved in the tyrosine degradation pathway are responsible for different types of Tyrosinemia. The tyrosine degradation pathway in mammals and C. elegans in conserved and the most severe is type I Tyrosinemia, which is caused by mutations affecting the last enzyme in the pathway, fumarylacetoacetate hydrolase (FAH). To study Tyrosinemia in C. elegans model, an RNAi was used that specifically silences the fumarylacetoacetate hydrolase (fah-1), as was previously reported (K10C4.2) [26]. RNAi directed against fah-1 causes severe phenotype of reduced size, impaired fertility and reduction of total number of viable worms (FIG. 32). Addition of tyrosine to the fah-1 silenced worms, leads to severe decline in all the observed phenotypes, while addition of EGCG is able to partially restore the fertility and the total number of viable worms (FIGS. 32-33).

1. A yeast screening system for candidate therapeutic compounds, said system comprises:
(a) a yeast cell and/or yeast cell line, and/or yeast cell population, that carry at least one manipulation in at least one yeast metabolic pathway, that leads to accumulation of at least one metabolite, wherein accumulation of said at least one metabolite is associated with at least one inborn error of metabolism (IEM) disorder; and optionally
(b) at least one reagent or means for determining at least one of, the accumulation of said metabolite and at least one phenotype associated with accumulation of said metabolite.

2. The system according to claim 1, further comprising at least one validation means for said candidate therapeutic compound, said validation means is at least one of:
(a) at least one unicellular organism that display accumulation of said metabolite;
(b) at least one multicellular eukaryotic organism that display accumulation of said metabolite; and
(c) at least one mammalian cell that display accumulation of said metabolite;
(d) at least one mammalian animal model that display accumulation of said metabolite.

3. The system according to claim 1, wherein said phenotype associated with accumulation of said metabolite is at least one of cell toxicity and formation of metabolite aggregates, optionally, at least one of:
(a) wherein toxicity is determined by measuring at least one of cell viability, cell proliferation, cell apoptosis, and any toxic phenotype on the organism or cell; and
(b) wherein at least one of, the accumulation of said metabolite and formation of metabolite aggregates is determined by at least one of metabolic profiling, microscopy, light diffraction, absorption or scattering assay, spectrometric assay, immunological assay, flow cytometry, liquid chromatography, nuclear magnetic resonance (NMR) and stereoscopy.

4. The system according to claim 1, wherein said metabolite is any one of a nucleobase, nucleoside, nucleotide, an amino acid residue, carbohydrate, fatty acid and ketone, sterols, porphyrin and haem, lipid, sphingolipid, phospholipid and lipoprotein, neurotransmitters, vitamins and (non-protein) cofactors, pterin, trace elements, metals, metabolites associated with energy metabolism, metabolites associated with peroxisome functions, or any intermediate product, derivative or metabolite thereof.

5. The system according to claim 4, wherein said metabolite is at least one nucleobase, any derivative, any intermediate product thereof, or any combination or mixture thereof, optionally, said nucleobase is at least one of purine nucleobases, any derivative or any intermediate product thereof.

6. The system according to claim 5, wherein said purine nucleobase is at least one of adenine, any derivative and intermediate thereof and guanine, any derivative and intermediate thereof, optionally, wherein said IEM disorder associated with accumulation of at least one of adenine and any derivatives thereof, is adenosine deaminase (ADA) deficiency and adenine phosphoribosyltransferase (APRT) deficiency.

7. The system according to claim 1, wherein said manipulated yeast cell and/or yeast cell line, and/or yeast cell population, carry a genetic and/or epigenetic modification in at least one of Adenine phosphoribosyltransferase 1 (APT1) and Adenine deaminase (AAH1) yeast genes, optionally, wherein said genetically and/or epigenetically modified yeast cell and/or yeast cell line, and/or yeast cell population, display reduced or no expression of APT1 and AAH1 genes, and wherein said cell and/or yeast cell line, and/or yeast cell population, display accumulation of at least one of adenine and any derivative thereof.

8. The system according to claim 1, wherein any one of:
(a) said metabolite is uric acid, and wherein said IEM disorder associated with uric acid accumulation is Gout disease; or
(b) said metabolite is at least one amino acid residue, any derivative, or any intermediate product or metabolite thereof, optionally, wherein said amino acid residue or any intermediate product or metabolite thereof is at least one of: Phenylalanine, Tyrosine, Glycine, Homocysteine, Arginine, Cysteine, Isoleucine, Leucine, Lysine, Methionine, Proline, Tryptophane, Valine, N-acetylaspartate (NAA), Homogentisic acid, and any derivatives thereof, and wherein said IEM disorder associated with accumulation of at least one of Phenylalanine and any derivatives thereof is Phenylketonuria, wherein said IEM disorder associated with accumulation of at least one of Tyrosine and any derivatives thereof is Tyrosinemia, wherein said IEM disorder associated with accumulation of at least one of Glycine and any derivatives thereof is Glycine encephalopathy, and wherein said IEM disorder associated with accumulation of at least one of Homocysteine, any derivatives thereof is Homocystinuria, wherein said IEM disorder associated with accumulation of at least one of isoleucine, leucine, valine and any derivatives thereof is Maple Syrup Urine Disease (MSUD).

9. The yeast screening system of candidate therapeutic compounds according to claim 1, wherein said compound is for treating, preventing, ameliorating, reducing or delaying the onset of at least one inborn error of metabolism (IEM) disorder associated with accumulation of at least one metabolite, wherein at least one of:
(I) said compound is for treating, preventing, ameliorating, reducing or delaying the onset of adenosine deaminase (ADA) deficiency associated with accumulation of at least one of adenine and any derivative thereof, and said system comprises:
(a) a yeast cell and/or yeast cell line, and/or yeast cell population, that carry at least one manipulation in at least one yeast metabolic pathway that leads to accumulation of at least one of adenine and any derivative thereof; and optionally
(b) at least one reagent or means for determining at least one of, the accumulation of said adenine and at least one phenotype associated with accumulation of said adenine an any derivative thereof;
(II) said compound is for treating, preventing, ameliorating, reducing or delaying the onset of Tyrosinemia associated with accumulation of at least one of tyrosine and any derivative thereof, and said system comprises:
(a) a yeast cell and/or yeast cell line, and/or yeast cell population, that carry at least one manipulation in at least one yeast metabolic pathway that leads to accumulation of at least one of tyrosine and any derivative thereof; and optionally
(b) at least one reagent or means for determining at least one of, the accumulation of said tyrosine and at least one phenotype associated with accumulation of said tyrosine an any derivative thereof:
(III) said compound is for treating, preventing, ameliorating, reducing or delaying the onset of Phenylketonuria associated with accumulation of at least one of phenylalanine and any derivative thereof, and said system comprises:
(a) a yeast cell and/or yeast cell line, and/or yeast cell population, that carry at least one manipulation in at least one yeast metabolic pathway that leads to accumulation of at least one of phenylalanine and any derivative thereof; and optionally
(b) at least one reagent or means for determining at least one of, the accumulation of said phenylalanine and at least one phenotype associated with accumulation of said phenylalanine an any derivative thereof;
(IV) said compound is for treating, preventing, ameliorating, reducing or delaying the onset of Glycine encephalopathy associated with accumulation of at least one of glycine and any derivative thereof, and said system comprises:
(a) a yeast cell and/or yeast cell line, and/or yeast cell population, that carry at least one manipulation in at least one yeast metabolic pathway that leads to accumulation of at least one of glycine and any derivative thereof; and optionally
(b) at least one reagent or means for determining at least one of, the accumulation of said glycine and at least one phenotype associated with accumulation of said glycine an any derivative thereof; and
(V) said compound is for treating, preventing, ameliorating, reducing or delaying the onset of Homocystinuria associated with accumulation of at least one of homocysteine and any derivative thereof, and said system comprises:
(a) a yeast cell and/or yeast cell line, and/or yeast cell population, that carry at least one manipulation in at least one yeast metabolic pathway that leads to accumulation of at least one of homocysteine and any derivative thereof; and optionally,
(b) at least one reagent or means for determining at least one of, the accumulation of said homocysteine and at least one phenotype associated with accumulation of said homocysteine an any derivative thereof.

10. A screening method of candidate therapeutic compounds for treating, preventing, ameliorating, reducing or delaying the onset of at least one IEM disorder associated with accumulation of at least one metabolite, the method comprising the steps of:
(a) contacting a manipulated yeast cell and/or yeast cell line, and/or yeast cell population, with a candidate compound, said yeast cell and/or yeast cell line, and/or yeast cell population, carry at least one manipulation in at least one yeast metabolic pathway, that leads to accumulation of said metabolite;
(b) determining in the contacted cells of (a), at least one of, the accumulation of said metabolite and the level of at least one phenotype associated with the accumulation of said metabolite; and
(c) determining that said candidate is a therapeutic compound for said IEM disorder if at least one of, the accumulation of said metabolite and said phenotype is modulated as compared with the accumulation of said metabolite and phenotype in the absence of said candidate compound.

11. The method according to claim 10, further comprising the step of validating a candidate compound displaying modulated level of at least one of, the accumulation of said metabolite and said phenotype as obtained in step (c), by:
(I) contacting said candidate compound with at least one of:
(i) at least one unicellular organism that display accumulation of said metabolite;
(ii) at least one multicellular eukaryotic organism that display accumulation of said metabolite;
(iii) at least one mammalian cell that display accumulation of said metabolite; and
(iv) at least one mammalian animal model that display accumulation of said metabolite,
(II) determining in the cells, unicellular organism, multicellular organism or mammal of (I), at least one phenotype associated with the accumulation of said metabolite; and
(III) determining that said candidate is a therapeutic compound for said IEM disorder if at least one of, the accumulation of said metabolite and said phenotype is modulated as compared with the level of at least one of, the accumulation of said metabolite and the phenotype in the absence of said candidate compound;
optionally, said method further comprising the step of administering to a subject suffering from said IEM, an effective amount of said therapeutic compound.

12. The method according to claim 10, wherein said phenotype associated with accumulation of said metabolite is at least one of cell toxicity and formation of metabolite aggregate, optionally, at least one of;
(a) wherein cell toxicity is determined by measuring at least one of cell viability, cell proliferation, cell apoptosis and any toxic phenotype on the organism or cell;
(b) wherein at least one of, the accumulation of said metabolite and the formation of metabolite aggregation is determined by at least one of metabolic profiling, microscopy, light diffraction, absorption, or scattering assay, spectrometric assay, immunological assay, flow cytometry, Liquid Chromatography, NMR and stereoscopy.

13. The method according to claim 10, wherein said metabolite is any one of a nucleobase, nucleoside, nucleotide, an amino acid residue, carbohydrate, fatty acid and ketone, sterols, porphyrin and haem, lipid, sphingolipid, phospholipid, and lipoprotein, neurotransmitters, vitamins and (non-protein) cofactors, pterin, trace elements, metals, metabolites associated with energy metabolism, metabolites associated with peroxisome functions, or any intermediate product, derivative or metabolite thereof.

14. The method according to claim 13, wherein said metabolite is at least one nucleobase, any derivative, any intermediate product thereof or any combinations or mixtures thereof, optionally, at least one of;
(a) said nucleobase is at least one purine nucleobases, or any derivative, or any intermediate product thereof; and
(b) said purine nucleobase is at least one of adenine and any derivative thereof.

15. The method according to claim 14, wherein said TEM disorder associated with accumulation of at least one of adenine and any derivatives thereof, is ADA deficiency and APRT deficiency, optionally, wherein said manipulated yeast cell and/or yeast cell line, and/or yeast cell population, carry a genetic and/or epigenetic modification in at least one of APT1 and AAH1 yeast genes, and wherein said genetically and/or epigenetically modified yeast cell and/or yeast cell line, and/or yeast cell population, display reduced or no expression of APT1 and AAH1 genes, and wherein said cell and/or yeast cell line, and/or yeast cell population, display accumulation of at least one of adenine and any derivative thereof.

16. The method according to claim 10, wherein any one of:
(a) said metabolite is uric acid, and wherein said IEM disorder associated with uric acid accumulation is Gout disease; or
(b) said metabolite is at least one amino acid residue, or any intermediate product or metabolite thereof, optionally, wherein said amino acid residue or any intermediate product or metabolite thereof is at least one of: Phenylalanine, Tyrosine, Homocysteine, Glycine, Arginine, Cysteine, Isoleucine, Leucine, Lysine, Methionine, Proline, Tryptophane, Valine, N-acetylaspartate (NAA), Homogentisic acid and any derivatives thereof, and wherein said IEM disorder associated with accumulation of at least one of Phenylalanine and any derivatives thereof is Phenylketonuria, wherein said IEM disorder associated with accumulation of at least one of Tyrosine and any derivatives thereof is Tyrosinemia, wherein said IEM disorder associated with accumulation of at least one of Glycine and any derivatives thereof is Glycine encephalopathy, wherein said IEM disorder associated with accumulation of at least one of Homocysteine and any derivatives thereof is Homocystinuria, and wherein said IEM disorder associated with accumulation of at least one of isoleucine, leucine, valine and any derivatives thereof is MSUD.

17. A method for treating, preventing, ameliorating, reducing or delaying the onset of at least one IEM disorder associated with accumulation of at least one metabolite, the method comprising the steps of:
(a) Obtaining a compound that modulates the level of at least one phenotype associated with the accumulation of said metabolite by the screening method according to claim 10, the method comprising:
(i) contacting a yeast cell and/or yeast cell line, and/or yeast cell population, with a candidate compound, said cell and/or yeast cell line, and/or yeast cell population, carry at least one genetic and/or epigenetic manipulation in at least one yeast metabolic pathway that leads to accumulation of said metabolite;
(ii) measuring in the incubated cells of (i) at least one of, the accumulation of said metabolite and at least one phenotype associated with the accumulation of said metabolite; and
(iii) determining that said candidate is a therapeutic compound for said IEM disorder if at least one of, the accumulation of said metabolite and said phenotype is modulated as compared with the level of at least one of, the accumulation of said metabolite and the phenotype in the absence of said candidate compound; and
(b) administering a therapeutic effective amount of the compound obtained by step (a) to a subject suffering from IEM disorder associated with accumulation of said metabolite.

18. A therapeutic compound for treating, preventing, ameliorating, reducing or delaying the onset of at least one IEM disorder associated with accumulation of at least one metabolite, wherein said compound is identified by a method comprising the steps of:
(a) contacting a yeast cell and/or yeast cell line, and/or yeast cell population, with a candidate compound, said cell and/or yeast cell line, and/or yeast cell population, carry at least one manipulation in at least one yeast metabolic pathway, that leads to accumulation of said metabolite;
(b) determining in the contacted cells of (a) at least one of, the accumulation of said metabolite and at least one phenotype associated with the accumulation of said metabolite; and
(c) determining that said candidate is a therapeutic compound for said IEM disorder if at least one of, the accumulation of said metabolite and said phenotype is modulated as compared with the level of the accumulation of said metabolite and the phenotype in the absence of said candidate compound, wherein said compound is identified by a method as defined by claim 10, and wherein said compound is at least one of a small molecule, aptamer, a peptide, a nucleic acid molecule and an immunological agent, and any combinations thereof.

19. The compound according to claim 18, wherein said compound is a small molecule compound of the general formula (II): 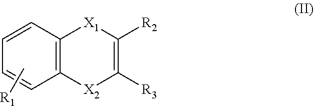
or a pharmaceutically acceptable salt, solvate, hydrate, any stereoisomer thereof or physiologically functional derivative thereof,
wherein each of X₁ and X₂ is independently selected from (CR⁴R⁵), O, S or NR⁴, each R⁴ and R⁵ independently is hydrogen, OH, C₁-C₆ alkyl, C₁-C₆ alkoxy, or CR⁴R⁵ is C═O,
R¹ is selected from hydrogen or —OH
Each of R₂ and R₃ is independently selected from hydrogen, an aryl optionally substituted or R₂ and R₃ form together with additional two carbons atoms, a six-membered ring, optionally said small molecule is any one of: 1,2,4-Trihydroxyanthraquinone (Purpurin) or any derivatives, analogs, salts and esters thereof, 1,2-Dihydroxyanthracene-9,10-dione (Alizarin) or any derivatives, analogs, salts and esters thereof, 1,2,5,8-tetrahydroxyanthraquinone (Quinalizarin) or any derivatives, analogs, salts and esters thereof and 5,6,7-trihydroxyflavone (Baicalein) or any derivatives, analogs, salts and esters thereof.

20. The method according to claim 10, wherein said IEM is ADA, wherein said compound is of the general formula (II), and wherein said method is for treating, preventing, ameliorating, reducing or delaying the onset of ADA deficiency in a mammalian subject, the method comprising the step of administering to said subject a therapeutic effective amount of at least one small molecule compound of the general formula (II): 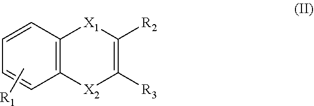
or a pharmaceutically acceptable salt, solvate, hydrate, any stereoisomer thereof or physiologically functional derivative thereof,
wherein each of X₁ and X₂ is independently selected from (CR⁴R⁵), O, S or NR⁴; each R⁴ and R⁵ independently is hydrogen, OH, C₁-C₆ alkyl, C₁-C₆ alkoxy, or CR⁴R⁵ is C═O,
R₁ is selected from hydrogen or —OH
Each of R₂ and R₃ is independently selected from hydrogen, an aryl optionally substituted or R₂ and R₃ form together with additional two carbons atoms, a six-membered ring, optionally said small molecule compound is any one of: 1,2,4-Trihydroxyanthraquinone (Purpurin) or any derivatives, analogs, salts and esters thereof, 1,2-Dihydroxyanthracene-9,10-dione (Alizarin) or any derivatives, analogs, salts and esters thereof, 1,2,5,8-tetrahydroxyanthraquinone (Quinalizarin) or any derivatives, analogs, salts and esters thereof and 5,6,7-trihydroxyflavone (Baicalein) or any derivatives, analogs, salts and esters thereof, or any composition comprising the same.
